SEQUENCE LISTING (52 sequences)

SEQ ID NO: 1 - DNA, 501 bp, Homo sapiens
gtgccgccaacccattgcccttgagatggagaccacctctttggtattgcaaagaaggtccttcgtaatcatctatccctcatcctcacccttctgctccatttgaaaggccctccagaacaacatgtcattcctgggaaccatctttggtattgcaaagaaggttcttcatgatcatcgtcccccatcctcaccctcctgcttcatttgaaaggccctccagaacaatgtgtcactaccagaaaccattcgctatctctgttccttgtttgtatttctgcactggaaatggccttacagctccacactcatccttcagacatgcagctccttggtgaccccatctgctgggtgctggcctatgtcacatccaggttgctttttattcattgtctcatctaccagcccatgcctccccaagggcagggctggtcctggttcactaacccatccccagcacccattggaatgctctagacttcagcccagtgagtcactaag

SEQ ID NO: 2 - DNA, 501 bp, Homo sapiens
aagaaccagccttcccacccgatattccgaccttggccatgggcccagcccattgaggtcaagctaattcatgccccttttcaaggcccagctccagccccagctcctccaggtagtttccatgactgttccagctccccaagggtccagctgcttgtgactgcctggctcatgctaggctgggattgatctttcactgacccctataaaggtgttgtactttcctaaccagcccaggttctctggagcagaagtttaccttattttgtacaaggctgagagcctctaaaggcatgtcctgggttgctgtcatctctggccttttcttaagcactgcacagagctgagcacacaggatctcaggatggaccaaagctgatgctgtttcctaggttacttgggaactcattgaaggtaggaggctcagagtgggccaggaggacagtctgccaccttgtatgcccatgcttcaccaagatgcatggcaaatacaaaacagtc

SEQ ID NO: 3 - DNA, 55161 bp, Homo sapiens
cagaggtccgtttctcgacaggtgccggctttccttagactcctccttggactgagaggaccagctcatcccttcctgcctgggctgctgcattcgcccacactgacttcttcactccagacttgccacctccaacccaagactaccctgctgcctgagcaaaccttctaaaatgcagaccctcccctacccctcccctgcttggggacacatgtgtccctaagtcctccagcccttcaaagtcaccgtttcctttcacaactacatacaatgaccgagccaccccatcctatcagatttacagaaggtctaggctccctgcaacagtgctggctcccaggcaggtgggtggggtgtcttacattttcctggtcactttttactctaaagtctactcctgttcaattcctgaacacttctgtgttggtcagaattccaggggtagtgagcaacagaaacaaactttaaacataaaggaaggtttattagaaagctcccttactggaggcaggggcaaggctagcctcaggtccaactggagttgaggccttaaaggctaccaggcccttctttcctcctctcttctctgcccatgctcttggcttggctctcaatgaagccgctacacttggcatctctggttcttcatgagaaaggaataggctctctctttggaacaaagtttaaaaattccagctgagcaagatggctcatacctgtaatcccagcactttgggaggccaagatgggaggatcgcttgagcccaggagttcgagaccagcctaggtaacataatgagactctgtgtctacaaaaaccattttaaaaattagctgggcatggtggcccatgcctgtagttccagctactcaggaggctgaggtgtgaagatcacttgatcctggaaggtagaggctatagtgagccatgatcatgccactgtactccagcctaggtgacagcataagacccggtctcaaaaaataaaaataaaaatcccagaggagggctatgattgggtaatttcagatcagctgtccattcctgtaccaaccaaccatggtcagcggggtgagacactctcattgtccagcttggtgaatagcatttggccaatccacagtggctggctgctagcctgatcacgtggctggaaaagagcatgcctgttctttgctcccaagggaacaaaatccgttcttaggaggcagaaccatctgatatgagaatggtctgtggccctccaaagaggcaagtacatgacacatatacagtatgtctgtggtattaaaatttcatggtaggagggaaggcaattaggaaaataaatatctaaaaagcctccttgtaaataataatgggaaaaaaaaggctgagcagcgttggagtagaggatggagcatttctcagaaggacagtaccatcttgggacaaaacaaagaaatgtccagttcagctcccgcttcctgcaacctctcacctctcctaaccctgtatccaacatgccccagcacctcctccttcactgctggcacaggctgttgtgtattgtagacttttctgaagatgaggcataacattcattgacaattcaccaccatagggaagctcctggttcatccatgcttggacctggcctgtgccagtccgtgtctcctcttctttttcctcctcctttctagagcccagctcctagcccttctacactgtctccatcttggaatgccatgctcaatctagtcatttcaatagctttgctttacaaagctagttaggtctctttccacacacttctcttttagatcatgctgagtttgtctttcatcaccaaaaaggcccatgaaggcattttcaaaaacataaagcctcctttccacactgaatctacccttagtaacacaaagcttataaacgagcaggttgttattaacctgctcagatcacgttgctaggcaaggtttattctaccaccagcagcctcagaaagtgaaaggctgtctagaggaaaccctgcaggctcaagttttttgtttttttattgtcgttgtttgttgttaacaccttccatttagagggatcaagtccagacagacttctttgaccatttaggctcccttagatggggttccccaaaggtgaggattcctgtgcagggatgtgtaaagaaaatgttcccaggagcgactggtaagagacgaggcaaggcaggacggagaatggaaaagatcccaggcagagtcccggcctctgcctggttccacaaggagttctagaatgtccattacacttcagagttccttccacctccaggcaaaggaacaccaatcactcaatgctaaggatgcccttagggggttgggaggatataaattcctgggtactttgtgcaaaaagtggttctcattgccccaggagagttctaagaaagttctaggttgtgaaaagggacctatttagaagcacaaggaaagccaggggaatggcactgacatggattgaatctgtgtgcccccaccccccagattcccaagttgaagctttaacccttgatgtgatgctatttgggaggtaattagggtttagggttaggtgaggtcatgagggcagaatccccatgatggaattagtgcctttagaagaagaggaagagacaagagtccaacatctctgccatgtgaggaaacagcaagaaggtgccacctacagacccaagagagggccctcatcagaacctgaccatgctggcaccctgatcttggatatcaccgcctccagaatagagagaaatcaatgtctgttgttaaggccacccagggtatggtactctgctacagcagcccaagctaagacaggcacccagaacacccccccaagagaggaacctgggctaagcaccacagtgcctactgctacccggaccctcctaaagctgcagagaatctggcttccctccagcttgccccacccagccaggctcccagctgcatgagaccttgctccctgcttctgctcaggagacttccccatttgcagtaagggtctcccattcctccatctgcttaaacccttcaaggtgctcctgcagtaccttcccaggaagtgccctctcactcccctgagctggcccagcacctgtgccatgtcccacctcccatcagttatggatgccaggtctcatgggttccccatgcctctccttcccatgtccctgcacactctgaggctggcctctgctcttggtgcctgctattaggctggcccctggagacctacagctgaaggctgctgaagggaactgtcccagccatctttcagagtctgcccagctctcaaagtccttcccagcttatgatgttttgtaggaggtatccaccttctacaaggagccctctctgaccaagcccatctggctgggtccgggaaagccaaatcctgccccaggacttgggatagggttttcaccctgccaccttcaaataaatgaggaatccgatcaattcttcctccatttttcctataaagatttccctgtaccactgagactaagctctgtacaaaataggagggaagggagaagaaaaaacaaggagaggaaggttggggcacatccacattcatccttatcctcccagagcttggagaacagtccacaccagcgaggcagagcttcagactcagacctgtgagatttcacaggagagggaggtgggacctggaacagccaggaaggctgcctggaggaaggggctgagggtggggaggaagcactcaaggacagaaaagtgtcccatgtcttagatagggagaccggaaggagcacagagtgtctgagttacttaaatcgggactgagttttccaaggtctctctccaccatgcctgcatctccaggagaaagtttggtgccccactaatcacattcctcacatgctttcccctcagatcctaacaacaagcaaggtgcaactgtggcttaagcttttaaaccccctgcaccaggcccacctgtcaggaacagctcctaaggaggcttctgaggctctggaagaacagacctcacctcatgagccctgacccagccccaggttgaacctgtccccgccaagaacagcactgctcagccacctgatatcaggatccctaaccatttcctgaaatggaacctgggccccatacagacagacacctgggccccatcccaagctacttcattggaatctctgggtgagccctgccatggctgtatttgctgtcggttgggttttttttttttttttttgaaagcattttattttgatagaatttcaaacgtacagaaaagttggaagaatgatccaaagcatatccatataccccttttccagatttaccaacaatttacattttgccccatctgctctcttgttctatctatgtatctatctgagtttatttctgaaccatttgaaagtatgttggaaacatggtgcccctttaatcctaaatgtgtattttccaagaatgagaacattctcttacctacccacagtacagtatcagaatcagcagatgtagcattgatacagtactatctaacccagcatccacactcccatctcagtcatcccagtaggcattagcacctgttaaaagctcctcagataggtgttaggcagtgctggttcagctgagacccagggcagcccaaccctgtgctgaagcagggaccacagcctaggcccctcctgcctgtctcaattcctctctcccacctggggagctacagccccgggcaatgctggcagccgatagaaggcattaaccccttctctgcaggctggatggagagcatagccccttggctggctgggtagcagcagctgtctgggaccagctataaataacctggaggctgtgggctgtggggcggggtcaccactgccctgtggaagaagtgggtggagaatacctcctcatgcccaaggccttaccttggcctcccctccagggccgggtggggtggggctggccagggtgtgaccaccgtgctgggcagcaggctccagtccctaacccccagccactactggcatgaggggtccctcagggcccccaggcctcctctacgtcccacacctcctcctctgcctgctctgcctcctcccaccgccgctccaatacgtaagtgcagggagtgggagtccacgaggtggggcgccaggagcacggagctcccagctctcatgactaggggcgggggtctcagggcctggtggctgggctggatcggggatccttgtgtctgatgatgaattggagatcccgtgcctgggtgagggggtctcagggcctggtggctgggctggatcggggatccttgtgtctgatgatgaattggagatcccgtgcctgggtgagggggcaggggaagggaagatgaaactgtgaggctggccaggatgagactgggagacccctgtggataagcagagggttctggggtgacatcctagcatccagaacttgactacctcagggtccaagattccctgtctcctatccattctctccccacccccatgacccccctccatctctcctgcaagatccttgacttctttccccagcccacccctagttctgacccttacccccagggacccgcagagagccacctcctgcccctgctgccttgtctccatccttcctctcagtgcctggaatcaggcagcccccagtaccagatgagcgatggggacagagtgggctctgggagaaccagcctctccaagggtttctcaagactcagagcttcatccagccacaccccgtgcctttgtccagagtcaggggccaccgatggaaggtggggagaagggacacagagcgaccatcaaaagagagctgaagaagtaggggagaggctggtgggagtggtccacctccagccttctccccattttacagatggggaaactgaggctcagagtggtagggtgacctgtcctaggcctcacaatctggaaagtagtgcctgtggaagctaagaggcttgtcgctgtcctgactctgccacagaccagctttggggaccattctctggggcttagtgtccctatacgtataaggaggataatacattcaacagtaattcaacagataattgcctacaaaaccaatccagatccctgctttgctgcttaatgtgtgaccttggacaggttactcaaatcctcagtttcctcatcagtaaaatgaggatcacaacggtgcttactgaacaagcttgttgtgaggaacattgaggtaatacaagtaaagcaagcagcatcatgcttggtgtggtgtgagcacgcagtgaatgccagctatgcttttgtgccagccgccacagatacagcatgaggacatgagggtccctgccttcctgggggcctggccaggtggaggagagaggtgggaggcaggtagtatgctgagctgtggtcagtgtccgataggcagatggcccccagacctgctgagtcctttgagtgactttgcagactgcaattgggatgggtgaaggttgtacagggggctcaccatccacccctctgcccccaagacatcttgggctttgagctctcttgccctctcccacttgttttccagataggtgccttccaggagatccaagtgttggggcaagggttgcccctcagcccgtccctctggggtccatgctgccagccgcccaagctggttagacggaggaagcttctgctggctgctccagaggtgccctaggattctcagggcaacactgtggtctcagcgtcccttgaggtcatcccaggccctgcgcctagctctcctgcggatcccggagtgggatagagaccagttacttacccacagccactcagcatgtaagcagctggagagcacttaaacctcggcctttctgacgccaaagcccagccttcctctcacggggccttcatggcaatgggactttttcccgggtcctgagacgatgagacctggagcaggcagtcgggagggaaggcagcccaggagagcccagccttggaggcttctcagttattcatccagtgtttaaggaggactgcccatggggaggatccagggtctgaacgctcatctggttcctgttcttgtggtgtttatagcccagaagcgggtgtggcaggaaagaaaggagtctgtgtttacaagttccagtaaatgggctcagaagaagcatgtgtaggtgtgagggcccttgacaggatgggggagcctggtgggaggtgggcagcatagcagagttggagatgggtagtccaagggaagggataggggagcaggttgggggtaaggccccagggccccaaccctgaaaagacattatgtgggagactgcagagctgtatgggtcctggtgggatctgaggacatggggagctctctgagcaggggaggtgcctggtctgatctgcaattgtaagggttccagctggtgcttttgagtgaagaggatgccctggcccagggcacactgagagcctggaggaacccacccagccttgaccctggtgctccctattccccgtggaggccttcgggaagctggtccggaggcaggtggtaatctagcagctgttaagtggtgttaggcgccttactcaaacaccgggaaggtgggaccacttatttacagacaggaaacaagtttaaagaggctcagcaatgccacttgcctcccactctgtgaagatcacaggagcctctcagcccctgctgactaagtccagctccttggagctttggacagcgagacaccccccagggtctcaggatggtgcctctgtgacctcttgccccaggtcctggcttctggtgggcccatctgccccaagaaggctcaacaaagagaacagaacacagagaagtccctggcagtccctggaccctccctgacctgtccggctcacaccaaagaccgagtccacagtgtcccctaacatacacacactgagggtctgcagagccggggctccagggccatttgccagcagctaatgcattgaatgacttttcccaaaaattcaactcccttctgagcgttttctagcctcactagctaatttagtctctagttttgccattttgttggtgtttcagtattttttttttaaatctgtctgagggcaatggctacttaattctgtattcctcctaaatctcaaatttgcagcactttaagaattatgggactactctttatcttcttagctgtcatattctacttcatttaatttaaaccattttatctacagctagtttatctacagtcacttcttccaaaattctaactttttaaattttaattttaattattttattttatttttttgagatggagtctcactctgttgcccaggctggagtgcagtggcacaatctcggctcactgcaacctctgcctcctgggttcaagcgattctcctgcctcagcctcctgagtagccaggactataggcgcctgccaacatacccagctaatttttttttgtacttttagtagagattgggtttcaccatgttggccaggatggtctcgatctcctgacctcatgatccacccacctcggcctcccaaagtgctgggattacaggtgtgaaccaccgcgcctggccttaatttttatttttaatttttgtgggtacatagtaggtatatatatttgtgggatacatgagatattttgatacaggcatgcaatgtgtagaaaatcacatcatgtaaaatggggtatccatcccctcaagaatttatcctttgtgttacagacaatctaattatacttttagttatttttaaatgtacaattaaattattgactacagtcaccctgttgtactatgaaatactacgtcgtattcattctatttttttttggtacctgttagcccatcccctactacccttcccagcctccggtaaccattcttccactctctatctccatgcgattgttttgaattttaaatcccacaagtaagtgataacatgcgatgtttgtctttctcaacattctaatttttagtcattgcaaacccatgggggacagattattcaataagcttattttattttacttagcagccctacagggcctatcatgggcactgatgcacctcacctatttttaccttcataccaacgccctgagttaaaaccatcctctccccattttgcagatgaggaagtgaggtgagctcacgtgcccagggccgctcagctggtatgtggcaggaccaggattccggcccacagcctgactccagattcctttcctttaacaaaattttaattttaatgttatttaattttaatgtcaagttatttttaggacattcggacagactccaaaacaaatcagaagagatgtgacttttatgcaaactggacattagatgtgttgataattaaggagatagctttaggccaagtggcctgctacgggacctcctgctggccagttcctgcccctatgttgggccaagtcacctctccctcccctccccttccccctcctactccagtcagtgttcagggcccagggctcggcacaggtgagacaagcaggactgaggggagagggattcatgggcgttcagagagaagagagaacagcagaatgaggagaggcctaaagcctcccgggggagctggaggggaactgggaaggaagagtggggcatcctaggaagacgaacttggccgagcacagactcaggaacaagaaagccgctgggagctgcccaggaggccaaggggattctcactcaacatccaacaccctacaagttagaactgagtatctaaagtctcctcttccaaggagcctgcctgaacacaccagaaaacaatgacccacttccctcatctgacaccccacccctcgcccgcccctcggccagtctcagctcccatttctgccccctatgtctgtgcagggcctgactgcctcgtcctccccctccttcctcatgtccaccttcctcccctcttccccctcctccccgttcctgtatccttccttcctcccccacctcctcccctttctcttcccagtctcctccacctccttcccccatactcctcttcctccccttccttccttctctcctccccattctccccctctcccttttctgaccccttcttctctcctcatcatcccctctcctccctgctcctccttcctcctttcttcccttcccttttcctactcctctgcactcctcctcccctcttttccagtcttctcctccaagccttctccttctcttccctctcctcttgctccttcctctaactctgtgctccctaaggggcagggcctgagctcaggctcaatgaaatccactgaatgaaagcaaacatggggtggaatgctgcccctcccagctgagtccttctctgaccttgggaagcagagggagaaagcagggagggagctcaccccattcccctccggtccctggcagcctgggcccaggacacatggtcctgtaaatctccccagcttataccatgggctaaatttagacagaagctttgtcgccaggccctggagggaacactagactgggccagacccccaggttccagggaggcgatacctttgaggcctgtcggggccccgggcacaggttctgtttggataggacccaaggtggaagaagagcagctggagtcttggctctcattcctgcatccttccccagccagggcctctcctttgcctcttctaaggtgagggcctcccatgccaggcactgagaggcgggaaccaggacctgggccctcacacaggctaggctcagagcaaagttcagagagaaggaaatataacaagcaggaaaagcccacaacctggtctcccgggattaggggtggggatgtagctctacctggctgagggggccatggtttcattaatccatccaagcagtcagtcagttgacaaacatttattgagcacctgctgtgtgccaggctctgaactggggactggggacacagatgtggaccctgtgctcccacagcctacagtcagtctcatgggctggggtgtgcatggtggagagctctctgccctagaacagccaagagctaatcagcacaggtgtccaagacgttcccctcagaagaaagaaaaatggaaacaggaaaacattccatttcttctctacactcttacacacctctgacaccaaaagtgtggattttctgccccaagcaattctccacttctctggggaccccaactgggtgtcctccaattcaattcaattctgatgctatcttcctggagttggcatcagcctccacaggtaaagcctcagtcccacaagactgcccccaattcagatgccagtcgcaagacagggcttctcattctcccaaccgacaggctatataaatcaagagttcccatgaccctctctttgggttcaatagtgtgctataacattttgcaaaactcaaggaaacatttctatttacaggtttattttaaagtctatcacaaaggatatagctgaacagccagatggagaggcacacaggccaagctgtggggagggcgcagagcaccgtgccctcactggatgccaccctccccgcaccaccaggtggtcaccaaccagaagcccccaatcccacctttgaggggtttcacggaggctccattacagaggcatattaacccaatctccagcccgtctcccttccccaaggtgtaggaaggtgctgaaagctccaaccttctaaccatggcttggtctctctggtaaccagctgcatttggcagcccaccaagagtcacctcattagaactaacgacactcctgtcacccaggaaattccaaaggttttagggattctgtgtcaggaactgggggctaagacctaatatttataattcctcatctctcttgtgcaaggtcctgcccactggctgggtctgcctcccgtgacccaggtgtctgcctccaaggcccctgttcctctgtggcaagagaggggcttctcacctgagcggcctcctgagccccacagccacccagtctggggctggggccctctcagcatccgtgacccacaggtcttcacttcctcacccaggatcagctcttcccatgggaacctgaccaagccataacctaccaactgactgggcttgaatcttgcctctgccccttacctgaagtgggacctttggtgggtgacttcgcttccctgggcctcagcagcctcatctgtgaaatggggttatttgcagctacctcagagactgccgtgaggattcaatccgctgtcttctgtagggcatttagaggcaggtctagcactagagtaagtgctgacttcacgtgaaagaagcctccccttcctgcctgtctcccctcttcttctcttggcacctccataccagatggtgcagtttcctgtgtgggccctgggctcgcctgcctctgagggtttgctttctcagcttctacctgggtcactttctcctcttcttactgctgttcctatggcccaagtccttccatccttttagattccgctccaatgtccagttcgccgaacggccttcggtttcctcaatgagcattcactgagcaccgctgaggggcgagctcagtgagggagatggaggctgcaccttcaggggcccattgtccgagggaaagctgctgagggcaaacggagtcccacactgactggagagctggggaaggggggcacagggtttccaggaagcttttctgggcaggaagagcttgagctgggccttgggagtcccgatgaggggactcgacctcccatcactgagctctctaactagccccctcctaaagtccttgccaagagtcaccattcagcgtgcgcattctacgtgccaggctcttgacctacagtaatttgttgaatgcacacaaaatcctgtgggatttgtattatccccattttaaagatgaggagatcaaggctcagagaacttaagtggcttgccaaggccacacagctaagcccagcatccccagaactctgcacactgggatgtgtgcactctacccactctgacagctcttctcaagtccttggaatgggattatccaagatatgtctgtccctgactttcctggaccatgtgttttctgaggacagcagcctctctttcctcctcctccctgcccacccatcaccaccaccactaggggctctgcccaaaattgacacctgcatggtagattacgacttcggactgagtcaaatccacccaaacaagccccttgaagagaatgagtatctttggctgtgtcagcttttggggccagcagagaagttataggagatgaagccaggatctgggattactccaaaaattacttctgatatgcctgtaccaaattttgcccagacccaaagagcaagcatcagttgcagagtatggtaaccctgtgcccactccccacaaagtgcttgctcagccagaagatggccacagcctgagaccacagaatccaaggcaaggaccatcccacattgcctgagtcctgggggaggatgccctgaccccatcatgggttcaaagcatgtgtcagaaaccccggtaaagtcaaacaggagatcacctcagtgatggggctacaaatgtcacagctcaggggctttgagagaaggcactaacatctgagatggaatggctgcatctgcctaagggcgcaccgtggacactgcatgcctctgggttgcccgtaccctgacctccagaggctgggattcactgggaaccctcccacagagggcattactagctgcttctcaaacaagggcgatgggcgccataaagagggtgatgggtcagacctagagccctcactgtccccagacccagccaaaagtatggcctgagaattcttcctggaaggtctcagtgtctggagtgttccctcccctccccagccccccaacccctggcagtggctcacggggtcagcaaggtcttcctgatgcccgagaagctggagctgaggaactctcagcagctcccttgatatgtggacagcaagaccatggcccccagtgtttgtcctgcaggccaagaggccagaactcctggctgacagctcaggaggcccccacagtgccgagacacacacacaacacacacacctgctcccctgcatttccctctctacctcacttcgtggggtgggagtttggcagaacacaggtgcagtgagtgcctccaagtggtgtctgcctcaccctaggcttccgggaggggtgcagggggcagcactctcctgtctgagcaggtggggtcattcattagaagctccaggctgacgtggtgagggctgagcatgcagcttccagtcccagcacaggaaatggactgagcccaatgtggagaacgtgatgtttgagggcttttgggctctggcccctggggacatcctggggcaaacattgacccagctgccaggaagagtacatctgacaggcagcaggaagagaaggggctgcacactgatggagcagtgtcttctggaatcccagaatcaggaatctcagggttgggaggggcacaaaagtcacacacgtgtgtgcatgtactcacacacacacacgctgaccatagagcaagaaaaggagtggtcttgggaggaatggggccaagcgttagaatttagagtgaattgctctcgttcagtcatacaccatgcattttccagcctctctgcctttgcacatgctgtgctctctaactagaatgcccttcctggcctgatcagcttggcaagctcctactcacccttcaaaacccacattagcatgacctcttctgtgatgctacccctgccctcctcagtaaatcgtacatgccgtaaaactgtacatcctatccatctttcccaccagaagatggtcccttcacgggtgcacatgcccacatctactttatccgtgcacatccagcacccactctgatgattttttaggagacgggatcctgggtgcttttctatcttttggatggaattgccattattgcaacaatcacatatcttccctagtgagtgcctaagctggccctatggtcccttcttttgtccctactcatctttctgggtgccccaaaatgctacctctcccaataagccttccctgtcttcctgaatccaggttaggcacttctctcacgtattaacctaatgctcttcgttgcaagtgcctgcttacctgtgtgacagggaaacaaagttaaatcatgcatttgcctcctcttccttctgcactttctaagtctagaggatccctacctggcctgggactacctgaaggacacccatcagttcccccagccagggcctgacacacagtaggtgttctgcaaatatttgttggttgaatgaacaactaatggaacccaccccccacccagctttgctgacagaaggacacaagccccatgacagtgacacatcccagcctgaggaggagacagccttgggggctgagggatggacagagcagacccctgcctaggaggcaacactgcattgggccacttctgaggcctttcttcggtccagcccaggccagactgctgctgctccatgtggccatgggcatctccctccttgttctccctctctgtactaagaggagtaagggatgagtgataggataggatgggggcagacaggcactgtcaatggctagcctgggcctgatctttgaccctcatccttgctgtcccctaggcagccgggcagagccagatgcccaaagaccagcccctgtgggcacttctggagcagtactgccacaccatcatgaccctcaccaacctctcaggtaggtaccctctgccaccaccctgcagcagcccctggagatgtgggtgggccctgcgggaggaagaggctttcctggggccagcccaggctcagcagggtggtgggtggggtgctggtttccagtcctggtttgatttctgccttgctgagggcccttacgcaggcccttccccattctaggctcaggcttgccactgataatgaagggcttggctgggaggccactccccattcccagctcccacgatggaagtcagctgggctggcattcaggagtccaagaaggggatgacccctcagctttccaactccagggaaactggcagaaggtggtaaacattctctggttgccgtggctacaggtgtgacttcctggtaaggacttgcaaagcagcagcccggccctgagttctggcccctcagaggtttccagtggctgattcacagggtcagggccagccactgcagagctgcacagagccctggattctgctgggttccagagagatgtctccagtgtcccaccctgtatcctggccttcccaggtctgcatccacaggatgggagtgggggtgacccttgctcgggcctggggccactgccagggcacaaggagacaagggtgctggccagcacccagacccagcctacaggcactgcccaagccaggcgtcaaagagaaagtcttgctgcctgttctccttccactggggcagtcaatagctccggtgttgggatcaggtagatccaagtctggatcctagctcttccacccagcggcctcgcccagcccctggaaactcacctaacccatctgtattagtgtcctggggccactggaacaaagcaccacagactgggggcttgagcagaagctgatttcatcaccattctggaggctggtaggccaagatcaagctgtgggcagggttggttcctcctgaggcctccctacttggcttgcagatggccatctttctccctatgtcttcatatggtctctcctctgcatgtctgtgtcccaatctcttcttcttctaagaacaccagtcctattggattaaagcccgcttaccttactttactttaatcacctcatcgaagaccctaaatccaaacaaggccacattctgaggtactgggagttaggacttcaacatacaaattttggagcaacacagttcagcctgtgacaccatctgaagctagtttcctcatctgtttagcaatggatctaagtccacctcggaggctcacagtgaaggctgaatgagagcccacggggacacctagcacaggccatgccagcatcagacaggaaatggaggcccctcccttcaggaaagcccaaccttccctcaccctcagaggcagcgtgggtttgtcagcagacccatagccttctccctgccttcctctgccctccctccagcaggtggcacagacccttccctgtctcccttggcggatggtccccgctctgagtgcagcggatagggtcccagcttctccaaacatacagccttgcctgttcagcttctaacagcattcatccatttgcaaaagggatccacatgatttctataaaattgggcacaaaaaaatttaactcaaaggagaagatgataagtactcattctcccacaacccaggaatttttataatgcctcatatccctcaagaatttatgcattcatagagcctcatgttcaattgtttttttattttctttttcttttcttttttttttaatgggataatagtgtgtccatagtttcgtcgcctgctttttcctcttataactcagagtgaacatttccccatgtcattaaatattcaaccaccgcatcgcttgtgacgctgcctagcagccactctggggatgcatcaccccacatgaaccatgaccggatggtggatgtagaggctgctagggagcccctcgagcttcaggaatcatttctcaagtccccaaattgataatgaccttcttttcagtgtccactgcttggggaaatgcatcatgtctaacgcagagttcaaaaccaagtaaaaccaaacactatatcttttagatgtacatacctgtgagaaaactgaaaaaaaatatgagccagagaatgacaaatgcaatagtcaagggaatgttccctctggggaaggagaatagaaataaatagataagtgactggtaatgatgtagtttccaattgggtggtaggcttattatattgtctactacatacaataaaaagactaaataaaataaagactaaaaaataaagttaagccatgcatgggctaatgcacagactaataatgacaaagggtcacgaatctatgatcaattcaattgtatgcccctgaaaaaggcaacgcatgcaaaatcacagagcaattatgacttcaggcagggtttaaatagattatttctattttattcatcacagaggcagctacatatatttgaaaaatattagggcatatattatgaggcatattatttatcctatccatagatgatataaaaatccaaatggactatttatttattttttattattttttgtaggctggtcttgaattcctgtgctcaagcaatctgcctaccttggcttccccaagtgctgagattatgggtgtgagccactgcacctggccaagaatccgaatggattcaaagataccttgaaataattcctcaatgcaacacacacacatatgccagggttggtcaaatgggaagagagccttggcctgaagacagggacctgggctttcctcagctcttctgccaaggtatctgtcctttcttagtgactcactgggctgaagtctagagcattccaatgggtgctggggatgggttagtgaaccaggaccagccctgcccttggggaggcatgggctggtagatgagacaatgaataaaaagcaacctggatgtgacataggccagcacccagcagatggggtcaccaaggagctgcatgtctgaaggatgagtgtggagctgtaaggccatttccagtgcagaaatacaaacaaggaacagagatagcgaatggtttctggtagtgacacattgttctggagggcctttcaaatgaagcaggagggtgaggatgggggacgatgatcatgaagaaccttctttgcaataccaaagatggttcccaggaatgacatgttgttctggagggcctttcaaatggagcagaagggtgaggatgagggatagatgattacgaaggaccttctttgcaataccaaagaggtggtctccatctcaagggcaatgggttggcggcactcctatgaggaagagtgacaggatcgtaattataaaacatcgttttatctgccatgaggagacagggagcccatttgggctgctgcagagatgaggatggcctgggagaagtaagggagggaaacctcttttgcttgtctgggaatcctattgtccatctgtaaaacaagaatgtgggattggatgtcactgagagtcctttctgtccccacagcttagtcccatggtttgacatgaagacaccaggccaattcctctgtcctgcagtggagtaggatagggtgaggagggggtgcctggcctgggacccctgtctccagtgctacagggcaaaagtgcagcaccttcaggcccctctggaacctctgtgccctcagcaaccccgtcgtaattcccaggaaacaccacgactattccagctgtgcctgaatagactccctctctatgtagtctaaccaaggacagtcccttagagaagcaaagatgcatcctgtccctttaaatcctgttttccagctgacatttgagtggtaggggatgaattagagagagaatgtgtgtgcatggtgaagatgcataactgtggctctgtgtgtgattctgttgtgcctacatgcctgtttgatgccatgtattaggctttgcaagtgtgtctgagctggcctggaactgtccactgctgctgcagctgacatcagaggtgggccatgggatgggatgcagggcaccagaggcacctgccttactctgctgctcatggtacacaggggtcttccaaagtacctgtcacacttccctgaacctatttgccaacctgtccccaacagcttggggacacacaaactgttccaaatacttatcccctaattcctggcctccagctgggatggggctggcctgcagcgctgggaacccatcactatcccaaagcctctaatctacctctgcttctttagttagcaaaaatgcccctgtctttgtttgtttgtacttggatttagtaaaattgagggaattttggggctcttcccaatttcttcctcatttgcctgtttgcagacactaaagtgagctgtaaaatcaatttgttcccaaactgctaccttttctagttttccctctgtcacatctcgaatgacaactgtgcctataataataagatccatgaagaaatggccccacatccaggggacctcgggtctgtgggtctgtgggtatgtgctcagaccccaactgctcattcaggatgcagaacagcctttgactctgccactgaaatggtcacttcccagaagaatctttggtatgtgagctctcccagttaagagcaatccagttcacccaggccaaccctctgttctgcagaaatctgcatgagatgagaagtccctggcctctggtcattgggagcacaccacctcttgacatagctgcatctataggtgctttaggcaccacctaggtggtggctgcagtaatgaacaaggataataacttaagcttggcttaggatttctcccttcctctgtgctgcaccgtgctgtgtgaagggctatcccttgtatatgagggacactattgccctgtgaatacatgggctgacctggccagccaccctctggcctgtgtgggaccctgggtaacagggctcagtctaggagcggagggtaggactctgcctaggtcctcccctgctcctggcactggataatatgaagaccaaggaacctctacctctgcagctgcccagagctgagcctgggctctcactgtccattcaggaggcagagggtagacctggctggctgctcagcatcttgttcctgccagtcagcaggccctggagagattccagggctcagccctggtcttagtgtggtctcactcgctctgcctgggcagtgacagcaccattaatatggaggctggtgagagcggagcacacaaaagcagcctgcctgctgctttgcctctctctgcccagggcatggtgctagttcatggtggtttcagcctttcctagcagcttaggtttatgtggaggatggcaggggaacaggactggtttctgagactaggttccagctctccttgccctctaaagatagaaacaaacaacacacaacacacatgtatgtcttccccagactctccgtctcataactcagaaccagagagtctcagagctgagaggggcctcggggaagatttcactgatggagaaagctccagaagagagggcaagcatcctgcctggggtaccatggcagggagagcagagtcagaggctggaccaaaatgcagcctgagtggaagcatgccttaccccaggccctgcttcccgagcccaatgccccactcagctggcagacacagcaggcccgccctgggcactctgagggtgcagccacagcagatcaccaaggaggcattctgggccagggtggggtggggggcctgggcaagttctctggggaggtttccagctcctccacacctgctgtggggcctgattctccccgcccctgccccgctactggtgtggaaaccagggtcaggtgaagccctgcccaagccttgaggaagagagacacattctcacttcttcctttattctttctggtgcccaaggcactaacaactgggtgtataaagccttccagataatttcagccaatttctcccctagacttactccatctgattaaatggccacccagtcactcaagcaggagacctgatgttatccctgcctctgggctcctacttaaaaccagccacccactccagttcctcctcaccaggtctggctgcccctccgaaatctctctccattccagcttccacggtctgatccaaggacttcaacccactcaggccacttgatctcattagactcatgggtgtctccctgcttccctccaggcctcctcgatacatttccttcccagaggctatggaggcttttggaggatgcagatctgcccatgggctctccgtctctgcctccctccagtgtcctcgctcagggaggggaagctcaggcggaaagctgccgaactttgggttgcggctgtccctcgattagcagagctgcggtgttctcctcgggcaggcgggcaggtgggcgcgctttgctgccccctgcagctcgggggcctgcgatccccgcacagagcattccgtcaccccaggcccacgctctccagcccaccgccctcctctggacgccgcgagtggaagagagctgcgaactgagaagccgtactttgggcagggtggagggcccgggggctggagactgagcccctccgagaggagccgcccggccccgccccccggcgcagccattggccgcggcggagcggctgtacccgcagctccgtgcactcggcggctcctctccgggaaggtccccacttgacagctctgggcgaccggaggtggcgcccaaaggctgcccgggagatcggggctgggctggcgggggccaggaccccgcgccctctcggccgctcactctcgcgtccactccctcgcagtcacgccgggcgcgcactcccactccctctccgcacgcggctgcgggacgcgatggacgcggcactgctccacagcctgctggaggccaactgcagcctggcgctggctgaagagctgctcttggacggctgggggccacccctggaccccgagggtaggcgggaggcgagcggtcggaggctgcgcgggctccttctctcgcgcgctgggtgcgcccctgaccccgcgctccgagagccccgcgtccctgcgggcccctctgagccctactgcgctctctcccgccgccaggtccctactcctactgcaacacgaccttggaccagatcggaacgtgctggccccgcagcgctgccggagccctcgtggagaggccgtgccccgagtacttcaacggcgtcaagtacaacacgacccgtgagtgcccccggccctcgctgcctgctctacaccggcctcctgagaaggcggtttagggagtaaagaggattccggacccagcggtacaaagctgctgacggccgcggtaccgcggaccgcgggcgttaaggaccccttacagtgttgggggcgcgccttctgcttctacccccaatacagcgtctaatctcagtaagccagtgatttgcccacaattcccgtgctccatgcggtgacagcccaaaaaggggagaaggaaatacttcctcctcttggagtgtaaacctccttccctcattgtaggttcccgttgcctgaagttccccattagggcttcattgtttagacaatgcttcccctgcccaggagggggacctatcccaggagggcggatatgctaagtcccaaatcccccagagtgagtgcagtgaccagaccctactaccagaggggaggaggagggtaggaggcaggagggctggggcgagcgcatgtttcgtggggaggacatgcaggcgtaggttctgctaagccagagggtgcggggtgttgcatcttccctaatccgaggtcccaggactgacctgctgggctgagtcaggggctgtgggcagcattattcctgcgcccccataatgagtggtgtctgaatcagcaccccaacaccatctctcaaggagaacaacctgtgcctgccttcctcctggcaaatcatggactggcttgaggctctggggaaaagggcagtagaggttgctggagttacacagcctccaggtggggtcgttcagtgtgggttctgggtgtgtgcgtgtgggtaggtgcctgtctgtgccctgtggaacatgggatgggagccaggtgtgtggtctgcgggaggctgtgggggcagcaatcactgccccacctagaacccccctgatatctccagccggaccccagttgtcaagcacgctgagaccctatctggattgtttgtctgaagcctctcatcctggaaacatcctcagagtgtctgtgtctgattggtagcccagctccacctccaccacacatacacacacacacacacacacacacacacacacacacaagctttcagaagctgaggctgagggctttgccatgagagatggttagaaggcaagagagatgtacctgcacctgatgccaagccgcactctcctggctgcaccagctgagggctctggtgggcccagggagcacatgcttcccatccatgattaaaacagatctccctagaaggaagtaagatccccaagctaggaggtgtgcaagcagaaggaatgatttccctgtgccttcctagtcctgaccttataaaactgtatttttaggattccgtgagctgtgtggggtggggcaatggagatcagcatgcctgattcagactgggcccggagattaagggccatccagggtaaagtggggtgatggagcatctcagcccacagcctgtgaacctcagggagctctgctttcgttcttaccccagcctgggctatgccttgttctccccgaccagcctgggggcccccccttcccagggcctgggtctcttcttcatagccaagctgagtacagtaatactcagtcaatttgtggaatacccactatgtgccagccactgcacaggcactgtattgaggctctcactcacaattgcaacagacaaaaagaacagaatgatgaatgccctaattaggggggaagcacaaggtctatgggattggggtgtgggggcggggtgacaaggaagacttcccaaaggtgctgcagacagcacatatcaggccattcatatcaattaagtctaatgagaagctgataaaaggttggggcaccacccaggctggtaaaagtagggccaattagcccctggtatctaatgaacaattttcttcatttagaccaaagtgctgcctgcactgcaactgctggagaatgagaggagcttgtggagggtggggagcacttaccactaaccagcacctggggtgtgaggtcagcctaggtgggaagagcagaggctgctaatggctgtggaaatgcatgattgatttccctcctcccctttcttcacatcagtgctgcttcctgcttctctgtttgccttttccttgggatccttcctttaaaaagaatgacagagggagaaacctccacgagaagcctaggggaagaacaggagcgttcaggctccacttcctgccccaattttgttatgctatagccaaacccgcattgaagcctcattccttagagagcaggaatcgcccgggtggggatagcagtttagatattagtctctgcgatttttcaactcagctgagcacagagaatctgccacaaagctaagtgttgatcattttccaggtgccagctaccactgagagagctttgtatccatatctcaggcatccataatcccactcgcccacactccctctgagatacatactgcttttatgcccatattgcaggcaagaacactgagtctcagacacatgaagtaacttattgaaagtcctaatcagctcctatgcaccagtgcaggcaggtgaatccacagccctcgctattaaccatggggcagtactccctccacaagaggcattcggtttgcgaggagagcttagaggtttggaaagaagactcatacctccggcctggaggatcagggaggacttagccctctgagctggacttctggggacaggttggattttagcaggtgagtgtaataggcacaggagaggccctctaggctgaggggactgagcaaaggcaaggagacaggctgggctttgtgccttgggaggagtgtgttgtatggagaacagggagtaggagaaagaaacaatgaggctggggaggggcatggaggtcaggtgatgcagggcattctacagggcttcaccatctggagagggagcctgggtgagcttgtgagcagagaagctcaattttgggagcacactgtcctctggagggaaaggagaagtaaggggatttctgcacagacccaggtagaccgcaggctgtaagagcaaatgaggttgaagacgccagcaaccttgggcatttgaaattgacaagatatggtgagagatggcaaggtaaaggttgggcacacaggaaacagctgagcacagttggggatgtgttgactttgaggagctggagggacagctgagtggagatgttcaggaagcagctggttgggtgggggaaaagctcaagagagcaccaagagtggagataaggcgtttagaagtcctcaggatgtggggttagcaaatagctacagtgtgtgagtttgggaggctgggtcagagccttgagccatcttacccagagttatttccagatgcagcagggccaagggaagggaagggactagcgcatggccacacatgttagtcagtggcagagccaagttaagcacacgagtctccttgggtgttctctacacaacctgtgctggcgcttcagtggctgagcttccacatgtttgtactgtggtttaactcatttaatccttgcaccatcgagtgacagagacatggcattatcctcattttacacatggggaaactgaggtgtggagcaatgaagtcacttgtaacagaacaaggattcaaacccaggttctgctgcctttcaaaaagcaactctgctaggttaagcagagaagaaattttttaaaaagatattggggagcttatggaatcactgagtgggctagagaaccagatttggggccccaagccaagaacacagggcagagctggccctgtgaggggacctccctctccctgaaaggccctgccaacaccaccagccctggccaccactagtggcatgatcaaagatcactgcaatctcaaactcctgtgcccaagcaatcctcctgcctcagcctcccgagtagctgagactgcaggcacatgccaccacacctggctaatttttaaattttttgtagagttgggatcttgccattttgccagagctggtcttgaactcccagactcaagcgatcctcctgtctctggctcccaaagtgctgggagtataggcgtgagccactgcacctggccaacctggagtcttaagcttcctgcagttatctctggagaggctttctccctgtctccatctgggggcctccagttcactaattccctcgggtaggtgcatctgatgggtgggggaatatgtggagccccaaatcccatgccaaagctctggctacaagggatcctgggaagtaaggatctggcattttagctctaggcggggaggtagcctcggtcttaccccaggattctccgaacccaggttcaaatcctgggtggctagaaacaatcctaaatgtccatatacaatttaattggggcaagtctcatcccttttgtgggcctgtctccctgtctgtaggccgaaaggaagtggattagcatggaagtggtttggccagaagtggggcacttatattccattttcctcccacctctccccattccacacggactcctctgagcccatctctgtgtgtacatctctgtctctgtgtctctctcattgtctccctgtctgtttctgattttctctatgtgtggttctgtttctgggtgtatgtttctgtctttctcagattctctcattttctacctctctctctctttctccatctctccctactgccttcttcatcccccgactccatctccaaacccactgtctccccactcccatccctccttctatctgccctcctcctgcacctcatcttgcctgtccctacggtccttttggctgcaaaggagggtcttgccacctccctcctggccatggtccattttccttctgtggaaccactaagtgcagcttggctgagcctcagcctccttgacctcaagctccgcaatgtcctactgggaatggctggggtcatctgtactgaggcatctgccaggacaggggaagaaggggcatcccatctaacctagccacatggggaggttaggggtccccacttcccagaaccccattctagagacatgcttaagcactgattgtgttacaatggaaacacataagctgagtggtctccagccagaggggtctctcccattgggtctgcctgcttatgcaggaatcccttcttcagccctaagagtcagaggtgataagaaaggctgagacctgccctcaggaagccccaggctgcttatcaaggaggcttgtgcaaatcaacagtcagagatcgggaagaggggcttggtggacactgggacattcctgggaggctttctggagggagtgggggtaaactttaaaggtcttggagcagttgggatgccacatttgttaaggacaggcgcagtgcaggggtgcaagcctcaggaggaccccagatagaccctgccctctgggttgaagaagtagaagctgtaaatccggaaactcaggtctgcattctcagactccagcgagacctgatcccccctgactctattccattctccaagggccacaagttactccatgtgtgagaacaacttccctgctgggagagagggaaggtgggacaggcatggccatcacagaggtttagctaagaaaagaaggaaggaagagaaatagagcagaagtggatgcccttgtggccatccagacaccccagagctgcccggctgcagagcgctctatgttccgagtccagccttcccctccccacttaagggtaggaaatgactggccaggagagggaaacacagctgctcagagttctacagcaagtctgggccctgataggcctctgctggctctgggaactcctggcttctgctctagcttctacccacctctgcatgcatcttgatctttcctgtctcctgctctccccaggaatcctccagccacctgatcgtcttgctcaggctcaccccttattggggttctgagtattttaccctttcccccagaaagaattgaggagcctagagacaagcttttcaaagatgcataagacctgagcctgccttccaggggctcctgttctaggaagagtgacagacaggcagactctcaaagagaactttttgatataggctcccaagtaaaatagcacagtgaagacagtcagagcacatggaagggagttattccaagagtaagggaaagtaaatttggaagacttcatggaggaagtgttgttgaacagtgccttgaaaaattgagctaatgatgtgtcagatggaagggagggtgggagcggaagtggaagaggagggagggaagaataagccagatcatggaggcctcaaatgttggaccagggcatgggcctcgattctgctggctttagagacagcggctgcttgtccgtagccctatggcacaggaccagaatactctggaaggatactgcttttcaaaatgtgccatctaaatctcaatcaagggctggctggggacctgggccttggccctttcctctcctcccacctgctccttaagtcagaggtcccagcatccttccctgtgcactccttaccaccagtgccacagcagagcctggagccctgaagacgggctgaaggaggagccccatctcagtgatcctcagggagctccccacagggcttcttggctgggccccaggtacaggcatttgggcccctctgactgcccaccctcaccaggtgcttcagagtcaggcaggcggtccctttcacacacagcacctgccaggagagcagctggggcaagggagagtgggaggaacctgggctcagatttagcagggctggatctaagtcctgggtttgccatgactcactgagggactggcaggccctccttgtccaagtcccagttttcttatctgtgtgaaggggacagagaacatgctgacctcgtggggccatggcacacaggggagatgatgctgcttggggctccctgtagacagctaaggcattgtcagcaaggggaggggagttgaaggtggcctggggcctggaaggaggggccatcatgtccttgggtaggggaaatcctgctcacaccaagaatggctatcctcattctggggtggtggtctcctgctatctctgagagcccccagaactctgtggcttctggggatgtcctgggagggaggattctgtctatctagattgagaaggtctggtacctaaaagcctgtagagaaaggcagccccttgcccccctggagggatgctgctggcatcctgccagccagctgtcttctccctgcattacttcataggaactaggaactgagagccagatctaatcccggccatcctactgactgcttttctccctttgggcaggtggtctcccctcttcagacctcagttcatctgcaccaggtgggtggagggcttgatgagttctagtgatgtttctcagttccaaggcagcttctgccacaggggtcctgtgttggtcccctccccactttcctccacaggctggcgtcatggcgacgagcccactgagtgttgctaggatggctggaaggagggagccctgtacagtagaggcgggagcatgaggtaggggcctcctcccactgcccctctctggggagggcagagcttacacagtgagctgggttccactcccccttccctttttcatagacccagtatggtctccattaaactgctcagtgcctctatgactcagtttccccactgtctcctgtaagtgtgcttacaggtcctgcttaaggatggcagggcaccatgagtgaaaagtcctgtggaaatagggagttgagggtctctgaaggtccagaacaggcagtcgaggctcagagagctgtgtccatataacactaagaaatggaatccaagttccctggcctgaatcccagacagcaccagcaaagggccctggggatgcctttcccacccagtggctggggttggcaatattagactcccaaactcaatctagcacttctacgtcagtctgggtctgagcttgggtgaacatcaccagctgcctgttcccctctgaccaccaatttgctgtgatcggatcaagacagaaatatgccaggtgggcctcaaagttggtcagtcctccttaacctgcagtggcctccaagcaatgcaggatgctgactcaaggagatgcacctcgtttgggaagctcaggaaaaccatgctgttagtactaggactgtacgtctgaccaccccagctctgtccaccttccccaaaagtctcttaactggggatgaaaagcaatccactccaggaagtggaggccaagctgggaggagacattgattgagaggtttttcccacagaaagcaagaaagatggccaggcatggtggctcatgcctgcaatcccaacattttgggaggctgaggcaggaggatcacttgaggccaggagtttgagaccagccaaaaccacatagtgagaccccgtttctacaaaacaaacaaaaaaataacaaatgatcatggcatggtggcacatgcctgtagtcccattactcaggaggctgaggcaggaggatcacttgagtataggagttcaaggctgcagtgagctaagatcacaccactgccccccaacctgggtgacagagtgagaccccatctcaaaaaaaaaaaaagtaggtaagaaaggtgactcaaagtcatggtcaaagatttctcaactaggccccaggccagacaagaagatggctggatctggagtggcccaactacaggcagtaaagggaactgagggtgggatatctggcccctaaggtattctcttctgaggatctggacaccaagtggtaatgacactgaagaggtggcataactggctgcatctgctgaaacacagggtcacagggagagtctgtctctcctaggtacaccaactccagggtctcccgtgaggaagcgtgtcccaagggagagagatcttctgcctcttcatggttgagtgaccccagatgtgtcagtttctctcttctggtcccaccagaccaaatgcagaagttgtcttccttcctaattcccaaggagatgcctgataactcagtttccaacttgcttcccttggaacagtcaaagctttcatgccaggaacctggagaatgctgcccacaggtttgctgtttgagactgacacccctcccccatgtggggttggactgcaatgtctgcagttttggcagctgggtcagcctacccatggtgtctttgtgtgagaccttgcttcctcctgcatgtatgtgtgactgttttgtatttgccatgcatcttggtgaagcatgggcatacaaggtggcagactgtcctcctggcccactctgagcctcctaccttcaatgagttcccaagtaacctaggaaacagcatcagctttggtccatcctgagatcctgtgtgctcagctctgtgcagtgcttaagaaaaggccagagatgacagcaacccaggacatgcctttagaggctctcagccttgtacaaaataaggtaaacttctgctccagagaacctgggctggttaggaaagtacaacacctttataggggtcagtgaaagatcaatcccagcctagcatgagccaggcagtcacaagcagctggacccttggggagctggaacagtcatggaaactacctggaggagctggggctggagctgggccttgaaaaggggcatgaattagcttgacctcaatgggctgggcccatggccaaggtcggaatatcgggtgggaaggctggttcttaggagctctccgaggtccccaaccttagagataggcagggatgaagaatggagttgtgctgtccgcggtgctgaaagttccgatcccatgtagtgacttcattccttctgtaaatattgcttctggccctccccacctcaatgtcatcaccttctctccctcaccaccactactactactagtactacctcttcttcttctcctcctcctcctcctcttcatcctccttcttcttctatcatcatcatcattatcatcatcatcatcatcatcatcatcatcatcatcatcacagggtctcactctgtcacccaggctggagtgcagtggcacaatctcagctcaccacaacctctgcctcccaggctcaagcaattctctcacttcagccttctgagtagctgagactacaggcgcacaccaccacgcctggctatttttttttttttttttttttttgagagatggggtttcatcatgttgctcaggctagtttcaaactcttgggctcaagcaattcacctgccttggtctcccaaaatgttgggattacaggcatgagccaccatatccagcctctccctcacttcctgtccttttctttgcaaatagccaatgaccccattgttgtgccttaccacagtcccattaactcttttccccatgcctacccctgtcccttgtactccttcccccaaaactaccaccaaaagtagtccctcatggtccctgtctcctatggtccatgtctttccagaaggtatcagagatagaagtgccaaccatttgatggtgatgtcaggatgcttggagttgtcacaatagcttgagggtgctaactggcatttagtagactggggagtctcacataacatataattgatccacctgagatgccaataacatcatagagaatcaatttactgtaatatttctaagaggcaaaactgagaccaagagggaaggggcttagtcatggtcacctagctatctagagacaaaactacagctagaatatagggtctcatgtcccagccaggcttcaggaccaccccaggtaactgcctgaaccaggaactctttgcaaagtgtcagcatctgaaacggggagagcccattgtaatgggggtttttggcaggcctgggccactctgggcatggaaaccaggcctcccagactacagagtcctgcaagagaagaccctgcctgcatcattcagaccctaatgcccctggaagcccacactccacatcatcagcactgatcctcagcaggggcacctgtgttgctatggtaatgggcacccacctctctgcatccagccccacaattctagccttgccatcagccctcagagtgtgtgcccatccgtctacgtttcccacggggtggatgagattctttctgtcttactccctcctgtccgaggctgtgcctgagggcccagtgccctcattggccacagcagacagctcccctgaagagtggggcacatctatcctccagtcctttgatacccagtgtctgtaaccagaagctcatctgcaggcagggctgggctgccagagagggaagtcccctcccgcttctgcatcccacctgctttcccaggggtcagcaggtctgagtcaattctgttcagccgacatttcctcaatggctctgtcctgagggagatatggtggctccaatatggcttctacttacttgtctatccccaaggagctcaagtccagtagcaaagaaaagaaggattaggtccaaagagatgaagttcatagtgaattatggcaaatgcactaaaacagaatttgtatttttcaaactatggattgtgacacaagagtggactgtggagtcaatttagtgggtcatgatcagtgtgtgtgaaatgaatagaagacattagaataggctggaaaggaaaatagtacagctggattctgtgtttgtgtgtgtgtgtgtgtactggattataacataaaatgtatgtgttattgtggttcaagggcaataaagtggaaaaactacgttacaagagaagggaagggatagaggaggaaacaaagaaaggactgcttcagacctaagggtgtcggggatggctccatgcaggaggcggcatttgagtgtggggttaaagatgggtaggagttcatccgtgagcagtgggtaagaaggaggaggtgctttaagtggtggatccacacaaagagggtatgtgccaaacctatgtgtgccaatgccagtggccatgtagaatgctggagatgggtcaggacagttaggctgggacctgggtgtcaagggccttgactatcatgctaagaaattggactttattcttgagcacaataaagagccactgatggctttaagcagaaatcaacatggtcaaggtggcaagaaaagaagcagggaggcaagaggagaggaggctgttgtagcagcccaggcaagaagactagagagcagatggagggatgggcaggaggggagttgactctggaaaaagaaaaggaggagacagaatccatggaacttgggaccccatgggcattggtgtgagggagaggaaaggtctctgatttgcagcatttctatcctgaccccctcccacctgcaagcccaccccatgatcagtgccaccatgagatatcatattcgggaaacaagtattccaaggccctgtcctcagggagcccccacattgatcagagtgaccagactcactcacctagacagctccaggctggtcatggggcaagggctggagtgtggggctcaggcggtcaggactggaggtgctcaggagagagagagctatagagtagaagcaatcaaggaggtcttcctgaaggaaggggcctagcactgagcttcaggaaagctggataatatctatgaacatatagaagataaatttttctgccctttgaagcacaggcaggatcctggtctgcctcacctcacatagactgttcagaggcaccgtaaacccccatcagggagggactgttcatttcacaaaagaacttaccctccttacatacttttgcttttttagttcagtgacatcactatcactgaccctcacgagttgaggtaacctttctctttcatgagcacgaatgttggtaaggaaagatagtgctaggtatcaaattagatcccacctggggagttgggcgccatggctaggggtggggagcaggcttcctcttcctgagagtcagaatcagcatcagaccagggcattccctgactcacttggcagggggatggcagggccctgccagggatgggccttggcccaggaggaaggaataggccagtcacgtccctgggtttgaagaatggatgagctccaggcaccaaactaaaaaacaattaagctaatagaatcttccccattgattttgctcttcggcaagtgctccttcccaattccattgcttccaactcatcgggctcaacggaagtgggtttttaagtaacttaattatttcagaacacactgcttccaaaaacaacctaaggttccccctccagctctgcaactctgtgctctgcaagctatttattttaattagctttacattgtacatctaacatatgctagaaagaaaaattcacagacaactgcccattagtcagagagctgaaccctggtacaaatcacccccaaacaatggaacgttcagcatttgtaacccacccggtaggtcagaaacacactagtgatgaaggccaagcagaggcaggcttcgagagctccgggccttctcaggtgggagggcttcctcctgattggagctggtgtatggagggggtggcttaggagccagaccttgacagttggagctgagatagagagaaaggcaaatgtgagcattttttaactttcctaccttcccccttgacaaaacctgcacttttgtgcacaggacattgctgcttgaaatgtcttttcacacttaaatcatgttccaacttaaatacagaggctggttcttgaacatttagaaacatctcgtggtagcctcccaggcagcagcagcccgagggcggtggcccagccctgagtccccagcattcactcattcctgtttgccactacttgatatggcctaaaggttaagaacagaagacccgaagccacaacttctgagtttgaatcctcttgtcatctttgtatccttggccagtagcttaatctctttatgcctcagtttcctcctctttgaaatgggttcatattaatgcctccttcatgggatggttgtggagattgaattgacatgtatatccatagcagttagaagaggaagtggtacatgggaaacatatgctatataaatggtagctattataaaaatggaagtgctgatattggtgaagtatggacctcataaatataaaacaattgagacaaggatgtaaattatgtggcatgctgaaagcagtgaatcttcaggcacaacccactcctcacagttggattcagttttgaagcaaatgaacaaaatgaaagtgtacaaaatgctagtcaaagattgtgtctaaaatgggttttagcatgcctttagctattttgtaagatgagccttttgtgctattttcacataagtgacttacatgtaaactttcagatgcacattgtatttgctgccctaggagagctgaggtgcgtacaccttatcctgtgggcaccagggagccagggagggttcttgaggaggggagtgacctgactcttggcagttggcattttttgtagggaatgcctatcgagaatgcttggagaatgggacgtgggcctcaaagatcaactactcacagtgtgagcccattttggatgacaaggtgagtggccctcacctgctgtgaccccaggcctcacacaggtatttggactcaggttgagcagagaagatcccaaggggggcagaccagtccttttgtcacccagtcactcccttctgtacctcctgcccctgcccccagtgtcctgccctgtgcttagtgacactgtaggggagtagagaagggggacatggagtcctaccttcaagagagcttgggctgctagaggacaaagccaaggtggcatgtgacacaaaaagggaataaccgtgtaatcaatgctcctaacatcttaggggttgtgtgactgcacaccttcctctccctctgggcctcagtttccccatctggcattctgattctgtgactccacatggaaacaaatgcagccccccaagggctgtccccatgagaagctgggtcctgctctgcctctctggagccctgctccagctctcgctctggaattcaattcagtgctgctgaggcaccgaactcctctccttgaggctacgcctcattcctgacctgaaaggatacatctcagctccatcccctcctcattacccagcatagtcctcagtggggctgggactttgccccagatgaagtccaaattgagagggaaaaacattcacccgccctgagaaagtgattctgcaaaatgtgagccacaggggttctgcagggaggccactcaatgggcaggggggcctccagcccttggtcctcacctccactctctctgcccctccagcagtggagaatcaacacaggggttgggtagggtggaggggtggctctccctggcaggaaggagtatttcatcactaactttgtttcaaattgccaacacttggtgataattaaaagtcaactgacttttattcagtttgaatagaatttctaaattctctatagacagtgcaccccctcgtgtggtctccagggcagactaatctcaatgcaccccaccccaggcctcccataggccattgcttcttaggtaggatggcaggttggccccattgtatagacatcaaaactgaggtccagaaataaaaatcgactcaccctcagtcactcagccagctgggatccgggctgagactaaactcaggagaagcattctagagcctagaattttaaaaaagaatctgtacccatacctctcaaaatgtgcccccaaaaatgcatgttggtgccaggcacagtggcacttgtctgtagtcccagctacttcagaggctgaggtgggaggattgcttcaggcctgcagatcaaggctgcggtgcactgtgatcacacctgtgaatagccactggaccccagcttgggcaacagagtgataccccatctctaaaaaaataaaaataaatgcatgttggaaggcctgaccacagccatttataacaacaagagccacccagggccaacaggaacgaaccctaagagccaggcacggcaatgacacctggagcacttctgcccccaaggtcaggacacagaggtggccacggctggaatacccagccccacccccacgtgcccatgcctccatctgccctgcccatgtctgtctgtccagcagaggaagtatgacctgcactaccgcatcgcccttgtcgtcaactacctgggccactgcgtatctgtggcagccctggtggccgccttcctgcttttcctggccctgcggtgagtctgcccttccgccttgctttctcctcatccctccccctacagcacccccttcacctctcccagacattctcacctccattctggggcccagcagataaaagggcccgtggagaaagtggggtttgggtggggtgccagggcatgaatcaccagcaggcatgtggggtgcagagtgggagctggactcagaggcccaactgtggccaccatgggtgggaagtggctcctagtgcagccctgccctcatcccaggctgagtctctacctggcctcagggggaacccagtcccagttgacccctgaacctggctgacccctgacctgcacctcccacaggagcattcgctgtctgcggaatgtgattcactggaacctcatcaccacctttatcctgcgaaatgtcatgtggttcctgctgcagctcgttgaccatgaagtgcacgagagcaatgaggtcattgctcggggagactcggggctatacctggggggctctgggctccgggggcacccatctgtctgtctggtctctgcttggacccctcctttgtcaggaagctcactacctgctaaagcagccctcttcttcttgagcagcttttactcttacaagctcttcctaatgttgaacttctgcaactccaccccattcttgcccccactctgagaacagatcccctctgctctctccaggctctgagacagaccccttggagatttgcacacagagactgatgttcaccagtccctgctactctcagctggtgacaactgactttctggctgctccttggctgacccatggccagaatgctgtcatctgggctgtggctcaggtgcaaaaccagacctgagctcacctccaccctagccattgtcccaacagagctgggctgggaggagcaggaagctgccaggctgggagcttcacaaagctgactctagacacatttatcctagtccactcctcatcttgtggattcaggtgccctcaaagacccctttgagaaaaggaagctagaactaacctctgttgagctttctctcacttaatcctaatgagaccagcagggatcattttccccattacatagacatggaaactgagactcacagaggttaagtgactcgccaaggtcacatgcagtgatgatacctctacataaggggctatcccacatgctaactgtccactctggatttctgtccattgctgtgtactgtgtccaagctattgtaaaactgtagactagtcttgggccatcacagagccctgtggcacaccgacagagacttctcagtacatttcctttgggtggcctgtgcctgagcagacaccagcctggtgtgatccttaacatggtcccacatagaacactgcctcagaatcaaacaggttcactcgagttgagggtgccttctagctccaacgtgggcttgtgctaatagaacatcatcaaccctggatgaaaagtagggttgggaggaggggcttggtagcttcccacctcagcaggactttctcagaaaaaaggagacagccagccacagggactgtgcagggaagcccagaagcagccaacagctcagcacaggagggttatctgagaactggggctgtcagagccagacagagaggatgggctgtgtgcagaagcagagcccctgacgcacagctccccggaccttgagctggctccatcattccccatgtcccagccttggctctcagggtgccctggggatgagcctgtggggaagatgaccaggtggggtctgttgagaggagctctggcctggaagcccaatgtctagaccctggccctgccatggcccactgcgtggctctaaaggggcagggtcttcccctccctgcctcctttgggaaggtagccttggtgcctggggaagggtggccttgctcagagggacagggacacattcccctctgctgggaaggcacggaaccacagcaggcacaaccacagccacaacacagctaccctcatgcctgtctttgccatcctctatctccttcaaccaccctggaaggcagggttactttttgaggttcagagaggtgcagtgacttgccgggggccgtgtactttgcaagtctccctccagagctagtaataattgtgccaagtccagaagttcccaaacccagagtctttggagaatcccctccctggccgcagccccacttcatacacccttgactcactttactcacttttattttttcatttttttctaaatgaaattccatatcccatccccaaaaggaagcaggtctgacttgctatggattgaaaatgactcaaaaaaactaaaatgaaaacacaacaagatggttgaattccagcctctgcctacagaaagctcagagccagactcttgctctctttactagaatgaaaaattagaaagagttagagagaagttaaaaaaaacacgttggccccagtggagaatggcaccttaacttaatctgaaagtgtgaaagagcactggagaaggatgaagcttttcagagtgaggttcgctggtatttgatggcatgtccacgaaccccctttggggagccctgtgtcttgttgccctgcctggggatgggtatgtcctacctgggccccaagctgcctcctgacagcctctctgccctgcacacaggtctggtgccgctgcatcaccaccatcttcaactacttcgtggtgaccaacttcttctggatgtttgtggaaggctgctacctgcacacggccattgtcatgacctactccactgagcgcctgcgcaagtgcctcttcctcttcatcggatggtgtgagggtcccaggggcagcagggcctagttggggggaggacttctcaccaggccatttcccttcgtcttgggtccaaggaccagagcctggccctgccctccacccccagcgcagtccggtggatgcagctacttccctccatgctggtgaggaacaagcaaaggatctctccaaaggatattcctcaaatagctcttctccccctgccaggaaggacctgtccaaaggcctttgtctctggctcagaaactggctggctctacttctctatcacaaaatcacgcagccctgcttggagagaggggcaggggcatgggccaggtgttgagggacagggcacccctggggttgtgggtggcctgctgttggaagagtaaaaacatgatcctagccctgttgtggtaccccacggtatgtcctacaagaaagtccctgtccatctgtcccctgcagcccaagtctttccttctttcaggcatccccttccccatcatcgtcgcctgggccatcggcaagctctactatgagaatgaacagtaagtggatgggccagccatggggggtcatggggaagggaatgggggtgttaggacaggtcagccttgggctctggccaagccgtggagcaggaattgtagccacacctagacagatccacgttcctgttgtccttaaattgaaaaaaatagtcctgcactcagaggtactcacatggtagggctcaaggaactttgctgaattaagttaggaattgtggttccttgaagtgccccccgacccaacgcatgacccctttggtctttgtaaggcctcctcagacttggatgtggcagctctggcccggggtatgtggaaggggccaaggccgcacatgacatccagagctgtccatgagcatttaaagagtccatgagcatttaacagagcttgtttttttttctgaagtctcagtctctgcctgggtgcagtgtcccatcctgaggttctccctatgccagaggctgtaatatgctgagccacagcccaaatgacagcaactctggccatgggtcagccaaggagcagccagaaagtcaggtgtcaccagctgagagcagcggggaccagggaacattagcctgtatgcaaatctccaaggggtctgtctcagagcctggagagagcagaggggatctgttctcagagtgggggcaagaatggagtagagttccaggagctcaacattaggaagagcttgtaagagtaaaagctgcccaagaagagggctgccttggtaggtggtgagcttcctgtcaaaggagggctccaagcagaggccagaaaccctgggagacagggagaatcctatctgaggcaggtctgcactggatgacctccaagatttcagaggaatgagttccccaaagttgatggcccctccctgtggtccagtagttgctggccgacccagaccctaaagcatttcccctgaccagcatccccggagaatgaattatgcctctggcagccagtgaggccagccaggcagcagctcccctcactgggtgcacaagcattggctcaaagacgcaaaatcccaggctggaggtaggaaggctccacttccacatctgctgctttgtgtggccgagtgagtgcctggcacacggaacagcgggtttcaagcaatatgggaacaggtggcctaaggcaattggccctgggccagcaagggggagcccagctctccctttattcctgtgcctcctgcatgcccagtcctatgcctagcagtctgcaatggggcaggtaaaagaggaggagaaagagacccatggcctgtcctcagtcgggctggggagatcagacccaccatgtagagcaaaagacagcctggtgggtaagggtggtcagtggagctggtatttccaccaaggctttctgggaaggggcagtcctggagattgggtctcaggaagggtaaagctggaggaccctgggcccaagtctgtgggggtcccccagttccagctcaggaggccccctacagccaccccatcttcacaggtgctggtttggcaaggagcctggcgacctggtggactacatctaccaaggccccatcattctcgtgctcctggtaagccctcagcggtggctgggatggggctgggtgccaagaggacagaagagggcccacccaggcagagacgctgggaaagatgtggcatgggcagccctgcgcacacactctatatgccacccccgtaccctgtccatgccaccctgtgccccagcaccacccggccttcccttcctcatgctcgatgccctggcgcagccctgcaccactctgctgacctcagggctctttctctgggaggagaaaggatctgatgggtagacttcctgtgccctcccggtctgacactgtacaatgctgagaaagccaggtgagcaggcagctggtgacaagagaagagtgtgcgggcttctgttcattccagacctacgttcccctcagcccccagtgaccctctccaggtccagcccaccacccccagcctgaagcggtgaataccgggatgcagcataatgaggaattgttccttgtccagctccctgccctttgccaaatgtatttgcatctaaggtataattgccacacatctttaatccacaatgagctttaattaagagagagttcaagtagaggctgcattctgcaaaaggaacccattatatatcatacctttaaaaaaaagagagagagataaagaaagaaagacatacaaaaaagccccgtaagaaattggcatttacgtaagacttatttcaggctcagtaatttcaggctcctgtatgtaccagcttccttaaagggccacagttccttctgggagactcagcgagagtgccatctcctcctgcacacagaaggtgcctggtggggctttcttttgtctaccttggagacaattattatttatctcttttattttcttactctcctttttctgtgacttcgggtttcatttgacctccatctttggagtgtccttgatatatgtgaactgatgaagactaggaaggtttttcacccactgctggaaattccaccgtcagggcttggttcgcttcagaggcagaagccaggttttgtcagcagctagtaggattgtccttggactcccagctgcagtgagtcactcacccctcagtgtgtatggaaaccccatgagatcctttccctcccaaccagctgccaagggccatcctggaggaataaggcttttgatgagcctggaactgagccaaggaaggctggcctggatgggggaccaatggtggcaaagagtaagccccaactgtaaagatgttggaagggtggtgaggaaatgccaagagaaagggaggcgaggaacgcaagagctcagtgtcaagacccagctccatgcttcctcttccagacccattctctggctccacccctgcgtctgtgctcacgagccctttccttctttcctttgggcccagctctttgcaccaacaggggcttccacattccaagctgagcccttccagaagctctatctacattatttccaacccttgcatgctcaagtcctaaaagaagtagggttatgctctccattttacagaggaagaagctgagagagatgaagtggcttgcccaaggtcaccactaatagctagcagagcagaggctcaaaggcaggtctcctgctttgtccccaccatctgcctgttgtcctcgaggttggacacgtgtttggtgttcctgccatccctacactccccagcagctcacagctgcatttctgctgctaagagacactcacttatttctggttctgagaagtgaatcaggtggttgaatttgcaggaagcctgacttcttgctggtgttgtcattgttggtgtcatgggaaaggattggcagtcactaggccagacacaggtcttagtggtctctgtgactatttcagcccatgctgatcttctaaggcaagctctgtggttaggcccctcgggggcagacaaggaagcaggagctcatgaggttaagcaacttacccaaggccacacaactgcaaattggaacagtcaaaatttgagccatgagactaggacttggtgcttcagttcatttgggaggggggcctcatcttcagttcttagcccatctgccatttctccaggtctttttgacaccttaggaaagatgtcatctcccccacccccaacccctccaggcccccagctaggccacaagccctctgcaagcaggggctgtgactgcctgtccacttcgccatcgctgatgcctggctcagagcctgacacatggtccattgattcattgaatgaatgaacaattaaataaaaccagaaacaggctgaacgagggcagggtacaatagacagttcttactaaagaactggagtgtgtttttagcagggaggtagagtccaagaatggaaagccggagagctcagtgtgggcagggtgaccagaaggctccctggagagagaggaaggtacctgtggtggtgtgaagggggaggctgggaaacaggctcaaaggaaagaaggattttcaggttacatagacccaagctgagatgagtggggccacataactaaatgtctgccaagtgtaaggatggagacaaggccttggttgctaggtagatgaatggatagacggatgggccagtggatgggcagataaaagagtaggtggataactgaatggattggttattgggtaggtagatggatgggtagatggatgggtacgtggatggatagataaaacagtaggtggataattgaatgaattggttgttgggtagatggatggatggatggaaggaaggaaggatgaatggatgaatggctggatggatggatggatggacggatggataaaagtaggtgggtaaatgaatgaactggttggtaggtaagtggatagatggatagatgaataaagtgggtggataattgaattgattagtgggttgatggatgttggaagattaatattgaattcaagaaaatgtggcagaaatgagaatatattttgattccaacagtttctactctcagggaagtccggaagaatcagaaaaggtcaaatactttgtattacttgtatgagaaaattaggattcacttatatgttaaggtgacttgtgtcccatgatccaggggaatatgccagagacccaggagctggaaattaggtgagccctaggagaaagctgggggtatcaggtcatggcagccccgctgaaggctttccatccccagccctccctgggacccccaggccataccactatgtggtaagagagcagtagacagtggctgaggtggggtgtgagctctccctgcccctagggctacccgaaagcttgtggggagcatccagaggctacaggttgacagtctcaggatatctgtctgtatcctgtgtccctcctctcccagccaggattcagaacttgggagggttagaagggcccagcccaagcttaagaagcaatggattttcttgacagagtgtcagaggtgtgtgtgtgtgtgtgtgtgtgtgtgagtgggtgtgtgcacacgagccagcagacacccctgggctaacatgtgtctccctctggcccacgagcactttgtcaggaaacagccccactgtgtcaggccgacacacactcccttgcctgcctgcctgcctatgctcggaggcaggtgaagagaaacgcagatgtgcccgggccccagatccatgctcacacatgcacacgctctgtgaggcaaacacacttgcacaagtgcttaaaggatagacccacagacccacactgcagctcccaggggacaacacagtcatagtcacagccaatacagatgaacagacactcgcaggcccatggggacccacacccagcagggagcagctggggagccacacaggccagttccagggccaggtgctgggtggggaggctctggtgctgagggaaaactcagaatgaagataaatgaggcccttcctggggcctcagagccagccccacagcccatccatcttccctgtctagggtgcctacgaaggctctggagttcctgggcacaggggtgggatgctggcagattaatcactcattcggcaactgagttcttaatccctccatctggccttgtccgccccaccatggcagtgctgtcctcagtcagaaggacctcaggccgacctagagccagtgacagtgcaagggactcagagagccccgcagtcaacaagtagggaccccgtggcagtgaggggaagggggctcccatagggtccctatactggggagagggttgggggctttctcagcctcagagggcatgccccgtgctctcggtcacgacccacagaggctgccacctttcctcctcagtcctgggctggagacacaggccctggaaaatggggctgtctccccatcctggcgagggaggagcacaggccttgggatgctggcaggtgctagggggacaggtaggataggtgtgtgcgtgggacatcggtttccaggggtctgcggggaagggggctgcctctcggctcatctcaaacacgtgctccttccttttccctctttcccatgcccgccctccctccagatcaatttcgtatttctgttcaacatcgtcaggatcctaatgacaaagttacgcgcgtccaccacatccgagacaatccagtacaggtatgtgagggggtcccttgaccccacaatggggacaggaggaggtcatcgggccgtccccctgcttcctggcagagccctctgccccaggcctcacccagggagctcgctcaagctcccggaagaaacaccaccactgtctcccgccgcccactgggtgaggccctgcctgccgcatgggctcagacctgtgtccacctctcgcccccaggaaggcagtgaaggccaccctggtgctcctgcccctcctgggcatcacctacatgctcttcttcgtcaatcccggggaggacgacctgtcacagatcatgttcatctatttcaactccttcctgcagtcgttccaggtggggcctgtgaccagatgggggccccacagtgagctttcctgaggctgctctggcagccatgccctcactaagggggtcccgtgggactggcacacccatggctcagttacatccacagagttagaagcccgtacatgctcagcctgagcctggccagttatccccatcccagcctgggtcttcatcctaccagcccgcacctactgtcccagcacctgttctgaggatgcaggcagcccaaccaaatgccctgggacaggtcacacaatattgaggagaccggagtctgcctggctccaggcctgtgaccactgctcactcagagtggaccgtggaatatctgcaccctgaggctaggaccccacccagctctgactccaggggttcctgggtcaaattgtcccccccccaccatcttgtccccagtgccaccccaccaggcagaacagctccgcaggtaggtcactgaccactccctcctttccctcctttccagggtttcttcgtgtctgtcttctactgcttcttcaatggagaggtatgaagctgggccctcggggtggcctgggatatccaggtcctcagaccccactccagggcagctccattggtgctgcccggccttgctggagtgccagggagaggaagccttgagataccacccggccttaggggaacaggaatccttctcacaggggctggagccagtttagaaggtcaactatgcagccccgaaataacatgccagcaccccacatgtgcactcagagctgggtgagcaaggaagcccaggccgcagggcctcatggaagagagaagccagagcagaggtggtgacagaaggaagcttccagaaagagatgtgtgccaagcaatcaggtctgtatggactcagggagcagaagagagaccagcacattcccccgctacctgccacagaaaagcccacacaccaagagcaggctctggggagtcaggcggccgtgaagcttggcatgtgcaagccaatcatgaggcccaaagccccagtgcagctgacgcagggaagaagcgcactcgtacctgggctggggtggatgaggacatggagcaaagagcagacacagcccctagggcctgggagcttccgaacactccagaggaatacacttgcctgtctctgcacagggcactggcgaggtgtctatgcaggcctgtgtctctgtggatgatagcacattccacttgcaaagtccatcagcagagacgggtcctgtgggctggatgagaagaagagagggggccagtaaggcaaacaaacaagggatgtttatggagcatttattctgttcgaaccatgtgcaagctaggatgtgagagacaccatccaatttccaaaaaaattcaattaccaaacaaatgaagcttaaatggcaccgggtagagccaactttagaaatgaccaagacagagagctttccctttcaggcagtaaatgccatggttagtcaggcaggggagtctagcaggaggtagagaaggtggaggggtgcagaggggacggtcttcaggagggaggaggaggcgcaccaggtgtgtgagctgggcgcacaccaccaacgccctgcaaggagcaggggcccaagaaggctgagggggccaagtctccaagctgcgctctctggtcccaagatgctaattttgaggtggcagagtgagtcagggaagggaaggcagtgtgtccagaggaaactggatgatctgggaggacagtgtggagtaaaggaggtgtggagtcaagcttctccaggaaaaggataaggaagaggcccctggagagaggcaacagcatcagggaaggacctgttttggaagcagacagttccttgtcatgagggtggcagtcgacccatgagagcagaaagcagaggaggggatggtcccagaagcccagggttggtccctgggagccagctgagcctctgccttccccacaggtgcgctcagccgtgaggaagaggtggcaccgctggcaggaccatcactcccttcgagtccccatggcccgggccatgtccatccctacatcacccacacggatcagcttccacagcatcaagcagacggccgctgtgtgacccctcggtcgcccacctgcacagctcccctgtcctcctccaccttcttcctctgggttctctgtgctgggcaggctctcgtggggcaggagatgggaggggagagaccagctctccagcctggcaggaaagagggggtgcggcagccaagggggactgcaagggacagggatgagtgggggccaccaggctcagcgcaagaggaagcagagggaattcacaggaccccctgagaagagccagtcagatgtctgcaggcatttgcccatcccagcctctctggccagggccttactgggcccagagcagagaaggacctgtccaacacacacagctatttatagtagcagacacagggctcccctgccctactcatggagccagcagccaggcaatggtgtggccctgcactggcccttggcctccacactcagtggtgcccctgcagttgggtggggtacgccagcaaaggatcagtttggctgccttatcccaggggctgtcacctagagaggctcacttgtaccccacccttgttcctgtgtcccctccccagccatcctcccgccttgggggctccatgaaggatgcaggcttccaggcctggcttcctctcttgggagaccccttctctgcctagtccacagattaggcaatcaaggaagacgccatcagggaagccacatccttagtcaaccagttgcatcgtgcggggcaaaatgaggagcagaggcatggaggagggaggcgtgggatgggaatagcagaaaccaaccatgtcttcagtgattgaaactcataccccattgccctttgccctccagtctccccttcagaaacatctctgctctctgtgaaataaaccatgcctcttggaagtggcctcccctcaggtcaagtcacccaggcacccgaagagtggtgtgggccaaggatcgtggctgggtctgagggggccacatccatcttccttagtgggctcgggccctggcctcgggccagttctctctctctctgacctctggtaggtttggagtctttatggtcaaagcacctgcaggcgggtctgtcccttatcactccaaccaggctaatacctggccccagcccccaaccaagggcagcttgattcagcctggaggaggtgtgcagctcatccagcaagccagggcctgtcccaaaggccaaggctctggaatctggtgggcagggtagacatggcccctcagttgaccaccagggcagcttccaggcttggcttccgtccctgtgtccctgcagcggagccgcaggagtcagggagagagaagcatcaggccatttcttccaaaagaaaccaatgctctaggtaccccctacaaaaataagttttgaaaaatcatgtatcccgtcttgcacatttttaagttgacatgtagtattttttacttaagttttaaatccttcaaaggtgtgatttctgccattttgtaaatatttacatttttaaataaaactgctgcaacaatttaaaatacatcaaatgaattctgatcaccatggtgatttaacacccaccctcatctattttaaaaacacctgacccagctctccttcttcttcttttttttttttttttttttgagacagggtctcactctgtcatccaggctagggtgcagtggcaagatctcagttcactgcaacctccacttcccaggctcaggtgatcttcccaacccagcctcccgaatagctgggactacaggcacgcaccaccatgctgggctaatttttgtatttttttgtagagacagggtctggtcatgttgctcaggctggccagctccgctttaattgtgggaaatgttacattgtttctttcctctttaatgtgtagcttcattccattgccctatagaattttatactaatgcattacttttatggctgaaagtgttttattgctcactttgtcatacttctctgccacacaatatatgtatgaatgaaaattaaaatatttcattttctatgaccaaagtttcaagggattttaaaataatcttctgggttgaattattacaattattttttgatacaaaatacagtaagccactgattaaaacaacatagatacctaaagtttttataagtaactagttaaacctaaaaaggaaaatgtattgaaaacacagcttaatgaaatggataggaagttaatggctcatgaatctacaggtgaatatttattgctagaataaatcaggactcagcatcaattcttttcttttttaatgttttattgtttatagtggggtgtgctgagaagccttttcaattcagtagatgatctggcactccagatagtcaattctctatactcttgctgagatgtacagtcaaaactgcatagtgatctgtcaccaaaaatgacctatcagctggctgtttaattaagtgtcctttcaatttggctgaaaaagatgaactgaacgttatctgacttgcagtaggctttgggcgcccatgtcatcagctcattccatttttcaccaccagctgtgtgggaatgcactgcaggttaagtgtgagccctgcttctcttgtcaagagggcagggaggttggggcaccctgattttattttctcaagtgacagaactcagaactcagggtatcttagaaagtcttactgcatacccagtggcagcatgatggccgcatccattagcatcatcagggctcttgtagaacaaggagccgcaaatggtttggcccccaagaggccacttggagatttctgtggccagatgaggctgtgaacaagtgcttatcggagcagacgctgcagagcctggcaccaagtcccaggcattcatcactgcttcttaatgcagcccccggctcattacgccaactgtcctttgctccctggagggaccaagggcctgtgatccctgctgggaggccaggcctgccaaggccaacaccaggaagagcatcaaagttgctaacctttgttcagcaggcacttcgatatgctctctctccagttcagaggaggctcctccttgatccaaccccagccctgaatccctcattactccgtctcctggttgtggctcccactgagctctatccttaaaatgccaccagagccattaggccatctctctgggtccctacttgttaagctcgttaggcacctgttccttaagccaagccagtcctcattagcatctgcttcggcgctggcagcacctcgtggagctggccttcctggaaagtggcctcattagcagactctccttctccgtagcaggcaagttccagcacccagatcctggaggcccttagggtccctgtttaaccctttcacccaccctgatttacacaactggggaagacctgaaccagccacaggtcctcacaccagcctcttggccccactccaagagagagtaacaggcagagccctgaccccagcacagctccaagtccctgggtcatcatcaaagtgcggctagaaagtacct

SEQ ID NO: 4 - DNA, 55161 bp, Homo sapiens
aggtactttctagccgcactttgatgatgacccagggacttggagctgtgctggggtcagggctctgcctgttactctctcttggagtggggccaagaggctggtgtgaggacctgtggctggttcaggtcttccccagttgtgtaaatcagggtgggtgaaagggttaaacagggaccctaagggcctccaggatctgggtgctggaacttgcctgctacggagaaggagagtctgctaatgaggccactttccaggaaggccagctccacgaggtgctgccagcgccgaagcagatgctaatgaggactggcttggcttaaggaacaggtgcctaacgagcttaacaagtagggacccagagagatggcctaatggctctggtggcattttaaggatagagctcagtgggagccacaaccaggagacggagtaatgagggattcagggctggggttggatcaaggaggagcctcctctgaactggagagagagcatatcgaagtgcctgctgaacaaaggttagcaactttgatgctcttcctggtgttggccttggcaggcctggcctcccagcagggatcacaggcccttggtccctccagggagcaaaggacagttggcgtaatgagccgggggctgcattaagaagcagtgatgaatgcctgggacttggtgccaggctctgcagcgtctgctccgataagcacttgttcacagcctcatctggccacagaaatctccaagtggcctcttgggggccaaaccatttgcggctccttgttctacaagagccctgatgatgctaatggatgcggccatcatgctgccactgggtatgcagtaagactttctaagataccctgagttctgagttctgtcacttgagaaaataaaatcagggtgccccaacctccctgccctcttgacaagagaagcagggctcacacttaacctgcagtgcattcccacacagctggtggtgaaaaatggaatgagctgatgacatgggcgcccaaagcctactgcaagtcagataacgttcagttcatctttttcagccaaattgaaaggacacttaattaaacagccagctgataggtcatttttggtgacagatcactatgcagttttgactgtacatctcagcaagagtatagagaattgactatctggagtgccagatcatctactgaattgaaaaggcttctcagcacaccccactataaacaataaaacattaaaaaagaaaagaattgatgctgagtcctgatttattctagcaataaatattcacctgtagattcatgagccattaacttcctatccatttcattaagctgtgttttcaatacattttcctttttaggtttaactagttacttataaaaactttaggtatctatgttgttttaatcagtggcttactgtattttgtatcaaaaaataattgtaataattcaacccagaagattattttaaaatcccttgaaactttggtcatagaaaatgaaatattttaattttcattcatacatatattgtgtggcagagaagtatgacaaagtgagcaataaaacactttcagccataaaagtaatgcattagtataaaattctatagggcaatggaatgaagctacacattaaagaggaaagaaacaatgtaacatttcccacaattaaagcggagctggccagcctgagcaacatgaccagaccctgtctctacaaaaaaatacaaaaattagcccagcatggtggtgcgtgcctgtagtcccagctattcgggaggctgggttgggaagatcacctgagcctgggaagtggaggttgcagtgaactgagatcttgccactgcaccctagcctggatgacagagtgagaccctgtctcaaaaaaaaaaaaaaaaaaaagaagaagaaggagagctgggtcaggtgtttttaaaatagatgagggtgggtgttaaatcaccatggtgatcagaattcatttgatgtattttaaattgttgcagcagttttatttaaaaatgtaaatatttacaaaatggcagaaatcacacctttgaaggatttaaaacttaagtaaaaaatactacatgtcaacttaaaaatgtgcaagacgggatacatgatttttcaaaacttatttttgtagggggtacctagagcattggtttcttttggaagaaatggcctgatgcttctctctccctgactcctgcggctccgctgcagggacacagggacggaagccaagcctggaagctgccctggtggtcaactgaggggccatgtctaccctgcccaccagattccagagccttggcctttgggacaggccctggcttgctggatgagctgcacacctcctccaggctgaatcaagctgcccttggttgggggctggggccaggtattagcctggttggagtgataagggacagacccgcctgcaggtgctttgaccataaagactccaaacctaccagaggtcagagagagagagaactggcccgaggccagggcccgagcccactaaggaagatggatgtggccccctcagacccagccacgatccttggcccacaccactcttcgggtgcctgggtgacttgacctgaggggaggccacttccaagaggcatggtttatttcacagagagcagagatgtttctgaaggggagactggagggcaaagggcaatggggtatgagtttcaatcactgaagacatggttggtttctgctattcccatcccacgcctccctcctccatgcctctgctcctcattttgccccgcacgatgcaactggttgactaaggatgtggcttccctgatggcgtcttccttgattgcctaatctgtggactaggcagagaaggggtctcccaagagaggaagccaggcctggaagcctgcatccttcatggagcccccaaggcgggaggatggctggggaggggacacaggaacaagggtggggtacaagtgagcctctctaggtgacagcccctgggataaggcagccaaactgatcctttgctggcgtaccccacccaactgcaggggcaccactgagtgtggaggccaagggccagtgcagggccacaccattgcctggctgctggctccatgagtagggcaggggagccctgtgtctgctactataaatagctgtgtgtgttggacaggtccttctctgctctgggcccagtaaggccctggccagagaggctgggatgggcaaatgcctgcagacatctgactggctcttctcagggggtcctgtgaattccctctgcttcctcttgcgctgagcctggtggcccccactcatccctgtcccttgcagtcccccttggctgccgcaccccctctttcctgccaggctggagagctggtctctcccctcccatctcctgccccacgagagcctgcccagcacagagaacccagaggaagaaggtggaggaggacaggggagctgtgcaggtgggcgaccgaggggtcacacagcggccgtctgcttgatgctgtggaagctgatccgtgtgggtgatgtagggatggacatggcccgggccatggggactcgaagggagtgatggtcctgccagcggtgccacctcttcctcacggctgagcgcacctgtggggaaggcagaggctcagctggctcccagggaccaaccctgggcttctgggaccatcccctcctctgctttctgctctcatgggtcgactgccaccctcatgacaaggaactgtctgcttccaaaacaggtccttccctgatgctgttgcctctctccaggggcctcttccttatccttttcctggagaagcttgactccacacctcctttactccacactgtcctcccagatcatccagtttcctctggacacactgccttcccttccctgactcactctgccacctcaaaattagcatcttgggaccagagagcgcagcttggagacttggccccctcagccttcttgggcccctgctccttgcagggcgttggtggtgtgcgcccagctcacacacctggtgcgcctcctcctccctcctgaagaccgtcccctctgcacccctccaccttctctacctcctgctagactcccctgcctgactaaccatggcatttactgcctgaaagggaaagctctctgtcttggtcatttctaaagttggctctacccggtgccatttaagcttcatttgtttggtaattgaatttttttggaaattggatggtgtctctcacatcctagcttgcacatggttcgaacagaataaatgctccataaacatcccttgtttgtttgccttactggccccctctcttcttctcatccagcccacaggacccgtctctgctgatggactttgcaagtggaatgtgctatcatccacagagacacaggcctgcatagacacctcgccagtgccctgtgcagagacaggcaagtgtattcctctggagtgttcggaagctcccaggccctaggggctgtgtctgctctttgctccatgtcctcatccaccccagcccaggtacgagtgcgcttcttccctgcgtcagctgcactggggctttgggcctcatgattggcttgcacatgccaagcttcacggccgcctgactccccagagcctgctcttggtgtgtgggcttttctgtggcaggtagcgggggaatgtgctggtctctcttctgctccctgagtccatacagacctgattgcttggcacacatctctttctggaagcttccttctgtcaccacctctgctctggcttctctcttccatgaggccctgcggcctgggcttccttgctcacccagctctgagtgcacatgtggggtgctggcatgttatttcggggctgcatagttgaccttctaaactggctccagcccctgtgagaaggattcctgttcccctaaggccgggtggtatctcaaggcttcctctccctggcactccagcaaggccgggcagcaccaatggagctgccctggagtggggtctgaggacctggatatcccaggccaccccgagggcccagcttcatacctctccattgaagaagcagtagaagacagacacgaagaaaccctggaaaggagggaaaggagggagtggtcagtgacctacctgcggagctgttctgcctggtggggtggcactggggacaagatggtgggggggggacaatttgacccaggaacccctggagtcagagctgggtggggtcctagcctcagggtgcagatattccacggtccactctgagtgagcagtggtcacaggcctggagccaggcagactccggtctcctcaatattgtgtgacctgtcccagggcatttggttgggctgcctgcatcctcagaacaggtgctgggacagtaggtgcgggctggtaggatgaagacccaggctgggatggggataactggccaggctcaggctgagcatgtacgggcttctaactctgtggatgtaactgagccatgggtgtgccagtcccacgggacccccttagtgagggcatggctgccagagcagcctcaggaaagctcactgtggggcccccatctggtcacaggccccacctggaacgactgcaggaaggagttgaaatagatgaacatgatctgtgacaggtcgtcctccccgggattgacgaagaagagcatgtaggtgatgcccaggaggggcaggagcaccagggtggccttcactgccttcctgggggcgagaggtggacacaggtctgagcccatgcggcaggcagggcctcacccagtgggcggcgggagacagtggtggtgtttcttccgggagcttgagcgagctccctgggtgaggcctggggcagagggctctgccaggaagcagggggacggcccgatgacctcctcctgtccccattgtggggtcaagggaccccctcacatacctgtactggattgtctcggatgtggtggacgcgcgtaactttgtcattaggatcctgacgatgttgaacagaaatacgaaattgatctggagggagggcgggcatgggaaagagggaaaaggaaggagcacgtgtttgagatgagccgagaggcagcccccttccccgcagacccctggaaaccgatgtcccacgcacacacctatcctacctgtccccctagcacctgccagcatcccaaggcctgtgctcctccctcgccaggatggggagacagccccattttccagggcctgtgtctccagcccaggactgaggaggaaaggtggcagcctctgtgggtcgtgaccgagagcacggggcatgccctctgaggctgagaaagcccccaaccctctccccagtatagggaccctatgggagcccccttcccctcactgccacggggtccctacttgttgactgcggggctctctgagtcccttgcactgtcactggctctaggtcggcctgaggtccttctgactgaggacagcactgccatggtggggcggacaaggccagatggagggattaagaactcagttgccgaatgagtgattaatctgccagcatcccacccctgtgcccaggaactccagagccttcgtaggcaccctagacagggaagatggatgggctgtggggctggctctgaggccccaggaagggcctcatttatcttcattctgagttttccctcagcaccagagcctccccacccagcacctggccctggaactggcctgtgtggctccccagctgctccctgctgggtgtgggtccccatgggcctgcgagtgtctgttcatctgtattggctgtgactatgactgtgttgtcccctgggagctgcagtgtgggtctgtgggtctatcctttaagcacttgtgcaagtgtgtttgcctcacagagcgtgtgcatgtgtgagcatggatctggggcccgggcacatctgcgtttctcttcacctgcctccgagcataggcaggcaggcaggcaagggagtgtgtgtcggcctgacacagtggggctgtttcctgacaaagtgctcgtgggccagagggagacacatgttagcccaggggtgtctgctggctcgtgtgcacacacccactcacacacacacacacacacacacacacctctgacactctgtcaagaaaatccattgcttcttaagcttgggctgggcccttctaaccctcccaagttctgaatcctggctgggagaggagggacacaggatacagacagatatcctgagactgtcaacctgtagcctctggatgctccccacaagctttcgggtagccctaggggcagggagagctcacaccccacctcagccactgtctactgctctcttaccacatagtggtatggcctgggggtcccagggagggctggggatggaaagccttcagcggggctgccatgacctgatacccccagctttctcctagggctcacctaatttccagctcctgggtctctggcatattcccctggatcatgggacacaagtcaccttaacatataagtgaatcctaattttctcatacaagtaatacaaagtatttgaccttttctgattcttccggacttccctgagagtagaaactgttggaatcaaaatatattctcatttctgccacattttcttgaattcaatattaatcttccaacatccatcaacccactaatcaattcaattatccacccactttattcatctatccatctatccacttacctaccaaccagttcattcatttacccacctacttttatccatccgtccatccatccatccatccagccattcatccattcatccttccttccttccatccatccatccatctacccaacaaccaattcattcaattatccacctactgttttatctatccatccacgtacccatccatctacccatccatctacctacccaataaccaatccattcagttatccacctactcttttatctgcccatccactggcccatccgtctatccattcatctacctagcaaccaaggccttgtctccatccttacacttggcagacatttagttatgtggccccactcatctcagcttgggtctatgtaacctgaaaatccttctttcctttgagcctgtttcccagcctcccccttcacaccaccacaggtaccttcctctctctccagggagccttctggtcaccctgcccacactgagctctccggctttccattcttggactctacctccctgctaaaaacacactccagttctttagtaagaactgtctattgtaccctgccctcgttcagcctgtttctggttttatttaattgttcattcattcaatgaatcaatggaccatgtgtcaggctctgagccaggcatcagcgatggcgaagtggacaggcagtcacagcccctgcttgcagagggcttgtggcctagctgggggcctggaggggttgggggtgggggagatgacatctttcctaaggtgtcaaaaagacctggagaaatggcagatgggctaagaactgaagatgaggcccccctcccaaatgaactgaagcaccaagtcctagtctcatggctcaaattttgactgttccaatttgcagttgtgtggccttgggtaagttgcttaacctcatgagctcctgcttccttgtctgcccccgaggggcctaaccacagagcttgccttagaagatcagcatgggctgaaatagtcacagagaccactaagacctgtgtctggcctagtgactgccaatcctttcccatgacaccaacaatgacaacaccagcaagaagtcaggcttcctgcaaattcaaccacctgattcacttctcagaaccagaaataagtgagtgtctcttagcagcagaaatgcagctgtgagctgctggggagtgtagggatggcaggaacaccaaacacgtgtccaacctcgaggacaacaggcagatggtggggacaaagcaggagacctgcctttgagcctctgctctgctagctattagtggtgaccttgggcaagccacttcatctctctcagcttcttcctctgtaaaatggagagcataaccctacttcttttaggacttgagcatgcaagggttggaaataatgtagatagagcttctggaagggctcagcttggaatgtggaagcccctgttggtgcaaagagctgggcccaaaggaaagaaggaaagggctcgtgagcacagacgcaggggtggagccagagaatgggtctggaagaggaagcatggagctgggtcttgacactgagctcttgcgttcctcgcctccctttctcttggcatttcctcaccacccttccaacatctttacagttggggcttactctttgccaccattggtcccccatccaggccagccttccttggctcagttccaggctcatcaaaagccttattcctccaggatggcccttggcagctggttgggagggaaaggatctcatggggtttccatacacactgaggggtgagtgactcactgcagctgggagtccaaggacaatcctactagctgctgacaaaacctggcttctgcctctgaagcgaaccaagccctgacggtggaatttccagcagtgggtgaaaaaccttcctagtcttcatcagttcacatatatcaaggacactccaaagatggaggtcaaatgaaacccgaagtcacagaaaaaggagagtaagaaaataaaagagataaataataattgtctccaaggtagacaaaagaaagccccaccaggcaccttctgtgtgcaggaggagatggcactctcgctgagtctcccagaaggaactgtggccctttaaggaagctggtacatacaggagcctgaaattactgagcctgaaataagtcttacgtaaatgccaatttcttacggggcttttttgtatgtctttctttctttatctctctctcttttttttaaaggtatgatatataatgggttccttttgcagaatgcagcctctacttgaactctctcttaattaaagctcattgtggattaaagatgtgtggcaattataccttagatgcaaatacatttggcaaagggcagggagctggacaaggaacaattcctcattatgctgcatcccggtattcaccgcttcaggctgggggtggtgggctggacctggagagggtcactgggggctgaggggaacgtaggtctggaatgaacagaagcccgcacactcttctcttgtcaccagctgcctgctcacctggctttctcagcattgtacagtgtcagaccgggagggcacaggaagtctacccatcagatcctttctcctcccagagaaagagccctgaggtcagcagagtggtgcagggctgcgccagggcatcgagcatgaggaagggaaggccgggtggtgctggggcacagggtggcatggacagggtacgggggtggcatatagagtgtgtgcgcagggctgcccatgccacatctttcccagcgtctctgcctgggtgggccctcttctgtcctcttggcacccagccccatcccagccaccgctgagggcttaccaggagcacgagaatgatggggccttggtagatgtagtccaccaggtcgccaggctccttgccaaaccagcacctgtgaagatggggtggctgtagggggcctcctgagctggaactgggggacccccacagacttgggcccagggtcctccagctttacccttcctgagacccaatctccaggactgccccttcccagaaagccttggtggaaataccagctccactgaccacccttacccaccaggctgtcttttgctctacatggtgggtctgatctccccagcccgactgaggacaggccatgggtctctttctcctcctcttttacctgccccattgcagactgctaggcataggactgggcatgcaggaggcacaggaataaagggagagctgggctcccccttgctggcccagggccaattgccttaggccacctgttcccatattgcttgaaacccgctgttccgtgtgccaggcactcactcggccacacaaagcagcagatgtggaagtggagccttcctacctccagcctgggattttgcgtctttgagccaatgcttgtgcacccagtgaggggagctgctgcctggctggcctcactggctgccagaggcataattcattctccggggatgctggtcaggggaaatgctttagggtctgggtcggccagcaactactggaccacagggaggggccatcaactttggggaactcattcctctgaaatcttggaggtcatccagtgcagacctgcctcagataggattctccctgtctcccagggtttctggcctctgcttggagccctcctttgacaggaagctcaccacctaccaaggcagccctcttcttgggcagcttttactcttacaagctcttcctaatgttgagctcctggaactctactccattcttgcccccactctgagaacagatcccctctgctctctccaggctctgagacagaccccttggagatttgcatacaggctaatgttccctggtccccgctgctctcagctggtgacacctgactttctggctgctccttggctgacccatggccagagttgctgtcatttgggctgtggctcagcatattacagcctctggcatagggagaacctcaggatgggacactgcacccaggcagagactgagacttcagaaaaaaaaacaagctctgttaaatgctcatggactctttaaatgctcatggacagctctggatgtcatgtgcggccttggccccttccacataccccgggccagagctgccacatccaagtctgaggaggccttacaaagaccaaaggggtcatgcgttgggtcggggggcacttcaaggaaccacaattcctaacttaattcagcaaagttccttgagccctaccatgtgagtacctctgagtgcaggactatttttttcaatttaaggacaacaggaacgtggatctgtctaggtgtggctacaattcctgctccacggcttggccagagcccaaggctgacctgtcctaacacccccattcccttccccatgaccccccatggctggcccatccacttactgttcattctcatagtagagcttgccgatggcccaggcgacgatgatggggaaggggatgcctgaaagaaggaaagacttgggctgcaggggacagatggacagggactttcttgtaggacataccgtggggtaccacaacagggctaggatcatgtttttactcttccaacagcaggccacccacaaccccaggggtgccctgtccctcaacacctggcccatgcccctgcccctctctccaagcagggctgcgtgattttgtgatagagaagtagagccagccagtttctgagccagagacaaaggcctttggacaggtccttcctggcagggggagaagagctatttgaggaatatcctttggagagatcctttgcttgttcctcaccagcatggagggaagtagctgcatccaccggactgcgctgggggtggagggcagggccaggctctggtccttggacccaagacgaagggaaatggcctggtgagaagtcctccccccaactaggccctgctgcccctgggaccctcacaccatccgatgaagaggaagaggcacttgcgcaggcgctcagtggagtaggtcatgacaatggccgtgtgcaggtagcagccttccacaaacatccagaagaagttggtcaccacgaagtagttgaagatggtggtgatgcagcggcaccagacctgtgtgcagggcagagaggctgtcaggaggcagcttggggcccaggtaggacatacccatccccaggcagggcaacaagacacagggctccccaaagggggttcgtggacatgccatcaaataccagcgaacctcactctgaaaagcttcatccttctccagtgctctttcacactttcagattaagttaaggtgccattctccactggggccaacgtgttttttttaacttctctctaactctttctaatttttcattctagtaaagagagcaagagtctggctctgagctttctgtaggcagaggctggaattcaaccatcttgttgtgttttcattttagtttttttgagtcattttcaatccatagcaagtcagacctgcttccttttggggatgggatatggaatttcatttagaaaaaaatgaaaaaataaaagtgagtaaagtgagtcaagggtgtatgaagtggggctgcggccagggaggggattctccaaagactctgggtttgggaacttctggacttggcacaattattactagctctggagggagacttgcaaagtacacggcccccggcaagtcactgcacctctctgaacctcaaaaagtaaccctgccttccagggtggttgaaggagatagaggatggcaaagacaggcatgagggtagctgtgttgtggctgtggttgtgcctgctgtggttccgtgccttcccagcagaggggaatgtgtccctgtccctctgagcaaggccacccttccccaggcaccaaggctaccttcccaaaggaggcagggaggggaagaccctgcccctttagagccacgcagtgggccatggcagggccagggtctagacattgggcttccaggccagagctcctctcaacagaccccacctggtcatcttccccacaggctcatccccagggcaccctgagagccaaggctgggacatggggaatgatggagccagctcaaggtccggggagctgtgcgtcaggggctctgcttctgcacacagcccatcctctctgtctggctctgacagccccagttctcagataaccctcctgtgctgagctgttggctgcttctgggcttccctgcacagtccctgtggctggctgtctccttttttctgagaaagtcctgctgaggtgggaagctaccaagcccctcctcccaaccctacttttcatccagggttgatgatgttctattagcacaagcccacgttggagctagaaggcaccctcaactcgagtgaacctgtttgattctgaggcagtgttctatgtgggaccatgttaaggatcacaccaggctggtgtctgctcaggcacaggccacccaaaggaaatgtactgagaagtctctgtcggtgtgccacagggctctgtgatggcccaagactagtctacagttttacaatagcttggacacagtacacagcaatggacagaaatccagagtggacagttagcatgtgggatagccccttatgtagaggtatcatcactgcatgtgaccttggcgagtcacttaacctctgtgagtctcagtttccatgtctatgtaatggggaaaatgatccctgctggtctcattaggattaagtgagagaaagctcaacagaggttagttctagcttccttttctcaaaggggtctttgagggcacctgaatccacaagatgaggagtggactaggataaatgtgtctagagtcagctttgtgaagctcccagcctggcagcttcctgctcctcccagcccagctctgttgggacaatggctagggtggaggtgagctcaggtctggttttgcacctgagccacagcccagatgacagcattctggccatgggtcagccaaggagcagccagaaagtcagttgtcaccagctgagagtagcagggactggtgaacatcagtctctgtgtgcaaatctccaaggggtctgtctcagagcctggagagagcagaggggatctgttctcagagtgggggcaagaatggggtggagttgcagaagttcaacattaggaagagcttgtaagagtaaaagctgctcaagaagaagagggctgctttagcaggtagtgagcttcctgacaaaggaggggtccaagcagagaccagacagacagatgggtgcccccggagcccagagccccccaggtatagccccgagtctccccgagcaatgacctcattgctctcgtgcacttcatggtcaacgagctgcagcaggaaccacatgacatttcgcaggataaaggtggtgatgaggttccagtgaatcacattccgcagacagcgaatgctcctgtgggaggtgcaggtcaggggtcagccaggttcaggggtcaactgggactgggttccccctgaggccaggtagagactcagcctgggatgagggcagggctgcactaggagccacttcccacccatggtggccacagttgggcctctgagtccagctcccactctgcaccccacatgcctgctggtgattcatgccctggcaccccacccaaaccccactttctccacgggcccttttatctgctgggccccagaatggaggtgagaatgtctgggagaggtgaagggggtgctgtagggggagggatgaggagaaagcaaggcggaagggcagactcaccgcagggccaggaaaagcaggaaggcggccaccagggctgccacagatacgcagtggcccaggtagttgacgacaagggcgatgcggtagtgcaggtcatacttcctctgctggacagacagacatgggcagggcagatggaggcatgggcacgtgggggtggggctgggtattccagccgtggccacctctgtgtcctgaccttgggggcagaagtgctccaggtgtcattgccgtgcctggctcttagggttcgttcctgttggccctgggtggctcttgttgttataaatggctgtggtcaggccttccaacatgcatttatttttatttttttagagatggggtatcactctgttgcccaagctggggtccagtggctattcacaggtgtgatcacagtgcaccgcagccttgatctgcaggcctgaagcaatcctcccacctcagcctctgaagtagctgggactacagacaagtgccactgtgcctggcaccaacatgcatttttgggggcacattttgagaggtatgggtacagattcttttttaaaattctaggctctagaatgcttctcctgagtttagtctcagcccggatcccagctggctgagtgactgagggtgagtcgatttttatttctggacctcagttttgatgtctatacaatggggccaacctgccatcctacctaagaagcaatggcctatgggaggcctggggtggggtgcattgagattagtctgccctggagaccacacgagggggtgcactgtctatagagaatttagaaattctattcaaactgaataaaagtcagttgacttttaattatcaccaagtgttggcaatttgaaacaaagttagtgatgaaatactccttcctgccagggagagccacccctccaccctacccaacccctgtgttgattctccactgctggaggggcagagagagtggaggtgaggaccaagggctggaggcccccctgcccattgagtggcctccctgcagaacccctgtggctcacattttgcagaatcactttctcagggcgggtgaatgtttttccctctcaatttggacttcatctggggcaaagtcccagccccactgaggactatgctgggtaatgaggaggggatggagctgagatgtatcctttcaggtcaggaatgaggcgtagcctcaaggagaggagttcggtgcctcagcagcactgaattgaattccagagcgagagctggagcagggctccagagaggcagagcaggacccagcttctcatggggacagcccttggggggctgcatttgtttccatgtggagtcacagaatcagaatgccagatggggaaactgaggcccagagggagaggaaggtgtgcagtcacacaacccctaagatgttaggagcattgattacacggttattccctttttgtgtcacatgccaccttggctttgtcctctagcagcccaagctctcttgaaggtaggactccatgtcccccttctctactcccctacagtgtcactaagcacagggcaggacactgggggcaggggcaggaggtacagaagggagtgactgggtgacaaaaggactggtctgccccccttgggatcttctctgctcaacctgagtccaaatacctgtgtgaggcctggggtcacagcaggtgagggccactcaccttgtcatccaaaatgggctcacactgtgagtagttgatctttgaggcccacgtcccattctccaagcattctcgataggcattccctacaaaaaatgccaactgccaagagtcaggtcactcccctcctcaagaaccctccctggctccctggtgcccacaggataaggtgtacgcacctcagctctcctagggcagcaaatacaatgtgcatctgaaagtttacatgtaagtcacttatgtgaaaatagcacaaaaggctcatcttacaaaatagctaaaggcatgctaaaacccattttagacacaatctttgactagcattttgtacactttcattttgttcatttgcttcaaaactgaatccaactgtgaggagtgggttgtgcctgaagattcactgctttcagcatgccacataatttacatccttgtctcaattgttttatatttatgaggtccatacttcaccaatatcagcacttccatttttataatagctaccatttatatagcatatgtttcccatgtaccacttcctcttctaactgctatggatatacatgtcaattcaatctccacaaccatcccatgaaggaggcattaatatgaacccatttcaaagaggaggaaactgaggcataaagagattaagctactggccaaggatacaaagatgacaagaggattcaaactcagaagttgtggcttcgggtcttctgttcttaacctttaggccatatcaagtagtggcaaacaggaatgagtgaatgctggggactcagggctgggccaccgccctcgggctgctgctgcctgggaggctaccacgagatgtttctaaatgttcaagaaccagcctctgtatttaagttggaacatgatttaagtgtgaaaagacatttcaagcagcaatgtcctgtgcacaaaagtgcaggttttgtcaagggggaaggtaggaaagttaaaaaatgctcacatttgcctttctctctatctcagctccaactgtcaaggtctggctcctaagccaccccctccatacaccagctccaatcaggaggaagccctcccacctgagaaggcccggagctctcgaagcctgcctctgcttggccttcatcactagtgtgtttctgacctaccgggtgggttacaaatgctgaacgttccattgtttgggggtgatttgtaccagggttcagctctctgactaatgggcagttgtctgtgaatttttctttctagcatatgttagatgtacaatgtaaagctaattaaaataaatagcttgcagagcacagagttgcagagctggagggggaaccttaggttgtttttggaagcagtgtgttctgaaataattaagttacttaaaaacccacttccgttgagcccgatgagttggaagcaatggaattgggaaggagcacttgccgaagagcaaaatcaatggggaagattctattagcttaattgttttttagtttggtgcctggagctcatccattcttcaaacccagggacgtgactggcctattccttcctcctgggccaaggcccatccctggcagggccctgccatccccctgccaagtgagtcagggaatgccctggtctgatgctgattctgactctcaggaagaggaagcctgctccccacccctagccatggcgcccaactccccaggtgggatctaatttgatacctagcactatctttccttaccaacattcgtgctcatgaaagagaaaggttacctcaactcgtgagggtcagtgatagtgatgtcactgaactaaaaaagcaaaagtatgtaaggagggtaagttcttttgtgaaatgaacagtccctccctgatgggggtttacggtgcctctgaacagtctatgtgaggtgaggcagaccaggatcctgcctgtgcttcaaagggcagaaaaatttatcttctatatgttcatagatattatccagctttcctgaagctcagtgctaggccccttccttcaggaagacctccttgattgcttctactctatagctctctctctcctgagcacctccagtcctgaccgcctgagccccacactccagcccttgccccatgaccagcctggagctgtctaggtgagtgagtctggtcactctgatcaatgtgggggctccctgaggacagggccttggaatacttgtttcccgaatatgatatctcatggtggcactgatcatggggtgggcttgcaggtgggagggggtcaggatagaaatgctgcaaatcagagacctttcctctccctcacaccaatgcccatggggtcccaagttccatggattctgtctcctccttttctttttccagagtcaactcccctcctgcccatccctccatctgctctctagtcttcttgcctgggctgctacaacagcctcctctcctcttgcctccctgcttcttttcttgccaccttgaccatgttgatttctgcttaaagccatcagtggctctttattgtgctcaagaataaagtccaatttcttagcatgatagtcaaggcccttgacacccaggtcccagcctaactgtcctgacccatctccagcattctacatggccactggcattggcacacataggtttggcacataccctctttgtgtggatccaccacttaaagcacctcctccttcttacccactgctcacggatgaactcctacccatctttaaccccacactcaaatgccgcctcctgcatggagccatccccgacacccttaggtctgaagcagtcctttctttgtttcctcctctatcccttcccttctcttgtaacgtagtttttccactttattgcccttgaaccacaataacacatacattttatgttataatccagtacacacacacacacacaaacacagaatccagctgtactattttcctttccagcctattctaatgtcttctattcatttcacacacactgatcatgacccactaaattgactccacagtccactcttgtgtcacaatccatagtttgaaaaatacaaattctgttttagtgcatttgccataattcactatgaacttcatctctttggacctaatccttcttttctttgctactggacttgagctccttggggatagacaagtaagtagaagccatattggagccaccatatctccctcaggacagagccattgaggaaatgtcggctgaacagaattgactcagacctgctgacccctgggaaagcaggtgggatgcagaagcgggaggggacttccctctctggcagcccagccctgcctgcagatgagcttctggttacagacactgggtatcaaaggactggaggatagatgtgccccactcttcaggggagctgtctgctgtggccaatgagggcactgggccctcaggcacagcctcggacaggagggagtaagacagaaagaatctcatccaccccgtgggaaacgtagacggatgggcacacactctgagggctgatggcaaggctagaattgtggggctggatgcagagaggtgggtgcccattaccatagcaacacaggtgcccctgctgaggatcagtgctgatgatgtggagtgtgggcttccaggggcattagggtctgaatgatgcaggcagggtcttctcttgcaggactctgtagtctgggaggcctggtttccatgcccagagtggcccaggcctgccaaaaacccccattacaatgggctctccccgtttcagatgctgacactttgcaaagagttcctggttcaggcagttacctggggtggtcctgaagcctggctgggacatgagaccctatattctagctgtagttttgtctctagatagctaggtgaccatgactaagccccttccctcttggtctcagttttgcctcttagaaatattacagtaaattgattctctatgatgttattggcatctcaggtggatcaattatatgttatgtgagactccccagtctactaaatgccagttagcaccctcaagctattgtgacaactccaagcatcctgacatcaccatcaaatggttggcacttctatctctgataccttctggaaagacatggaccataggagacagggaccatgagggactacttttggtggtagttttgggggaaggagtacaagggacaggggtaggcatggggaaaagagttaatgggactgtggtaaggcacaacaatggggtcattggctatttgcaaagaaaaggacaggaagtgagggagaggctggatatggtggctcatgcctgtaatcccaacattttgggagaccaaggcaggtgaattgcttgagcccaagagtttgaaactagcctgagcaacatgatgaaaccccatctctcaaaaaaaaaaaaaaaaaaaaaaatagccaggcgtggtggtgtgcgcctgtagtctcagctactcagaaggctgaagtgagagaattgcttgagcctgggaggcagaggttgtggtgagctgagattgtgccactgcactccagcctgggtgacagagtgagaccctgtgatgatgatgatgatgatgatgatgatgatgatgatgataatgatgatgatgatagaagaagaaggaggatgaagaggaggaggaggaggagaagaagaagaggtagtactagtagtagtagtggtggtgagggagagaaggtgatgacattgaggtggggagggccagaagcaatatttacagaaggaatgaagtcactacatgggatcggaactttcagcaccgcggacagcacaactccattcttcatccctgcctatctctaaggttggggacctcggagagctcctaagaaccagccttcccacccgatattccgaccttggccatgggcccagcccattgaggtcaagctaattcatgccccttttcaaggcccagctccagccccagctcctccaggtagtttccatgactgttccagctccccaagggtccagctgcttgtgactgcctggctcatgctaggctgggattgatctttcactgacccctataaaggtgttgtactttcctaaccagcccaggttctctggagcagaagtttaccttattttgtacaaggctgagagcctctaaaggcatgtcctgggttgctgtcatctctggccttttcttaagcactgcacagagctgagcacacaggatctcaggatggaccaaagctgatgctgtttcctaggttacttgggaactcattgaaggtaggaggctcagagtgggccaggaggacagtctgccaccttgtatgcccatgcttcaccaagatgcatggcaaatacaaaacagtcacacatacatgcaggaggaagcaaggtctcacacaaagacaccatgggtaggctgacccagctgccaaaactgcagacattgcagtccaaccccacatgggggaggggtgtcagtctcaaacagcaaacctgtgggcagcattctccaggttcctggcatgaaagctttgactgttccaagggaagcaagttggaaactgagttatcaggcatctccttgggaattaggaaggaagacaacttctgcatttggtctggtgggaccagaagagagaaactgacacatctggggtcactcaaccatgaagaggcagaagatctctctcccttgggacacgcttcctcacgggagaccctggagttggtgtacctaggagagacagactctccctgtgaccctgtgtttcagcagatgcagccagttatgccacctcttcagtgtcattaccacttggtgtccagatcctcagaagagaataccttaggggccagatatcccaccctcagttccctttactgcctgtagttgggccactccagatccagccatcttcttgtctggcctggggcctagttgagaaatctttgaccatgactttgagtcacctttcttacctactttttttttttttgagatggggtctcactctgtcacccaggttggggggcagtggtgtgatcttagctcactgcagccttgaactcctatactcaagtgatcctcctgcctcagcctcctgagtaatgggactacaggcatgtgccaccatgccatgatcatttgttatttttttgtttgttttgtagaaacggggtctcactatgtggttttggctggtctcaaactcctggcctcaagtgatcctcctgcctcagcctcccaaaatgttgggattgcaggcatgagccaccatgcctggccatctttcttgctttctgtgggaaaaacctctcaatcaatgtctcctcccagcttggcctccacttcctggagtggattgcttttcatccccagttaagagacttttggggaaggtggacagagctggggtggtcagacgtacagtcctagtactaacagcatggttttcctgagcttcccaaacgaggtgcatctccttgagtcagcatcctgcattgcttggaggccactgcaggttaaggaggactgaccaactttgaggcccacctggcatatttctgtcttgatccgatcacagcaaattggtggtcagaggggaacaggcagctggtgatgttcacccaagctcagacccagactgacgtagaagtgctagattgagtttgggagtctaatattgccaaccccagccactgggtgggaaaggcatccccagggccctttgctggtgctgtctgggattcaggccagggaacttggattccatttcttagtgttatatggacacagctctctgagcctcgactgcctgttctggaccttcagagaccctcaactccctatttccacaggacttttcactcatggtgccctgccatccttaagcaggacctgtaagcacacttacaggagacagtggggaaactgagtcatagaggcactgagcagtttaatggagaccatactgggtctatgaaaaagggaagggggagtggaacccagctcactgtgtaagctctgccctccccagagaggggcagtgggaggaggcccctacctcatgctcccgcctctactgtacagggctccctccttccagccatcctagcaacactcagtgggctcgtcgccatgacgccagcctgtggaggaaagtggggaggggaccaacacaggacccctgtggcagaagctgccttggaactgagaaacatcactagaactcatcaagccctccacccacctggtgcagatgaactgaggtctgaagaggggagaccacctgcccaaagggagaaaagcagtcagtaggatggccgggattagatctggctctcagttcctagttcctatgaagtaatgcagggagaagacagctggctggcaggatgccagcagcatccctccaggggggcaaggggctgcctttctctacaggcttttaggtaccagaccttctcaatctagatagacagaatcctccctcccaggacatccccagaagccacagagttctgggggctctcagagatagcaggagaccaccaccccagaatgaggatagccattcttggtgtgagcaggatttcccctacccaaggacatgatggcccctccttccaggccccaggccaccttcaactcccctccccttgctgacaatgccttagctgtctacagggagccccaagcagcatcatctcccctgtgtgccatggccccacgaggtcagcatgttctctgtccccttcacacagataagaaaactgggacttggacaaggagggcctgccagtccctcagtgagtcatggcaaacccaggacttagatccagccctgctaaatctgagcccaggttcctcccactctcccttgccccagctgctctcctggcaggtgctgtgtgtgaaagggaccgcctgcctgactctgaagcacctggtgagggtgggcagtcagaggggcccaaatgcctgtacctggggcccagccaagaagccctgtggggagctccctgaggatcactgagatggggctcctccttcagcccgtcttcagggctccaggctctgctgtggcactggtggtaaggagtgcacagggaaggatgctgggacctctgacttaaggagcaggtgggaggagaggaaagggccaaggcccaggtccccagccagcccttgattgagatttagatggcacattttgaaaagcagtatccttccagagtattctggtcctgtgccatagggctacggacaagcagccgctgtctctaaagccagcagaatcgaggcccatgccctggtccaacatttgaggcctccatgatctggcttattcttccctccctcctcttccacttccgctcccaccctcccttccatctgacacatcattagctcaatttttcaaggcactgttcaacaacacttcctccatgaagtcttccaaatttactttcccttactcttggaataactcccttccatgtgctctgactgtcttcactgtgctattttacttgggagcctatatcaaaaagttctctttgagagtctgcctgtctgtcactcttcctagaacaggagcccctggaaggcaggctcaggtcttatgcatctttgaaaagcttgtctctaggctcctcaattctttctgggggaaagggtaaaatactcagaaccccaataaggggtgagcctgagcaagacgatcaggtggctggaggattcctggggagagcaggagacaggaaagatcaagatgcatgcagaggtgggtagaagctagagcagaagccaggagttcccagagccagcagaggcctatcagggcccagacttgctgtagaactctgagcagctgtgtttccctctcctggccagtcatttcctacccttaagtggggaggggaaggctggactcggaacatagagcgctctgcagccgggcagctctggggtgtctggatggccacaagggcatccacttctgctctatttctcttccttccttcttttcttagctaaacctctgtgatggccatgcctgtcccaccttccctctctcccagcagggaagttgttctcacacatggagtaacttgtggcccttggagaatggaatagagtcaggggggatcaggtctcgctggagtctgagaatgcagacctgagtttccggatttacagcttctacttcttcaacccagagggcagggtctatctggggtcctcctgaggcttgcacccctgcactgcgcctgtccttaacaaatgtggcatcccaactgctccaagacctttaaagtttacccccactccctccagaaagcctcccaggaatgtcccagtgtccaccaagcccctcttcccgatctctgactgttgatttgcacaagcctccttgataagcagcctggggcttcctgagggcaggtctcagcctttcttatcacctctgactcttagggctgaagaagggattcctgcataagcaggcagacccaatgggagagacccctctggctggagaccactcagcttatgtgtttccattgtaacacaatcagtgcttaagcatgtctctagaatggggttctgggaagtggggacccctaacctccccatgtggctaggttagatgggatgccccttcttcccctgtcctggcagatgcctcagtacagatgaccccagccattcccagtaggacattgcggagcttgaggtcaaggaggctgaggctcagccaagctgcacttagtggttccacagaaggaaaatggaccatggccaggagggaggtggcaagaccctcctttgcagccaaaaggaccgtagggacaggcaagatgaggtgcaggaggagggcagatagaaggagggatgggagtggggagacagtgggtttggagatggagtcgggggatgaagaaggcagtagggagagatggagaaagagagagagaggtagaaaatgagagaatctgagaaagacagaaacatacacccagaaacagaaccacacatagagaaaatcagaaacagacagggagacaatgagagagacacagagacagagatgtacacacagagatgggctcagaggagtccgtgtggaatggggagaggtgggaggaaaatggaatataagtgccccacttctggccaaaccacttccatgctaatccacttcctttcggcctacagacagggagacaggcccacaaaagggatgagacttgccccaattaaattgtatatggacatttaggattgtttctagccacccaggatttgaacctgggttcggagaatcctggggtaagaccgaggctacctccccgcctagagctaaaatgccagatccttacttcccaggatcccttgtagccagagctttggcatgggatttggggctccacatattcccccacccatcagatgcacctacccgagggaattagtgaactggaggcccccagatggagacagggagaaagcctctccagagataactgcaggaagcttaagactccaggttggccaggtgcagtggctcacgcctatactcccagcactttgggagccagagacaggaggatcgcttgagtctgggagttcaagaccagctctggcaaaatggcaagatcccaactctacaaaaaatttaaaaattagccaggtgtggtggcatgtgcctgcagtctcagctactcgggaggctgaggcaggaggattgcttgggcacaggagtttgagattgcagtgatctttgatcatgccactagtggtggccagggctggtggtgttggcagggcctttcagggagagggaggtcccctcacagggccagctctgccctgtgttcttggcttggggccccaaatctggttctctagcccactcagtgattccataagctccccaatatctttttaaaaaatttcttctctgcttaacctagcagagttgctttttgaaaggcagcagaacctgggtttgaatccttgttctgttacaagtgacttcattgctccacacctcagtttccccatgtgtaaaatgaggataatgccatgtctctgtcactcgatggtgcaaggattaaatgagttaaaccacagtacaaacatgtggaagctcagccactgaagcgccagcacaggttgtgtagagaacacccaaggagactcgtgtgcttaacttggctctgccactgactaacatgtgtggccatgcgctagtcccttcccttcccttggccctgctgcatctggaaataactctgggtaagatggctcaaggctctgacccagcctcccaaactcacacactgtagctatttgctaaccccacatcctgaggacttctaaacgccttatctccactcttggtgctctcttgagcttttcccccacccaaccagctgcttcctgaacatctccactcagctgtccctccagctcctcaaagtcaacacatccccaactgtgctcagctgtttcctgtgtgcccaacctttaccttgccatctctcaccatatcttgtcaatttcaaatgcccaaggttgctggcgtcttcaacctcatttgctcttacagcctgcggtctacctgggtctgtgcagaaatccccttacttctcctttccctccagaggacagtgtgctcccaaaattgagcttctctgctcacaagctcacccaggctccctctccagatggtgaagccctgtagaatgccctgcatcacctgacctccatgcccctccccagcctcattgtttctttctcctactccctgttctccatacaacacactcctcccaaggcacaaagcccagcctgtctccttgcctttgctcagtcccctcagcctagagggcctctcctgtgcctattacactcacctgctaaaatccaacctgtccccagaagtccagctcagagggctaagtcctccctgatcctccaggccggaggtatgagtcttctttccaaacctctaagctctcctcgcaaaccgaatgcctcttgtggagggagtactgccccatggttaatagcgagggctgtggattcacctgcctgcactggtgcataggagctgattaggactttcaataagttacttcatgtgtctgagactcagtgttcttgcctgcaatatgggcataaaagcagtatgtatctcagagggagtgtgggcgagtgggattatggatgcctgagatatggatacaaagctctctcagtggtagctggcacctggaaaatgatcaacacttagctttgtggcagattctctgtgctcagctgagttgaaaaatcgcagagactaatatctaaactgctatccccacccgggcgattcctgctctctaaggaatgaggcttcaatgcgggtttggctatagcataacaaaattggggcaggaagtggagcctgaacgctcctgttcttcccctaggcttctcgtggaggtttctccctctgtcattctttttaaaggaaggatcccaaggaaaaggcaaacagagaagcaggaagcagcactgatgtgaagaaaggggaggagggaaatcaatcatgcatttccacagccattagcagcctctgctcttcccacctaggctgacctcacaccccaggtgctggttagtggtaagtgctccccaccctccacaagctcctctcattctccagcagttgcagtgcaggcagcactttggtctaaatgaagaaaattgttcattagataccaggggctaattggccctacttttaccagcctgggtggtgccccaaccttttatcagcttctcattagacttaattgatatgaatggcctgatatgtgctgtctgcagcacctttgggaagtcttccttgtcaccccgcccccacaccccaatcccatagaccttgtgcttcccccctaattagggcattcatcattctgttctttttgtctgttgcaattgtgagtgagagcctcaatacagtgcctgtgcagtggctggcacatagtgggtattccacaaattgactgagtattactgtactcagcttggctatgaagaagagacccaggccctgggaagggggggcccccaggctggtcggggagaacaaggcatagcccaggctggggtaagaacgaaagcagagctccctgaggttcacaggctgtgggctgagatgctccatcaccccactttaccctggatggcccttaatctccgggcccagtctgaatcaggcatgctgatctccattgccccaccccacacagctcacggaatcctaaaaatacagttttataaggtcaggactaggaaggcacagggaaatcattccttctgcttgcacacctcctagcttggggatcttacttccttctagggagatctgttttaatcatggatgggaagcatgtgctccctgggcccaccagagccctcagctggtgcagccaggagagtgcggcttggcatcaggtgcaggtacatctctcttgccttctaaccatctctcatggcaaagccctcagcctcagcttctgaaagcttgtgtgtgtgtgtgtgtgtgtgtgtgtgtgtgtgtgtatgtgtggtggaggtggagctgggctaccaatcagacacagacactctgaggatgtttccaggatgagaggcttcagacaaacaatccagatagggtctcagcgtgcttgacaactggggtccggctggagatatcaggggggttctaggtggggcagtgattgctgcccccacagcctcccgcagaccacacacctggctcccatcccatgttccacagggcacagacaggcacctacccacacgcacacacccagaacccacactgaacgaccccacctggaggctgtgtaactccagcaacctctactgcccttttccccagagcctcaagccagtccatgatttgccaggaggaaggcaggcacaggttgttctccttgagagatggtgttggggtgctgattcagacaccactcattatgggggcgcaggaataatgctgcccacagcccctgactcagcccagcaggtcagtcctgggacctcggattagggaagatgcaacaccccgcaccctctggcttagcagaacctacgcctgcatgtcctccccacgaaacatgcgctcgccccagccctcctgcctcctaccctcctcctcccctctggtagtagggtctggtcactgcactcactctgggggatttgggacttagcatatccgccctcctgggataggtccccctcctgggcaggggaagcattgtctaaacaatgaagccctaatggggaacttcaggcaacgggaacctacaatgagggaaggaggtttacactccaagaggaggaagtatttccttctcccctttttgggctgtcaccgcatggagcacgggaattgtgggcaaatcactggcttactgagattagacgctgtattgggggtagaagcagaaggcgcgcccccaacactgtaaggggtccttaacgcccgcggtccgcggtaccgcggccgtcagcagctttgtaccgctgggtccggaatcctctttactccctaaaccgccttctcaggaggccggtgtagagcaggcagcgagggccgggggcactcacgggtcgtgttgtacttgacgccgttgaagtactcggggcacggcctctccacgagggctccggcagcgctgcggggccagcacgttccgatctggtccaaggtcgtgttgcagtaggagtagggacctggcggcgggagagagcgcagtagggctcagaggggcccgcagggacgcggggctctcggagcgcggggtcaggggcgcacccagcgcgcgagagaaggagcccgcgcagcctccgaccgctcgcctcccgcctaccctcggggtccaggggtggcccccagccgtccaagagcagctcttcagccagcgccaggctgcagttggcctccagcaggctgtggagcagtgccgcgtccatcgcgtcccgcagccgcgtgcggagagggagtgggagtgcgcgcccggcgtgactgcgagggagtggacgcgagagtgagcggccgagagggcgcggggtcctggcccccgccagcccagccccgatctcccgggcagcctttgggcgccacctccggtcgcccagagctgtcaagtggggaccttcccggagaggagccgccgagtgcacggagctgcgggtacagccgctccgccgcggccaatggctgcgccggggggcggggccgggcggctcctctcggaggggctcagtctccagcccccgggccctccaccctgcccaaagtacggcttctcagttcgcagctctcttccactcgcggcgtccagaggagggcggtgggctggagagcgtgggcctggggtgacggaatgctctgtgcggggatcgcaggcccccgagctgcagggggcagcaaagcgcgcccacctgcccgcctgcccgaggagaacaccgcagctctgctaatcgagggacagccgcaacccaaagttcggcagctttccgcctgagcttcccctccctgagcgaggacactggagggaggcagagacggagagcccatgggcagatctgcatcctccaaaagcctccatagcctctgggaaggaaatgtatcgaggaggcctggagggaagcagggagacacccatgagtctaatgagatcaagtggcctgagtgggttgaagtccttggatcagaccgtggaagctggaatggagagagatttcggaggggcagccagacctggtgaggaggaactggagtgggtggctggttttaagtaggagcccagaggcagggataacatcaggtctcctgcttgagtgactgggtggccatttaatcagatggagtaagtctaggggagaaattggctgaaattatctggaaggctttatacacccagttgttagtgccttgggcaccagaaagaataaaggaagaagtgagaatgtgtctctcttcctcaaggcttgggcagggcttcacctgaccctggtttccacaccagtagcggggcaggggcggggagaatcaggccccacagcaggtgtggaggagctggaaacctccccagagaacttgcccaggccccccaccccaccctggcccagaatgcctccttggtgatctgctgtggctgcaccctcagagtgcccagggcgggcctgctgtgtctgccagctgagtggggcattgggctcgggaagcagggcctggggtaaggcatgcttccactcaggctgcattttggtccagcctctgactctgctctccctgccatggtaccccaggcaggatgcttgccctctcttctggagctttctccatcagtgaaatcttccccgaggcccctctcagctctgagactctctggttctgagttatgagacggagagtctggggaagacatacatgtgtgttgtgtgttgtttgtttctatctttagagggcaaggagagctggaacctagtctcagaaaccagtcctgttcccctgccatcctccacataaacctaagctgctaggaaaggctgaaaccaccatgaactagcaccatgccctgggcagagagaggcaaagcagcaggcaggctgcttttgtgtgctccgctctcaccagcctccatattaatggtgctgtcactgcccaggcagagcgagtgagaccacactaagaccagggctgagccctggaatctctccagggcctgctgactggcaggaacaagatgctgagcagccagccaggtctaccctctgcctcctgaatggacagtgagagcccaggctcagctctgggcagctgcagaggtagaggttccttggtcttcatattatccagtgccaggagcaggggaggacctaggcagagtcctaccctccgctcctagactgagccctgttacccagggtcccacacaggccagagggtggctggccaggtcagcccatgtattcacagggcaatagtgtccctcatatacaagggatagcccttcacacagcacggtgcagcacagaggaagggagaaatcctaagccaagcttaagttattatccttgttcattactgcagccaccacctaggtggtgcctaaagcacctatagatgcagctatgtcaagaggtggtgtgctcccaatgaccagaggccagggacttctcatctcatgcagatttctgcagaacagagggttggcctgggtgaactggattgctcttaactgggagagctcacataccaaagattcttctgggaagtgaccatttcagtggcagagtcaaaggctgttctgcatcctgaatgagcagttggggtctgagcacatacccacagacccacagacccgaggtcccctggatgtggggccatttcttcatggatcttattattataggcacagttgtcattcgagatgtgacagagggaaaactagaaaaggtagcagtttgggaacaaattgattttacagctcactttagtgtctgcaaacaggcaaatgaggaagaaattgggaagagccccaaaattccctcaattttactaaatccaagtacaaacaaacaaagacaggggcatttttgctaactaaagaagcagaggtagattagaggctttgggatagtgatgggttcccagcgctgcaggccagccccatcccagctggaggccaggaattaggggataagtatttggaacagtttgtgtgtccccaagctgttggggacaggttggcaaataggttcagggaagtgtgacaggtactttggaagacccctgtgtaccatgagcagcagagtaaggcaggtgcctctggtgccctgcatcccatcccatggcccacctctgatgtcagctgcagcagcagtggacagttccaggccagctcagacacacttgcaaagcctaatacatggcatcaaacaggcatgtaggcacaacagaatcacacacagagccacagttatgcatcttcaccatgcacacacattctctctctaattcatcccctaccactcaaatgtcagctggaaaacaggatttaaagggacaggatgcatctttgcttctctaagggactgtccttggttagactacatagagagggagtctattcaggcacagctggaatagtcgtggtgtttcctgggaattacgacggggttgctgagggcacagaggttccagaggggcctgaaggtgctgcacttttgccctgtagcactggagacaggggtcccaggccaggcaccccctcctcaccctatcctactccactgcaggacagaggaattggcctggtgtcttcatgtcaaaccatgggactaagctgtggggacagaaaggactctcagtgacatccaatcccacattcttgttttacagatggacaataggattcccagacaagcaaaagaggtttccctcccttacttctcccaggccatcctcatctctgcagcagcccaaatgggctccctgtctcctcatggcagataaaacgatgttttataattacgatcctgtcactcttcctcataggagtgccgccaacccattgcccttgagatggagaccacctctttggtattgcaaagaaggtccttcgtaatcatctatccctcatcctcacccttctgctccatttgaaaggccctccagaacaacatgtcattcctgggaaccatctttggtattgcaaagaaggttcttcatgatcatcgtcccccatcctcaccctcctgcttcatttgaaaggccctccagaacaatgtgtcactaccagaaaccattcgctatctctgttccttgtttgtatttctgcactggaaatggccttacagctccacactcatccttcagacatgcagctccttggtgaccccatctgctgggtgctggcctatgtcacatccaggttgctttttattcattgtctcatctaccagcccatgcctccccaagggcagggctggtcctggttcactaacccatccccagcacccattggaatgctctagacttcagcccagtgagtcactaagaaaggacagataccttggcagaagagctgaggaaagcccaggtccctgtcttcaggccaaggctctcttcccatttgaccaaccctggcatatgtgtgtgtgttgcattgaggaattatttcaaggtatctttgaatccattcggattcttggccaggtgcagtggctcacacccataatctcagcacttggggaagccaaggtaggcagattgcttgagcacaggaattcaagaccagcctacaaaaaataataaaaaataaataaatagtccatttggatttttatatcatctatggataggataaataatatgcctcataatatatgccctaatatttttcaaatatatgtagctgcctctgtgatgaataaaatagaaataatctatttaaaccctgcctgaagtcataattgctctgtgattttgcatgcgttgcctttttcaggggcatacaattgaattgatcatagattcgtgaccctttgtcattattagtctgtgcattagcccatgcatggcttaactttattttttagtctttattttatttagtctttttattgtatgtagtagacaatataataagcctaccacccaattggaaactacatcattaccagtcacttatctatttatttctattctccttccccagagggaacattcccttgactattgcatttgtcattctctggctcatattttttttcagttttctcacaggtatgtacatctaaaagatatagtgtttggttttacttggttttgaactctgcgttagacatgatgcatttccccaagcagtggacactgaaaagaaggtcattatcaatttggggacttgagaaatgattcctgaagctcgaggggctccctagcagcctctacatccaccatccggtcatggttcatgtggggtgatgcatccccagagtggctgctaggcagcgtcacaagcgatgcggtggttgaatatttaatgacatggggaaatgttcactctgagttataagaggaaaaagcaggcgacgaaactatggacacactattatcccattaaaaaaaaaagaaaagaaaaagaaaataaaaaaacaattgaacatgaggctctatgaatgcataaattcttgagggatatgaggcattataaaaattcctgggttgtgggagaatgagtacttatcatcttctcctttgagttaaatttttttgtgcccaattttatagaaatcatgtggatcccttttgcaaatggatgaatgctgttagaagctgaacaggcaaggctgtatgtttggagaagctgggaccctatccgctgcactcagagcggggaccatccgccaagggagacagggaagggtctgtgccacctgctggagggagggcagaggaaggcagggagaaggctatgggtctgctgacaaacccacgctgcctctgagggtgagggaaggttgggctttcctgaagggaggggcctccatttcctgtctgatgctggcatggcctgtgctaggtgtccccgtgggctctcattcagccttcactgtgagcctccgaggtggacttagatccattgctaaacagatgaggaaactagcttcagatggtgtcacaggctgaactgtgttgctccaaaatttgtatgttgaagtcctaactcccagtacctcagaatgtggccttgtttggatttagggtcttcgatgaggtgattaaagtaaagtaaggtaagcgggctttaatccaataggactggtgttcttagaagaagaagagattgggacacagacatgcagaggagagaccatatgaagacatagggagaaagatggccatctgcaagccaagtagggaggcctcaggaggaaccaaccctgcccacagcttgatcttggcctaccagcctccagaatggtgatgaaatcagcttctgctcaagcccccagtctgtggtgctttgttccagtggccccaggacactaatacagatgggttaggtgagtttccaggggctgggcgaggccgctgggtggaagagctaggatccagacttggatctacctgatcccaacaccggagctattgactgccccagtggaaggagaacaggcagcaagactttctctttgacgcctggcttgggcagtgcctgtaggctgggtctgggtgctggccagcacccttgtctccttgtgccctggcagtggccccaggcccgagcaagggtcacccccactcccatcctgtggatgcagacctgggaaggccaggatacagggtgggacactggagacatctctctggaacccagcagaatccagggctctgtgcagctctgcagtggctggccctgaccctgtgaatcagccactggaaacctctgaggggccagaactcagggccgggctgctgctttgcaagtccttaccaggaagtcacacctgtagccacggcaaccagagaatgtttaccaccttctgccagtttccctggagttggaaagctgaggggtcatccccttcttggactcctgaatgccagcccagctgacttccatcgtgggagctgggaatggggagtggcctcccagccaagcccttcattatcagtggcaagcctgagcctagaatggggaagggcctgcgtaagggccctcagcaaggcagaaatcaaaccaggactggaaaccagcaccccacccaccaccctgctgagcctgggctggccccaggaaagcctcttcctcccgcagggcccacccacatctccaggggctgctgcagggtggtggcagagggtacctacctgagaggttggtgagggtcatgatggtgtggcagtactgctccagaagtgcccacaggggctggtctttgggcatctggctctgcccggctgcctaggggacagcaaggatgagggtcaaagatcaggcccaggctagccattgacagtgcctgtctgcccccatcctatcctatcactcatcccttactcctcttagtacagagagggagaacaaggagggagatgcccatggccacatggagcagcagcagtctggcctgggctggaccgaagaaaggcctcagaagtggcccaatgcagtgttgcctcctaggcaggggtctgctctgtccatccctcagcccccaaggctgtctcctcctcaggctgggatgtgtcactgtcatggggcttgtgtccttctgtcagcaaagctgggtggggggtgggttccattagttgttcattcaaccaacaaatatttgcagaacacctactgtgtgtcaggccctggctgggggaactgatgggtgtccttcaggtagtcccaggccaggtagggatcctctagacttagaaagtgcagaaggaagaggaggcaaatgcatgatttaactttgtttccctgtcacacaggtaagcaggcacttgcaacgaagagcattaggttaatacgtgagagaagtgcctaacctggattcaggaagacagggaaggcttattgggagaggtagcattttggggcacccagaaagatgagtagggacaaaagaagggaccatagggccagcttaggcactcactagggaagatatgtgattgttgcaataatggcaattccatccaaaagatagaaaagcacccaggatcccgtctcctaaaaaatcatcagagtgggtgctggatgtgcacggataaagtagatgtgggcatgtgcacccgtgaagggaccatcttctggtgggaaagatggataggatgtacagttttacggcatgtacgatttactgaggagggcaggggtagcatcacagaagaggtcatgctaatgtgggttttgaagggtgagtaggagcttgccaagctgatcaggccaggaagggcattctagttagagagcacagcatgtgcaaaggcagagaggctggaaaatgcatggtgtatgactgaacgagagcaattcactctaaattctaacgcttggccccattcctcccaagaccactccttttcttgctctatggtcagcgtgtgtgtgtgtgagtacatgcacacacgtgtgtgacttttgtgcccctcccaaccctgagattcctgattctgggattccagaagacactgctccatcagtgtgcagccccttctcttcctgctgcctgtcagatgtactcttcctggcagctgggtcaatgtttgccccaggatgtccccaggggccagagcccaaaagccctcaaacatcacgttctccacattgggctcagtccatttcctgtgctgggactggaagctgcatgctcagccctcaccacgtcagcctggagcttctaatgaatgaccccacctgctcagacaggagagtgctgccccctgcacccctcccggaagcctagggtgaggcagacaccacttggaggcactcactgcacctgtgttctgccaaactcccaccccacgaagtgaggtagagagggaaatgcaggggagcaggtgtgtgtgttgtgtgtgtgtctcggcactgtgggggcctcctgagctgtcagccaggagttctggcctcttggcctgcaggacaaacactgggggccatggtcttgctgtccacatatcaagggagctgctgagagttcctcagctccagcttctcgggcatcaggaagaccttgctgaccccgtgagccactgccaggggttggggggctggggaggggagggaacactccagacactgagaccttccaggaagaattctcaggccatacttttggctgggtctggggacagtgagggctctaggtctgacccatcaccctctttatggcgcccatcgcccttgtttgagaagcagctagtaatgccctctgtgggagggttcccagtgaatcccagcctctggaggtcagggtacgggcaacccagaggcatgcagtgtccacggtgcgcccttaggcagatgcagccattccatctcagatgttagtgccttctctcaaagcccctgagctgtgacatttgtagccccatcactgaggtgatctcctgtttgactttaccggggtttctgacacatgctttgaacccatgatggggtcagggcatcctcccccaggactcaggcaatgtgggatggtccttgccttggattctgtggtctcaggctgtggccatcttctggctgagcaagcactttgtggggagtgggcacagggttaccatactctgcaactgatgcttgctctttgggtctgggcaaaatttggtacaggcatatcagaagtaatttttggagtaatcccagatcctggcttcatctcctataacttctctgctggccccaaaagctgacacagccaaagatactcattctcttcaaggggcttgtttgggtggatttgactcagtccgaagtcgtaatctaccatgcaggtgtcaattttgggcagagcccctagtggtggtggtgatgggtgggcagggaggaggaggaaagagaggctgctgtcctcagaaaacacatggtccaggaaagtcagggacagacatatcttggataatcccattccaaggacttgagaagagctgtcagagtgggtagagtgcacacatcccagtgtgcagagttctggggatgctgggcttagctgtgtggccttggcaagccacttaagttctctgagccttgatctcctcatctttaaaatggggataatacaaatcccacaggattttgtgtgcattcaacaaattactgtaggtcaagagcctggcacgtagaatgcgcacgctgaatggtgactcttggcaaggactttaggagggggctagttagagagctcagtgatgggaggtcgagtcccctcatcgggactcccaaggcccagctcaagctcttcctgcccagaaaagcttcctggaaaccctgtgccccccttccccagctctccagtcagtgtgggactccgtttgccctcagcagctttccctcggacaatgggcccctgaaggtgcagcctccatctccctcactgagctcgcccctcagcggtgctcagtgaatgctcattgaggaaaccgaaggccgttcggcgaactggacattggagcggaatctaaaaggatggaaggacttgggccataggaacagcagtaagaagaggagaaagtgacccaggtagaagctgagaaagcaaaccctcagaggcaggcgagcccagggcccacacaggaaactgcaccatctggtatggaggtgccaagagaagaagaggggagacaggcaggaaggggaggcttctttcacgtgaagtcagcacttactctagtgctagacctgcctctaaatgccctacagaagacagcggattgaatcctcacggcagtctctgaggtagctgcaaataaccccatttcacagatgaggctgctgaggcccagggaagcgaagtcacccaccaaaggtcccacttcaggtaaggggcagaggcaagattcaagcccagtcagttggtaggttatggcttggtcaggttcccatgggaagagctgatcctgggtgaggaagtgaagacctgtgggtcacggatgctgagagggccccagccccagactgggtggctgtggggctcaggaggccgctcaggtgagaagcccctctcttgccacagaggaacaggggccttggaggcagacacctgggtcacgggaggcagacccagccagtgggcaggaccttgcacaagagagatgaggaattataaatattaggtcttagcccccagttcctgacacagaatccctaaaacctttggaatttcctgggtgacaggagtgtcgttagttctaatgaggtgactcttggtgggctgccaaatgcagctggttaccagagagaccaagccatggttagaaggttggagctttcagcaccttcctacaccttggggaagggagacgggctggagattgggttaatatgcctctgtaatggagcctccgtgaaacccctcaaaggtgggattgggggcttctggttggtgaccacctggtggtgcggggagggtggcatccagtgagggcacggtgctctgcgccctccccacagcttggcctgtgtgcctctccatctggctgttcagctatatcctttgtgatagactttaaaataaacctgtaaatagaaatgtttccttgagttttgcaaaatgttatagcacactattgaacccaaagagagggtcatgggaactcttgatttatatagcctgtcggttgggagaatgagaagccctgtcttgcgactggcatctgaattgggggcagtcttgtgggactgaggctttacctgtggaggctgatgccaactccaggaagatagcatcagaattgaattgaattggaggacacccagttggggtccccagagaagtggagaattgcttggggcagaaaatccacacttttggtgtcagaggtgtgtaagagtgtagagaagaaatggaatgttttcctgtttccatttttctttcttctgaggggaacgtcttggacacctgtgctgattagctcttggctgttctagggcagagagctctccaccatgcacaccccagcccatgagactgactgtaggctgtgggagcacagggtccacatctgtgtccccagtccccagttcagagcctggcacacagcaggtgctcaataaatgtttgtcaactgactgactgcttggatggattaatgaaaccatggccccctcagccaggtagagctacatccccacccctaatcccgggagaccaggttgtgggcttttcctgcttgttatatttccttctctctgaactttgctctgagcctagcctgtgtgagggcccaggtcctggttcccgcctctcagtgcctggcatgggaggccctcaccttagaagaggcaaaggagaggccctggctggggaaggatgcaggaatgagagccaagactccagctgctcttcttccaccttgggtcctatccaaacagaacctgtgcccggggccccgacaggcctcaaaggtatcgcctccctggaacctgggggtctggcccagtctagtgttccctccagggcctggcgacaaagcttctgtctaaatttagcccatggtataagctggggagatttacaggaccatgtgtcctgggcccaggctgccagggaccggaggggaatggggtgagctccctccctgctttctccctctgcttcccaaggtcagagaaggactcagctgggaggggcagcattccaccccatgtttgctttcattcagtggatttcattgagcctgagctcaggccctgccccttagggagcacagagttagaggaaggagcaagaggagagggaagagaaggagaaggcttggaggagaagactggaaaagaggggaggaggagtgcagaggagtaggaaaagggaagggaagaaaggaggaaggaggagcagggaggagaggggatgatgaggagagaagaaggggtcagaaaagggagagggggagaatggggaggagagaaggaaggaaggggaggaagaggagtatgggggaaggaggtggaggagactgggaagagaaaggggaggaggtgggggaggaaggaaggatacaggaacggggaggagggggaagaggggaggaaggtggacatgaggaaggagggggaggacgaggcagtcaggccctgcacagacatagggggcagaaatgggagctgagactggccgaggggcgggcgaggggtggggtgtcagatgagggaagtgggtcattgttttctggtgtgttcaggcaggctccttggaagaggagactttagatactcagttctaacttgtagggtgttggatgttgagtgagaatccccttggcctcctgggcagctcccagcggctttcttgttcctgagtctgtgctcggccaagttcgtcttcctaggatgccccactcttccttcccagttcccctccagctcccccgggaggctttaggcctctcctcattctgctgttctctcttctctctgaacgcccatgaatccctctcccctcagtcctgcttgtctcacctgtgccgagccctgggccctgaacactgactggagtaggagggggaaggggaggggagggagaggtgacttggcccaacataggggcaggaactggccagcaggaggtcccgtagcaggccacttggcctaaagctatctccttaattatcaacacatctaatgtccagtttgcataaaagtcacatctcttctgatttgttttggagtctgtccgaatgtcctaaaaataacttgacattaaaattaaataacattaaaattaaaattttgttaaaggaaaggaatctggagtcaggctgtgggccggaatcctggtcctgccacataccagctgagcggccctgggcacgtgagctcacctcacttcctcatctgcaaaatggggagaggatggttttaactcagggcgttggtatgaaggtaaaaataggtgaggtgcatcagtgcccatgataggccctgtagggctgctaagtaaaataaaataagcttattgaataatctgtcccccatgggtttgcaatgactaaaaattagaatgttgagaaagacaaacatcgcatgttatcacttacttgtgggatttaaaattcaaaacaatcgcatggagatagagagtggaagaatggttaccggaggctgggaagggtagtaggggatgggctaacaggtaccaaaaaaaaatagaatgaatacgacgtagtatttcatagtacaacagggtgactgtagtcaataatttaattgtacatttaaaaataactaaaagtataattagattgtctgtaacacaaaggataaattcttgaggggatggataccccattttacatgatgtgattttctacacattgcatgcctgtatcaaaatatctcatgtatcccacaaatatatatacctactatgtacccacaaaaattaaaaataaaaattaaggccaggcgcggtggttcacacctgtaatcccagcactttgggaggccgaggtgggtggatcatgaggtcaggagatcgagaccatcctggccaacatggtgaaacccaatctctactaaaagtacaaaaaaaaattagctgggtatgttggcaggcgcctatagtcctggctactcaggaggctgaggcaggagaatcgcttgaacccaggaggcagaggttgcagtgagccgagattgtgccactgcactccagcctgggcaacagagtgagactccatctcaaaaaaataaaataaaataattaaaattaaaatttaaaaagttagaattttggaagaagtgactgtagataaactagctgtagataaaatggtttaaattaaatgaagtagaatatgacagctaagaagataaagagtagtcccataattcttaaagtgctgcaaatttgagatttaggaggaatacagaattaagtagccattgccctcagacagatttaaaaaaaaaatactgaaacaccaacaaaatggcaaaactagagactaaattagctagtgaggctagaaaacgctcagaagggagttgaatttttgggaaaagtcattcaatgcattagctgctggcaaatggccctggagccccggctctgcagaccctcagtgtgtgtatgttaggggacactgtggactcggtctttggtgtgagccggacaggtcagggagggtccagggactgccagggacttctctgtgttctgttctctttgttgagccttcttggggcagatgggcccaccagaagccaggacctggggcaagaggtcacagaggcaccatcctgagaccctggggggtgtctcgctgtccaaagctccaaggagctggacttagtcagcaggggctgagaggctcctgtgatcttcacagagtgggaggcaagtggcattgctgagcctctttaaacttgtttcctgtctgtaaataagtggtcccaccttcccggtgtttgagtaaggcgcctaacaccacttaacagctgctagattaccacctgcctccggaccagcttcccgaaggcctccacggggaatagggagcaccagggtcaaggctgggtgggttcctccaggctctcagtgtgccctgggccagggcatcctcttcactcaaaagcaccagctggaacccttacaattgcagatcagaccaggcacctcccctgctcagagagctccccatgtcctcagatcccaccaggacccatacagctctgcagtctcccacataatgtcttttcagggttggggccctggggccttacccccaacctgctcccctatcccttcccttggactacccatctccaactctgctatgctgcccacctcccaccaggctcccccatcctgtcaagggccctcacacctacacatgcttcttctgagcccatttactggaacttgtaaacacagactcctttctttcctgccacacccgcttctgggctataaacaccacaagaacaggaaccagatgagcgttcagaccctggatcctccccatgggcagtcctccttaaacactggatgaataactgagaagcctccaaggctgggctctcctgggctgccttccctcccgactgcctgctccaggtctcatcgtctcaggacccgggaaaaagtcccattgccatgaaggccccgtgagaggaaggctgggctttggcgtcagaaaggccgaggtttaagtgctctccagctgcttacatgctgagtggctgtgggtaagtaactggtctctatcccactccgggatccgcaggagagctaggcgcagggcctgggatgacctcaagggacgctgagaccacagtgttgccctgagaatcctagggcacctctggagcagccagcagaagcttcctccgtctaaccagcttgggcggctggcagcatggaccccagagggacgggctgaggggcaacccttgccccaacacttggatctcctggaaggcacctatctggaaaacaagtgggagagggcaagagagctcaaagcccaagatgtcttgggggcagaggggtggatggtgagccccctgtacaaccttcacccatcccaattgcagtctgcaaagtcactcaaaggactcagcaggtctgggggccatctgcctatcggacactgaccacagctcagcatactacctgcctcccacctctctcctccacctggccaggcccccaggaaggcagggaccctcatgtcctcatgctgtatctgtggcggctggcacaaaagcatagctggcattcactgcgtgctcacaccacaccaagcatgatgctgcttgctttacttgtattacctcaatgttcctcacaacaagcttgttcagtaagcaccgttgtgatcctcattttactgatgaggaaactgaggatttgagtaacctgtccaaggtcacacattaagcagcaaagcagggatctggattggttttgtaggcaattatctgttgaattactgttgaatgtattatcctccttatacgtatagggacactaagccccagagaatggtccccaaagctggtctgtggcagagtcaggacagcgacaagcctcttagcttccacaggcactactttccagattgtgaggcctaggacaggtcaccctaccactctgagcctcagtttccccatctgtaaaatggggagaaggctggaggtggaccactcccaccagcctctcccctacttcttcagctctcttttgatggtcgctctgtgtcccttctccccaccttccatcggtggcccctgactctggacaaaggcacggggtgtggctggatgaagctctgagtcttgagaaacccttggagaggctggttctcccagagcccactctgtccccatcgctcatctggtactgggggctgcctgattccaggcactgagaggaaggatggagacaaggcagcaggggcaggaggtggctctctgcgggtccctgggggtaagggtcagaactaggggtgggctggggaaagaagtcaaggatcttgcaggagagatggaggggggtcatgggggtggggagagaatggataggagacagggaatcttggaccctgaggtagtcaagttctggatgctaggatgtcaccccagaaccctctgcttatccacaggggtctcccagtctcatcctggccagcctcacagtttcatcttcccttcccctgccccctcacccaggcacgggatctccaattcatcatcagacacaaggatccccgatccagcccagccaccaggccctgagaccccctcacccaggcacgggatctccaattcatcatcagacacaaggatccccgatccagcccagccaccaggccctgagacccccgcccctagtcatgagagctgggagctccgtgctcctggcgccccacctcgtggactcccactccctgcacttacgtattggagcggcggtgggaggaggcagagcaggcagaggaggaggtgtgggacgtagaggaggcctgggggccctgagggacccctcatgccagtagtggctgggggttagggactggagcctgctgcccagcacggtggtcacaccctggccagccccaccccacccggccctggaggggaggccaaggtaaggccttgggcatgaggaggtattctccacccacttcttccacagggcagtggtgaccccgccccacagcccacagcctccaggttatttatagctggtcccagacagctgctgctacccagccagccaaggggctatgctctccatccagcctgcagagaaggggttaatgccttctatcggctgccagcattgcccggggctgtagctccccaggtgggagagaggaattgagacaggcaggaggggcctaggctgtggtccctgcttcagcacagggttgggctgccctgggtctcagctgaaccagcactgcctaacacctatctgaggagcttttaacaggtgctaatgcctactgggatgactgagatgggagtgtggatgctgggttagatagtactgtatcaatgctacatctgctgattctgatactgtactgtgggtaggtaagagaatgttctcattcttggaaaatacacatttaggattaaaggggcaccatgtttccaacatactttcaaatggttcagaaataaactcagatagatacatagatagaacaagagagcagatggggcaaaatgtaaattgttggtaaatctggaaaaggggtatatggatatgctttggatcattcttccaacttttctgtacgtttgaaattctatcaaaataaaatgctttcaaaaaaaaaaaaaaaaaacccaaccgacagcaaatacagccatggcagggctcacccagagattccaatgaagtagcttgggatggggcccaggtgtctgtctgtatggggcccaggttccatttcaggaaatggttagggatcctgatatcaggtggctgagcagtgctgttcttggcggggacaggttcaacctggggctgggtcagggctcatgaggtgaggtctgttcttccagagcctcagaagcctccttaggagctgttcctgacaggtgggcctggtgcagggggtttaaaagcttaagccacagttgcaccttgcttgttgttaggatctgaggggaaagcatgtgaggaatgtgattagtggggcaccaaactttctcctggagatgcaggcatggtggagagagaccttggaaaactcagtcccgatttaagtaactcagacactctgtgctccttccggtctccctatctaagacatgggacacttttctgtccttgagtgcttcctccccaccctcagccccttcctccaggcagccttcctggctgttccaggtcccacctccctctcctgtgaaatctcacaggtctgagtctgaagctctgcctcgctggtgtggactgttctccaagctctgggaggataaggatgaatgtggatgtgccccaaccttcctctccttgttttttcttctcccttccctcctattttgtacagagcttagtctcagtggtacagggaaatctttataggaaaaatggaggaagaattgatcggattcctcatttatttgaaggtggcagggtgaaaaccctatcccaagtcctggggcaggatttggctttcccggacccagccagatgggcttggtcagagagggctccttgtagaaggtggatacctcctacaaaacatcataagctgggaaggactttgagagctgggcagactctgaaagatggctgggacagttcccttcagcagccttcagctgtaggtctccaggggccagcctaatagcaggcaccaagagcagaggccagcctcagagtgtgcagggacatgggaaggagaggcatggggaacccatgagacctggcatccataactgatgggaggtgggacatggcacaggtgctgggccagctcaggggagtgagagggcacttcctgggaaggtactgcaggagcaccttgaagggtttaagcagatggaggaatgggagacccttactgcaaatggggaagtctcctgagcagaagcagggagcaaggtctcatgcagctgggagcctggctgggtggggcaagctggagggaagccagattctctgcagctttaggagggtccgggtagcagtaggcactgtggtgcttagcccaggttcctctcttgggggggtgttctgggtgcctgtcttagcttgggctgctgtagcagagtaccataccctgggtggccttaacaacagacattgatttctctctattctggaggcggtgatatccaagatcagggtgccagcatggtcaggttctgatgagggccctctcttgggtctgtaggtggcaccttcttgctgtttcctcacatggcagagatgttggactcttgtctcttcctcttcttctaaaggcactaattccatcatggggattctgccctcatgacctcacctaaccctaaaccctaattacctcccaaatagcatcacatcaagggttaaagcttcaacttgggaatctggggggtgggggcacacagattcaatccatgtcagtgccattcccctggctttccttgtgcttctaaataggtcccttttcacaacctagaactttcttagaactctcctggggcaatgagaaccactttttgcacaaagtacccaggaatttatatcctcccaaccccctaagggcatccttagcattgagtgattggtgttcctttgcctggaggtggaaggaactctgaagtgtaatggacattctagaactccttgtggaaccaggcagaggccgggactctgcctgggatcttttccattctccgtcctgccttgcctcgtctcttaccagtcgctcctgggaacattttctttacacatccctgcacaggaatcctcacctttggggaaccccatctaagggagcctaaatggtcaaagaagtctgtctggacttgatccctctaaatggaaggtgttaacaacaaacaacgacaataaaaaaacaaaaaacttgagcctgcagggtttcctctagacagcctttcactttctgaggctgctggtggtagaataaaccttgcctagcaacgtgatctgagcaggttaataacaacctgctcgtttataagctttgtgttactaagggtagattcagtgtggaaaggaggctttatgtttttgaaaatgccttcatgggcctttttggtgatgaaagacaaactcagcatgatctaaaagagaagtgtgtggaaagagacctaactagctttgtaaagcaaagctattgaaatgactagattgagcatggcattccaagatggagacagtgtagaagggctaggagctgggctctagaaaggaggaggaaaaagaagaggagacacggactggcacaggccaggtccaagcatggatgaaccaggagcttccctatggtggtgaattgtcaatgaatgttatgcctcatcttcagaaaagtctacaatacacaacagcctgtgccagcagtgaaggaggaggtgctggggcatgttggatacagggttaggagaggtgagaggttgcaggaagcgggagctgaactggacatttctttgttttgtcccaagatggtactgtccttctgagaaatgctccatcctctactccaacgctgctcagccttttttttcccattattatttacaaggaggctttttagatatttattttcctaattgccttccctcctaccatgaaattttaataccacagacatactgtatatgtgtcatgtacttgcctctttggagggccacagaccattctcatatcagatggttctgcctcctaagaacggattttgttcccttgggagcaaagaacaggcatgctcttttccagccacgtgatcaggctagcagccagccactgtggattggccaaatgctattcaccaagctggacaatgagagtgtctcaccccgctgaccatggttggttggtacaggaatggacagctgatctgaaattacccaatcatagccctcctctgggatttttatttttattttttgagaccgggtcttatgctgtcacctaggctggagtacagtggcatgatcatggctcactatagcctctaccttccaggatcaagtgatcttcacacctcagcctcctgagtagctggaactacaggcatgggccaccatgcccagctaatttttaaaatggtttttgtagacacagagtctcattatgttacctaggctggtctcgaactcctgggctcaagcgatcctcccatcttggcctcccaaagtgctgggattacaggtatgagccatcttgctcagctggaatttttaaactttgttccaaagagagagcctattcctttctcatgaagaaccagagatgccaagtgtagcggcttcattgagagccaagccaagagcatgggcagagaagagaggaggaaagaagggcctggtagcctttaaggcctcaactccagttggacctgaggctagccttgcccctgcctccagtaagggagctttctaataaaccttcctttatgtttaaagtttgtttctgttgctcactacccctggaattctgaccaacacagaagtgttcaggaattgaacaggagtagactttagagtaaaaagtgaccaggaaaatgtaagacaccccacccacctgcctgggagccagcactgttgcagggagcctagaccttctgtaaatctgataggatggggtggctcggtcattgtatgtagttgtgaaaggaaacggtgactttgaagggctggaggacttagggacacatgtgtccccaagcaggggaggggtaggggagggtctgcattttagaaggtttgctcaggcagcagggtagtcttgggttggaggtggcaagtctggagtgaagaagtcagtgtgggcgaatgcagcagcccaggcaggaagggatgagctggtcctctcagtccaaggaggagtctaaggaaagccggcacctgtcgagaaacggacctctg

SEQ ID NO: 5 - DNA, 33654 bp, Homo sapiens
  misc_feature (26)..(26): n is unknown
gggaatgagtctccatttcttctgcnatntgctgaggttgcctcctcggccagagaacataaaagtgttattcagtaactctctccttttcccttggatgtgggaaggaacgggcgcaccacctttcctccctcttccatttccatgctgcctttttgaagttgcaagagatgctaaaagaaggtccaaccccaagtgagagcctggcctttgcctggaggggtaggggcaggtggcctatatcgccagggtcctggaaaggaacctcttacccactcactcccaattcctatccaactcaagtcactccagtaggctgcttttgagatgcaaatacaaaatgctttcttctccccctctactttttcattgaaatgctttgcatctcagaaaagaggaaaaaatgatgtgtttaagacaggagtgtctcacccgccatcaagcctgataaggaattgttaaattttaattagacaatttgattccttcaagtgccgaactttcagggaaggcagcaccaagacttaagctgagacggaagcataggaaacacgttgctggggtttatgctgcatccagggcttctgtcccccacctttctagctctgtgccccagatgaaatagaaatgaggtggtggggatagagggagggagagagagagagagagagagagagagagagagagagagagagaaagagaggatcagagaaaatcctctctgttctcccaggacctgccagctttcttcctggatcccaggctgtcctaacagattgacccttttcaccccactccatcagcctggggactaagtgttgctacaccttccaagtttctcctgagagaaaatgtcaggtttgggtgtcagcctcaaactgctgagccttaaaggagcttttttcccttccaatacactttataggttggggcgnctgccagttttggggcgtgactgggaaagcagncccttccatacctcatctataccccagaaaggactgcccactccctgtcccctcctctactcctggggggcagtgcagtgggtggcctgagcagggtggcctgcctggggcagcctgggtgggctagcttagccccgggtatctgggctcttccagtacttctcaacttgagagctagctaatttccaaattagggcacgcacagaaaatagtagcatttgtaaggcatatagggtgaatagcagaagctgctgaggttggaagtgcctgcctctacatcccaattgacctaatatttctatgcaccttgatgcattctagacctctgattggggaagttccactctgaagcttctgaagagagccactattatagaaggctggagttaggctagatgttaggaagaacttgacatgcaggggtcctgagcactagaatggatgactctgaaagatatttctgtagatctttgcccttctgagctagggcagagcaaagagtcctggagacagatgaggagatctttggctctttgatgaccagtagcttgcaggtcaccacacaaatgcagtcttctctttctcctggcttccaccagggcagctgagaaagaaaagcctaggaggcctcagaggacccatggaaccttgtctgcaagcatctgcatggtgagggtggaatgaggagcagcaattacccccactttcctgcacagaaaggtgccttaggggagaggcagccacccctggagtgaggcctggcagctacaggattagattgttcatcctccacccaggaaacactgtcaagtagctcaacctgtaacagctggaaaataacagtgtccatgcatgtatttggtgctcactaagttcttgttgactggggattgtgatttggagttttgtaagcttttatgcatgcccaggggccacctgggagaaagatggagatgacatttggagggtggtgagcaagttgatcatattttcacaatcaaaaatcaaaatgcttgttttgataacaatagatttttaaatatactgtgccactttatttgtctcaaaaaaatttctcatgtaagaataatgcaccttgggttatatatttagccantctccaytttaaaaaaagaacaagaaattgggaccatcatggcaaatacaaggtgccatgtgttatgtggctttgcattttccctcaacaagctccagagtggacaaagaactcaccaggcttctttggagccagaaccagcccagcgtaactggagcagcactgagaacctttcctcactccgctaccatgaacacaagctccagccaggatggagaagctactcacttgtgtattcttttgggatgccccttggagcccaacaatctgtgcagcccggagacagaaggacaaaagggagggatgggctggagctgcagcagggaagcctggggaccagcaggttttctcaggcctcttgagaaaataccagcttagaaatgctgggaaggccctgtattgctatccactctacaatgcacttcccmctgcccccaccattttaatgtctctgaaatcagaatgcatttkacaatggatgacatcttggagctctactggggttggatggcattwttttctttcctagtgatatatraaatagtggtgcatcgtcattcgattttgtcttagagtctatgaaatgtggtgattagtatgacagtcatcgrctatagcagctgggctgggagagctgtatcttccaggccagctgctacctcatctcaagggtcacttgagtttgtcctacagccagggcccagggacaggctttgtcttttccttccwttacttcctcccaaggagttggtggctgcatcttgaaatccatctcccttcatctagccacctatttttcaggatgggccataggaggcagtgggcccaggaggacctcagaccctagtgagctgcatttgaacagcaacagtcacttcatcttgctgagccgttggcttgtggtctgattgcttgtttcatttcattcttttgcttggtttgatttttaaagttgacattctcaagggcttacctgtgcctggcacatggccatgagtcatcttacttaatactcacaattgttgttatctctattttgtgtatgggaaaactgggctcaggaaagccaaataatctggctgaggccccaaagttagtaaatggtagagwtgggacttgaacccaggttttttagtcctcaactcgaatgttatactagcacctctcaggctagagtaccttctctgtggcagtggatttgctgatgtctgaattcctgctcagcttgatgtttacatggtgagagccagctaggctgcccctgcttgtgagaggtgaaagccccaagtagataaggtgctgaagacacgatagaaaccaggactgacaccaggtgttaaaagatagccctgtactggccgggtgcggtggctcacgcctgtaatcccagcactctgggaggccgaggcaggcagataatgaggtcaggagatcgagaccatcctgactaacacggtgaaaccccgtctctactaaaaatacaaaaaaattagctggacatggtggcgggcacctgtagtcctggctactcgggaggctgaggcaggagaatggcgtgaatccaggaggcggagcttgcagtgagccgagatagccccactgcactccagcccgggtgacagagcaagactccatctcaataacaaaaaaacccccaaacaagcaaacagacaaaaagatagccctgtaccaaggtacttataggggttacaggtatttaggcatttgccatgttttcccttttttttcctatggtgtaaatatactacgtttgtattagcaacaaaaagcaacgagaacaaccaataataaataatagattagaaaatcaaatgtgtgaggcctttctcagaacttgggtacagggactctcccttaccctgcagaagtaaccagcactccagagaatgaatgctcaccctcaaccccacctgacctgcagtgctcacatttgccatctacattgtactcataattttaaatgctagactagtgcctgtaatcccagcactttgggaggccaaggcaggagtatctcttgaggccaggagtgtgagaccagcctgggcaacatagtgagacccccatctctacaaaacatttttaaaaattagccaggcatcgtggcacatgcctgtatttccagctactcaggaggctgaggtggtaggatcacttgaggccaggagttgaggctgcagtgagctatgattgtgccactgcactccagcctgggtggcaaagtgggattctgtctctattaaaaaaaaattaaatcccagtttacatcctccctcctccaggaagttctccacaaacgccccagccacacacatgtgcatatgctcctgaacatcaactgtagtggacacggtggagtgccacccagatccccctgcaggaccacagccctcatccccaactactgggagtgttggcaactgacagtttttggcaattgcccactgctgaggagagatgcctctttcaaggcctcgctgctttccctgggacagcagaagcttgtgacatctatgagaggggattaaaggccctaccctgttgccctcatttaggcaactggcagggtgccctgtggggtcacctgatgcctttcttgtgacttctccctctggcctgtcctgcttcctcacttctttgcacatcttgatcctgagagcattcccctgtacatttcctgcaggctgatttcctcttcagagtctacttctagggacctgtattagtctgctctggctgctatgacaaaattccacagattgggtggattgaacaacagaaatctgtttcctcatagttctggaggctgggacgtcccagatcaaggtgtcaacagggctgattttgcttaaggcctctctccatggctagcagatggccgccttcttgctgcattcccacttggtctctcctcttgtgcgcgcatccctgatgtctctttttatgtccaaagctcctcttataaggactccagtcagattgggttagggcccaccctaagcgtctcattttaacataatcaactctttaaaggccctgtatcctaatacagtcacattctgaggtactgggggttaggacttcaacatatacattttgtgggttgggggaagagcatagtgcagactgtaacaggacccaacatgcagcacctatggcacccttggggtgccttactttttcatcacttgttaatctggtagcctatgttagcaggtgcctttcaagtgggaaggggttttcttcccagttgcactaacagaacctttgattcagttcagcaaacatctgttgaatacgtactgcccacaaaccagagtcaagagttgggaaagaacaagatagtatggtttttatttgttcattccaagaacaagaggaggaatgagcaagacatgttgtaactcttgtgatgtctacaccccagtgaaaaaaagagactcctaaacaggtgtctcctgcagcactggggtgtgtttcagtgctgcaggaccctgaggagggtgggtgatggctcaggcaggaggatatgggtggattctaagacaggaggattctaggcaagactcactttgaaggatgcatagggactggaaaagcagagggaagggaagtgaacagaagagttttccaaatccctttcttattaaagtagggataataattagacaagatcaagcatgttccaggtagtatggccatagacagggataataattaaaagcctccgagggttgttttgaggataaatgagacagtatatccaaaaaacttagcaaactatcaaaccctttacaagtatatgatgctatcatcagcagcagggagtggggggagaagaagagagcgatggtcaggatgggggcagtaagtgtgcctcacacgcctcaggcccctgacactgctttgcacagtgctttatgcccagcaggttcaataaatgccgattgagtaaatgaatgtcagaagcagccccaggcacctgatcaatacatacatgcaggcctcccgctgtgggctccaaatgggaagagggctcagttggttttgcagaatacagcatcaatatgacatatgcttcattaccttagaacaggaagatttgctgcgtggtggcattccatgtcaccccacatggaattaggcactttgcttttccccgaggctccagagtgatggtggttggtaggacatgatccatattcttctactaaagctggaagagtgggtgtgtcagacagggatgagagtgtagagattgggcagaccctcttccaccatcctctctgacccattgctggaggtgtgccctgttcattgattggacatggcaccacattcatcccaaatggcatcaattggtggttttcaatttgcagtagcaaagtacctggaagtcatgtgctttgtatgaaacgccttggaatgctgataagtttaattctattctgtaaaagaggaagacttttgttagctgaaaagccaactatattatccatgcattatgcttcagtcacaaccaaaactccggctgtcaagttcatcaactcctgatgcctcccaagtctgctcctgagcccctctggtcccttttctctgtgaatatgcgacaccagcctcccagagccccacgcagaaatcctagcattaccctcaactccttatctccctgcttgtcccatacccacttttgccccacacctatccaaggaccaaggaccatgaaattgacttgcagtttagctcctaattctgaacaggtctctccatcctcgtttccactatcttcgttcagatgaccacaatttctaacctaaattacagccaaaacatcactctcctctgagctctcttctacaccatacactttctagaacaccagtccaatagggtcatgtctctgggcccaacctctcagggactctccagctgacaagacacaatctggcctgtaaatgtccctgcagtttaattatcctcaacattactgccataccctacatttttggccaaacagaattgtttgctgtttgatgatttgataccatgtcatattgtctgtctggaacatccccccacccctctcatttagcaggctaagtcctccttctacttcatcccagatgatacccacttcaggaagtctttcccttctgtgggatgagatgcccattctgcagggctttcactccctgcatcctgccaaacctcatcatctcttacctggattcctgctacagcctcccagttggtgtcccgcttccactctgggcccctcctctccgttctccacagtgctgtcagaatcacctattcaaaaggcgaatccgatcatgtggttcctgctgcccttaggatcatgtataaactcctagcatgacttttaaggccctctatgatcttgcctattgcaacctccccagactcaacccttgccaggtccctctgcatcagctatccagaatctctttgaggccctccacctgctgtctacctctctacctctgtgctttgcatatactgcttccaatgtctagaccttctgctgactcctagttctttacctggctaattcctacacatccttcagttgtctgtctgttgaacatcacttcctctagaaagccttccctgaatacctgaactaggttatgtatctctctccctgttccatttcctactccctatcacccttagatgtaattgcttgattaattggctgttgtttctcctagaatgtgagctacagaaccatgtatattttgttcctgcctatatttctagaactatatagaacaatgcttaataaatatttatagaatcaagaatgaatgaatacccatttctgtttcgatgactagaaggtagcaagcctggttaactgaagtgtgtggtgcatgggtcgtgcttaagaagtgtttgttgaatgaataaataaatgatggatcagctctgttcaagctcatcttattatgcatttttaaaaaattgtgtttgctcatttgtcctaccagctgtaaaaacttgctagcaaaagagtggcagaaggcccaacatttttagaaaattcttagccgtaaaccttaaaatcccgagttggaaaattctcattattaaatgccaggagttggtcttcttcctggagacctctgcaagaggccctctcactgacaccttgtgtccatttttcctggccagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtaggtgggccccctgcttgctccagcactttctccagcagggccctgcactggacactggggactctagctccccactggcttttaccaatgagtttcctggtgcttccccaggtggtttttccttcactctgggcttactttttctccttacgaaatgggtagattgtttccttaataatcccaactaccatttgtaaagtgcttactaagtgctgggcctcacgtagggactttaaatatagcattttcctatataaccctcacagcagcttagaggctggcattattgccaccattttgcagttgataaaccaggtggtcagagatgttaagtaactgctgcagcatcacacggctggcaagtccaagctggaattctggcctcagagttagttccttaggtcatattggaaataggagtaaccggcaaggatccccaaggaggggcatgtttatgctcccaggcccctgaaaccttcactcccatctccccacttcagaaatgggtctgctgctttatgctccccatatcccctctccttcccacagcttatcctggggccctgtccaggacctgcaggtagaggctgccatggactgtgctgtagcccttttgttaggaagaattgtgtagtcactcatttcttggcccagctctgcacctcaaaatggggagtaggcagcacagtctagacactgtgcctggtgcattgctgacacagacccagtgaatactcttggtaaccctgggagtccatgctactcttctcactttacaaaataggaaacaggccgagcacggtggctcacgcctgtaatcctagcactttgggaggccgaggcaggcagatcacgaggccaggagttcaagaccagcctggccaacatggtgaaaccccatctctattaaaaatgcaacaattagccaggtgtggtggcatatgcctgtaatcccagatacttgggaggctgaggcaggagaattgcttgaatctgggaggcagaggttgcagtgagctgagatcataccattgcactccagcctgggcgacagagcaagactctgtctcaaaaaaaaaataaaattaaattaaaacaaaaacaaaataggaaatagaagctttggagaagttgcctttcttcacccaggtcacagggagaaatgctgatggagaaatcccaaagcagatattattctccaggaagacaccttcagagccagcgagcagatgtgggagcatttagggatcatcggtgggcacaagctggatcctgcccctttctctgcccagctttctaggatggcatgtccaggactcaagcaatctgggagtctaggtgcatgggaaggcatgggatcaggcatttaggaaagtgccggtgctctgcccactgaaccgttaccttccttccctgagcaggagaggaattgaccagtgcctctgaggccatccctgcctgagagggaaggggttgttgaaagaaaatgagaaagctttgtaggtttaaacaggggagaaatctagatgaggacgctcaggtgaggaggcgagactggtggaaagtggcacacctccctgtccctgggcccccaaggggctcgtgcgcttactgttctcaccaccaccccgggcccaagggagctctgatccatcaccccttggcttcctttactacagccaatgcaggctgcaacttccaaaatgacctgactggatattaaggaagaaccaaaaggaaacacaaatacaaaaccacataaaaatattcagttgacacaggccaggagtccaattccccctggatccatgtttatgagggsccctagactctgtccatgccttcttaattcttacgcttgatcccctccttcttccctcttccaaaggagggtcctaggtctcagaccatagggraaaatggtagagcaaacaaactctctgtgcctcagtttcctcatctggaaaatgcagataataatagtatcatacatcatagcattgttgaggatcgaattagttgatgtatgaaaagtgattagaatgatacatggctcacagtgagcactgtgtaaatgtcagccatggcgatgatgataaagatgaagatgacaatagacatccagcactgctccacatagggaggactctgtctttcctattcaccgcccccttgggataaagaggaaggagagagtactccctatgttcgccccaggcaagtaggtaccctcttcccagaggaacgtgtgttctacccagggctctgggcatggcttctgttagcttccagaccccctgtccctacccaggcacttgggtagggacaaatccatacatgggctatagggggttcccaggaatctgtaagtctttcgaacagatcctttaaacatgcttgttcaccttattttaaaggtgaagctgagtgtgtcaggggaggagggtgcagaagccattctcagagggcagggstcagacttccccacagcatctcatcaaactagagctgggcactgagccttgtttaatggacattatctcagcgatttgttgtccctagagaacgtcccaggtgacgtctcaccctgccctgatgtcctatcagcgcaccctcacaaccacgcactgtcccctctctaatacatgctcttgtcatccagattttttcctcctgttccatttccagcaatgccatgccaacatctgtctggcttaggactaagtgtgcgttatttatcttgctaagtgtatgaaaagcacgctggttttggagtgagaggaccaggttcaattcccagctccattcctttccatctagatgctattggaagagttcctgatgtccacatgtgccattccatgtcagcctcacaaccctttaaggagcggcttcctgtgtgcatttattttctagatgaggagattgagaagatgaacaaatatctcagagttattcattcactcagtcagcactgactgagcactccgaggtcttaccccaatccttgtgcaatgggctaaggcactgacctgctggggatgcagaggttgacagggcccctacagtgcagtctgctgccggtgacagtggagcagtccccataggaggtgtggggcataaggcccagcatgagggtgtcagggaagactttcagggagaaatgatgctttcggaaaaattatgcaggagagggagggagacacttgcattctcaagaaagccacccagcagggagagggagtagctgbctggaggtatggaggagaggtggttatgtcatttgcctctgaggcttactgtctgcattctaagatataagcatcaagtgtttggaacagtgcctgacacatggtaagtccttagtattattacagttattaggacttagctgagccagctcagggcctgtactgcaggtctcagctttatgtgagcaagagcattaaggaatgatgcctggatgcctgggggtgtgaagaaaagagccttgggttcgactagggaacctggggccactccttcctctgctactaaatcaccaagtgatcttgttctgttttcttctctgaccctccctagttttgtccacccttgaaataatcatctttccttttcacatttcatgcttaccaagtacttgtcacctaattatctcctctcttgataagctagatggtyccttccagggcagcttagtagagagcatgggatgtgatgtttcagattccagctctgctgcacacctgccaggtgaacttggccacgttacatggcctctctgggcttcagttccctcacctatgagtgggataagcaagcccttcttgtaaaagttttaagaacaatacatgagataaagtgcaatgcccacagtagatgtcttatggacagtggccaccaatgcattttcttgagtctttaagttagcagccctaactactctccaaggcagcttcccctgggaccacagaggaaatctctttgttattctgggctccagataggccagtgcaaggagagatttaagatgtccatgaaggcaggactgtgtctgaattgctcacaactgtgtccaggagcctaggatagaggctggaacacagtaatttgctcaataagtgtttgtaggaaagaagggatggcagaaaggagggaaggagaggaggaagacagggaggaaggccctgatatctcaacctagatccccagtggctcccaagtactggtcccaggaggggcttcaaagtgggatgtaaatcaggtagtgctggttttctgcccagtggtaccctaaaggcacactctgtttatgccaggaaagccctttacgtaccctcctgcctccacccgcaaaatgggcatccagctgttagctcctgccaacctggtcagcgcagcagcacagggagctgggagagaggccctggtacggggcccccatgtgagctcccaggaagacagtgccgcatgagaggcttgctgtgttctggggcaagctgctgtttctgccacacaggatgacagcagctgcattgcccatcattgagcttaaatgccagcatttggttggatgcctaggagcagaagagagggcagtgatcagaatgagcaaggttataattagccctgctgccaagctacacggtgacacgaagtctgccctactccgggttgcgggagggggctttcacgctcctctctgctcctattggtagaagccaaaggtgcatctccctgtcctgactccatcctcagggaactggggctgctccaagcttccagagcgctagggacagctgccgtgagtgtgtgcctgcgtaatctgccatgagtgtgtgcctgtgtgtgaatgggtgttacgtggggaggggggagggcggtgcgttcatgtgtgtgtgtgtgtgtgtgtgtgtgtgtgtgcctgtgtgcatgtgtgtatgagaaacgtgctcctgccaggagctagactcttcatttcccatcctaccaaggacacagctcatcacagcaacccctccgcctaccccagcccactcgggcgttcctcgagcactagcccagatctgccttctttcgttctctgggtataagaaggagcagaaatttcttctctgtttcatggcgagtcttcatcttcaccattattcttaacagatcattttggggccccgactctgccatgtactgggccaaccactgaagggcttgcctggctcagccagagcctgggccagagcaggactaggaactgagcccacaggccttggggaaatcactgcactttttggaggcttggtttcctcattggttctcagtttcctcacgggcattctaaaattttacctggaatctaatttacagaggtatggctgagtgataacacagggaagccagtgccattgggaagagtttaacgtgatacacaatgtccctagaaaccagatgctcagcccgccatgtggggtgcagggggaagcagcaggcttgggtcaagagtcagaatcaggggtgagggaaagcccaggctacagccctgttggggctcccacaggaagtgcaaggcagggcagattaagcaacctagggttggctagtctcaacatcctggcaggctttgggctacaggggtgatctctagttgtctctctggtacctgagataatttgggacaggggaaatattgtcttggtgtgtgtgtgaattagagaaacggggtggctggcttgcatttgagaggcatccttccaagtcagtcattttctgtcttcaggaattaactagctctgggacggtaagtctctctctgggtctgtgaggctcccaagtgccagaatatcaggatcagagaagatagaaaaatatagtcaatataggtgtctctgtgatgaatgggtgccaaatacacaaatacagaatctaagaaaacacatggggttaacaaaggttgcagagattaaggtttaaagttgaggccctccgtataagttggctgtctttcttctagcacagtaattggcaataagtggtcttatgtatctgggagaagataagtgagggscaagggcctcagacacccatgggcatcactccccacaccccagctggatgcccacaagacttgcagctgcctcctgagtctgtggcctcatcggctccaggagtaccaggctacaggacctaagctaatctcccactcctgctgtccatccattatgttgctttgtccccaggtggtaggtgagtggaaattcagcaggattcactgtgacatcttcgtcactctggacgtcatgatgtgcacggcgagcatcctgaacttgtgtgccatcagcatcgacaggtgagcccagccagggtggaagaggttgctctggccactcaccattctggctgccccagcttttctcaaagccaggctgtgtctcgtgtctgtgatgctgtgcagctggctgtgtgcatgggggtccatgtgtttgtgtgtatgggtgtgcatgtgtgtatgtttggctaggagagcacacactccctaaagagaagccatctatggacagtgatgctgccaagcatactcagacagtgactggtacaaaaggggaaagattgtgattctgacggaagccctagctctagcacatcttactcatgtgaccttgggcagatgatggacgttttcagagctctcagtttcattttctagaaacctacattgatgacacctgtggcctggagctgcagtatctggttatgcattttatgtccttggcacatggccaagggtcccaggccatgcacaccttgctaagctgcatccttggagccttccactaatttgcacagatatgaccctacctccctcacaccattcttggaagatgaaataagttaatgratgagaagggctgggtaaaaaaaaaaatgtgatgaaggattgtagacaattgccattgttatttcctgctaactaaggcttaaatcttgctctgaggggcctggctaacaatttatcaacaaacatttccaccttatgggctcaacacaagtgcagtgggtacagtttcaaaagtgcaaagatgcaggccatggttcttcgaaaagcctgaaatccaggtgggsccggttagaataatagatatgagctaagagtaaacaatagctcaccagtgattaagtgtttgggctttgccaactgtcctcgccatctgagtttggggacaggagggagggttgagttagcatctcaggcaagcttcatagaggtgtgtcaactgtttagccccaaggagtgagggtgtctctggtgtgtgcatattgtggagtgaggggtccctgggcctgcaccccagattcagggtcccccgcccttgcaggtacacagctgtggccatgcccatgctgtacaatacgcgctacagctccaagcgccgggtcaccgtcatgatctccatcgtctgggtcctgtccttcaccatctcctgcccactcctcttcggactcaataacgcaggtacattctgcttttgtttgcctgaggctcagctggccctgggcccctgctccctcgaagggccctgagggagcagagtccctggcacagatatgggtggaggcccaatggaggcctatcactaccctggactgagtccaggctacgtgtcctgggccaagccccactcaagagttgttctaggttggcagagaggaaacagtggcccaggacacaacggagttggatgaggagtggggataatggttcaggggtatgtctgggaattttcaactttggttattaagttgaagaggcaagacgtgcttcacaaactgcatgtgtgtatatgtgcacatgaacatgggatggcggaggctactgggcttccatccactcctgacaatagtatttttgtaatgaagctgcagccctcagggcctgctaaacccaggtcaggccttagccacctggagcaggaaagctagaagtgaataggaggtaagctgaccacatgcccatttctgtgggaggcagagtcaggagtgtctcagacagaggcaacacaaagcccccgccagcgtggaacttcctctcatcagggaggcaagagcgatggcaggtgtgcacctctggagatcgtaacaacaaaagatacagtgaagccttgaaaatgtgtggttcggtatctattataagcatcacaggaattctaagtgggaataacgctaaacatacttggattaatgcacaaggccctgagatagaggacaggcgtctggtagacctagaagggcacgcaaacatatgaaacacataggaacacaagtgagttcaacagacagagccaagttatcttgctgcaaacattaaaaggtggccaacctctcccaatacacaggtcagactaaaaagatggtttactcttttaaaagttttcttgtgtcattctttctggatacatcggcttcacttgttatgcccagacatggcaaaactaatgaccaagtaatgagggaatagtaatggaaagacttgggagcagtccatcatcacagcttaactttttgctcacaaccgtgtttttaatactctggtatctgctgtgcgtttgtgtatatctaagatgaccaggcagccttaaacatctagttgcgttcatattctctgtaaaatcgctccttgttcctggaaggacatgaatgggctcttgtggaattatggccggtgggcccgctgactccctgcctgcccgggctctccctcccccagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggtctcaagacaccccccaaccaactccaagggtccccacctaaccattaccaagagggctcatcttatgctcaggtgggggcttgggaaacctcagcaagggttaggtctaggctaaaggaattcccaggagccgggcaagggcagattttgaaggcatggacctcagtggagcaatctgagtctccaggagagggaggcagggtccatgacactaaataacaaggggaagtctttgcttggagatctttgtgactgaaggcggcaactcttgcttggtacccccgtgggctcctctcttcctcttttctggtttctctgtctcactttagctccctctgactcctccatcctctttcctttaccttccgtactctctgtcctcccctccaaacacacatcacttttcctgacttcctctccactatgtctctcctgtgcttctctttcatttcccccctgatgtcttgtgaattctccccttcactcagtccttcacaaggaagacagtgtgtgggcacagaaggaacaagagctcttgggctagacgcatcaggttcagatcctgtcactgacactttttttgctgagtgaccttaggcaagtttcttaccttctatgagcctgtttcctcatctgttaaatgggaatcaaaataccagcctcacagggtggtcttgaggattccacgtgagaagggacgtgagtgtgcctagcacagtgccaggcccttagcaggtgctccataaaaaaccaggtccttgtgttcctcgtcatacccaccactttttgtctcattccagccttcccccttgccccaactgcctcctctggcccccacccttctgatctctgagcccttctgcccagtctgagttccatggactgctgcactttgggtttctgtcccccctccatccccaccactttgtgtgacccatgtgctggctcactccacagggcaactgtactcaccccgaggacatgaaactctgcaccgttatcatgaagtctaatgggagtttcccagtgaacaggcggagagtggtaagtgctcaggccaggacccagagccaggtctttctgcccctagggaagcccactggccatggttctgagacctcagaagctggccaatgggagaagcaccccagaaacccccaccttgcctcagctgaaggcagactcaccgtgcacacctccaagcaggcatgaagtgagacacctcggttctgcaaggcatggatgtgtacgagaaaatggttggccataccaacgtaataaaaatgataataatggctattcacatttctcaaacatctaccatatccctattatctcatcaaatcctcaccacgaccccgggaggtaagtctctttgatcgaaccgatcttcagttgacagaagaggaaacaggctcagaaagattaggcaactcacccgtctcaagagttggtgacactaagcccagacctgtgtgactctgaaatccacacctgtgttctttccactgacatgagctgccttatggatgggcaggttctggggtaggacgagcagagcagctgcggggactggtggcggassagtttgtgtacatagagccctcaggtgcggaagcmmagcagaccccagcctctgccaggtggtagctgtaccaacatgcaagcagcaggcattccatcctccagagggatggagaacagggccagagaacccacagagggccgcatacaaaatccaggtctggtgtcctgccttcacctgcactgcaagggcaggactctaagaagctgtttatgaggcaggtgccaaaacagagcctcagagtcagggccaaggcagcagcccagtcatgccacctaggcacatagtgaggctgcactttagaagttcagactaacacctccaaggcctcaaacaagagaacctatggaagaacccaggaggccatgagtggatccatgccagggctctctaggtcaccccagcagggaagatgtggggccccaggggtcagccttttggcacctagattagtctatccaggagaatatggagcccacgtgtgtacgcaggtgcaggtacccatgaagtgggagcactgggccccttgtttgacagggagaacaggggtgggcaccctctttgcagtcggaacatgagtttctggaggcagggccaaacatcatcagctcttgaccccagcagccactgtggcacctggcactttgctaacagtaagtgttgctcaggccatgagagacaagtccctggtattcagccctggcagaacagaagtggggtattgaggctgcatgaggattgccatgggaaaaaggacaggggcaatcctgcaggggctgccatgggtcctgggtcccatgcctcagtgacatccttgcctccctggcagggtgaccctgtggtgtttgcaggagtcttcagagggtgaaagggaggggccagtgagatgggtggctgatgcctgggaacttgtccggctttacccagagccctctgcctctggtgcaggaggctgcccggcgagcccaggagctggagatggagatgctctccagcaccagcccacccgagaggacccggtacagccccatcccacccagccaccaccagctgactctccccgacccgtcccaccayggtctccacagcactccygacagccccgccaaaccagagaagaatgggcatgccaaagaccaccccaagattgccaagatctttgagatccagaccatgcccaatggcaaarcccggacctccctcaagaccatgagccgtaggaagctctcccagcagaaggagaagaaagccactcagatgctcgccattgttctcggtgagtcggccctggctgctggccacagcccggtctgtgaaaggtcccattccctgccatgtccttcctaaccatggctgcaggatcatggggntagcatttcctcaggcacaggccaagcccattgacatacagacagccctattaggtagattctgtaattgtgctcactttacagatgggcaaacagagttttagagtggttataaaacctgaccaagaggacccatggtttgaactcaggcagtctgactccagagtctggaaatttgactagtctattatgctggcaccagaacctcatgggcctatgtgcccaggaaagtcactttctctctctaggctcctcatctgtagagtaggttacttctcagggctgtcatggcaatcagttggtgaaagcattttctaaactacaaagcactatccatatgtaagtgtcactattatggtttttattactatggctctttttgaggaattgggaaattcagttcacttgcactcaaacaagactccatgggaccagagctgtgaaaaataaatgagatgttggggagtggcccccaatctaaatgaaaagcatccctgtccataaagcaggatttgtcaatagaagtctagaaaaccacatagtaaaggtcaaaacccaaaaggtggactgatatgggtggctgggaaaaggagggtcccctgtgactctgtcttccaggaagcacctgaatcccctttgactgccggatgatcctggggcgtgagtggggcagctgttacctcccttcttttatgaatgagagcacttgatcccatgggactactcacgatcatgggactgggatatggtaggctaggattggagccaggccctctgctgcagtccataggggctcagctctgtggccctactcttcccagacaccattcagtctccagggatgcctcctgtgcctgtagggcattccatgtacacattgcttccaggctaaatatcacacctgtgcaggaggtcctggaaaaagtgcacctgagttaggtgagcagagcaggtaggtggctgccctgggtgagagggcatggttcaaggcagggactagcaagggtgtacccaccaagactggggaaattgggggaaggctagagcgagatcatctggggacctggatagaccggagttcagctctcagccttgctgctcaagagtgaatgaccttgggtatattgcagagctgttgtgagtgtttttcttatgtaccttatgaggatgctataagaactaagtggcatgggtctatagccctcaaagatgacctggaagtagagtagatatttccatttctactggctaacctgggtcagaatggtaaccttttggatttattgttaaaccatgtacccttttctaggaggtgggaaagggacaaatgagggaagtgaggctgaaggagctgaagggatacttcactgcaaatgggtgtcagaggcaggtctaggatccaagaccgggactcctcctacagtgcttctgtggctacagcccaccgtcttggcatacgagccagggcgcactgggtgtgggtgttcccagccgtgcctccccggctctggggaccagcctgaccatgccctctcccccaggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccrgccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccagscaacttcagtcctgggagacccatgtaaataccagactgcaggttggaccccagagattcccaagscaaaaaccttagctccctcccrcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtagaaggcagcagtgccacttctgtgcttggcattcaagtataggaagacccctgtgtctgcagggtctaacccaagggaagccaggttgcccccactgntccacctcccctgttgcagctcctgcttcctctgaaggactcatcctttgccctcttacccaccagggcagagaaggctctgtggaaaaggtggccttggatgcactggcattgcctgtgtctgcctatgtcccttgncctgtcttctgtcccatgtcaggatccccttcctctagggcaggctgggagaagcagggaaggccctgaccactgcggcctggacagttctccctcctctcagcttccagggcggtcccaagctccaagccttccgggggaaaaacttggtactgccccaacaacagaaacttggctttctacaaatgaagcgtaaatcancccagtgagggaggaatattcttaccaccttgagaataaccgcagtgatgacaaacaaggtgccagcacccacgggctcaggcgctggggagctgtcagggcgtaatttgcatgctaaatacaatatttttagcaccaaagtttggagcacttaacttgccctgaacagttaattatggactttgatcttctccttaaacctaaaggtagcactaagccctgggagaggctcctgtccccaggagcaccctgattctggaaagtgagcaaaacaggcccctagtctaactcggactgggtcataacaccaaggacccagtgaccatctcctctggaaagcatcaggtccccaaggggtctagaagccccagggacccaacccatccccattgnacacataccatgctcaatgtctgtgaaagatcttggcctggatggacgcttaaaagtatatcccacaattaggaatcttatgagggtatacaggcttatcagatgtgangattggaagagatgacaaagagaagcagaggaaagaagaaggaagggagggagggagagagggagggacggaagggagacccagagcagagtgagaagagcattgacagggagcagaggggaagagggcagngcaggggcggnaggcggtgcaggggaaagttgcccacagttgtcacgaggcttcatgtctttctgccagacagcagattgacagctagagtgggcaggggagggctgggctccaccctctccccccctcagcacttcaggggcaaagaaatgggaggagtaggacccgacacgacacgggaacacaaggtggaagggggtggcccaggctctgactctctccagagaggtcctgacgatggtctcttgctcttgacgacaggatggaaaggaagcctccagttttcactcctctttgcctttccctaggtttctgtctgtgnctgtgccgctctgtagagtgccttttaatcaaaggatcattcatttgcctttacagcaggtagagtctgcacccttgtcccctgccctgccctttcctagagcacagcccaggatcaccacctaagggccaggcacagtggggcactttgcatatgtcgtcttagatgggaatggaaatcacacagtcacaaaggagcagacccagaggtaactgcaagtcttcaaggcttcaggccctcgttcttcctcctgggtcctgaaggactctcaggtggccctgggctggggaaagttcctgggaattagaagacgagttgtctagcagactaagaagttgcattgcttcgtccacccatctgctgtcttccttagggaatatttattgagcaattcttgtgctccaggccctgggtgaggtgctgggattaaaacagtcaacaatggtcaccggccctgccttcgtgagtccagtctagtgggatcacaacaaagtgagactctgcttcccccaaggcagagcccagggacggcagacacagcacaggcacatgctgcgcacttccaaggcacctttctcctagatgacgtgtgacacagcagccctggtgtgccttggccttgaacaattaggtagggcaccagtaggggcaggaagactgacagcatggaccactacttcctgcgtggctggggcaggaggccctgaaagaagcagctacagatcctgctttccaggtggtctcaccacaggcacagccagttgcctgcaactaagaacactgaagcccggcctgctttgggggcatttccagcatcccctcctatctggaatctcttcccaaaacatccctgtccccagggactgcatagctcctttcacctggtgggccagccctctctgtgtgccactggctccctgccntcctgggtcccaggacctgggctgagtgtgcaactttagcttccatctgcacagggccctctctgcgggctttgccctccctgatggcatgttcttctcctaacccactggagtgaggggcattcttatcgtctccacttacagaagagaagcctctcatacagaaagggtagtttccctttcaggctaaatcttctgattttacctcttatagtgtccattccagccctggcttctgaccagcgtgagtgcagctgaaagaggctaggatggtcagaagattttttttaaaagagaaagaatacttgttttggaccagacctgtagggtaacagcctggggtgcatgtaaacagaacttgggcctctccagggagcaagtgggtaggtggtgggggcagagcagttattagtggggcttcaggagtgagagggccagtctgggcatcggtgtgccagagggtcctctcctgccaaccaggtggacctggctctgctacaytagtttytgggggtgcaggggaagcaggaagctggtggggggagagtggaccatctttggagtagggagacctatcctggctttgtggggttagaggggacgtgggaagggggtacctggtaggaacaccttcctgcttcctctctcatcacagttctgctacttgscaaataaggcctcattggagcagaggcccagagaggcgcaggagagggaatggcaaggagggatgtggagagacctttcactcacagctggagccagagacaaccccaagttcataggaagtgctgtgtgaccgctccacttgcatgtggctttctcccactttcacatctggcttctgacacccagagctgcgtgtgaggccagggagagggggccacctccctggcaccccaggnccaggggtgtcagagctgcacaggaagtgaaggaatcctatcaggaaggggtgaggtggtcccattccaggaatgggagggcaggcctgggccaccgactgtgcagagacaaagctggccatccataccttctggattttcctgatcaggcccaactccaaacactctgtcctgttncccccaggagtgtgtaagttgggagggtcatttgggggccaggctctgtnctgcgtccagggcaggggaaagcatgggaggggcaagagaggcaggacactcccctggagaagacaagtcccttgttggttttctcccagggctgacagcacagacctgacccaggctctgggtagaggaggcatgactggctagtggggctagcctttgccctataactgacctgatccgtaggtgtttcccaggggncctcggtgtctcagcctacaccaaggcactgaaggagagtctccctttcctttcctgaatgtatcggcaaagaaaaatgggcagaggcttctaagagcatccttgcaaaaggctcccaagggatgggctgtctatgtcttattgcaagagaaacattcctctgatttaggaggaactgcaagaaatgaatgtgttgagtgcttattaactgccttgccctctgactggcagggcatcaggtttaattcttacaatgacccagagataaacattattgctgctagatgataccatcaagctaagatagttttagcaacctgagatctncaactanaaatggctcagcgaagacatgcacccaggtctgcctgcccgagtccactagacccagccacccgcagtttccctcnccgacctcagcccagcctggttcctgccctctgtccctaacattaccttacaccaaaaacccctgctgcctgtaatccgattcagcaagcagggaggaagcaccctctgagctccagacaccagggcactaagagaggactggatggaacacagaaacaagtagggcgtggtgatgtggcatgcaatgagcaaggaaggtggacccaagagaagctctgtcaacctcactgcactgggcatcgggacagaatgcccctgaggaaggagtgagcatctccagggggcaagcagagacgtcttcgtgggcaggaacaggcaagttggatctggaagggcagatcggatcccggcaggcaccggtggaagaagaggacacagcattagcaggaagaagggcctggggttggtgggtgctgcagcaatgctggctgggtttcagagtgggctnggagccctaactcactggaggccctcacttattggtgagatgtgttctataggtaagcacagggggnatcaggaaaggaatgttcctgaacacaagatcaccttggagctgycatctgtgtgttcaggaagtcctggggntccctccttcctctcctwcttgccacaggcctggagccctgtttcctccccttctcagacctgcccattatcattctcttcctggggctcagtgncctcaggttggctttggagagggacacggtctccccagtctcctcatttcccgtaggccttttcagccgngaggagttttctgctgatccctgcagtcctgcctcccttcaagacagtcgcggcagnctgtttaggcctctgccctacactttgntgtggagacagnttgmcaagnctccctgytcctttaggattcccagcctcataactggagggttccaccaacagcttccatctcccagcacaagaagggatccaggtggaacaatctcagctatctgcatctctggggatggctaggctaggggatgccatctcccaggacccaggaatgtcctccttgaggtgggctggattggatcaagcacagtttaggatgggaaaggaggctgtcatatgagtaaagggtaatgaaggggttacctgagtaacactggtgagaaaggacaggaacagaaagctggaagaggagagaagtgatggtgagtaccaatgtgtgctggagataggcgtgatctaattgcatccccaagataacaaatctgttataaatacagatgccgaaatctggaaagtctgggtaaagtccacagctagcaaaaggcagaattgggatgccaagccaggtgtgccttgctctgaagccccatagggcagggtccccatctcccaccactgtcccccatttcctcatcctgcacacagatgaagaggtctttaggatgcatggngtgttctgattgnatcctccttctgctgggaaactgacaggaggtcccaactgtccctcatcctgcatgggagntcccaaatcagaatgaacattaaggtctctccattgaatacgcacctcccacacctgcacctcaacagnactgtccctccttcagntgggcactctctgctccccatagcagntgtgtntntttcacattccccagccaatcctctccccttntatccagtccaggctttgctcctcttntgctctgtccttgtcctcattcaacatatgcatcttccaggaaaatctttcctgatcgaccccacgcaacatggcaactgcctggcggaggcaagacaggtatagactgtgctgtccactgtccccatgtggctagatacccacatttaatttattgaaataaaaaaacttgattccttggttacactagccacatttcaagtgctcaggagcatctgtggctggcggctatcctactgagcagtgtggatgtagaaggcttccatcctcccagtgctgatccagaatggctcctaagttaggagtttggataccttcaactaaaatagggtataaagggtgaagtcaggttctccctccaagtggagatgcccagtaagaaattggggagctctttccccccgatccccaatctcccctaaatagggcttgaacttctactggagaccctctccttgggaatttcaagggcctcattcaaacccaattttaaacatccctagggaagctctagaaggcagctgtgttgaaatgggactcaaggtggataccgtgtgtgtttgtgtgtgtgtctgtgtctggctaggagtggcttgatagggcagaagaagacactggtaggcaatgaagggccatgttgtccagggctggcctgaagtcacctctaagagttatctcttagaggaaggaagtggaggaagcaggggtgtgagaatctctgcctagactcaggatcaccagagctgaaatgattcccagggacagtcctgtccacatctccacagcatccctgccattcattggacactaggtgcaaaaggttcatctactttttctaactcttttcttcaaggccttttctcagttctcagctctttccaataggatcagccagccactcttctccctctttggttccctcctgatcactcccactgatggatctgagcacccttggctggaagtatgtagccccagagccttctctggctctcatagagtcctagatttggagttagatctggaagggcagataggatcctggcagacactggtgggagacttcagagtgcaaagagaagcaaacagccctgtgcctcctccacttccctcctcttgctcccgcatgttctgggaaagcttagaactcctaggggaatctttaactacaagttttgcctaaggcttagtctcagttgagactaaagagatttgagcctgggcagctagcaggrggagtagggaagatggaacagtcaggtggagtgtcccctggaaacccagttcgtcagggagtccaaatgggaagaggaaaaaggacttctggacaacccaagctgctgggccaactgatgtctgcaaggttatggctaaggaagggcacagagagaaaccatgggatgctccagtgcttctcagccctgggtgctcttcagaatcacttgggaattgttaatgatgctcacaccaaggtcctaccctggaggttctggtttaattggttgagggtagggccaggcgttggtatattttaaaagccccttgcccaggagattttaacgtgcaggctcggtttataaccatggctctaggacacactccagcttcctgggctcacatctgactttccatggtggccataagctatggcaaggtggtggtaaagggcccagggcccaacaggctggagctgtggtccagaggccaaaggatggaggacacaaagctgctagctctactgaaagggacttgttaattcaacaggattgcacgtgggcaacacattttcntctgccccacctctctcaccccatgctgatgccaggctgctagctgctgtccccatgcccagactggagtgcccagcccaggagaatgacacgcagaggtgggcctgccaatcagcttggctgagagctggccaatagttgggacagatgattccttttctctgacaattgcaaaacatccttgagtgggaggagctgcttctactatgctgaccagcagtgtagggagacagaaggagcaggataggaccccaggaaactcatcctgtccctactccctccaaattgttggctgctctcctggaagctgaactagggccctggtcacataaagttagtgtgaacagctccaagccacaaccagcaggacaggacaggacagctgatgggtggcctgggtgtggtcacaaaagtgagccctgcaagctccatactgagtccagacaacccacccctcaccatcatcaccccctccaactccctctgctgcctctgcctcctgtctttatgtgaactcacacacacatatgaacacacatataccctacattagacaaatcgcatacacacacaagcacaccatactaacgctgctggcctctatatgcaacttggttccacaggccaatccattctctaaacaggagcctcaggagcctcagctttcttgacttcaggagtttgaaatctggctgtaaaactggacacgatgtaggatgaagggtggtgctggggaccagggacctcacatctcacatagggtgacttatccacagttggagtgcacccttcctatatcctcaaaatgccatgagggagacccaggcattgggaccaggggcactggattaggtgtggtcagtgtggcaacagatggctggctgggactttagtgagcagaaacctcctttgaacttttagcttctgccccttgagcaaagcacatgtgtctggctctccccgccccagcctgaggtaaggagccaagagcccaagacgtgagatgggaggatcctgcttccctcaccgctgtgcctccctccatctccagagggggtctgctaggatgcaagggcccctgtgaactcagaagcgagtggcaaaaggtggtaggtgtcttggattggagagggtattcatttggggagcttgcatccccataaaggaggaggaggaggcctggggtctgttggaagtgactggaggatgtgttgaaggcagctctccaagccagagccccttcctcagctacagccacagtgatacctctccccatgcccccatcacccaggttcctgtctcctctcaaggtggaaggggcaggtgaagactgcagacagggaagatgccctgccagaagcccagctgcactttcaccaattcntactccatccaggcggagaggccccaagtagtctaaatttctttctttctttctttttnatatggagtctcgctctgttgcccaggctggagtgcagtggtgcgatctcggctcactgcaacctctgcctcctgggttcaaggaattctcctgcctcagcctccctggtagttgggattacaggcacgtgccaccatacccagctaaattttgtatttttagcagagacagggttttgccatgttggccaggctggcctcaaactcttgatctcaggtgatctgcctgcctcagcctcccaaagtgctgggattacagacgtgagccaccacggctggccaagttgtctaaatttccatctcggctcctggcttagaaccacccagagtggccactgacggctccttgccctctaggaaggacatgatgccctgctttcggctgcggagggccagttgcaggggtgtgcagctcactccatcctggacgtccagctgggcgcctgcctcgaccagcactttgaggatggctgtgttgcccttgagggcggccaggtgggcgggtgtccagcccaccttgttgcgggcgtggacatttgcgtgatgttctakgaggttgatgacactcaggaaggtgctcctctggaccgccaggtggaggggtgtccagcctgactgctctgcagcattggggtcagccccacactgcagcagtgctgacaccaccgcctcctccccgtggcgtgcagctaggtgcaggggagtccagttcacagctccaagagcacccatgtttgcgtggctctctgccagcagatggatgatctccargtggcccttgtangctgctagatgcaggggtgtccaaccctggtgggtgggcagctcaagctggctccgtacctgagcacatcttgcagatcaggtatttgcccctggcagctgcagtgtgcagtgggccatagccgctctggtcaagggcatcagggaccgctccactcttcagcaggtgttggatggccctcactttgccccgctctactgccaggtgcagtggtgttctcaggtttctctgctgancatccaactcagccccctggctggtcagcatcttgaccaggctaacatggccaaagtaggcggccacatggaggggggtcttgccctcagcctcacgcaggttggggtcagcctgacgggagaccagaagccgtgccacattctcaaagttattctgtgcagccagntgaagangggtccacccttcacgttcctgggcatccacacaggccccgtggtccaagaacangcgcgcantgcggtcatccccattctgggctgcaaantgcantggggcc

SEQ ID NO: 6 - DNA, 501 bp, Homo sapiens
gtgccgccaacccattgcccttgagatggagaccacctctttggtattgcaaagaaggtccttcgtaatcatctatccctcatcctcacccttctgctccatttgaaaggccctccagaacaacatgtcattcctgggaaccatctttggtattgcaaagaaggttcttcatgatcatcgtcccccatcctcaccctcctgcttcatttgaaaggccctccagaacaatgtgtcactaccagaaaccatttgctatctctgttccttgtttgtatttctgcactggaaatggccttacagctccacactcatccttcagacatgcagctccttggtgaccccatctgctgggtgctggcctatgtcacatccaggttgctttttattcattgtctcatctaccagcccatgcctccccaagggcagggctggtcctggttcactaacccatccccagcacccattggaatgctctagacttcagcccagtgagtcactaag

SEQ ID NO: 7 - DNA, 501 bp, Homo sapiens
aagaaccagccttcccacccgatattccgaccttggccatgggcccagcccattgaggtcaagctaattcatgccccttttcaaggcccagctccagccccagctcctccaggtagtttccatgactgttccagctccccaagggtccagctgcttgtgactgcctggctcatgctaggctgggattgatctttcactgacccctataaaggtgttgtactttcctaaccagcccaggttctctggagcataagtttaccttattttgtacaaggctgagagcctctaaaggcatgtcctgggttgctgtcatctctggccttttcttaagcactgcacagagctgagcacacaggatctcaggatggaccaaagctgatgctgtttcctaggttacttgggaactcattgaaggtaggaggctcagagtgggccaggaggacagtctgccaccttgtatgcccatgcttcaccaagatgcatggcaaatacaaaacagtc

SEQ ID NO: 8 - DNA, 58567 bp, Homo sapiens
caggcagatctaggtgaatagtaacaatgacaatgacgatcattcataatattgaataataaaaatagcaaacatttgaagcatttactatgtgctaggcactgcgctaagcactttacgtattttaattcctctatcaaatgccggttggccacatgggattaaacacctgcctctgcctccaaaccccagcacaagcatcacctccttggtagagtcttccccattggtctcaggcagaattaatattcctgcctccacacccgcagtgcctggtctattgttgacctaatttcactagctcttaattatttatcaaagtggctgcttcccattccagcctgtgaacatcttgagggccaggaccctgttatatttatctttattagcctcagtgtccaacagtgactggtacctagttgctcctcattaaatgtttgttgaatgaatgcacagatgaatggagtaattaatacaatatcccccaatgttcatgggattttaaagaggccactttcctagcctcatgcttcacaaacattccagaccagtgctactccaagcatgatctacagaccggcacctgtctgcaaactgtgttgccagtttatgacctggtaagtgcagaaattgagcataagcatttagacttttttttttttttttttttttggagtgagagttttgctcttgtcacccaggctggtgtgcaatggcgtgatctcagcgcactacaacctacgcctcctgggttcaagcgattctcctgcctcagcctcccaagtagctgggattacaggcatgcgccaccatgcccagctaattttatatttttagtagagatggggtttctccatgttggtcaggctggtctcgaactcccaacctcaggtgatctgcctgcctgggcctcccaaagtgctgggattataggcgtgagccatggcacccgacccatggctcatatttaatctccacaactattgctatcatcctatttttcagggggaactgaggcttattgaagttaagcaactccccaaggtcctagagagcagtggtagggcagaggtggtgacccaagcactttgatccacagctcacgcctcagccacatcactggcagagagtcaagggactcaacagaaacattcatgctaacttctcgggttctggaaagctctatgcaggaggtgatgtttgttctgagccttgaagaaggcataatggggaggggcattccaaggcagaggcacacaagaggcaaccgcgtgaggggagtctggtgaggttgggccgggtgcggtggctggtttcaaactcctgacttcaggtgatccacccttctcagcctcccaaagtgctgggattacaggtgtgagccactgcacctagctacattttgacatttttaagccattagacagaatagttttatgtccatttgatgtgctagtgtttaaaacagggctttcattttgtctttctttctttctttctttctttctttttttcttttttttttgactgagtctcactctgttgcccaggctggagtgctgtggcaggatctgggttcactgcaacctcctcctcccaggttccagcgattctcctgcctcagcctcccaagtagctgggattacaggctcgtgccaccatgcccggctaatttttgtacttttagtagggatagggtttcaccatgttggccaggctagtctcaaactcctgacctcaagtgatccacctgccttggcctcccaaagtactgggattacaggcaatgagccaccacatctggccttttattgcatttttctaataattcattcccgcagtgttttgcaatcgcattggcctgtgacagattagaaattttaaaatacagaaccagttctttgccacagttgtctgagaagtatggttccagaccagcaacctcccggggatggttaagccagtggaaccccacaggtaggaagtgcctattcagccctgcctccctgggtctcacccatcattacatcaaatctgtctctcctactgttgggaaggacatgtgattgtcttttattcctgtttggaatcctcacatttctcctgtcttccaaatctggcagattaaatgaatgaaagctgattctgctgctggctggacaggaaggggtgagaaggcaagggagggatgggatgagaagagagtttggaaaaggccatcctgggaaaagaatgctggaatggagtaggagcctatcctgaggagggtgtgcagaggctgctctggctggggctgagggaaggaagggtgtaggagggagggacagggcagacactagcaccttcaccaagcccagcagatgagagccaggcccttgaaggtgaaaggaagtagtttcaggacaaatgaaaggggatattgaacctttttacagaaggaatgttaaacgccaaactttcctaacccccctgaggctgccaaggccaaaactataaataagtaggtcgaggaattaggcagaagcctcctcccctgacctgtccgtcagcgggatgtggtggggtggaggttaagtcagtgaaatcaacacagacaggatgaaatgagctatctgtagacgccacttcagaaagcgagaaggtctgactcctgggcaaaaaagtcaggaagagcaagacttgattgcagtccaatcaatggcattagcatatttaaagatcatgtcaaggggaggataatggggctcagagaggcaaaatttgatggtgtttattaagcagagagaaacacagaagctggagccgtctagtccttagagagtgtgctccaaggtgggaccagggagggagcaaggccttggctgggcctgggcaaagctaccagaggaccaaccggcttcctctcctctgattccacagcctactgctgctggtgaatttctgatcaagaattctgctccccacaaagaactgcctagagggtgggaccaagccacccctcacacttgagcatccagtccactccatcgtgatgatgatgatgatcatagtattggtgactacagctaaccctcattaagctctcactatgtgccaggctccgttccaagctctttacaaataccaactccttcaaactccccaaccaccctcagaagtagcgtttctaccccgattttatagatgagaaaattgaggcattgttaaataactctccaagtttatacagcttgtaagtggaaaagccaagattcaaactcaggccatctgactccagaacctgcacatcagactgccccgcacttaactgttgttggtaacagttagttgtccatctgcttagcatgtccatcccttctggggttggctggggcaagtcagggcccaactcggcacagccagctccggagcactaacctaatgggggttttgcaagactcgggactgtgtgcttcacctggaaagtgccaggtgtgtggataagccatgtgagcagctggcttcctgggcaatgcccctccccacttcagtgtcccaggcccctttagggcccagccctccaggcactgtgagctgggaatgaggtgaaggaggactaggcttaccctctgtgtgcctccattttctcaccagtaaactgtgtgtgttggatcttacgatcttctaaaataactccagctccaaatgaaggccagtggacccatgaacacgctggctcttgcccagtgtgctgagaagagggcaggagggaggagtagggtgggcacggcagggcacagggtgcagggcaggtgggttcagtttgatttcaaagccgactgcccgccgccccaggtgctctggtgtacccaataggggctgcattggccaggcggcctaggctctgcgggtggagtgaagatttctctacctcccagtttctcccttttcagctctggtaggagagaggaagagtgaagaggaagtcaaaagttctgttcatctggaagggctgggagttgcttcctgggatggggagctccgaagccaattcagaagacggccccggggtctggcggggaggcccaccgctgcccagagggctcggccggccgggagggggcgctgtgtcctccccacgccctgcccgcgggctcctagcagcgttcgccgcgctgcaacgcaatgcgcgcccggctttcccctgggcagcgttcagtgcccgcttgtcctgcagggcaccgccgtctccctttcatcctggggctcccaggtccaaagggttcccagatccagcctcttgcctgaaccaaagtggccaggcactcagtttgtaagatgaacaaatgaaggaaagaaggaacgaatgaataagggctcatcttgcagagcgttcgcgtctcagcgcgcaccaggcacatcagtgccaaaggtgctgggggtcgggcccagcgcccggcgcggctggaatgggggcggcgaggagctcctcctagccagccgcagggcgggaactgcccaaacgcaggtcgcagcctcactgggttccgcggcggctccgctcagtagggaccgccctgttccctagcccccaatagcctcgccctcataaattcacctgagcaccctggcccaagtcaccccagatccctgggacttcccaggccccgacctcggccgggcgggaggctgatcctttaccgcggcccccttcccccattcccggcttctccccgggctgcggcggccgccactgctgccagcgcgccccgaggactgccgcaaaccccgggcgcagggaaaggggatcctggcggcgggacacagagagcgggccgggggaaggggagggaacggaggaggcgaggaggagctgggagggagccgaggggcgaggcgcggaggagggaccgaaggggagcggaggggagagggagggggaggaaggggaggggacgagctgggcgagggggcggggagccgaggcgaggagcggcgcggaccgtgggggaggtggcgggggcgaggccggcaagaggcggccgcgggccgggctgcgtcgggaaacggcggccagacttccccgggaaggggcgagcgagagccgggccgggccgggccgggccgcggggccgggaagcgccgagccgggcatctcctcaccaggcagcgaccgaggagcccggccgcccaccccgtgccgcccgagcccgcagccgcccgccggtccctctgggatgtccgtaggacccgggcattcaggacggtagccgagcgagcccgaggatgggagggcacccgcagctccgtctcgtcaaggtaacagcccgccggccatccctcgagcgctggcgcccccggcccctggcggacgcgggacggggctgggctgtgggtgtgatgggggcgggggcgctgggagagccgtgcttaggtcggggaaggctgggctccggggcagcctaactctctggacctttggagccagggttgggtaggcagggggagactcaggtgagaaaagaaatcgtggcgaagccgccgggatgttggcggaggagggggtccgcccacccgggtagccggctccgcgccaagaatcgctctaggctctcgggcagacgcctaggggaggggaggttccacctcccacgcccttcctgcagacctcggccccgggactggagactctgaagcggggttcccacctcgccccagcgcccccaaacagctccccgactccgcacgcccccgccctagtcttgggagcgcgtcctgccccttcccctccgcggcgagctgcgagtccggagagatgaatgccgcccgcgcctgcgagcccttgatcaatatcgcagtctcgggggaggagacggtgcctctggcggcagttcctgttccccgaccgcggctgggagcgcgacgctcgttctccgagcctgggcacccgcgatccccgcgctgcgcggccgaccctgcgacgcggatcgggcgctggagtttaggaaagtagccagaccgcacgtccagcgtctgcccgctgaagttcgtgggcggtgggggcggcccgcgggttgtgtgagccagggaagggccgggaggccggcagtccggcccgccgctcgtgtcaggcgtcacttgcagccccaagcgctttccagagcgttttggttttgcagggtataggcgcgcccgctgcagccggccggagaggcgggtgggcctctgcgctgctgccgggacaccccgcttcgcgccagtctgggcatcgcctcacccggggctcgggactccgcagggtccgctccagccacaaacatagacaccagcttgacccggctcagggcattcagagcgattacattcaagagacttcggtggatttgggggaggggtgcggattggcggaatttcatttttccatcagttattaaaccaactgtacgtataccggcttaccaagtggtttgagttaaaccaccaagatttttaaatgcagcagctgctgcttccttagttgagacgttacaagttactgagatttttctctaatcctgcccctccccaaatatttataaattgattcttaaaatgatcaagggttggtcttactgaacccacatcccctcccccatccgttacctgagggctagggatgaactcagattattttctgggcgttcgaggagacagacaggtgaggaaggtagaagaaaaacatccagagaggtcgagtagttggcactgaatgggaatagtttccaaaaggctcgcgtgtaccgaagcctggtctaaatgcacaaaccagttagaccaaattcagtcttttaaatggattcaaagaactgaactgcgggtgttttcttttcttttttttttttttcaggtttacgcggaaatagatcaatgttctttttctggcctttcatagaattgtgtgcaaatctctgtttagaatgatcatgttttccttaaaaaagtctttttatttcctttgttcctactagatggtggggtgtttgccctttatgcctggcctaataaactcagtaggcattcaataaattccagatgaatgtggggcagtgagtgaatggccagcgaatgctggtttcaaacgttggttacaattaaatagttgcttttcttcttttatttatgtagcatgtacaattcactgggcattattctacctgctttgcaaatattaagtcatttagtcctcctaacagccctaggaagtaggaattattcctgagacacagagaggctaagtaactggttcaaggtcacacagccagtgagtagtgaagtcagaattcaaacccagacactctgataaaaggctttgttttgtttgcttttaccagaaaatatcctttttttttttttttttttttcaagacagggtcccactctgtcgcccaggttggagtgtaatggtgtgatcatggctcactgaagcctcaacctccctaggctctggtgatcctcccaccccagcctccagaatagctgggaccacaggcatgcaccaccacacccagctaatctttgtattttttgtagagatggggtttccccatattgccctggctggtctcgaactcctgggctcaagcgatcagccagcctcggcctcccaaagtgctaggattacaggcatgagccaccatgcccggccattaccagaaaatattcgaagccacctaaatctcactctagagctcagccagatgagagctcaggtagttttgtcagcatgagagtacaaatctaggggatttagggttaatcccaaatccagctccatccccaacaagtcagccagtgttgggaaagggtccactctggggcctatattgtgggcctcacactataacagttacagagtcctgggggaaaatgtgtgtgaatggggagagacagggtgtgtgaaagtgtgtgtgtgagtggggagagacgatgtgtgtacaagtggccatgagtcaggagaagacagacaagacattgtctctatccccagggaggggcaggggcctgagcacctcggagtcagccacacacccactgcatctcctcgtagggagatggagtgtggctgtaaacatgggtcccagtgtctgtgaggggtgcagaaggcaaggcctctggatggattctaacaggagagtgagctcccaggagggagctggctattgttattcttatataagccaatttagcagcaataggcaaagtttgaaaaccagaatggaaaatgattgcaacctcactcttttaatctggctactgtataattcatttaaaaaataacccccacaggtccccttctcaagcacctgtctgggcacagctgtggccattgggcagccctctcctcagtgtcacagcccaagtctgtttcacgttactcccgaggctctgcaggagactgtcaatgtctgcatgttgcctgtgagtgaatggactttgccatccctatatcggtgcatatctgggccatttccaagttgtttctttgattaaacgatatagagccaggttttcgaggggagcgttggcaagaggcatgaggaacgcgaaacagtgagcccagggtgggccagtgggctgctgtggcgtgcgggactgctggcctggccacagactgggggcaaagacagggaggggccaaaagccaggccaagccccttggatttgaaatgaccagccatagagaggagtgtgtagattatggaaaagttgaacactcacccagagccagtgaggacgagctcaagtggaaaatccatttctcaccctctctaacatttggctcctcaaaagcatcaacctccaggttcagccacacataaggagtttgggggtcaaggtgccaccaccccaagtgcccgtgtggcctagaaggaagcagttaggtgccgggctgataatttcagcagctattgaaggggaaagaccatcagcctgggagtcagtgaacctgactctgatcccagctctgcctccaatcagctgtgtgatgacaggtaggtcaccttccctctctgagccacagtttatctgtctgtaaacaggggtattggactggattctctgttatctctcatgtctctgtgaaaggtctgtgttttcaaatactccttagggacatgacctcacatagaaagaggggggtgccacgaactggattcccagcactcccccttcccagctctgctcaagctgagggaccttggaacatggtacttggacctaagtactctctctgggcttcagtgtccccagctataaaatgggggaaatgcctaactcaggaggttgaggtaaggacaaaaggaattaatacatggaaagtgcctggaacagtgcctggcacagtctgctttcagaaaaagctgcaaggtggctactctcagcagatataagttaatgcaggaacagcaactagcagctcttggacatgcaatatattatatattttttgcatcttttacccttcacggcactgagcctcacaatccgttgcacagagtaggtgtttaacaaatgtttgtcgacttcatggatgagtttattgttgcttcttccatttggtattgtacaattggaaagaacccgtttctgttcccaccagatgcagatgtcaggtcgaaacagaaggctgagagatgcttaaaccaccctagcagaaaagccatctctgatgcagaaaggactagaactcccccttaagcccactgtggttattaaaaaagagaaaacaacacacaaccagtgcagcaccccagtgatctggagaatcccttgagggtagagcctcagccagtgaaactccagcgcttttgccgtgtctggaggagggcaggtcactaacagtgatctgggccgtttcttcctcagtgaaaagagggagtagggtgatctctcccagctctgattctaccctaactgctcacggtatcaaccaaagaagggtgagcccattttgaacgtaaggccaggtcgagatttcagctgcagttttaatagtcaaacaatgaatttgttaactacaaatcattcttgtctacctaaattcagggaggtccctgtcagcagagtccctaggaattttttttttttttttggagacagggtctcactctgttgcccaggctggagtgcagtggtgtgaacacagctcactgcagcgtcaaccttgtgggctcaagtgatcctcccacctcagcctcccaagtagctgggactataggtgtgcatcaccacacctagctaattttaaatttttttgtagaaacggtcttgccgtattgcccaggctggtctcgaattctcaaactcctgggctcaagcgatcctcccacctcagcctcccaaagtgtagggttacaggcgtaagccacagtgcccagccttcatccttttaaaatgcttgtctttggacttgttgctctaggccagccatcccttaccccacccagtgtggcagtcaacatcttcttgtatgggtgggaacagcagatgaagcaaatgtgggcttgaatccttactctgccctgaagctgtgggcaagtcacctcttcatctgtctaaagcgggggaaataatacctgctttatggcataatcataatgaccatgagatgccaggtgtgaaattatgcacagttgcacagtgtctggcacacagtaagtgcataataaatattagctatagtagtcacttaataaatcttaccaacttcctcagtctaagaaaggccatcaaaaggagaagtggtgaggggatgggggtagaggaagggtatttggcgtatagaatctcttcctgcgtcagagcacttttcacagtccctggggaaaagggatcagctaccagccatcagctgagcttgaaggtcatagacagacctgaacatctgggatatggaaaaacctgacagtaaaagtagatatgtagggaggataatacgaaatgattcactcactgatgcaaacaatcattttgaaaataactagctggattctattagacaacagattcttaatcacttgttttctgagtatgtggtatccgcttccacaaaattgaatggatggcaggaagggagggaggcagacttcatgaaacaggcatgtcagaatctggctttggaagacgggttccccttcacgtagaactgcggattcaaatgctttagccaaacatgttctttggcaactgtccacccagcagccataaaactgtttctgggacaaatgaagatgtttgcggctgttctttctgccctataagcacaggcaatttgtccatctcacccgcctggcactgttgggcccagagagaagcctcagcgaggtgctttgagatgggatttgtggggtgtgcatctgcccccctccgcatgtcagctggtttccaagccccagccagtctggttgggagccagtgcgcctaggtcagagcccagaaaaactggctagactggtccagtcacttcgtggctgcagaaccagggaggcagggcttgttctagactgaccgctatactttagagtctacccactggagttttaagaggtagtgggggctatcaggaagcacagtgagaatcttagttgaacctttgagtgctgggatgtcaggggctttaggagaattgggacaatatattggtcatcatccctggtcagtaccaaagcccacagagctgaaaggcacctgcccagggtgaaaggctgggacaagtgatggccgcagcattgccatttccagtggggagtgccctccctctgtgatccatccacctctccctccctgttttcccagcctctttggcaaggtgtgtgaagggctcatttctagaaagcccttcctagctcagcagaggccagaggtaccaggctgggtggggagcctgtgcacagagacagggaaaaggagtgtggctgggggacaaggggctgggctgggtgggctggaaaagcaaaccgggtccaggcggggttttaccctggggaaagggctttaggagagtccagggccatctgaggcacagtccttacaggtccaggccccctgcactggggacccaactgcactcagagcacgggcctggcagggtgcccaagggctcgctcagggtggatgatttcactcttacggaagatgcggagacagacagacagtgagagaagcaaaaggggagggagaagaaaaagacctgcctgcctgccttggtcattcttgcttttaactttctccctagcatccaagagagcctatgcccgggttctgaggccagcggtggcatctcctggaaacttcctctctgtttctcacaagaattcctccacccatccctatcctggtgcctgggtgcccgaagccgtcctcagctgttaggagagccttcggcgcagagatgccccagatgagctcccgccatgatcccttccaccacccccagggctgctcctcctccccagaagcagggtgcagaagggacaggatctgttgcctctgtgggcctgtgtgtttctcagtgggcatttttttctctctctctctttctctctctctctctctctctcacacacacacacacacacacacacacacacacacacgctggtaccctggcgagtgagctactttgcagtgtttgtaacacccgcacctgccacctccctcagaagccactgtgtggtcaccacctctcctctgagcatagccagacctgtacggcaggtgtgcaatgccacctcaaggccagggttagaggctgacttccagaggagcagctgggcacctggggaccacgggcttagctgggtgagtgactagcaggttagacagcctccccggcatccctcagctgcccgtgcctggctgggagcccgagagactcagccactccttcatcttcccagacgggtagggcaaggctggtccttggagggaggctggtccttgcgtcctgggcttggtcttggtctaagccacgcccttccccttcctctttctgccaactctgctcctgtcccttgcctacgcctcccaggttcttttaagcaggtacactgtttactggccaggtgcgccatttactgagctgctgttgcactaaggcccacacaagtgaccccattgagtcctcccatccccttctagaggggaaagaacctgatgctcccctaatctcttgctcctcactcctggccctctgcctgagtcgcactgttctccttcctcccctgccaacccctacccagcagaatatacatatatgacacttctcttacaggagcttctgcccccaagtctttctgtgccaatcttcatctgtcatgtccaccaaagtcttcctgaactggccccacctcctcctccaggaagccctccagtctaaatctgtctctcactggttgttcctttgttctcacctcatcccgtcccctggtgtgagcctctagctcatcgtggatcctgggctgtcccacaacatggggtcttacagttctttgatgtatccccccacagggggctcaaaaacacctactaaccagattccacatggacccctggtgacccacatctaacctgagcccttgttcccaccgtagactatgagctctgcctagaccagaaagattcccttacaaaacaaaacaaaacaaaaaaataacaaaacctctgtgtgcccagaacttagtccagaactgtgcacacagtaggtgcacagtagatgctgaactaaatggagggtctctccctgctcccactccccaatgaacgtctgtctgagtgtggaagtgctatggcgacagaagacacaagtggcaagccagacagataggtccctctctgcctccgcgtgtggatttgtgactcaacggcttatcaagggctacgcggcagcgcggcttagtaacattaaaagattccagcactgccctgtgttccgcctccggtccccatagcactgggggcgttgtccacgtgcacgcaagggcagcatgttgcttcatgagcggttctggacagggatggtgggagatgttgctagagggaattgtggccctgggcttagaaacaaaggggcaagaaggtctcagaagctggggcctccagagagcagggtcagggcactctagcctccaaatggggactggggaggcggcctaggtggcatcctggggttccctccctggtgctgtcctggttctgctggtcatgaggcaggagtagccaggcaaggcaggggcgccaagagcactggcattcctggtaccttcgtgagatcccgtccgttggcatcccacccctccagctccctgtgagtcggtgtgggggtgcagcctggggcgtcttttctctcctgatggctggggcctcatggcctgactgtggaagctgctgcaaggctgtgaaatgcagggcaggggggcctaggagtggtggggctcacatgcaccctggaggaaggaccactgtccctaaagctgacagggcaggagacctggggttggagctgactcagccacttcttggatgggtgaccttaggcaaatcacatgacctctctggacccttgtgtcctctttactggaccgaccacaatgcagaatgaaagaggaaatgcctgtgctgctgcaaggggcctgctcccaccaaaggccagtgagacctgctgttttcatgggtgggagtcccaccccaggactcccctcagaactaagcccaactatcactcaagacccgcctcctccatgaggccttccctggtgagccaggcatcacactctgtcctccagccaaagcagggctctcaaaccagctgtggggaaggaccacatggatttttccaatccatggccaactggcgtgtggtcctgctgtgtagcctgcaccacgtgattcacctggcaagtctgacagcatctgaattggtctagaccctgtttgacgagatgagtcttgactgcatgctcggatgtcaccgcagggatggattcacatgaaatgcttactctccagttttgtacttctcaagttgtagaccaggaaccaattgttggtaggttggcaaacttgaccagtcggaccagtcctcaccaccctttgaatagcacgggcctaaagccctcctttggggccagaattcactgcctggcccagatctctggttcacccttcacacagcctgcccctcctgccatgcttcttgcacttgtgtgtctccattgcatggatgtgagtgatgtagtggctctttctggcagccaaggctgggcgacggaagaaggaagaacccttgtccaggaggaggaaactctttggtttactcagaccccttacccagtggcgggttctgagcaggccccttccccctccttgtcacactcatcaatctgcacttggtgacaaggtgagctgaggacccatgatcagtggtcgcagcccctagtggggcaagggcagccttgctctgtgtgggttccccaaagctcacaggacttagccccatgctgttctaccccttgtccccgcactcatccaaggtaaaggaatcccacttctccagggcccagggcccaggacccggctctgcctcctgcctctgaggtgccggccctggagaggctctggtggagcctcagctgggtgagctcagctcgctgactctgggaagaggctgctgttcaagctctttctgggctgcctcattcctcaactttggctgcatcagcaggcagaccttcctgcatctcctgtgcaccccctgcaggaggagatctggaggagctgacccagcagggccaggacagagtgagtcaggggaaggagttcccatctgcccagctggccaggaaaggtcgtttccaccagtccgctgccttcagactgaggccaggaggtgggagactgactgatccaaggtcgcacagggcattcaccaagaagagtgcaatccaggtcctcttcctgccttgaaggtgcctctctcttgtaatcccacactcttcccaaggtccactctacagccaccttcctcccctgccccgcccccataatgggcaggcaggcaagtgtcttcaaagctgggatggagcattgaaaccctccaacccagggcttcccaacctcagcactattgacatttggggccagataatccttcactgtgggggctggcctgtgcattttaggatgtttatcagcattcctggcctttacccattaaatgccagtcgcacctcccccttcctgattatagcaaccaagaatatcttcagacatcaccaaacgtcccccgggaggcaaaatggcctccaggtgagaatctctgagctggctgagttatcctcacttctgttgatgcttcagaatcactggaacgcatttaaaaaacataagggtccctgggctccccacaggcctattgaaccccaattttgggggtgagttgtgagaaatgggattttctgtctcccaggtcaggtgtcaagcactgtctggaaattctgattttgtctaagtcactgcctttacaagcaaagaatcagaggacggggccgttgcttgttttatgtctggttagtccttatttctgccctaaatattccctgttcggtggggtttggcggtccagcagccctctttaaactgacctttggccttagaaggcagtcgtttttccaggaaaaacagccccaaagcctttagcatttttaaataaagtagaacctaattttcagccaccagtttcactcttcctttctggccctctttaaatcccttttaaaacgcagctccataaaggacatcttttctctgccgacaccgaaatccccctcactttacacttgtcagagagcggctgagccgagagcacagctgatagtgtcacctgcgtagctggtgtctctgcggctgtccccacacctcggctgtgcctcccacccactcagggacagtttcccagtgtctgggagccactcacagctaacagctcccaggggcaggcactggatcagaaccctgggctgggctcccctcttctgtgagagccaaacagagcccttcctgagtcccatccattggcagggtcctactgttgtccgccccctcgttcccactgccagctctgggggagctgggctgcagccaggagccagagtttgaaaggcaaaggaaacctgtacctatgtgtcttttggaggatccaggcttgtgatgaggaagaggagggaagggaaggcaggcagggcaggcagggaaacaggctgtgctgcccaggggcaggagcatggctccaatcccagtccctccagttactgcagtcaaaaccctgggtaagttactgtctccgggtgcaccagtctctcgtttgaacagtggggagatagtaaaggctctgacttgcaggggtcgagaaggattaaatgaagaaattcacagggcctggcactgtgcaagccttcagtaaaggctgaagaaagaaagaaggaagaaagaaaggaaggaaagaagggagggaggaaaatggaagccagggaagactgagaaaggtagggtggttaagacagggagagagcaagatggggaggagagaggcaggaggccgagaaggcacgggctacaagaacatatccatgagtcaatttgagagcaaatcatacaaagacggggagcaagtgaatgcatgagaggcagaaatgaagggaagacaaggaggatgggagctacgctccgggcaccccctgcccccatgcttagcaaatccatatcattgacctggatgtacactccattcctgtaggtgaccactgctggcttctgggaaagagtggagctggaaagtggggtacatgccctgagtgggcagggggctgggcaaggggcacttctgggaagaaaagccaagtcaggaaggaagaacgtgcaaagttgagtaacagagtggtgaccatttgtgcggcctgcgcacctagtcccatgctgttttttctttttctttctttctttttttttttttttttttttttttgagacggagtctcgctgtgtcacccaggctggagtgcagtggcacgatctcggctcactgcaagctccgcctcccgggttcatgccattctcctgcctcagcctcccgaggagctgggactacaggcgcccgccaccacgcccagctagttttttgtatttttagtagagacggggtttcactgtggtctcaatctcctgacctcatgatccgcccacctcggcctcccaaagtgctgggattacaggcatgagccaccacgcccggccccatgcagttttatatgcaccatattgttgaatctctccaggagctttgggatgtgaatattttatccctatcagccccatttacagatggggaaaatgaggcttggagaagttcggtgattctcccaaggtcacacaggtaggagggggcagataaaagattccaacaagcatctctccagccccaagtttagagatttgaatttctactctcctgtacatcctggagccagggcatattcagacctggaaaggaatgtagagaacacagtgacctccccattttacagatgaggtaactgaggtggggaggtgacttctctaccatcacggagcaagttcagcagagtcagccctgtgcttcccacccagagtgctttttcctctagcacatccttctggagtccaaggtgcggggctatggcgggagcaaggagaggagagggagctcctgctgagcaggaggaatagagaggaaaatgagaaaaaggaaacagccccctacaatctcctgctgggaatcatcatgagaccccatggtcagagggggcagaaagtgaaaacccatctgttcacttattcattcagtcatttattcattcaacaaacatgttggggcctgctctgtatcgggccccactgaggatacaaaaatagccaaacacgggctgggcagggtggctcacgcctgtaatcccagcactttgggaggccgaggtaggtggatcatctgaggtcaggagttcgagaccagcatggccaacatggcgaaaccccatctctaataaaaatataaaaattagccaggtgtgatggtgggtgcctgtaatcccagctactcaggaggctgaggcatgagaatctctttaacccaggaggtagaggttgcagtgagccaagatcgcgccactgcactccagcctgggcgacaaggcgagactccgtctcgggaaaaaaaaaaaaaaaaagccaaacacaggccttgcctgcagggagcttacaactcaagcttataacagtacatctggaaccttggaggggcgtgtgtgactctggagctggggtctcatggcccttgctgccctggggtgggagaggcaggtgacccactatttctagccctgctgcaaggttttcttaggggagtagaggcgtgaagtagcagggagggcgggcgggggcaccctgagccggggccccagggagaaaggccgggggcggggaacacccaggcttcccccctcccttaactgtaggcaggagattttcctttccattttttcttctctgtacaacagcaatttaagttaattaaatgtaacaataaattatttctcgcctgcctggaataatctttcagtgctcacccactttgaggggaagcactggctctttgccttgagatgggaactggttgtggctgtcacagaaagaggctgtgggaggagggccaggcccgaggcgggatggggagaagcctgggcagtcaagagggctgagaaaggacccctggcatctctgtcctctgtgcctagtctagttttactctggctggtctccacctgtgggtggatgagaaaaaggtgccccatgagggtgcccctctgctggtacacatcttgcaagtctatcccccactgacccctccacttggcacttctgtagcaagaaattctgcagaaaccacacaattgcgtgtggggcctgccctgctcttgtattcaattagtccgtaactacagatatatcccccgtaggtacagagacgtttctgaagagttgattgcactgacgctttatttctgcaaaccagatcccagtatttaaatgcaacggcggacccagctttttacagaaatcttcatttatgacctcatttctttttcgcgtttagattttcttctcttgcctttgcttaaaataggctccacctgaacacagtgcaaagcaaagcagagtcagaagggtcggatattgggattgtggagcctgcaggaccccaggacccaggaccttggccagcagatggaaagtgtgggctggggcccttgcctctggggtcaccaggtacatcttcgatcttggccacactggagagtcaagggcttctctgggccccgagtcagggtccgccagagtctcccaggccagggaaggcaacaaggcctggtttcactctgacctgctgaaccgcctccgtggaacaggtgactgtcggctgtctcgctgggatgatgagggtgacaagctgggataattcactgctcaggaacctgccatggtgctccaggccgactggatccatctcaacccagtgtctgaggccctccgcaggcagcctgccacacccctacaagctcaggaccctccccttgccgggaaggccctctccctccctctgacagccacgtccactctgccccgacctccagctcacctcctccaggaagcctgccctcatggccatggtgctgaccccatctgaaactccatggtccccctgccctcagtctgtgcctcatcttgccatcgtcatgtatgtgtgtgtctgtctgtccttccccatctcttctgagttcctttccttttttccaaatcatgtacttagtcctggactctttgaatgaaggatcccagaccaccaagggggttggagacaccactcaatccagtggatttcaaacctaaaaagcaacttaccttttaaatggaaaaactccttcccaaagcaaggcttcctgagaaggcagaaggacctggcggaagatgaggagaggccaggtccagcctgcggggcctcccccagggcccggaagggacatcaggaaagctgctccaggccagccctctgcagtggaggtgcattctggggcccagaacagccaagctgggctgatgaccctgccgatgcttgtagggactttatattgcagatgcaggtctcctggttccccacaaatggtccatctcccagtgaagtgggtaagaaagaccacattgcccccacttcacagatgaggatgttgagtctcagaggagtcaggtgactcaccgaaagccacacagccagtgacaggactgccttggggctcagcagaagggcgagggaccgtgttgaaggatggctgtccagctgtggctctgccgaaggccagtgcggtccctgacccctgggggctctggggactctcccaagctgagcatcagtgctagctgtgaaaggagagagaaaacaggctcccttttccctgttccccaggaacttgtaggaaccaggcctgagatgcctcagctgcctccaggccacccctccctccctccagtggccttcaggaccctccccaaaaggttaattaccgaatcatcacatcaaaggaaattgcacttttgtgccttaaagaccaaactgagcttcaacaacacggttatttatggtctctgataagcattttctgggtgtgattaacctttgtaccaaaatcacactgggaattgggatggcagcttcattccagccccattctgtgctggccctgggggtgggggtgtgggtagcagctgctgcaggctggagcaggccatgtcagaacctggccctccaccccctccacaggctcctgcaccaccttcctccccactgcccatcccttcttccaaggcgctgtcttgacctgcgcctggccagccagaaggcgcgactgtcttcatggttcatctccttgaaagacagcaggagcacctgcttactgaatcccagctcctaaaaacagttatgtgacattgggtacatcaactaccccagacttaaaatgggagcatgatacccgcccagtctgctttcccttccatcccagggacccactagatctttggaaaatccttttacgcgatgctataattgagccgcacgcacccttggcctcagactttaaccttgaaggtttggctctttggtgagtggagagatttgtagctcagctgcaagttttatttggagccttggggctgccaggctgtgcacggaagtgaggcattagccagtgagtgaacctcgtgctctgccagcttcagcttcagtgccgttttgattttctctactagttggaagatagtaaatcacatgaagtcttgaaaacttggttctgaaaggagcgccagtggctgggactggtgatggagtggaggagcaagaggcatctgagaaaggccaaaagcactttggtttgatttcagagaagatgacatgttcagttcaccccatttaccatatgcttcgactgtagttcccactgtttcagggtgctagttgttggtgagaagtggaggaagccaagaaccctccccgggaaaatggttttcatcacacacaccaactgcatttatttgcaaatcttcacactgcccgctccccaggttaagaattctccgtgtttttttttttgtttgttttgttttttgttgttttagcccagcatggtggctcactcctgtaatcccagcactttgggaggccgaggcgggctgatcacttgaagtcaggagtttgagaccagcctggccaacagggtgaaacctcatctctactgaaaacacaaaacttagctgggcatggtggtgcaggcctgtaatcccagctacttgggaggctgaggcaggagaattgcttgaacccaggagacagaggttggagtgagcggagatcgcaccactgcactctagcctgggcgacagagcaagactgtctcaaaaaacaaacaaacaaaaaaattctccattttaagggcaaacaaacaacatccttgaaactttgggggcctgttcctgccagtgcagtaattgcctattgatggagatagaaaaccacagtggagcttgaagaggagatcagcggatggtgaagaggagatcagcggatggtgggaggaaaaatgcaggaaatctctggacttttcatggaagtatgattcaggaataaggcagaagccctcacaaaccttccacagagcaagaggtggcacaggcacagattctgctacagagcagacctttccagagaggaaaggttggtttgggaattttaagaagcatttttctttgcataacacaacaccagtcctctgtgtttagaaaatgcctgtgtgaaccatcacattcaagagagggacacaagtgtcagggttctaggcagccaagggaagactagccctttgcctggaatttggcttcattttctgacgaatcaagatttgctctgctcctctgtgcacgccaggacattaagatgcgagaataagaacttatagcctgtatatttgccatctaattagtgtcttgggtcctaagtgctttgtgccgaggtggttttcgtgcacttaggctggtctaagtgctctacttgtgagcctcacgggcacagggtccagtgagtgtacagagtgcactgggaaaaggggtacattggagggagaactgagctgctttgctttggagagttgggcaggtactactaagcagacgggccctggaactggcctctgaagaatgcagagttgaccaggtgaccaggggtggggggtggcacagactgcatgggcagagggttgcagacatgagaaggatcccagatactgggagataataagtaattggatggaaatagaacgtgatgtgccttcaaggtgggggatatgaaagaccggagcacgaagcacagcttaaggattagatggataagtggaatgtcaggcaatggggctgatattgtcaaataggatgcctccagtttcttttctttcttttttatttattttatttatttatttattttgagatggaatcttgctctgttgcccaggctggagtgcaatggcgtgatctcggctcactgcaacctccacctcctgggttcaagcaattctcctgcctcagcctcctgagtagctgggattacaggcatgccaccatgcccagctaatttttgtatttttagtagagatgggggtttcaccctattggccaggctggtctcaaactcccgacctcaggtgatccgcctgccttggcctcccaaagtgctgggattacaggcgtgagccactgcacctggccagtgtctccagtttctttaatttgcctgtggctgagctggctctgggagccctagtttgagagagagagtgcgtgtgtgtgtgtgtgtgtgtgtgtgtgtgcgcgcgcgtgcgcgtgtgtgcatgtgtgtgtgttgggaggtggcgtgggcagatgtggggtgggaaacgaggagtgatgatttctagtccccttctagaaggggcctctggccctagcatctgaactgcccctctccggggacagagccgagcctgggtccttgctgattgctgagcgggcagtgcagctttgtcatgaggacactttgctttctgcttagctggcaagagccaccggctggcctggtggcctcctcctctctctacttctgcattttcacttggaaaatgagggtaaagatatttctccatctctctggagactgtgaagtttaattactgattaataaagtgttggaagttccttgaggccagagagatgaatgggtttgttatcatcactcctgagtcccaggcgcgagttggctaaccccagtgccatcttgctgagtgtcccgtgtgcagagcacaggatgggggtgtgaggctggggttatgcttcagaacctggagcttggtccagggcagagagacccctatgagagtatcagcaagggaatggagtgaacaaaaccactacaaactcaggcactaccgactcgggggaccatgtgggtgagtcatgggtagtggggaccaggctctgacgaggagtgggcactcttcccctggaccttggaatgtgggtgggagagaggggagcagagtctgccttcatcatcgggacaggactgggagggagtgggtaccgtgaaagggggtggccctggtggggtggggtgagataagggagaacattcccggcaggtgaacatggaaaaggcaggtcagtttgcttctaaggacagggagaaggtcctgagatccgaaaagggtgagtggagaggagtccagggaagagaggatggacacccctgaaggatatggggcaggcagggaaggagggatggcgtctggcttgggaagatggcgcatgagtggggcccgactggagtgaagcaagtgaggcatttcctcaaacctcagtaatcgcaatagataatttttttttttttgagatagagtattactctatcgcccaggctggagtgcagtggcgtgatctcggctcactacaacctcaacctcccgggttcatgcaattctcctgcctcagcctcccgaatagctgggattacaggtgcctgccaccatacctggctaatttttgtatttttagtagagacggggcttcaccatgttggccaggctggtcttgaactgctgacctcgtgatctggcctcccaaagtgctgggattacaggcatgagccactgcacccggccatgatagataatattttaatatcattactgactttttccctttttgccccaagctccaacatggcccagcactgttactgagcctatctttcctttaaaaagtttttgatattttgctcatcatggattttttggcgttaatctttattttaaaaaaataattatgttaaaatattccgtatcttgacgactaagattttcgatgctcccttcacttttgcactcaccacgtggagtcccggccccttgtgagaaggctgcgtgctgcctgggctggccaccgacctgcttctagcctacaccctagaagtctttgctcaccaagggacttgacaagcccctacctctctcccgatcttattttcctgctgtgaaatcccaagctggcttccagagctaagggtctgtgtctgaggctctgtagtgaaatttgcagttggtacagaaatgggctggggctgagacacctggccatggggttgggggcctcagaaggagcagaccccaagaagaaacagcagagaagtgatgaagagagcagcctcccatgaggcctcttgggcttcaggctcaccaccctgtctgggaactctgctggtggccgggcacccagctccccacagccagaggggaggaccggttacaatgctcccctctccctgcccctcactcatgcttcttaaccccttttgaatcaaatcccaggggaaacagccacctctcctccaacaactaaataaaaaccactaagttcagatcagttaacatatccttcttgagtgcctattgtataccagaaacacaatgtcattcattcatttcttcagcaaaattgagtgcaatgaacaaaatccctgcccctacagagcatgcagggctatttaaaggacttcaatatacatacatcatgtaagcttaaagggggtgggacagttagacccatttttacaggtgaggacactgaggcacacagaggtcaagtcccctgcccatggcaacacagcgtaacggggggctggacactcggcctctctcttgactctaaccctgtcgggaggggggtaatgcgatggttgtgtgggtgggtttagttgaagaaggggcagccctccaggtatagggcctatgccactcccttcccaactgggctaaaattctggaatcagagtgacacggagccttcagggaggttaaagggcaaaggtctgccctagaatagggtgtccatctttccagacacatttatcccttacctctctgtgcctcagtttcctcatctgcaaataggcacagccacaccaaccccagggaaaatttagtgtcacggcagatgtacaagagcttgataaatggtgaggcacaatttgtgatgacggataattaggcaggcggggatgagggcccaggaaactgttcacaaaacaagaggccagctctggaggaagggagggaagtgggtgcacgcatggggatgtcagcggccatggtggtcagcgggccggtgggagggtgtgtgtgtgtgtgcacatgcacacgtgtatatggtgggacaccgttggaccatgttcaggattccatggggatggaggtggcttcctctggccgcactcagcccctaggggtaaaattcgcctcttccctaggagcctgggctcctgatcctgtcagcttccctgccaggcccctccaagagcagggcaactccagaccctccaggcagagaggacccagaaggaggagagaggagatgaggggcgggtgtctgggaagcgactggatgtgtgacagggtgggaggggaggcaggcaggtgcagtgtcacccctgcagttttcctgggtgatggagcaacatgctcatggctcatggcacctgcctaatgtgtccttcgcctcctgtgcctgcaggcccttctccttctggggctgaaccccgtctctgcctccctccaggaccagcactgcgagagcctgtccctggccagcaacatctcaggtgagtcccctcaaccccctcctgcaagattcctggtcaccacaatgccccctaccccaggtatcccagagcctgcacacagaaccatgccctaaggcaggatttgcagagtcatttcctctccagggctccaagcaattcttcacatctccaccgagcaccgtgttgagaaggatcctgaggttgggtgtaaggttggtggggaagagctgcagttggttagtagcacctgccgtgggcacaggagtttgcagcaaggattctgagtagttatcttccctgctttagggtgggcacaggggagggcatcccagcccttgctgctgagttgtgactcggggaggcatcaagggacggaggaaaggctgccacagtctgtggctaaaaatgcccagagtgtaatactccacggtgctaacccgtgccaggcactgatcaaggactggagctgtattaacccaatttgcacaatgtccctctgaagtaggtgctgttgcccccattttacacataagaaaactgaggcacagagaactagtaagcgccagagccctgagtggagcacgggcagcccagcttctggttgtggtctccagcgccgtgctctgtatctctccaatagaaaagacagcatagctgcaaatattattaatatctgtgtcactttaccatttacagagtgcttttgcataatcatctcgtttcatcctcacaagagtgtaaagccgaacgtttgtacagttgaggaaactgaggcatgggagagttacataacccccatggcaccatgcagccagtacatggcagtgactgggacccaaatctaggtgtttgggctgcctagggcatgtcttttaaaactttgggtgtggtcctgggcatggctgctgctgggcaaaaatgtagagggtccctgcacctgagtgtctttggaaggcttgtggaccttctatttaagtcttcaagcctctccttgattcacaccaactctttcatgaagcacctactacacagcaggccccacgcagggcactgggggaaacaagggggctcacaggctagtctgggcatcgaatccacaaatgcatgtgcgatgaggcagagtggaagacgcatgcttgcaaagaaacagacgcaagtgtctgcaggcgggcaggaggccttcatttctgaaggtggctctgtgggaacaggaggcctccacatctcatggtagctgcttcctggccagctgttccttcactagggagagcctgggggcagaggaacaagccctggacttggagaaggcacactgggtccctgtgctggctcacagaaggtgcctggtcatgtcccggctaggggtgggtactggtagccacagggatgagcataccctcaacccctcttgaacctcaccttgcccaagtgtagagggataggggggctgttacctgccccaaggatctcttatgagattcaatgtactaagttttatggcaatatttcacaaactgtaaactgtagagtgtcatgccagtactaggtttggtttggtttggtttggtttggtttggtttggttttagagatgggatctcactctatcacctaggctgaagtgcagtggcacaatcatagtttagtgcagcctcgatcttctgatctcaaggaatcctcctctctcagcctcctgagaagctaggactacgtgcacaccaccatacctggcctaatttttaaaattgttttgtaaagacagggtctcagttgcccaggctggagtgcagtggcacaatcatagctcactgcagcccagatctgggctcaggtgatcctcctgtctcagcctcagcaggcttttaaaaggcttactgtatagctggaatgaagggaaggtacttgccagtaattgctcataatgatggagggagcagggaggctaagcaggggtggtgcaaatccagggaggcttcctgaaggaggaattttcaaggtgaatattaagagacactgttagtgtgaactggcagaagagagccagtgctgtgagctacagcaggaggtgagcaaggaccagtaggtgggtttggcggaaggaccaggaagaagtgaggagggaggagtgggaaactgagaaggtgctaggagcccagagcatctggctgcaaagttgattaagctgtgcttggggaatggggaagtaagttcttgagcatgagtctggagttctaggagagagagatggggagggaggagagctgctcagaggctgggcctgagaggaaaggccctatccccagctcccccatgctctggggccaggttaggctctatcaacttgttcgttatgctcaggaggagagacatccctgagcccacagaggtgtcacctgtccttggagccagcaggccatgatgaatagtgttcattccctagtgatttgtactctgtgcccgtgcccccagcccagcccagcctgcctggctgcatggcacgcgatccatcaccctccacgatggcctctgataatgggccatttgtcttctgactcgtagacctacccagaccgacgggcactggtagagacagatggtgagggtggggcggaggggcgagacagatggatgggcgtctggccggggcggctggcaccgagccagggcctttggggggccttttacgtgcctcatgccaggaactgggagagagtgggtccccagggctgaggcctatcatgtggcagaccacagacttggcgactcataaaatcatggcatgagtatgacaatgccaggaggtaatagaatcatttactaatgacatcacagacaaatagaatctgagttgacaattacctgggttcatcagttctcatctctggtcttaaggtagtgaatgataattaataatagctaggccaggcacggtggctcacgcctgtaatcccagcgctttgggaggccgaggcggtcggatcacttgaggtcaggtgttcgagaccagcctggccaacatggtgaaaccccgtctctactaaaaataaaaataaaaaattagccgggcttggtggtgggcgcctataatcccagctactcaggaggctgaggcatgagaatcgcttgagcctcagaggccgaggttgcagtgagcccagatcacgccactgcactccaatctgggcgacagagcgagactctgtctcaaataataataataataataatagctaatgtttatttagtgtccactgtgtgttggacgccattccaagtgctttaatctcattcaatcattacaatactgtagtcaggaatatttttatccacatgttacagtagaggaacctaaggctcagagaagttaaacgagttgcccaaacacactaagcagttactaagcagtggctgcaagatatgaaccatgtgggcctgacttcagattttgccaccagcactgcgttatgcggccactcagagccagggtgcccagggcccggtgtagtcagccgtctaggggtgcagggccacccctgcagttttccgtacccacactaggtttgcacacggccccatattctcctccatgcctcccgtatgcctggcactgtactagctgctgggaaggcaaaagtgaagagcccacagacccgcaggacaggagggccatccacttgtcctgatggatcacacaccctacagaggcagctttcttctctgcccagccaaacagaaggcagctcagccatgaggtggccgagcaaagaccaggtttctgccaccctctccatgccaggggacccaggcagaggccctgcccattcatcccacagcacattcctctgactgggcaagaacgtgtgtttcacaggctgcccttgggccacctggctgctccccctctggacagcctgggctgagtccccctcctgggtttaagccaaatagggggaagggcttcccagcctcagggctgctgccaagtgcccacctccagaagccgctgatgggagacccgacgtaactgggacagagccaggttccagcttcctctgaagttatctaccatgggcggatctgccaagcacacgtggaccctgagcctgatgggaggcaaactgtgtaacctacctgggggaagaagccagtgcctgattgtgcacagcactaaataggccaccttcaaagggctcttacctgttcactaaagcagccctcccaggaccctgaccccgcagggtttcttccctggcaggtgggagccctccacacctctcctctgctctccctttgcagggatttgaggcagtctgaccagaatgcgggccccacgcatttgttctgtgcatgtatggggaaggaggaagatataggaccggtttttgcattactttagtccacttgactccagcagctcaccctcaacctaaaggcctgtggtgacgtgcgctggtgagtgggttggttctactcccacatccccaaccaggaaagaccctgaaggcgtctacctgccccgtgaatgagggagtgagccacagagtgataggaacgttgacacctcagacacaccaagcacacacctgctccgggggtccttgtacttgcagttgcctctgctgagaacactttccccgcaggttttctccctcacttcctttaggtatctgcttaaatggtagtactttagaagccaccacactaaaagagcaccttggtcagcctttatctctttgcactgcttagtttttctccatagtatttctcactggttaacagaaatgtgtgtgtgtgtttgctgcctctccactagaatgtaagcaccatgaggacagggcatttatttgattcattattatatcccaagccccttgaacatttcttaggtagtcagtaaataacagctgtctcttggtatccatggggcactggttccagaactccccatggatatcaaaatgcagggatgcccaagtcactgatataaaatggagtagtatttgcatataacctatgcacatcctcctgtatactttaaatcatctctagattatttctaatacctactacaatgtaaatgttatataaatataaatagctgttatactgtactgtttagggaataatttcaagaaaaaagtctgtacgtgttcagtacacttttccccaaaacctatggatatggagaaacaattgtacttgttaaataaagagcaaagtcactattccttaatttaccaggagaggcactgtgggacagggagctgtcaggaggcgttggcacctaacagcacctctgcctttgtgcagctggttggtaaattggcttggctatttgcaaggttggattactctggtccaaattcctcctcatcccaggactatgacggctgactaggaaaatacctccacctgccaggagagcagaagaggtcaggcttttgtggcactggaactggagggaggggacattggcccaagaatcagaaggtccttgaggtccttatagccatcaagctgagtgacacagaaactgacttcctgccttctccccttcccagctaggggatggacaccctctctgtccccagcacctgtgaaatgatctttctctctgcctccttctcctgacttcccgcctgcctctctctatttcttccttagtgtcttcttctccatcttttttcctctgtctccccacctctctcctggcccacttggcctcccacctccctccctctccgcctgttttctcctcctcattgtagtcgggtggcttaagtgagtggcatgaattaagtcgtgcaaagcgcctttcacagtgcctggtttatattatagtagtgtccaacaaatgatccccctcaagtcctatacacaatttcccttctgaggccttgtccctacagggtcccagggaccccccactctcctcctccctggactcggtgtccgcaggacgcaggatggcgatctgaaatccttgtggggatttcagacacagagacactaaggcccagagagggcaggaccccgaggccttatgggaaggcaccgagcccacaaggcagtcccaggccgggaagtccagtggacagtgccaactggccaggagcgtggcgcctgaggccctgtcctgctgcctcctccctgcccttgtcctctaggtcccctcccttctaactccactccgcggctgaggcccttgtgcctttggccacgcccccccaacccccccaccccaagcccctgacccagcaggagtgctgacctttcactcggggatgggggaactttgtctcctcgccccttttcatttggcttctcctccccgttcttgcagcctctctctccctttcttcccgtctcacacagactttcaattccttccaggattttctgggctcttaatgtaaaggttgccactgatgctgtgtcaccagcgccccctctgtgcatccttaggagctgcgggggccaggagggagggggaggcgcggcgctgctggggaactggagtcgcagctctgcgatcgaggggcagctgcccagatcagggccagctgtgcagctgtgggcagcaccctgagctggcatcaaaagatgagcattgaaaatccactcccgtctcagtatctttggttactgattgtcttggaataagccatttggccactttggcctcagttttcttatctgtaaaacaagagagaattagcacccatggtggcatagtaggtggctgggaaccataccacagaggagaggcaaatggcttctgacctgcccaccccttgaaggtctccacgtccctggccagctgtggacggggtcaggcctttggttagagcccttagagctcccagcagtggccggtgcaggtcctctaggccagaaggcctatcctcagtggctggcctctgcctcggctctggcctcgtcctcgctgtggctggtgcccagtcctttcctgccctccgtgccttgcgaaagcaaggcgcaagaagactggccggggaagggaagaaggaagggcaggggccacactgatggagagggtgtgttccgtgtgttcattccagacgctgccacatggaacattacatttgagcctagctccctagatactgtggatgaatgagtgaggagtgaatgagcaagctactgaggaaggtacaatggtgccatcaaatgagatggcatatgtcatgtgtgcctggcacacagtcagccctcaacaaacgctgctcccttcctgctgcccccttaactgtccctcaccccctgtgactgactcacaatgtggcgtaatggaaagagcactagatttagagtccaaaatacctgcctatgagtcttggttctttcacccattgacccatgaccttggaaaagtcacctaacttccctgggcctcaatttcttcatctgtagaaaggaaataatcacattgccagatgtgtgaagatcaaacgaaatagatgaagagctttgaaacagcagggcatggcctctatacgatcctcccctccccgcggccacatgcccccagctctccccccaagtccctgtccccgggctcaacactgtcagcaggatccaggacaggttaaaatcccagcctggtacgccgttgcctcggatggccctgcttcctcactccactgcttccactgccactccgtgataacattcacagagactccgctgtggcacagcacctgggcttggcccactccagccgcctggcccgcagtcacctcgtgtccattaagccccaatccagatttctttctctttcaattaagccacttggcctgaaggcctctgccccttgagtcctgatttgcacaaatgccctatgaatagctttgattagaaagaaaaaccagcatttggtacaagtgctgctcggcactgccctgagtccagcagagaaagggagccaatttaacccagtgctgtttcctgagagactggggacaagctaatttggggaggagatggcaggcagccccagccagggcttggcagctgctaaggcctcggcccaggcctgaagaggctgcccccacccgcgctggttcatggttcctggccctccgtggcttgtctgtccactcttgtgccaattctaggttattaacctggggacgctggggctcaaaaggactggtgtgccagcctcactcagagggaggcactaaaaggcagagaaagaaggggcagcctcaggggggtgccaggactgtgccagcccccaccctttggaggccatatccaccctcccctcctgaagtgtcactgggcctcaactctgccccaacatcctctctctctcgcgtgctctctctctctcctccaaaaagcaaatatttggaaggggactttgacatcttctttctagttcagatgtttatatctctttgagtcacagacccctttgagaatctaatgaaagccgtgggcccatttccccattaaaatgcatgtatacaccaatattttcattacattcagggtctctgtgaactgttgaagtttccagtgggctgagattaagaatttctgatctggttcaaccacttgattttattatagatgaagacaagacactaaggcccagagagggcaagtgacttgcctatgatctcacagcaaattagtggcacagcccattctgaagcagtgggcccagactcccagttcagtgtgtcctttccactcccccagatggtgtctccctccctttcctctccatagctcacttctccatcagtcctgtcctagggccctgcacagaacaaagtacacgtgacgtatggtcagtaagtgtggttaacaggccagccggaagaaggaacaggccagccggaaggagggccaacttcatttagctggttcttccctgcagggccagggtagatgtggtttgattgcagagacgtgaggggctaggtcaatgattggatgggtctgttccagggtgttcctgtgatgtctcagagctccaggctgtcaccatgcgagggaccaagagaaatgtccttaggctacagagcaggggaccagaagcatgcccaggcttcctctaaatgccaggaccttagcttttaacaccacctccagccatctctcttcctcctctgctctggaacccatgctggctccctagtaccatagcatcagattcagatttcttccccttgtgtcttctttgcataggatgagctcatttctgcactggtcattgttgtcatcctgtccctagctggtgtgagtgggaacgagtggggaggtgggcagaactctggaatgtcctctgcctgcctggggccttggccggaatatcttgctgtgccttaccagccgtctctgcccccaggactgcagtgcaacgcatccgtggacctcattggcacctgctggccccgcagccctgcggggcagctagtggttcggccctgccctgcctttttctatggtgtccgctacaataccacaagtaaggaagaagtggagggtggaccatctgctgggaggtgggacaggatggggagagcttggaggtgggggaaggaagaatgacgatgacaataacagtaatacctcttgtggggatcgcccctgttttccaaaggtcagcgctgtattctcctgggtgccctgtgctcattactcgtgattctcacaatacccttgcaagacactatcacgcctcctataactaaggcccctggggctcagggaggttagggaacttgccccattggcagctggtaaggggtgaagctggaatttgcatccacgtctgtctggcactttcttctgccattaaacttcaggttagtctgtttgagcctactgtaataccatgaagcagggcaggaatcatcagagccccagctgtgtggataagaaccttgagtcgagaggttctgaggcctaccccgggccctccagccaggaggcaggggacccagagtttcagatgcacaggcacaggctcaacttgcatcctggatttgtaggtggagtggggcaggacggggttgacccagggtccgtcggatacttgatcctcatgtaaacacctggctccccggaggcccgaaacccccagagccgggctttgcagctccctgctattccgcctgccactctcagaattaatttgctctcccctgtcaatgagtcaggtccctgcttatcaagagagcaacagctgtgggccaggagccctaacctacctgaggggaggctgggggcctgatccctgcaattggggcctcctgtgtgcacccttgggtccttgtggtcttgaactcagagtcccaagagggcacaggggtgaccccagacaccatgtagtttactccaagactcactgtgtgacctcccagcacattgttgcccctcatctggccctcagcccctcatctgaggatggagagggctggatggcttggcttctaagatgtcttccagctcaaaactcccagattccttctcctgcccctctttcctctaccagatggatttggggtgttaaggttgggggctacagcagaggagtaggaagacccagccagaaagtgactccccagggagtgacttgggaggccagggcagggcaggaggctggggcagccagatctagcagcctcgtgtgtctgtaccatgtcctggccatgggagggactcgggagagggagaagacacactgggaggggcttgggggccaaggggaggaagtgcagaaaggaagaagggcctcttggccaggtcagtccaaggggtgcacagttggccagcccccaatatagtcaggcccattttgtaataaggaaattgaggcacagagaggctaataacctatcggaggtcgcagagcctggaggcagggtcacagcccagggacgataactctcacgatgctgggggctgagtccttctggagcccctaggaaggaggcagcatctgcacatcagaggagcttccaggaggaggcaccagttcctgtcatgtccacttccagagtgatcctcgtgtctgggcctacctgcccccactccccagcgccaccgcccagcctcaggtttcagatctgagttggtcactccttcacttggaatccactcttgtgtggcctccgtgttcaggctgctgggtggggccggccaggctgtctgtggtcatggccctgtcaccttttgcagccagccctggccaccccccatgagttccctctgctcccgttgcaaagggttacttgacatgctccagataaacattttcagccctcagtgcctgggcacacacttcacttgctggccctgcctgaagtggcagtcaggagtcctaagttctcattccagctgcatctgcagctggttaagtagcccttggcacagaccttctgtgggcctcagtttctacacctgcagaatgggtgggaggattgagtgtggtgctctctaggctcccccgcgcttccctctgtgatcccaggaaaggagctggggtgatggcctctcgacatggggtcagtctgtgagggcagggctggaagacccctttccaggtgcctccagcgccagatgaggtggagcacgcccgtgcagtcagtcacaagtgggtacccagagctgcacctcatcattaagcactaattaatccatttactgatgagttgctgccaggacttctaggccttctccctaatgccctccctcctgctgcatctgatcttcaaagggatgggaagcatttccaaaaatatctattttcccgtgattctttgggaggactgctcagctcagcaattaaagacaaggaggtcgcatcctccctggcaacggctccctgcttgttgggcctgctttctttcggatcacctcctttctccccatcaaagcctgctccctctgggtgacccagcctctgatgggactcctgactctcccccacatgtcaaggggggcaagggtgttgagcaaaggggctcctcccacaatatccaccggtaccctttgatggggacaagggacacgctctgagtctcacgtaaggttatataaatacagctccaagacaataataataaaaataacagccaacatcacatcccaggtacagttccaagcaccttgtgcatgttaactaaaaatattcaactcttgatgtaggagctgctattcagatgctctcacttacagaggaggaagctgacaggtttcgggatttgcccaaggtcacacagctagtggaaggtagtgccaggatttgatgctaggctgtctgggtccaggggccacgctcttagcactactccatgctgcctcttaactcaagaaccaccccaccccatcccaccacccccgggccccttccagccggatctctgtcccaaagcccctatccctggaggtatttcagagcagcctctggtcccgggacttcccagccatggcagagacactgggttctcctggttcctcgtctcaccctcccctcagacctccatgaaaacccactgggtgagcctcagccctccggtaactcatccctcatttggcaattggagagaatcagcagcctcttcaccaactctgcccaagggcctgaggctccctgagactgcggtccactgacatggaagagctttaaaaatccatttagccggatctcccactggccagctgtagctgagcagctcctcagcagggggaccagggaggggaatggaggggagtagtgcgagagaggagaggccacatctacttttcagccctggaggccaggacgggctccctatcatcaccttcttgtcctgatgctgccagcagaggcagctcccaggctggatggtcccttggggatcagccagtccagcccctccttttataaatgagaaggggtccccagaggtcagtcatgcagcacggcagcagctcagctggtcccagggccccagcctctagatcctcacagggttctgccccatggggctccagactcaaggactgggactccagcagctttcccagccctgttccagccccaagggccactgggagccacaggaaggcctacagtgagggaggctccaggtgagatactcagggcttcatttctctgccaggcagttctgcctgaagtcagagccagaattaactcaaggaagcatgtttagagctactgatgccagagccgggtacagggtggggtggggcaacctctgccagcagagaagatgagatggaatttagaactcatgctgtcccttcccagctaggttttaggaccctggagctcaagcctggtaggcatgaaggagggtaaggggaacacgttgggcagctctgcccatcccagggccctaaagccaggcatcaattcagctcacagggcccctccccatctccttagaaggtgacaggttctgggccacagtccaggccggtggctgcacaaacctccatatgtccctgggccactctcctgccttcctccttgagtgacatgggccaggctaggagcagctgtgacagacctgagtgatgacatctccccacacacgcctgtcacctgtcattttctgtccaagagacctccatcatggatgttccccccacccacagaccacgaccttccaacacaggcccgctatggggaagtgccaccatgggaccttccccaagatctgccccatgaactctgagcccccatgtgcagcacagccagtgaggacctcccacggcagcctctgtgagatggctctaccacaagaagccctgtcatgccacctgccacgcgagctgtcaccgcatacccttccaccgtgtcatgcccagcctcgtgaaaattgactcatgaggtctcaccatttttttgctgtcttgcaagacccctgaaaaggaggtttctcagaagagcacctacctgtcatctgccaccacataatgacttgcaaaggaacagcccagggagctggtcaagcctgtccacgggccagtcctgtgggcatgatcatgccagcttgagtgggcagagccgggggagccaccaacccctgctggaggccaatgggcagaaagaggcaggaagatgggggtggtaaggaagcccctctccccagcttcactacacaacccccagccagcaggtgcctgtactggccatctacagatccatctacgcatccatctgggccaggatggtcaggcaggggccggggctgccccgccatcactgcctctctcttccttttccagacaatggctaccgggagtgcctggccaatggcagctgggccgcccgcgtgaattactccgagtgccaggagatcctcaatgaggaggtgaggctgagccgaacaaggctgcccatatggaggggagtccagggtccccagcagagttttgtgcctgctacttggagtcagggagccagaggctaatgtcaaggccctgctctagtgaagctgactggggagctggagctgtcaacttggaaaaggcccatgagcaggcgccctcgagggcaggctggaccaatggggcactgggcaggctctgatggtgatgagatgggtccttgttccttccttccttctccctgtgctcatcgggcagccgcttgcactgggcatgggactgtcctgggggtgcaaagggagaccagactcggtcacaggagtcccacccttttccacaacacatgcctgagagatacatccagttccagccacagggctgtatgggaaccagggacgggatggaggtagcaatgcagtttgaaaaagcccttggaaagccttttaaaatgttaaatgtttttgagcagatattgcttacacagaactcagaaggtacaaatgggaatacaatgtcccctcccaccccgtccccagccactggattccctcccagaggcaaccattttgccaatttcacaagtgtccttccagagacattctccgcatacacgagtaattttgtatacgtattcttttttgtttttacctgaatgttgcatgttatacacactgtctacaccttgcttttttcatataatcatctatcttagagatggttccatatcagtacataaagagcatcttcattctttttgcatttgcataatatcacaaaatgtaccataacttatttaaaccagtctttattctcagtctttagttattacaaatgctgctgcaatgaataatctttgaagggtgatatttggcagaggcacaaatatatctatcctggagctgctactgccctcggtctctcccaccctgtctcctttcccacccgacgctttctcccttggtgctgaggaagacgggggtcaaagggcagaaggggggacaccccaggctgtccgagggccagggattctgattgtggaagagagagactgacctaagcagtcccaagaatcccgtcgcctaaagatcagagctggaagggaccgtggagactctccttcccatcttgaggttcccagacttgcctggttataagaatcgtccagaggcagatctcctgaggtcaggagttcaagaccagcctgtccaacatggtgaaaccccatctctactaaaaatacaaaattagccgggtgtggcgggattacacctgtaatcccagctacacgggaggctgaggcaggagaatcgcttgaaaccaggaggcggaggttgcagtgagccgagattgtgccattgcactccagcctgggcaaaaagagcgaaattccattaaaaaaaaaagtggggccaagtgcggtggctcacgcctgtaatcccagcactttgggaggacgaggcaggcagatcacgaggtcaggagatcgagaccatcctggccaacatggtgaaacctcatctctgctaaaaatacaaaaattagccgagcatggtggcgcatccctgtaatcgcagctactcaggaggctgaggcaggagaatcacttgaaccagggaggcggaggttgcagtgagcccagatcgcgccacagcactccagcctggcgacagagcaagactctgtctcaaaaaaaaaaaaaaaaatggaatctcccagagcacttgtccaaatacaggtcccagggccacacccaacacctaagaaatcacactgtccaaggaggacctgggaatctgtgtctttcatgagtgccccaggggagccttctgatggggcaaaagtgagaaactctaatctagtgcgacccgttcacactacagatggggagactggggcccagaaaaggggaatgattcacacaaggtcacgtggctctgggacattccctttttttttttttttttttttttgagacagagtcttcctctgtcgccaggctggagtgctgtggcgcgatgtcggctcattgcaacctccacctccctggttcaagtgattctcctgcctcagcctcctgagtagctgcgattacaggaatgcaccaccatgctgggctaatttttgtatttttggcagttaagtcagagcccggaacccagggctttggagcccaggctccggagcacaggctctgcagcccaggctctgctttgcccactgccaggtatctggcgtgaaacaaagttaacggggaaagaatcactttccttcacctgtagctcccaccccggcctggcaagctttggttagccccacccctggcttcctggcctcaagtcactgagctaatgcggggctctgctgtctccttccggaagctgcagctaggtcaatgcctagcttaaaagactcacgagttcttccacggtgctgctctggcagggcgaggggctgcctggcatctcagatcccacaggccagacctttgggtggcactcaaggctggggtgggttggtcaggctccctgatgatctgatctgagcagggaaagccctcagcttgctaagcccccacacagagagcccacctgggaagtcctgggattgggaggagggctcctcctggactgggggaaggaggtggggttccaggttaggagacttagttgggccagaggagatggccttggccttggctggtggggtgggagtgggcaagaccgttcagggatgtgaggagcccgtagcctggcacacagtagaggaggtgggaggaaaggaaacagggctggtgctcagaggagcgggtcagtgctgtcagtgactcaggaccacacgccattgcagagagggatggtgtccaggaggcacagctaagccatgaggtcaggctgcaggccgcactgtctgtcccagcttcacgccctgcactcaaccctcctgagggtcagcgcggggtcttcgtggttcacctgtctctcctgctctattgcaagccccttcttttcagttggctgatggggacactcggcagcccccattttccccagcacccttcaaaggcctaagggcagtaggttagccaccctcagcctgccctgcaacacccaaccctgccaggacaggggtctctacctctgtccaccagcagggttaggacaaggaagaggatcgggagcccggtctcatcagccccctctttgcattgcagtgggaatagcacggaccttagggtttgggtttcaacgggaacctgctgcatgaccttgaggaggcaacttaacctcaccaagttcccaaaaatggtggccaggaattcagatctctgccttctggggatggaagggtggtgttggcctgtcttggcctatgggagacgttccattcacctgccgccccctgtctctcatctcccctgtgaggtcaggggaggttgtagtgtacacctgggggagtgacccgccccaccccccagcccatccgtgcctggctctgccatctctttcctctgcagcccctgctggcctggtgcctagcactctgggtaatcgattagtttaattagtgaaaatgccattcccttctgccagcccccagcctcgccagacccctcccagaactgcagaggaaagtatccaattaattgagtggtaggtttctcagctctgggcctgggctaagccctaattaagctccagcgccctggggtatcgcagataatggattcgcagaagtctgcctgtgaaatgggacttgcgagggcacctcaaggccaggcaccccaggagatctgcccgcagccagcaccaccaggggacaggcccccaactgttgcatgcatggctggccgggggatggcactgagcccccagcaccacccctacacctgctgcctgtatcagcaccctctcctccccccaccacctcccgctactactgttcactcccttccccaccgtccagccttcccccacccacccaacacttgcacacactctatcccctttccccacgttctgctgcgcacaggagcctgggcctcaggcacagcctgggagagcacaccgtggtgggacatgaaacggattctgggggtctggtttgtggaccaaggttcactgctcaccgtgtggggagaggtgagtggtggttggaccagggcttctgaactgcagaggtgctttttcctaaaaccaagctccgattccatgggcctggcgtagggcatacattccactttcctcaagatctctgcgtgctcctctgcgtgctgttgctgggccaggggccaccctttgaggatcgaggggctggagtgagtgcccactgcagggtaagaggagtagctctggaagcctcggtggagaggacgtgccagaatggagtgggcaccagtggggagcttggaagggaggtctcattgccaccaacccagagaggcatcaggacggatctggcactgcagcgcctgggacgaggtggtgtcctgcagagagtccagtcagagtcagccgggcacaaattgcttattcaattcagatcactgagggtacagcggagtggcctctgccaagtaccacgctgtgccaccctccttagggcggggtgcctgctggtcttaggtctccagactggatggagatggagtgctggtcagggcccgaggggtagctgtgcccatttgtccttcggacatcccagctgctttgctgttatcgtggccatcggtcggggtgtcactggctgtccctgggggtgctgctgactctcctctccaggtatcactggccacctctcagggtgttcctgggtgcctcttaaggccttgctgtctctctaaataatgctggccagaactctggttgttattggaaatgtcacagtgtcactggcttctgtctgggtgtcgcaggatgtatttgtctcagggtatcagcagccatccctcaggctgtctctccagctgtcttctcaggttgcatgatgctgatgtggccgatgagagacagggcttgaacccggcccaggcccgactgctcagggaggcacactgagactttgtcccccgggaatggtttggcctgattctccctcaggctcttggaggaaagccctcttgggcgctattgtcccagcaggaggtcccccgaggctcctgggcccaaagtggcgtgagaccaccccagagagtgcctctgctttcaattcctgcttgtcccccaagaaatgtcgcagggggccggacacggtggctcacgcctgtaatcccagcactttgggaggccgagacaggtggattgcctgagctcaggagttcgagaccagcctgggcaacatggcaaaaccccatctctaccaaaaaatacaaaatattagctgggcatggtggtgcatgcctgtgatcccagctactcgggaggctgaggcaggagaatcacttgaacccaggaagcagaggctgcagtgagctgagatcctgccactgcaccactccagactgggcgacagagtgagactccatccctcccccacccaaaaaaagaaatgtccctgggaaacagggaaaagagggagcttaaagccaggcagacccaggttccagataccggctgcgtggcgtggccaattattcagtctcttctacggaaagaactgggatgagaggagcagtctccaagggcagctgtgggaaagaaaagcagtagtgtctgcagagctcctggcacagtgctcagcatacagcaggtgcttaacagataactcctccccactccaaccccaagggcccggtacggcctgactcgggagtgaggcaggggccctggaaggcagcactgaagggtctccctgcacactgcgctcctcccgcccagcctgggcctgtgaacaaggccggggtgttgtgccatgctcggctccacacatgttgtcattacccaggggcttctcagcatggatttgtggaaaatttaataaaaggatattaaataccagctccagacagcctgtgatgtgtgcatatcttccagagcctccggattggggcatctgccgccaggcgctcagagaccctccccacttgcacgggtgataggtaccctctcctcactcccctctctgtctctgtctctcctgccttcatttacacctccctggatcccagtcctttggaggctggagttgctaatgtaattacctgccatgttccagaaagcctctgcctggaaaccagctccaagaacagagcttctgtagcagcctctggagaggttccccaatgagggtgctccttcagaagggccttggtggccgggcggaactgggcatagggtcttggcttctcctgggatgcctggtggatagggctggacttgatctctggtttcaagtgggttctgtaattccaacaggaaggcctatggagtagctccttggtggccagtagctccttggtggctctgagaagatgatgagggccgttgtgagctcagtgcttctcagcacttgcatgtcacccacctttgtgcctcatgatcattcagccccaccctagagcctagaaacggggaggaagggaggtgctgccagggacccctcccttctcaaagtcacctgcttttcaacacccgtctttccttccttcactcacacactgctttggagtttgactgcgtgtcaagctcttggtttctaggccctgcttcttgggacctataaaagtgtggatgggggctgtgctcttgggcaggagtctgcagagagctgggcatggggactgaccagtcccctattatgggctggggacctgtttgcagccttcgtatcttgccaatctacatgtgcaggcaagaggtggttttggagttgcgcgacctccagcgcgtcagcataacctctgtgctgttttcctacaaaataaagcccagatgcctttccggctccccccacggggttgccctgatggtttaagacgataacagatatgaaaatcctctgtaaacaggaaggcttcaccactcttggaactcaagattgctgtaaagaggagggcagtgacctccatcagtatattaaattcattaagaaataaacccggggaatgtgttacagtcctgggagaggatcctgctcactctgcacagatccccttgggccagggatggggaaggccccggaaagtgagttatggagcctaaggagacttttctgtggtttctgctgattctgtggatgccagagcccagcagctgcccggcccctccttgctcacaggagcaggcggcattgcagcaggagggatttaggggtggctgagaaggtggtgactcagttcagaaatgggtggagaagagaaatcccgggagtttggtgtctctgcagctgagaggtccacctggtgttctgactgggtggcagcatgggtagatgcgagtgtgagggcgcacctgtgagccggccacatgtgcttgtgcatgcaggggcggggggtccatgtggagtggggaatcccacgtgatgtggagattcagttcgacacaacacggagcagtgaacacagcatgggattcagggacaagcagagcctcaaactggacggcgttgctctgcagcgaggcctgaccctcagcagatgctcaggaagggggagtggcccttgagatcatgacccgctccatcccagccacccctaggcgatgtcctcacagagcagctcccatccagcagaaggctcacctctgcccctctctcctgctccagaaaaaaagcaaggtgcactaccatgtcgcagtcatcatcaactacctgggccactgtatctccctggtggccctcctggtggcctttgtcctctttctgcggctcaggtgagaagaccccagcactgcctcctcctgtccccaggacctagagcagaagcagggtggagaagtgagaggagcagctctaggttggggtgggggttgctgggagagggtggcaggggctggcttatctgagagtctcaggtctctgggaacctctggcagagccgctctgccctctccccagtagctgctggaatggtggggagggacaaaacttgtcttatgtcacccatacccaggccaggctgcacccattggggtgaccaggcagatggagccctggaggtgggggctccatggagtggtgccccatttcaggttcgaaggtacctgggccccagcactaccgccaaggatgcaggtggcagagccggggatggggatggttgggatcaggagccgagcatcagggctggaggctgggagcagggctgacccctgtgactgtccatcctggaatggcagggagtagaaggcacccccaggggaagcgggcatcgccaggaatccagctgccctggtccatggagcacaggctccatggagtgccaggctctgcctggggcttatgtctgggtgggctgcaggcaggaagggctccatggggcattaggagagcctggctgtcacctccctgtgtgacttggccagtcagccccaccctgtgcctcagtttcctcatctacacatctgggctggggtgatggaggtggcctacccctcatcctctctctcctatcgctcccatcatccacccgccctgctgcaccaggagcatccggtgcctgcgaaacatcatccactggaacctcatctccgccttcatcctgcgcaacgccacctggttcgtggtccagctaaccatgagccccgaggtccaccagagcaacgtggtacgtcctggcaggggagcggggagcaggtcaggccaaacccaggtcagaggaggggcccgcctgccctgcagaggaggagcccacagaacaggagtgggatcccagggtatgccctgtcctgccctggggaggcccaggcccagggtttggtgcctcccctgccccccatcatcatctctggttgggggtggggtggcagggctggtgcaggttggtgacagccgcctacaactacttccatgtgaccaacttcttctggatgttcggcgagggctgctacctgcacacagccatcgtgctcacctactccactgaccggctgcgcaaatggatgttcatctgcattggctggggtgagctgggcagccacctccgcagcctgggcagtggcggccgccgggctgccctctcctccagactcaggccagcgggctggggggcctgagggatggaggtcgggttggggcggtaaggtgtgcacgatagccctctgctcctcttgggggtgggcggcagtagaagcaccttgaaggaggtgtgtgagtttgagatccaccctgagtaaccccagaccccctggagcctgggctgcactggggttctccaggcccacatcctccagcccccgctgagggctctgtgacagcccatctctcccccaggtgtgcccttccccatcattgtggcctgggccattgggaagctgtactacgacaatgagaagtaagtcatctcctttcccttcctgaccccaaggtttaggctcccagcccagcttggtgacactccccacgggcattggccatgctggctttttcccctcaggaccatggtttctgcatctataaagggagagatctgggggctggactcattcagagggtccctgcctgtgctctgcctgggtggggctggacaagcaggacccagcctcctttatctgccttgagcttacacaggaagcagcctggagccagactgcccaggttcaaatagctgtgtgaccttggacagattactgcagtgtgcctcagtttcctcatctgtaaaatggacatgctactgcctcaaagggctgttgtggggtgatatgagtccatctatattaagcatttacaatagctcctggcacatagagtaaatgctatataaatgtgttctattaatattaagacatcttaataaaagagtccatgaatttaatagaagtggacctagatgatctcagaggcttcttccagctctgaagttctttggttccttatgaaatggactcagatgtttccaatcgtgcctggaaacttgatttctgctgcagtggagctcaactttggctcacattagaatcacctggaatgcttggaaaagtaccaatgcccaggcttcaccctcagagaatcaggcgcaattggtctggggtgtggcctggcacccagaagtcttgaaaactgcccaggtgattctaatgtgcagctcagattgagaaccactgagagagagcggggcagaaaaggtggacatggatggcaggggagccccaggaccttctttgccaaacaagtgttcccgggaacctatctgactgacggacctggcgtccaagtggcctccaccacagtgacaagccctctcctcccccatggaggggagggggcctgcacggcttcattgggctgaaagaggagcctggaagattatcttgcgtgtttatgtggttgactccctcctgactccctcctgactcaggctggctgaggccttgggaagccgaggcaggcggatcacttgaggtcaggagtttgagaccagcctggctaacatggtgaaatcccgtatctactaaaaaaaaaacaaaaaaacaaaaattagctgggtgtggtggtgcgcacctgtagtccagctactcgggaaggctgaggcaggagaatctcttgaacccaggaggcagaggttgcagtgagccaagatcgtgccactgcactccagcctgggcaacagagtgagactccatgagtctcaaaaaacaaagcaaaacaaaaactgtttgggctaaaggagaaagtgagtctgagcggctctgaagctccacctggtggtgagaattcaacactgcaacagagaggatctgggtgtgagaaaccagaaacgaacttgcagaccagaggcccgaggactggacccggccctcagaagctgtgtgtggccagcatggtgttttctgaaaactcgattttgtagccaacctttaaaaaatccaaaggtttcatttacaaatctcaatttctggcttatgagatctagccactgggggtcttcatacccacgaggacataagggcttccagtttgtctggtccccacccactccccaggcctccctggagcttgctggagggacactcgggaaaatgcaaagcaattacctcctgcagcaggtagaacctgtgcaagtattcctctcaaggttgttcaggctgtgactggacagattcaggctgagattgcgaactgctgggtgcccactgggtgacaggtgtggtgccactcccaccagcagccccccagagggcatgctgggcctcagccactgccaggggactgtgaggaggagccgaggatgcagcctcgtgttgggcccatgcccgttcccagccactcagtctttcccactgtctacctcctgccccggtgcttgcttccaactttgcatctcatcacagaggatgctgtccgttctgcagatgggacaattgaggcatggcagtgggatcaagtgacttgaccttctgccagggttggaattgggacatctacctcttggcctccagccccagtcctgtcctggccaagcactgtccctccccatgccatcaaggtggacgcagatgacccttcctcccctttcctctgtggccttctaggtgctggtttggcaaaaggcctggggtgtacaccgactacatctaccagggccccatgatcctggtcctgctggtaagaacctgggtaggggcaggagacagggcccagtggggaggggcaatcagtgccaaccgtggacagaaaggacccctctacctagaggtgggggccacccaaagagggggcatgggtcagagatgtgcaggtgctcatgaggaggagggagaacagcaggggcactgaggccagagctgagaagcctgggtcccaagcctcttgcacactccggcccgctggtgtgctcaaattgcagatcaatttcatcttccttttcaacatcgtccgcatcctcatgaccaagctccgggcatccaccacgtctgagaccattcagtacaggtaaccgggtaccaccttcctcaggcctccccctgatgaaacccctgctccccatgcctctcacgtgccagagacctgccactccctcccccgacctggccctctttgccgagccagcgggcagcccgtcctggggtgggctgtgactccgagcctccccacccgccccaccccaggaaggctgtgaaagccactctggtgctgctgcccctcctgggcatcacctacatgctgttcttcgtcaatcccggggaggatgaggtctcccgggtcgtcttcatctacttcaactccttcctggaatccttccaggtacagccctggagggacacatcagcacctccttgggtggggattctgccaagcagaggcctggagggcaggaggccagggagaagcaaggggcagcccagaggctgggtgggcaacacctgcagccgacctttgacgcctcctctctcctccccagggcttctttgtgtctgtgttctactgtttcctcaatagtgaggtgaggacccgggggccctgcagcggggttcagggctgtgaggcctgttgggactggcgattgtctagagccttctcctcccctcccagggctgcctctctccctccctgctcctaggtccctaggggtatgctgctgggagccccagggtggcccctcccacctgtccactcccacagtgacagccccctcctttgctctcagaggccgctggcaccaggctggaagccagagctccagtatctttgatgagcctgtgaaaaccaggaagggctgagccctgggcaggggatacatgtggtttgagggcagggagccttcatggcaaagggcatatggtgccttcgtgtgggttagaaaagggtgccccttccccaggacatttgagaaacctgttccgacaaatatgcaaagcagtcgtgttaaggctgtgagtgtcatcccctactgaggacttccatgtactcagctgacctgcacagccgcttacctgcacagctgctcgtggcggcccaggggagggagggggtcctgagccacaggctcagatgtcgtgctcctccctgtgcccacaggtccgttctgccatccggaagaggtggcaccggtggcaggacaagcactcgatccgtgcccgagtggcccgtgccatgtccatccccacctccccaacccgtgtcagctttcacagcatcaagcagtccacagcagtctgagctggcaggtcatggagcagcccccaaagagctgtggctggggggatgacggccaggctccctgaccaccctgcctgtggaggtgacctgttaggtctcatgcccactcccccaggagcagctggcactgacagcctgggggggccgctctccccctgcagccgtgcaggactctagctcatgagtggaaagtcacctacaggactgggccgggcccagggcctctggcttccctgcccaatcctccctggagaagggacatgggaatgaattgaaatggggcgctggacacctacagcagcacgcatgtccctccaaggctgtcttctcccagagcacaagaaggccagcccactgggccctggggctgccctcggcaaccgtggggaggccatttgctgccctggggcatcatgggcaactcgtgacagcctctgactcaccacgatgacgcctctggacctcggtgatgccttccgacaccactgggaaccaagggccctcactcaggaaccctggagacagaagtcaggtgtcatcatcagacttgcggccacagcactagagtcacccccccaggcctccagaaccttactggcactgtggcactgccaccagcaatgccctgccttgctgccttcaccctgaacatttagtaccctgcaggccaggccagcttcccctcacttaaccaccccataccagtcacctcctgctccttttcctcttttgtgagaagatgggggctggagggggcagagtggcctgtgagcaagagccaggggtgtcccagtcccagcctctggggcagagcttgtagccctggatggcctctggggcaggaccactagctaagcaagccaggagaagacccctgcccaagtggctcttgggacaacgtgctgcttacactccaggtgtggaccggccgcagcccccactgacctgcccatgtccagagggactggacagccagggcagggctttggggggcactagaagatgagggtgtcggctgtgaggcgggtggctggtataaataatatttatcttttcaaccagcatttgtgaaggcctggactccacagggggctggatggagagggtgtctcacagaggtgggggctgcttcccagctttcaggggctgctgtgttgctgagaaacctgaccccactctcaccccggcaaggatggtgccaggagagcccctgagggccgggagaggggtgagggcaggagggagggcttcgaggaacaggtgggtctggccagggcccccttagttagggacagcttgttgtgaggaggtgaattagagggagaggagggccacggttagggatgcagaggagggacaccatgggtcaggggaaggaacaaaggagacgggagtgtgtcccagccccaggcatgagctgtgctgattggtgggaagcagaattaggatgctgggccactggacaggctgtgaatgcagctgtctccactagggccgccacaagaagccatccctgagaccctgaggtttcttgtgcaaactcgagggaaggggagtacagagagggcaacagggagtgaagccaggcaaggaaggcaggggaaaggtgggggctagagggggacacggtagcctgtgagcaagagacaggtatgccccagccctggccagtgccgctctgaggtgggtggcacactcaggctcccctggcttgctggcttcagtgcccccaactccgggccagagcttgtatagccctgggtggcctctggggcaggactgacactccaacactgttgtgggcgcctggagctcccttccgctggggaagccctccctcatcccacccaacaggtgagggagctggggtatttatccaccatctcccatcagctgcgggttgagggctgctcccagggggacatgaactcttctgcattcccacatcaggctacagagggccagagagagttctccggcacagtgatgtgcgggtgggtggaagccagtgtgtgctgcattggaggggcagggggtgtgggtggaacaccagaactggacacagggccccactgcagcccgggccttgtctctgggtctctctaaccagggaggagagccccgtgggtgggacgtgtgtatacacatgtgcacttgtgcacccaggcgcacacaagagcttgcagtgtcaggacaccaggaacagacaaaggtgacacagtgccgaaagccccgcagctcctgctgagggagggcacgtttcgttggggagggcctgggcccgggtgtagctaaggatatccttgaaggccctatggggtactctgctctgccaattctccaagctctgtgccctgaagtagagagatggggtgggggacccagggagcaccgtgacagaggtaggctggaggcttccagagcaacgtgggggatattgtggggagtgggaagaagcccagtaaagggtcctggctgcctggagagactagaagtgacctgagtctctaatggccacaacggagacagctttcccaactctgcctctcacccagtgaaggtggagaagtgagaaggcgaggctttcccgagacaatgccagtggggatgggatgagggtcgcaccgcaggggtggaggagccgagagggtgggggtgaggggaagacagggaagggacggtacagaccaggaagtgtggataagcccagccaggccgcaccccgctttcctgagtgggcttcctggctgctctcactgccaggcaccttctgggcacctactgtgtgctcagcactgtgctaggtcttgggtgagagatgagcagccacccaggggggaccccagggaccccccaaaaaacacaaagctcagagcctctggctctggaaggccaccaagaggacagctccaagacaagtctgagggtcatcggagaagctggaggcggaatccctgggcaggcagatggagcctggggctcccctcccattgagagaatctgagggcggaggggctgcttcagggaacagccaggaacatctgagcctcagaacctgcagccc

SEQ ID NO: 9 - DNA, 21 bp, Artificial sequence
  Synthetic nucleic acid
agaaaccatttgctatctctg

SEQ ID NO: 10 - DNA, 21 bp, Artificial sequence
  Synthetic nucleic acid
cagagatagcaaatggtttct

SEQ ID NO: 11 - DNA, 25 bp, Artificial sequence
  Synthetic nucleic acid
ccagaaaccatttgctatctctgtt

SEQ ID NO: 12 - DNA, 25 bp, Artificial sequence
  Synthetic nucleic acid
aacagagatagcaaatggtttctgg

SEQ ID NO: 13 - DNA, 21 bp, Artificial sequence
  Synthetic nucleic acid
agaaaccattcgctatctctg

SEQ ID NO: 14 - DNA, 21 bp, Artificial sequence
  Synthetic nucleic acid
cagagatagcgaatggtttct

SEQ ID NO: 15 - DNA, 25 bp, Artificial sequence
  Synthetic nucleic acid
ccagaaaccattcgctatctctgtt

SEQ ID NO: 16 - DNA, 25 bp, Artificial sequence
  Synthetic nucleic acid
aacagagatagcgaatggtttctgg

SEQ ID NO: 17 - DNA, 21 bp, Artificial sequence
  Synthetic nucleic acid
ctctggagcataagtttacct

SEQ ID NO: 18 - DNA, 25 bp, Artificial sequence
  Synthetic nucleic acid
ttctctggagcataagtttacctta

SEQ ID NO: 19 - DNA, 21 bp, Artificial sequence
  Synthetic nucleic acid
aggtaaacttatgctccagag

SEQ ID NO: 20 - DNA, 25 bp, Artificial sequence
  Synthetic nucleic acid
taaggtaaacttatgctccagagaa

SEQ ID NO: 21 - DNA, 21 bp, Artificial sequence
  Synthetic nucleic acid
ctctggagcagaagtttacct

SEQ ID NO: 22 - DNA, 25 bp, Artificial sequence
  Synthetic nucleic acid
ttctctggagcagaagtttacctta

SEQ ID NO: 23 - DNA, 21 bp, Artificial sequence
  Synthetic nucleic acid
aggtaaacttctgctccagag

SEQ ID NO: 24 - DNA, 25 bp, Artificial sequence
  Synthetic nucleic acid
taaggtaaacttctgctccagagaa

SEQ ID NO: 25 - DNA, 23 bp, Artificial sequence
  Synthetic nucleic acid
gtcactaccagaaaccatttgct

SEQ ID NO: 26 - DNA, 24 bp, Artificial sequence
  Synthetic nucleic acid
tgtgtcactaccagaaaccatttg

SEQ ID NO: 27 - DNA, 25 bp, Artificial sequence
  Synthetic nucleic acid
caaacaaggaacagagatagcaaat

SEQ ID NO: 28 - DNA, 26 bp, Artificial sequence
  Synthetic nucleic acid
atacaaacaaggaacagagatagcaa

SEQ ID NO: 29 - DNA, 23 bp, Artificial sequence
  Synthetic nucleic acid
gtcactaccagaaaccattcgct

SEQ ID NO: 30 - DNA, 24 bp, Artificial sequence
  Synthetic nucleic acid
tgtgtcactaccagaaaccattcg

SEQ ID NO: 31 - DNA, 25 bp, Artificial sequence
  Synthetic nucleic acid
caaacaaggaacagagatagcgaat

SEQ ID NO: 32 - DNA, 26 bp, Artificial sequence
  Synthetic nucleic acid
atacaaacaaggaacagagatagcga

SEQ ID NO: 33 - DNA, 20 bp, Artificial sequence
  Synthetic nucleic acid
caggttctctggagcataag

SEQ ID NO: 34 - DNA, 23 bp, Artificial sequence
  Synthetic nucleic acid
cagcccaggttctctggagcata

SEQ ID NO: 35 - DNA, 22 bp, Artificial sequence
  Synthetic nucleic acid
acaaaataaggtaaacttatgc

SEQ ID NO: 36 - DNA, 23 bp, Artificial sequence
  Synthetic nucleic acid
tgtacaaaataaggtaaacttat

SEQ ID NO: 37 - DNA, 20 bp, Artificial sequence
  Synthetic nucleic acid
caggttctctggagcagaag

SEQ ID NO: 38 - DNA, 23 bp, Artificial sequence
  Synthetic nucleic acid
cagcccaggttctctggagcaga

SEQ ID NO: 39 - DNA, 22 bp, Artificial sequence
  Synthetic nucleic acid
acaaaataaggtaaacttctgc

SEQ ID NO: 40 - DNA, 23 bp, Artificial sequence
  Synthetic nucleic acid
tgtacaaaataaggtaaacttct

SEQ ID NO: 41 - DNA, 20 bp, Artificial sequence
  Synthetic nucleic acid
ttcctgggaaccatctttgg

SEQ ID NO: 42 - DNA, 20 bp, Artificial sequence
  Synthetic nucleic acid
ccatctttggtattgcaaag

SEQ ID NO: 43 - DNA, 20 bp, Artificial sequence
  Synthetic nucleic acid
tcaccctcctgcttcatttg

SEQ ID NO: 44 - DNA, 20 bp, Artificial sequence
  Synthetic nucleic acid
gggtcaccaaggagctgcat

SEQ ID NO: 45 - DNA, 20 bp, Artificial sequence
  Synthetic nucleic acid
catttccagtgcagaaatac

SEQ ID NO: 46 - DNA, 20 bp, Artificial sequence
  Synthetic nucleic acid
gcagaaatacaaacaaggaa

SEQ ID NO: 47 - DNA, 20 bp, Artificial sequence
  Synthetic nucleic acid
ctgcctggctcatgctaggc

SEQ ID NO: 48 - DNA, 20 bp, Artificial sequence
  Synthetic nucleic acid
tgggattgatctttcactga

SEQ ID NO: 49 - DNA, 20 bp, Artificial sequence
  Synthetic nucleic acid
cccctataaaggtgttgtac

SEQ ID NO: 50 - DNA, 20 bp, Artificial sequence
  Synthetic nucleic acid
ctgtgcagtgcttaagaaaa

SEQ ID NO: 51 - DNA, 20 bp, Artificial sequence
  Synthetic nucleic acid
ggccagagatgacagcaacc

SEQ ID NO: 52 - DNA, 21 bp, Artificial sequence
  Synthetic nucleic acid
caggacatgccttttagaggc

(19) United States
(12) Patent Application Publication
(10) Pub. No.: US 2013/0244990 A1
(43) Pub. Date: Sep. 19, 2013

(54) GENETIC POLYMORPHISMS ASSOCIATED WITH DEPRESSION

(71) Applicant: The Board of Trustees of the Leland Stanford Junior University, (US)

(72) Inventors: Alan F. Schatzberg, Los Altos, CA (US); Laura C. Lazzeroni, Redwood City, CA (US); Greer M. Murphy, Stanford, CA (US); Jennifer Keller, Los Altos, CA (US)

(73) Assignee: The Board of Trustees of the Leland Stanford Junior University, Palo Alto, CA (US)

(21) Appl. No.: 13/690,928

(22) Filed: Nov. 30, 2012

Related U.S. Application Data
(60) Provisional application No. 61/566,314, filed on Dec. 2, 2011.

(51) Int. Cl. C12Q 1/68

(52) U.S. Cl. CPC C12Q 1/6883
USPC 514/178; 435/6.11; 536/24.31

(57) ABSTRACT

The present disclosure provides methods for determining risk for depression, as well as compositions for use in such methods.

### BACKGROUND

Depression is typically diagnosed as major depressive disorder (unipolar major depression, bipolar disorder (manic-depressive illness), and dysthymic disorder (dysthymia). There are a number of subtypes of these major categories of depression. Diagnosis of these mental disorders is based on the Diagnostic and Statistical Manual of Mental Disorders, fourth edition (DSM-IV) (American Psychiatric Association; Diagnostic and Statistical Manual of Mental Disorders, fourth edition (DSM-IV), Washington, D.C., American Psychiatric Press, 1994).

Single-nucleotide polymorphisms (SNPs) are small variations in genomes. They are among the most common forms of human genetic variations. Many monogenic human diseases have been associated with genetic polymorphic variations such as SNPs in the so-called susceptibility genes. Genetic polymorphic variations are also associated with varying response to drugs and natural environmental agents.

There is an ongoing need in the art to identify additional SNPs, including those associated with psychiatric disorders such as depression.

### LITERATURE

Papiol et al. (2007) J. Affect. Disorders 104:83; Hiroi et al. (2001) Mol. Psychiatr. 6:540; Binder et al. (2010) Arch. Gen. Psychiatry 67:369; U.S. Patent Publication No. 2010/0105062; U.S. Patent Publication No. 2010/0119625.

### SUMMARY

The present disclosure provides methods for determining risk for depression, as well as nucleic acids and compositions for use in such methods. The present disclosure provides a method of detecting a single nucleotide polymorphism (SNP) associated with depression in an individual; and a method of detecting a predisposition of an individual to develop depression. The present disclosure further provides nucleic acid reagents and kits for determining a subject's genotype with respect to a single nucleotide polymorphism associated with depression.

### BRIEF DESCRIPTION OF THE DRAWINGS

FIG. 1 depicts depression-associated CHR-R1 gene SNPs.

FIGS. 2 and 3 depict features of the analysis of CRHR1 SNPs.

FIG. 4 depicts receiver operating characteristics (ROC) based on CRHR1 genotypes.

FIG. 5 depicts depression-associated GR SNPs.

FIGS. 6-8 depict features of the analysis of GR SNPs.

FIG. 9 depicts depression-associated DRD2 SNPs.

FIG. 10 depicts features of the analysis of DRD2 SNPs.

FIG. 11 provides a nucleotide sequence of a human DRD2 gene.

FIGS. 12 and 13 are tables listing SNPs in linkage disequilibrium with CRHR1 and GR SNPs.

FIGS. 14-16 depict SNPs that are tagged with R2 0.8 or greater by the genotyped set.

FIG. 17 is a table of data relating to various SNPs and depression.

FIG. 18 depicts association of SNPs in the human CRHR2 gene with depression.

FIG. 19 depicts the nucleotide sequence of rs4723003 (SEQ ID NO:1).

FIG. 20 depicts the nucleotide sequence of rs2284216 (SEQ ID NO:2).

FIGS. 21A-K (SEQ ID NO:3) depict the nucleotide sequence of a human CRHR2 gene (GenBank Accession number NG_{—}029169.1). Sequences corresponding to rs4723003 and rs2284216 are shown in bold and underlined text.

FIGS. 22A-K (SEQ ID NO:4) depict the reverse complement of the nucleotide sequence depicted in FIGS. 21A-K. Sequences corresponding to rs4723003 and rs2284216 are shown in bold and underlined text.

FIG. 23 provides a nucleotide sequence of a human corticotropin-releasing hormone receptor-1 (CRHR1) gene.

### DEFINITIONS

As used herein, an “allele” is one of a pair or series of genetic variants of a polymorphism at a specific genomic location.

As used herein, “genotype” refers to the diploid combination of alleles for a given genetic polymorphism. A homozygous subject carries two copies of the same allele and a heterozygous subject carries two different alleles.

As used herein, a “haplotype” is one or a set of signature genetic changes (polymorphisms) that are normally grouped closely together on the DNA strand, and are usually inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including, e.g., single nucleotide polymorphisms (SNPs) in which a particular nucleotide is changed.

Linkage disequilibrium” refers to when the observed frequencies of haplotypes in a population does not agree with haplotype frequencies predicted by multiplying together the frequency of individual genetic markers in each haplotype.

The term “label” or “label containing moiety” refers in a moiety capable of detection, such as a radioactive isotope or group containing same, and nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent). A nucleic acid probe or primer can be bound, e.g., chemically bound to label-containing moieties or can be suitable to be so bound. The probe or primer can be directly or indirectly labeled.

The term “direct label probe” (or “directly labeled probe”) refers to a nucleic acid probe whose label after hybrid formation with a target is detectable without further reactive processing of hybrid. The term “indirect label probe” (or “indirectly labeled probe”) refers to a nucleic acid probe whose label after hybrid formation with a target is further reacted in subsequent processing with one or more reagents to associate therewith one or more moieties that finally result in a detectable entity.

The terms “target,” “DNA target,” or “DNA target region” refers to a nucleotide sequence that occurs at a specific chromosomal location. Each such sequence or portion is preferably at least partially, single stranded (e.g., denatured) at the time of hybridization. When the target nucleotide sequences are located only in a single region or fraction of a given chromosome, the term “target region” is sometimes used. Targets for hybridization can be obtained from a biological sample that includes genomic DNA.

The term “hybrid” refers to the product of a hybridization procedure between a nucleic acid probe and a target nucleic acid.

The term “hybridizing conditions” has general reference to the combinations of conditions that are employable in a given hybridization procedure to produce hybrids, such conditions typically involving controlled temperature, liquid phase, and contact between a probe (or probe composition) and a target. At least one denaturation step can precede a step wherein a probe or probe composition is contacted with a target. Guidance for performing hybridization reactions can be found in Ausubel et al., Current Protocols in Molecular Biology, John Wiley & Sons, N.Y. (2003), 6.3.1-6.3.6. Aqueous and nonaqueous methods are described in that reference and either can be used. Hybridization conditions referred to herein are a 50% formamide, ×SSC wash for 10 minutes at 45° C. followed by a 2×SSC wash for 10 minutes at 37° C.

A “nucleic acid,” “polynucleotide,” or “oligonucleotide” is a polymeric form of nucleotides of any length, may be DNA or RNA, and may be single- or double-stranded. Nucleic acids may include promoters or other regulatory sequences. Oligonucleotides are usually prepared by synthetic means. Nucleic acids include segments of DNA, or their complements spanning or flanking a polymorphic site in a CRHR2 gene, as described herein. The segments can be between 5 and 100 contiguous bases, and often range from a lower limit of 5, 10, 12, 15, 20, or 25 nucleotides to an upper limit of 10, 15, 20, 25, 30, 50 or 100 nucleotides (where the upper limit is greater than the lower limit). Nucleic acids between 5-10, 5-20, 10-20, 12-30, 15-30, 10-50, 20-50 or 20-100 bases are common. The polymorphic site can occur within any position of the segment. A reference to the sequence of one strand of a double-stranded nucleic acid defines the complementary sequence and except where otherwise clear from context, a reference to one strand of a nucleic acid also refers to its complement. For certain applications, nucleic acid (e.g., RNA) molecules may be modified to increase intracellular stability and half-life. Possible modifications include, but are not limited to, the use of phosphorothioate or 2′-O-methyl rather than phosphodiesterase linkages within the backbone of the molecule. Modified nucleic acids include peptide nucleic acids (PNAs) and nucleic acids with nontraditional bases such as inosine, queosine and wybutosine and acetyl-, methyl-, thio- and similarly modified forms of adenine, cytidine, guanine, thymine, and uridine which are not as easily recognized by endogenous endonucleases.

“Hybridization probes” are nucleic acids capable of binding in a base-specific manner to a complementary strand of nucleic acid. Such probes include nucleic acids and peptide nucleic acids. Hybridization may be performed under stringent conditions which are known in the art. For example, see, e.g., Berger and Kimmel (1987) Methods In Enzymology, Vol. 152: Guide To Molecular Cloning Techniques, San Diego: Academic Press, Inc.; Sambrook et al. (1989) Molecular Cloning: A Laboratory Manual, 2nd Ed., Vols. 1-3, Cold Spring Harbor Laboratory; Sambook (2001) 3rd Edition; Rychlik, W. and Rhoads, R. E., 1989, Nucl. Acids Res. 17, 8543; Mueller, P. R. et al. (1993) In: Current Protocols in Molecular Biology 15.5, Greene Publishing Associates, Inc. and John Wiley and Sons, New York; and Anderson and Young, Quantitative Filter Hybridization in Nucleic Acid Hybridization (1985)). As used herein, the term “probe” includes primers. Probes and primers are sometimes referred to as “oligonucleotides.”

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer but typically ranges from 15 to 30 nucleotides. A primer sequence need not be exactly complementary to a template but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

Exemplary hybridization conditions for short probes and primers is about 5 to 12 degrees C. below the calculated Tm. Formulas for calculating Tm are known and include: Tm=4° C.×(number of G's and C's in the primer)+2° C.×(number of A's and T's in the primer) for oligonucleotides (“oligos”)<14 bases and assumes a reaction is carried out in the presence of 50 mM monovalent cations. For longer oligos, the following formula can be used: Tm=64.9° C.+41° C.×(number of G's and C's in the primer-16.4)/N, where N is the length of the primer. Another commonly used formula takes into account the salt concentration of the reaction (Rychlik, supra, Sambrook, supra, Mueller, supra.): Tm=81.5° C.+16.6° C.×(log₁₀[Na⁺]+[K⁺]+0.41° C.×(% GC)−675/N, where N is the number of nucleotides in the oligonucleotide. The aforementioned formulae provide a starting point for certain applications; however, the design of particular probes and primers may take into account additional or different factors. Methods for design of probes and primers for use in the methods of the invention are well known in the art.

As used herein, the terms “allele” and “allelic variant” refer to alternative forms of a gene including introns, exons, intron/exon junctions and 3′ and/or 5′ untranslated regions that are associated with a gene or portions thereof. Generally, alleles occupy the same locus or position on homologous chromosomes. When a subject has two identical alleles of a gene, the subject is said to be homozygous for the gene or allele. When a subject has two different alleles of a gene, the subject is said to be heterozygous for the gene.

As used herein, the term “isolated” as used herein with respect to a nucleic acid, refers to a nucleic acid separated from macromolecules or other contaminants that may be present in the natural source of the nucleic acid, or that may be present during recombinant or chemical synthesis of the nucleic acid. An isolated nucleic acid can be purified, e.g., at least 80%, at least 85%, at least 90%, at least 95%, at least 98%, at least 99%, or greater than 99%, pure.

“Depression,” as used herein includes, e.g., major depressive disorder, dysthymic disorder, atypical depressive disorder, bipolar disorder, and subtypes of the foregoing. The various forms of depression are defined and are separately diagnosed according to criteria given in handbooks for psychiatry, for example in the Diagnostic and Statistical Manual of Mental Disorders 4th edition (DSM-IV) published by the American Psychiatric Association, Washington, D.C. (1994). In the DSM-IV, depressive disorders are classified under mood disorders and include major depressive disorder, dysthymic disorder and depressive disorder not otherwise specified (or “atypical”). In general, regardless whether the depressive syndrome is melancholic, atypical, or some admixture of the two, a diagnosis of major depression is given when depressed mood is present, or loss of interest or pleasure in all activities is present, for at least two weeks. The term “major depressive disorder” (MDD) is understood in art, and refers to a diagnosis that is guided by diagnostic criteria listed in DSM-IV or ICD-10, or in similar nomenclatures. (DSM-IV-TR., 2000, Kaplan, H. I. et al. 1998.).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a single nucleotide polymorphism” includes a plurality of such polymorphisms and reference to “the primer pair” includes reference to one or more primer pairs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for determining risk for depression, as well as nucleic acids and compositions for use in such methods. The present disclosure provides a method of detecting a single nucleotide polymorphism (SNP) associated with depression in an individual; and a method of detecting a predisposition of an individual to develop depression. The present disclosure further provides nucleic acid reagents and kits for determining a subject's genotype with respect to a depression-associated single nucleotide polymorphism.

Detection of Polymorphisms Associated with Depression

The present disclosure provides a method of detecting a single nucleotide polymorphism (SNP) associated with depression in an individual. The method generally involves analyzing a polynucleotide sample from the individual for the presence of a depression-associated SNP (or collection of such SNPs) in one or more of a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene, wherein the SNP is associated with depression, and where the presence of the polymorphism is indicative of a polymorphism associated with depression (e.g., increased risk of developing depression). In some cases, the depression is major depression. In some cases, the depression is major depression with psychotic features. In some cases, the depression is major depression with non-psychotic features. In some cases, the depression is major depression with endogenous features.

### CRH-R1 Gene SNPs

In some cases, a subject method involves analyzing a polynucleotide sample from the individual for the presence of a SNP in a CRHR1 gene, wherein the SNP is associated with depression, and where the presence of the polymorphism is indicative of a polymorphism associated with depression (e.g., increased risk of developing depression). In some cases, the depression is major depression. In some cases, the depression is major depression with psychotic features. In some cases, the depression is major depression with non-psychotic features. In some cases, the depression is major depression with endogenous features.

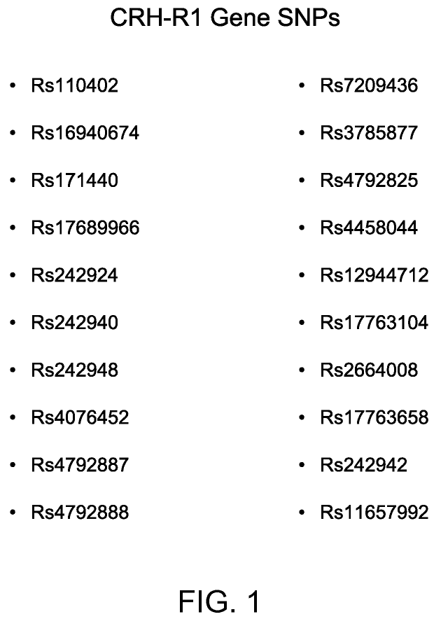

Depression-associated SNPs in a CRHR1 gene include those depicted in FIG. 1, e.g.,

Rs110402;

Rs16940674;

Rs171440;

Rs17689966;

Rs242924;

Rs242940;

Rs242948;

Rs4076452;

Rs4792887;

Rs4792888;

Rs7209436;

Rs3785877;

Rs4792825;

Rs4458044;

Rs12944712;

Rs17763104;

Rs2664008;

Rs17763658;

Rs242942; and

Rs11657992.

In some embodiments, all 21 of the above-listed CRH-R1 SNPs are detected. In other embodiments, 1, 2, 3, 4, 5, 6, 7, 8, 9, 10, 11, 12, 13, 14, 15, 16, 17, 18, 19, or 20 of the above-listed CRH-R1 SNPs are detected. In some cases, rs17763104 alone is detected. In some cases, rs3785877 alone is detected. In some cases, rs4458044 alone is detected.

In some embodiments, the following CRH-R1 SNPs are detected: rs110402; rs16940674; rs171440; rs17689966; rs242924; rs242940; rs242948; rs4076452; rs4792887; rs4792888; rs7209436; rs3785877; rs4792825; rs4458044; rs12944712; rs17763104; rs2664008; rs17763658; rs242942; and rs11657992.

As noted above, in some cases, all 21 of the above-listed CRH-R1 SNPs are detected to diagnose patients with major depression with endogenous features and to discriminate them from healthy control subjects. In some embodiments, a subject method involves analyzing a polynucleotide sample from an individual for the presence all 21 of the above-listed CRH-R1 SNPs in a CRHR1 gene, where the presence of the polymorphisms is indicative of a polymorphism associated with major depression (e.g., increased risk of developing major depression, e.g., major depression with endogenous features).

In some embodiments, a subject method involves analyzing a polynucleotide sample from an individual for the presence of a SNP at rs17763104 in a CRHR1 gene, where the presence of the polymorphism is indicative of a polymorphism associated with major depression (e.g., increased risk of developing major depression). In some embodiments, a subject method involves analyzing a polynucleotide sample from an individual for the presence of a SNP at rs3785877 in a CRHR1 gene, where the presence of the polymorphism is indicative of a polymorphism associated with major depression (e.g., increased risk of developing major depression). In some embodiments, a subject method involves analyzing a polynucleotide sample from an individual for the presence of a SNP at rs4458044 in a CRHR1 gene, where the presence of the polymorphism is indicative of a polymorphism associated with major depression (e.g., increased risk of developing major depression).

In some instances, all 21 of the above-listed CRH-R1 SNPs are detected to diagnose patients with major depression with psychotic features and to discriminate them from healthy control subjects.

The various SNPs can be found at: http://www(dot)ncbi(dot)nlm(dot)nih(dot)gov/projects/SNP/. For example:

CRHR1 gene SNP rs17763104:
ACGGGGTTGCCCTGATGGTTTAAGAC[A/G]ATAACAGATATGAAAATC
CTCTGTA;
CRHR1 gene SNP rs3785877:
GCACCTGGGCTTGGCCCACTCCAGCC[A/G]CCTGGCCCGCAGTCACCT
CGTGTCC;
CRHR1 gene SNP rs4458044:
AGAGCCCTTCCTGAGTCCCATCCATT[C/G]GCAGGGTCCTACTGTTGT
CCGCCCC;
CRHR1 gene SNP rs110402:
TTTCTAAACACAGAGGACTGGTGTTG[C/T]GTTATGCAAAGAAAAATG
CTTCTTA;
CRHR1 gene SNP rs16940674:
CCCTTTCCTCTGTGGCCTTCTAGGTG[C/T]TGGTTTGGCAAAAGGCCT
GGGGTGT;
CRHR1 gene SNP rs171440:
GTCCCCTGCTCTGTAGCCTAAGGACA[C/T]TTCTCTTGGTCCCTCGCA
TGGTGAC;
CRHR1 gene SNP rs17689966:
CAAGCACTGTCCCTCCCCATGCCATC[A/G]AGGTGGACGCAGATGACC
CTTCCTC;
CRHR1 gene SNP rs242924:
AAGACACTCAGGTGCAGGGACCCTCT[A/C]CATTTTTGCCCAGCAGCA
GCCATGC;
CRHR1 gene SNP rs242940:
GGCACACCAGTCCTTTTGAGCCCCAG[C/T]GTCCCCAGGTTAATAACC
TAGAATT;
CRHR1 gene SNP rs242948:
CTGCTTCCCACCAATCAGCACAGCTC[A/C]TGCCTGGGGCTGGGACAC
ACTCCCG;
CRHR1 gene SNP rs4076452:
CAGGAAAATGATGCTCTGGGGTTAGT[C/G]TTCCCTTTTTGCTTTCCC
CAGTGGA;
CRHR1 gene SNP rs4792887:
GCCTCTGGGGTCACCAGGTACATCTT[C/T]GATCTTGGCCACACTGGA
GAGTCAA;
CRHR1 gene SNP rs4792888:
AAGAAAGACCACATTGCCCCCACTTC[A/G]CAGATGAGGATGTTGAGT
CTCAGAG;
CRHR1 gene SNP rs7209436:
CTGTCCCACAACATGGGGTCTTACAG[C/T]TCTTTGATGTATCCCCCC
ACAGGGG;
CRHR1 gene SNP rs4792825:
CTAATGCTTTGAAGGATACTTTACAA[A/G]TGAGTCACCAAAAATATT
GGCAAAT;
CRHR1 gene SNP rs12944712:
CTAAAGCTGACAGGGCAGGAGACCTG[A/G]GGTTGGAGCTGACTCAG
CCACTTCT;
CRHR1 gene SNP rs2664008:
GGGAGAGGGGAGCATTGTAACCGGTC[C/T]TCCCCTCTGGCTGTGGGG
AGCTGGG;
CRHR1 gene SNP rs17763658:
TGCCCACGGGCTGCTCATCGTGAGCC[A/G]GGTCAGTGACCAACAGTG
CTCAGAC;
CRHR1 gene SNP rs242942:
cacacatgacatatgccatctcattt[A/G/T]atGGCACCATTGTACC
TTCCTCAGT
CRHR1 gene SNOP rs11657992:
GCCCCCAAATCCCTCCCTTCTGGTTC[C/T]GGAGGCCCCAGGGTTACT
CTCCCGC.

### Glucocorticoid Receptor (GR) Gene SNPs

In some cases, a subject method involves analyzing a polynucleotide sample from the individual for the presence of a SNP in a GR gene, wherein the SNP is associated with depression, and where the presence of the polymorphism is indicative of a polymorphism associated with depression (e.g., increased risk of developing depression). In some cases, the depression is major depression. In some cases, the depression is major depression with psychotic features. In some cases, the depression is major depression with non-psychotic features.

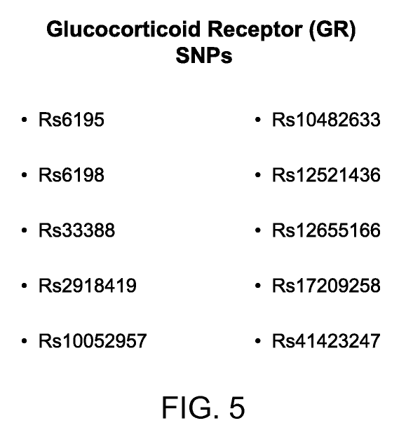

Depression-associated SNPs in a GR gene include those depicted in FIG. 5, e.g.,

Rs6195;

Rs6198;

Rs33388;

Rs2918419;

Rs10052957;

Rs10482633;

Rs12521436;

Rs12655166;

Rs17209258;

Rs41423247.

In some cases, all 10 of the above-listed GR SNPs are detected. In other instances, 1, 2, 3, 4, 5, 6, 7, 8, or 9 of the above-listed GR SNPs are detected.

In some cases, the following GR SNPs are detected: rs6198; rs33388; rs2918419; rs10052957; rs10482633; rs12521436; rs12655166; rs17209258; and rs41423247.

In some embodiments, a subject method involves analyzing a polynucleotide sample from an individual for the presence all 10 of the above-listed GR SNPs in a GR gene, where the presence of the polymorphisms is indicative of a polymorphism associated with major depression (e.g., increased risk of developing major depression, e.g., major depression with psychotic features).

In some embodiments, a subject method involves analyzing a polynucleotide sample from an individual for the presence of a SNP at rs33388 in a GR gene, where the presence of the polymorphism is indicative of a polymorphism associated with major depression (e.g., increased risk of developing major depression with psychotic features); e.g., where the presence of the SNP predicts cortisol from 6 pm to 1 am. In some embodiments, a subject method involves analyzing a polynucleotide sample from an individual for the presence of a SNP at rs2918419 in a GR gene, where the presence of the polymorphism is indicative of a polymorphism associated with major depression (e.g., increased risk of developing major depression with psychotic features); e.g., where the presence of the SNP predicts cortisol from 6 pm to 1 am. In some embodiments, a subject method involves analyzing a polynucleotide sample from an individual for the presence of a SNP at rs10052957 in a GR gene, where the presence of the polymorphism is indicative of a polymorphism associated with major depression (e.g., increased risk of developing major depression with psychotic features); e.g., where the presence of the SNP predicts cortisol from 6 pm to 1 am. In some embodiments, a subject method involves analyzing a polynucleotide sample from an individual for the presence of a SNP at rs10482633 in a GR gene, where the presence of the polymorphism is indicative of a polymorphism associated with major depression (e.g., increased risk of developing major depression with psychotic features); e.g., where the presence of the SNP predicts cortisol from 6 pm to 1 am. In some embodiments, a subject method involves analyzing a polynucleotide sample from an individual for the presence of a SNP at rs12521436 in a GR gene, where the presence of the polymorphism is indicative of a polymorphism associated with major depression (e.g., increased risk of developing major depression with psychotic features); e.g., where the presence of the SNP predicts cortisol from 6 pm to 1 am. In some embodiments, a subject method involves analyzing a polynucleotide sample from an individual for the presence of a SNP at rs41423247 in a GR gene, where the presence of the polymorphism is indicative of a polymorphism associated with major depression (e.g., increased risk of developing major depression with psychotic features); e.g., where the presence of the SNP predicts cortisol from 6 pm to 1 am.

In some embodiments, a subject method involves analyzing a polynucleotide sample from an individual for the presence of a SNP at rs2918419 in a GR gene, where the presence of the polymorphism is indicative of a polymorphism associated with major depression (e.g., increased risk of developing major depression with psychotic features); e.g., where the presence of the SNP predicts cortisol from 1 am to 9 am. In some embodiments, a subject method involves analyzing a polynucleotide sample from an individual for the presence of a SNP at rs10052957 in a GR gene, where the presence of the polymorphism is indicative of a polymorphism associated with major depression (e.g., increased risk of developing major depression with psychotic features); e.g., where the presence of the SNP predicts cortisol from 1 am to 9 am. In some embodiments, a subject method involves analyzing a polynucleotide sample from an individual for the presence of a SNP at rs41423247 in a GR gene, where the presence of the polymorphism is indicative of a polymorphism associated with major depression (e.g., increased risk of developing major depression with psychotic features); e.g., where the presence of the SNP predicts cortisol from 1 am to 9 am.

GR SNP rs2918419:
TGTATTTTAACTGGCCTTTTCCTTGT[A/G]TGTCTCCTACAATGAGCT
GTTGAAG;
GR SNP rs33388:
GAAAGTCATGGATGGATTATGAGTTA[A/T]TCACACACCTAGAGAAGC
ATGTAAA;
GR SNP rs2918419:
TGTATTTTAACTGGCCTTTTCCTTGT[A/G]TGTCTCCTACAATGAGCT
GTTGAAG;
GR SNP rs10052957:
AAATGAAGGTGATGTATTCAGACTCA[A/G]TCaaggcaaggacctgat
ctatctt;
GR SNP rs10482633:
TACTTGACTTGGCTATGGTCTAGATA[A/C]TCCATGAAAATTTAAAGG
ACAGATT;
GR SNP rs12521436:
ctctgagcctcctcctcactcatcat[A/G]cttcagccactgtcctcT
CCCTGGC;
GR SNP rs41423247:
ACACCAATTCCTCTCTTAAAGAGATT[C/G]ATCAGCAGACATAACTTG
TCTACTT.

### Dopamine 2 Receptor (DRD2) Gene SNPs

In some cases, a subject method involves analyzing a polynucleotide sample from the individual for the presence of a SNP in a DRD2 gene, wherein the SNP is associated with depression, and where the presence of the polymorphism is indicative of a polymorphism associated with depression (e.g., increased risk of developing depression). In some cases, the depression is major depression. In some cases, the depression is major depression with psychotic features. In some cases, the depression is major depression with non-psychotic features.

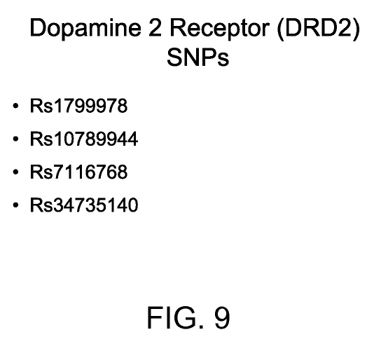

Depression-associated SNPs in a DRD2 gene include those depicted in FIG. 9, e.g.,

rs1799978;

rs10789944;

rs7116768;

rs34735140.

In some cases, all 4 of the above-listed DRD2 SNPs are detected. In other instances, 1, 2, or 3 of the above-listed DRD2 SNPs are detected.

In some embodiments, a subject method involves analyzing a polynucleotide sample from an individual for the presence all 4 of the above-listed DRD2 SNPs in a GR gene, where the presence of the polymorphisms is indicative of a polymorphism associated with major depression (e.g., increased risk of developing major depression).

DRD2 SNP rs1799978:
GAGGACCCAGCCTGCAATCACAGCTT[A/G]TTACTCTGGGTGTGGGTG
GGAGCGC;
DRD2 SNP rs10789944:
ACTCTGGCCGCGAGCCCCGGGTCTCG[A/C]GCTTGGGTGCTGGGGGAA
GTGCCCT;
DRD2 SNP rs7116768:
GCTTACCTTCAAGCCATAGGGCGCCC[C/G]GGGGCAGAGACGGCGCC
GGCTGCTT;
DRD2 SNP rs34735140:
TCACCCTCCGCTACTGCCGCCACCGT[-/C]GCCTGCACTGCCGGGTA
GAAACGGG.

### CRH-R2 SNPs

In some embodiments, the method generally involving: analyzing a polynucleotide sample from an individual for the presence of a SNP at rs4723003 and/or rs2284216 in a CRHR2 gene, wherein the SNP is associated with depression, and where the presence of the polymorphism is indicative of a polymorphism associated with depression (e.g., increased risk of developing depression).

### Linkage Disequilibrium

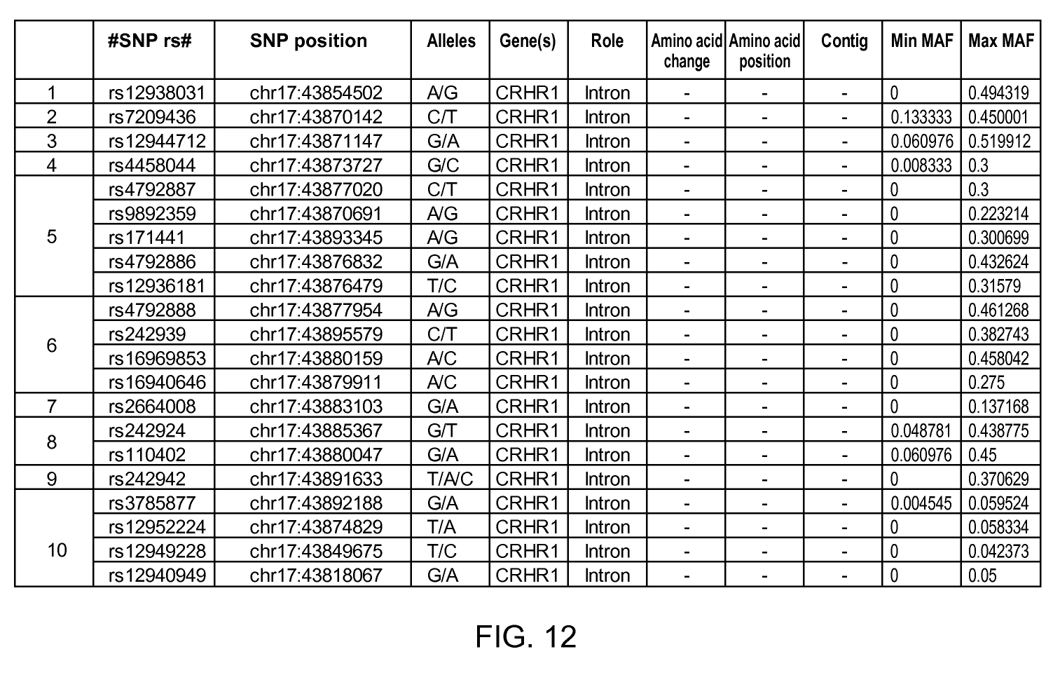

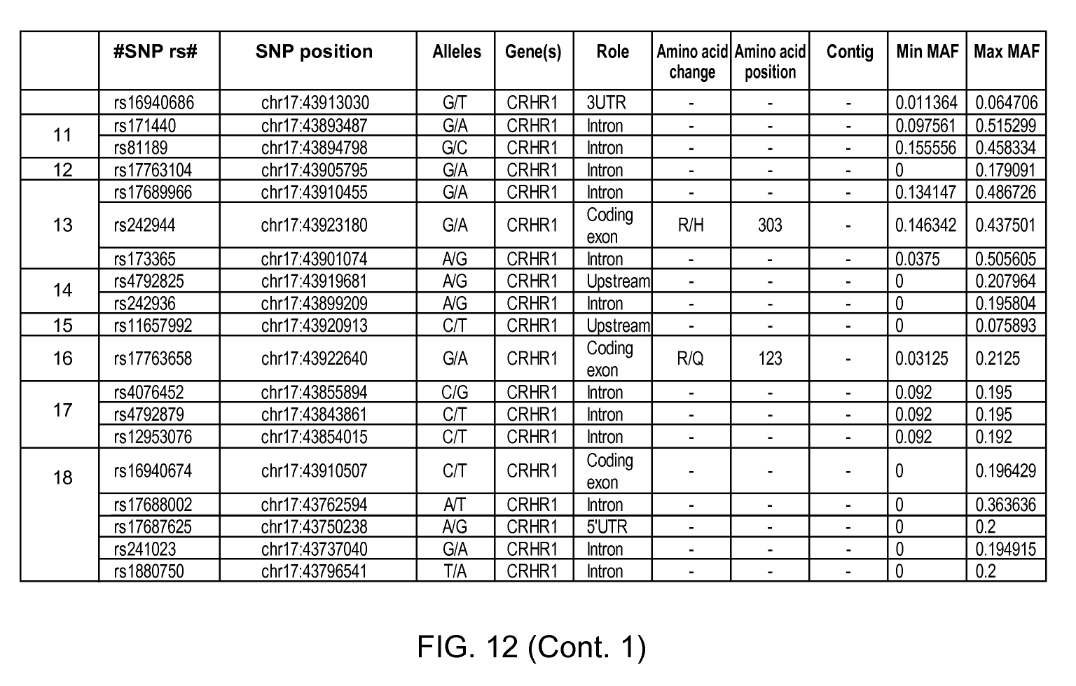

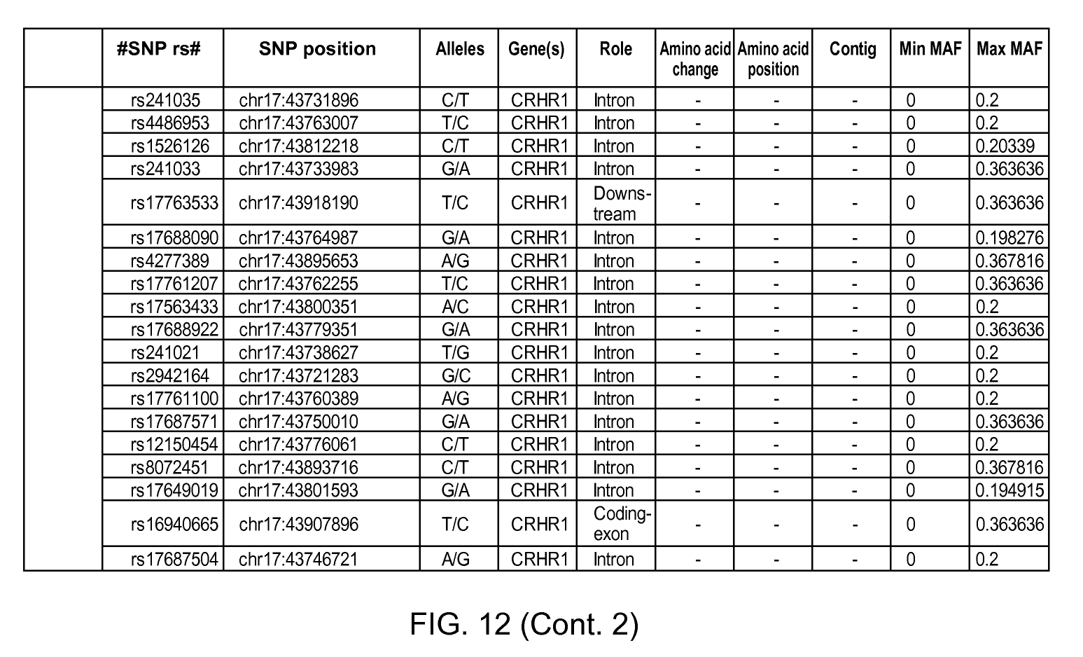

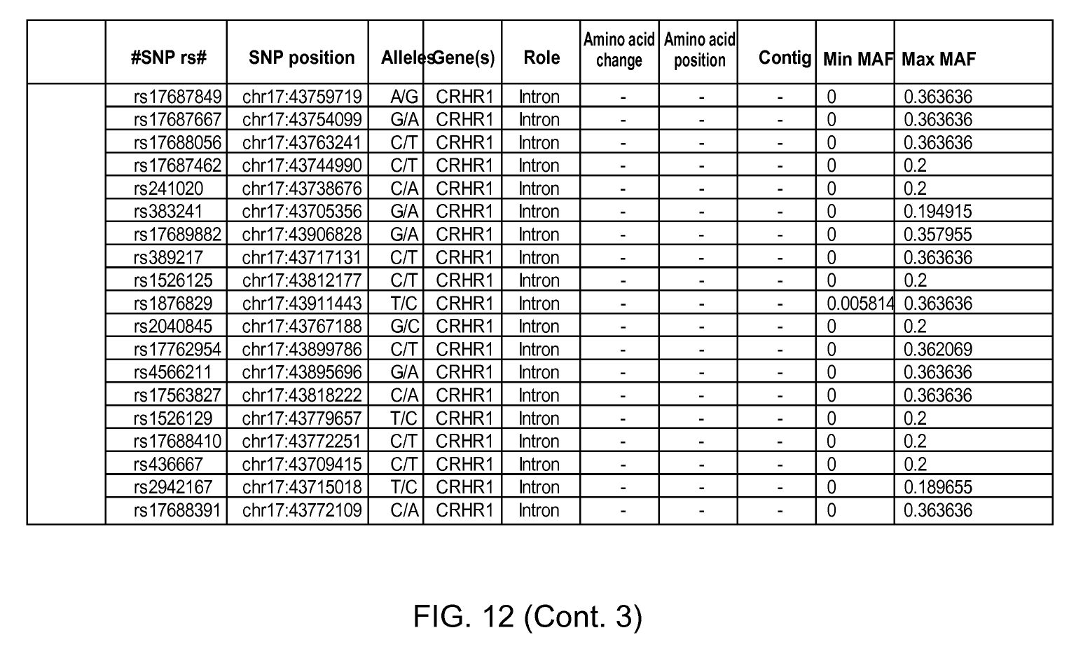

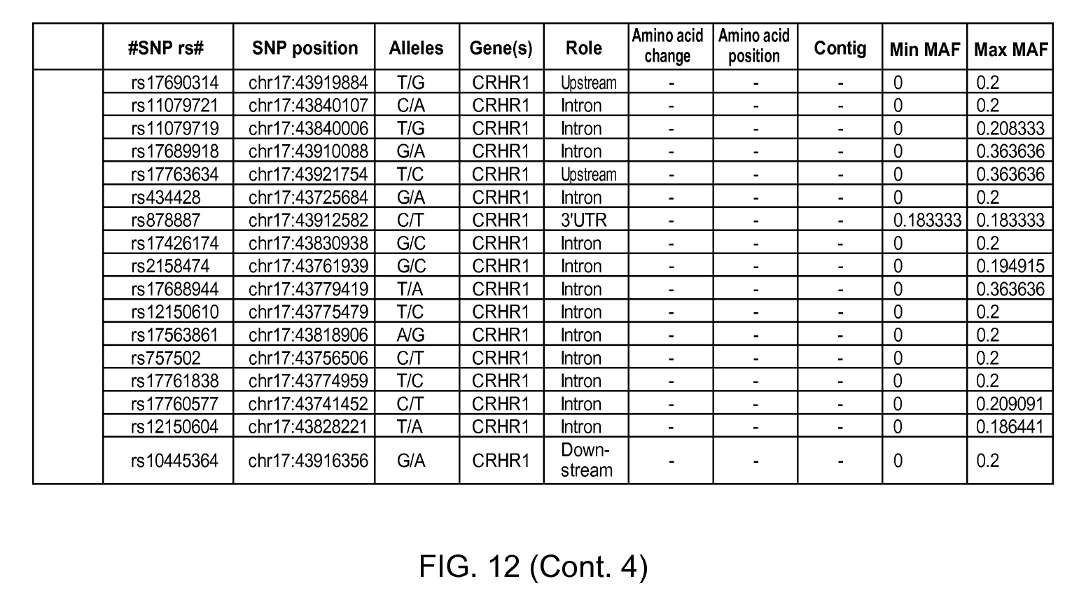

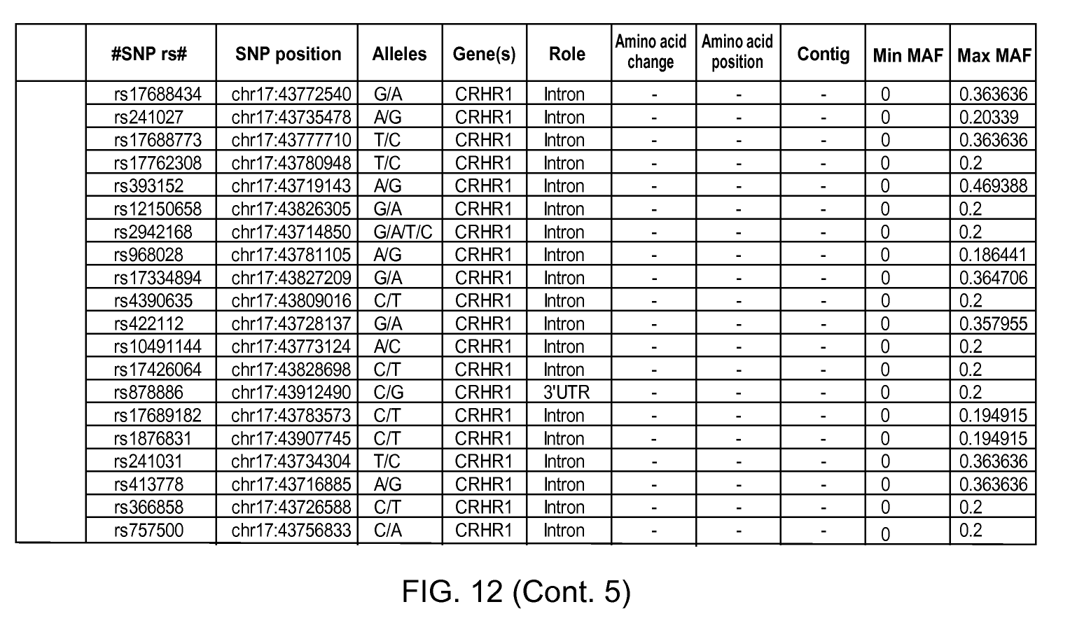

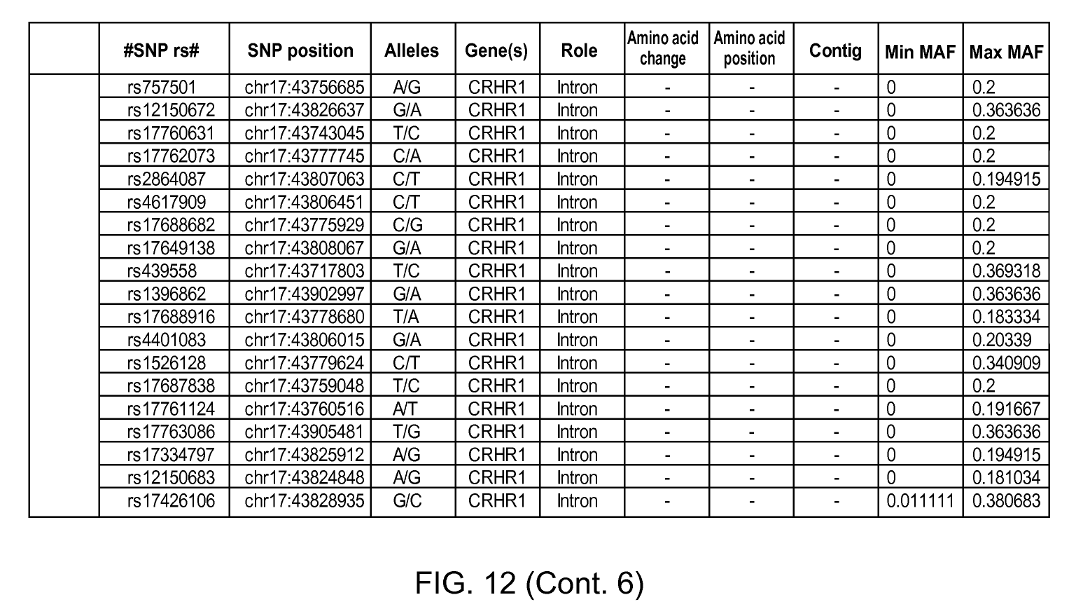

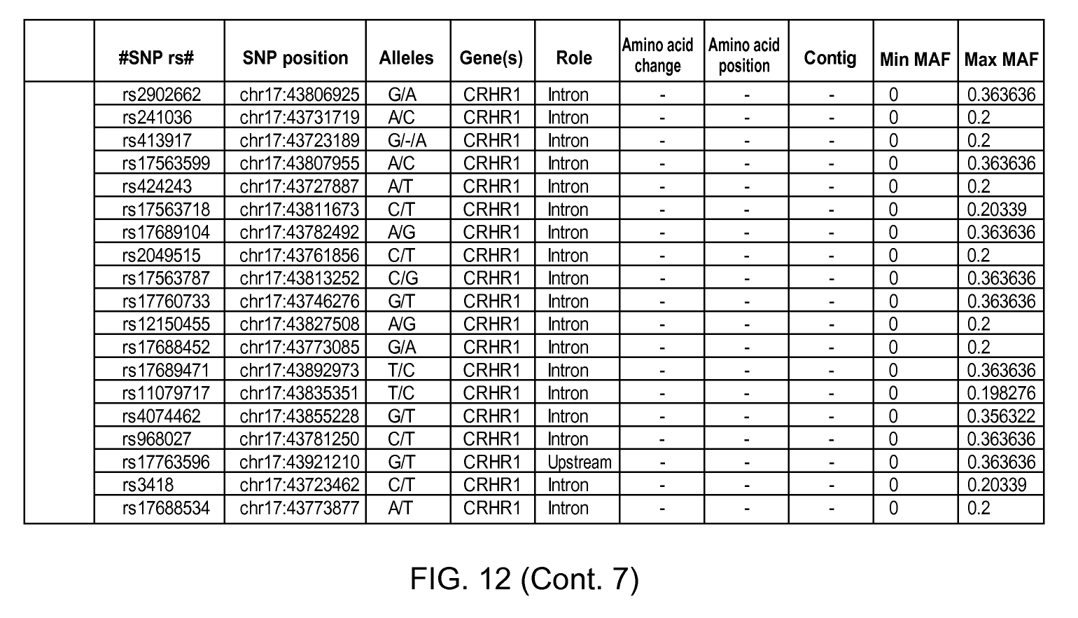

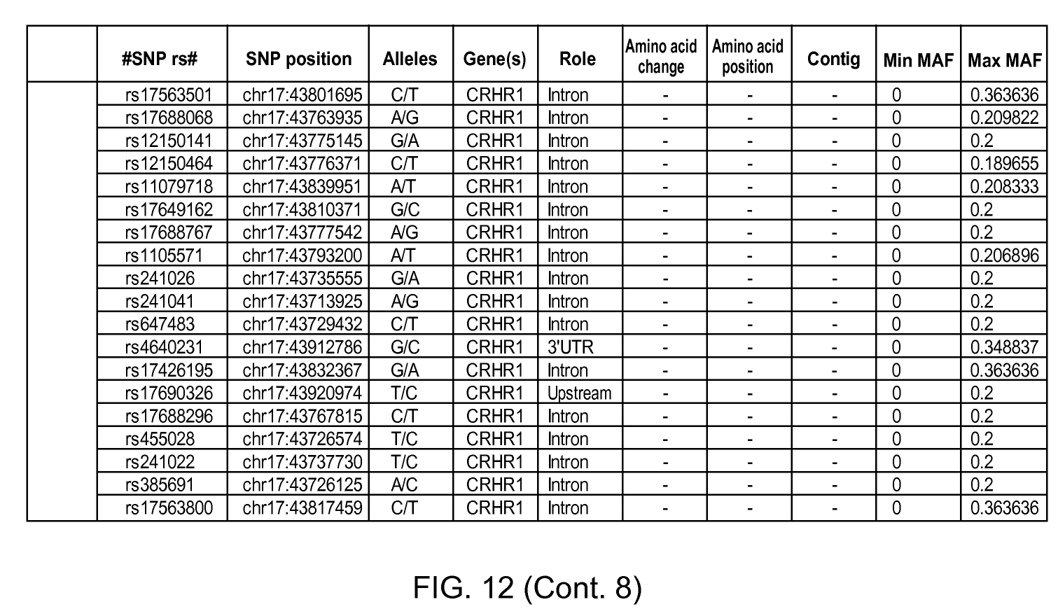

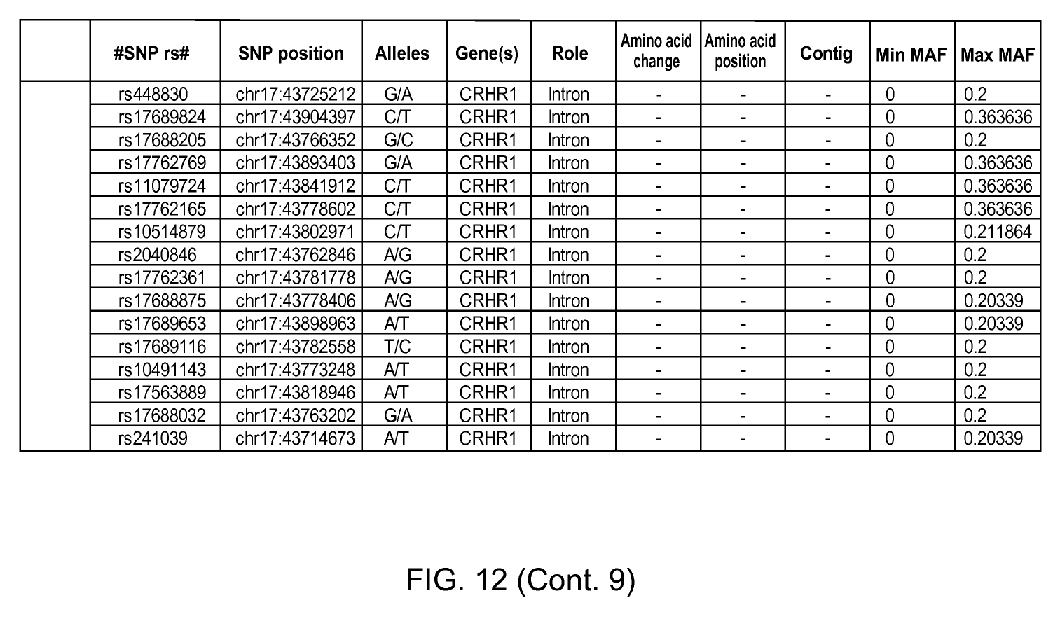

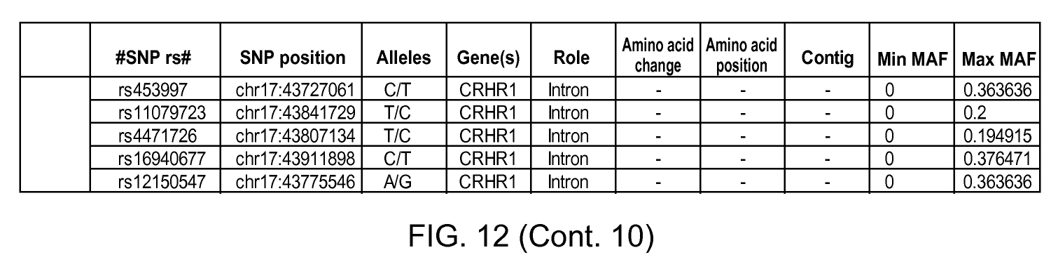

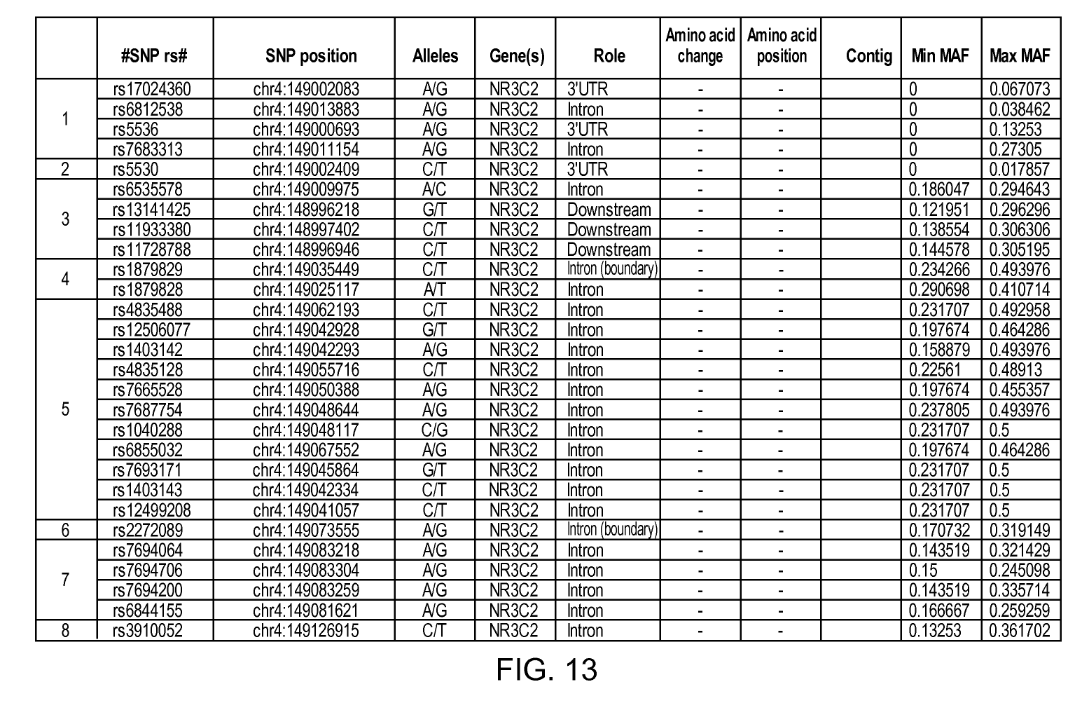

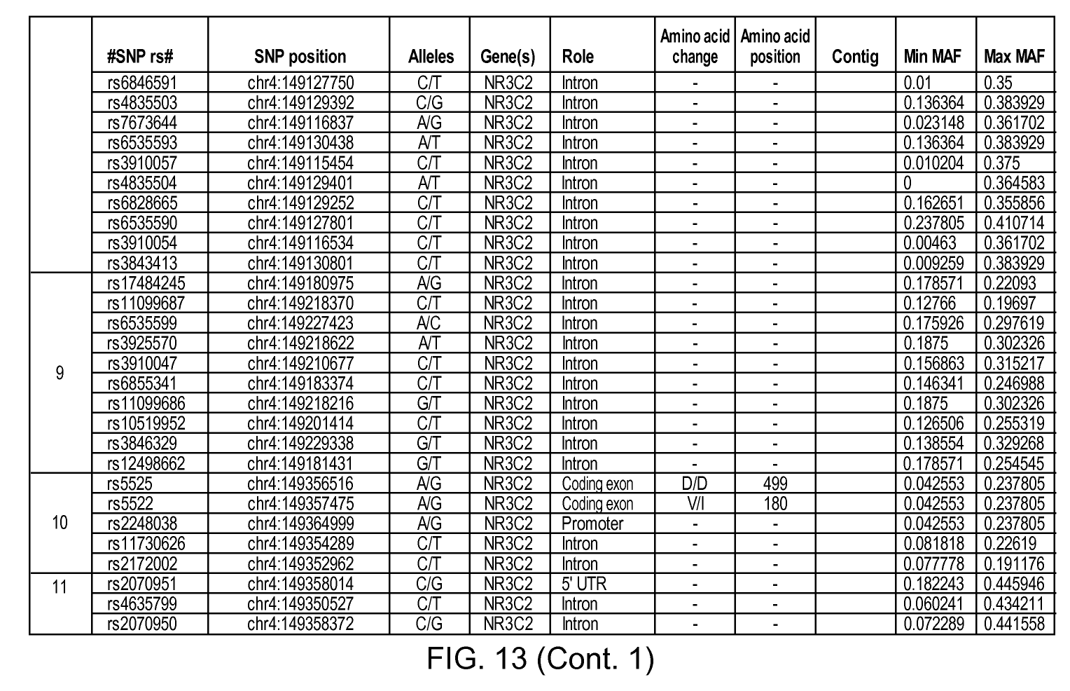

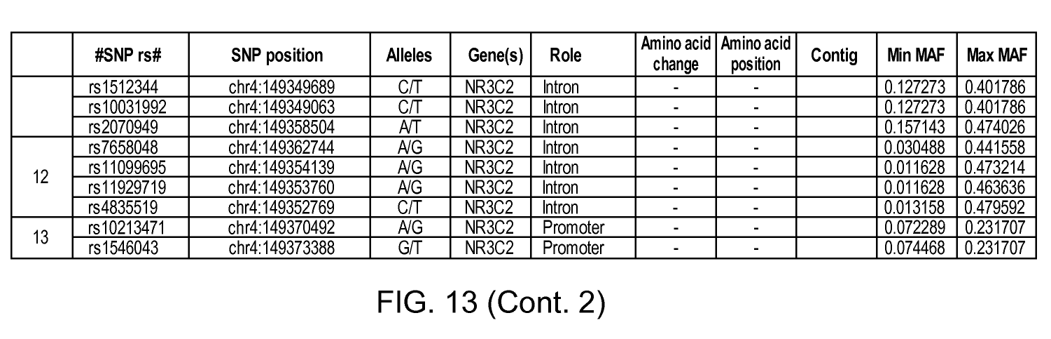

In some embodiments, a subject method further comprises detecting a SNP that is in linkage disequilibrium with one or more of above-listed CRH-R1 and GR SNPs. SNPs that are in linkage disequilibrium with one or more of above-listed CRH-R1 and GR SNPs, and that are suitable for detection using a subject method, are shown in FIGS. 12 and 13.

### Detection Methods

The SNP(s) can be detected by one or more of the following techniques: (a) restriction fragment length analysis; (b) sequencing; (c) a micro-sequencing assay; (d) hybridization; (e) an invader assay; (f) a gene chip hybridization assay; (g) an oligonucleotide ligation assay; (h) ligation rolling circle amplification; (i) a 5′ nuclease assay; (j) a polymerase proofreading method; (k) an allele specific polymerase chain reaction; (l) matrix assisted laser desorption ionization time of flight (MALDI-TOF) mass spectroscopy; (m) a ligase chain reaction assay; (n) enzyme-amplified electronic transduction; (o) a single base pair extension assay; and (p) reading sequence data. Such methods are well known to those skilled in the art.

In some cases, the SNP is detected by hybridizing nucleic acid from an individual with a nucleic acid probe that includes the SNP. A nucleic acid probe that includes the SNP is also referred to as an allele-specific probe.

Polymorphisms are detected in a target nucleic acid isolated from an individual being assessed. Typically genomic DNA is analyzed. For assay of genomic DNA, virtually any biological sample containing genomic DNA or RNA, e.g., nucleated cells, is suitable. For example, genomic DNA can be obtained from peripheral blood leukocytes collected from a patient. Other suitable samples include saliva, cheek scrapings, organ biopsy samples, whole blood, buccal samples, tissue biopsy samples, and the like. Alternatively RNA or cDNA can be assayed. Methods for purification or partial purification of nucleic acids from patient samples for use in diagnostic or other assays are well known

Detection of a SNP that indicates a predisposition to developing depression can be carried out using any of a variety of well-known methods. Suitable methods include, but are not limited to, use of allele-specific probes; use of allele-specific primers; direct sequence analysis; denaturing gradient gel electrophoresis (DGGE) analysis; single-strand conformation polymorphism (SSCP) analysis; and denaturing high performance liquid chromatography (DHPLC) analysis. Other well-known methods to detect polymorphisms in DNA include use of: Molecular Beacons technology (see, e.g., Piatek et al., 1998; Nat. Biotechnol. 16:359-63; Tyagi, and Kramer, 1996, Nat. Biotechnology 14:303-308; and Tyagi, et al., 1998, Nat. Biotechnol. 16:49-53), Invader technology (see, e.g., Neri et al., 2000, Advances in Nucleic Acid and Protein Analysis 3826:117-125 and U.S. Pat. No. 6,706,471), nucleic acid sequence based amplification (Nasba) (Compton, 1991), Scorpion technology (Thelwell et al., 2000, Nuc. Acids Res, 28:3752-3761 and Solinas et al., 2001, “Duplex Scorpion primers in SNP analysis and FRET applications” Nuc. Acids Res, 29:20.), restriction fragment length polymorphism (RFLP) analysis, and the like.

The design and use of allele-specific probes for analyzing polymorphisms are described by e.g., Saiki et al., 1986; Dattagupta, EP 235,726, Saiki, WO 89/11548. Briefly, allele-specific probes are designed to hybridize to a segment of target DNA from one individual but not to the corresponding segment from another individual, if the two segments represent different polymorphic forms. Hybridization conditions are chosen that are sufficiently stringent so that a given probe essentially hybridizes to only one of two alleles. Typically, allele-specific probes are designed to hybridize to a segment of target DNA such that the polymorphic site aligns with a central position of the probe.

Allele-specific probes are often used in pairs, one member of a pair designed to hybridize to the reference allele of a target sequence and the other member designed to hybridize to the variant allele. Several pairs of probes can be immobilized on the same support for simultaneous analysis of multiple polymorphisms within the same target gene sequence.

### Probes

Exemplary allele-specific probes for analyzing a CRHR2 SNP that is predictive of depression include:

1) allele-specific probes for detecting rs4723003, where exemplary probes include, e.g.:

a) 5′-AGAAACCATTTGCTATCTCTG-3′
(nucleotides 36,229-36,249 of SEQ ID NO: 4);
b) 5′-CAGAGATAGCAAATGGTTTCT-3′;
c) 5′-CCAGAAACCATTTGCTATCTCTGTT-3′;
and
d) 5′-AACAGAGATAGCAAATGGTTTCTGG-3′.

Such probes would be expected to detect the C-to-T substitution that characterizes rs4723003.

Probes that would be useful for detecting the corresponding “wild-type” or “normal” sequence corresponding to rs4723003 include, e.g.:

a) 5′-AGAAACCATTCGCTATCTCTG-3′;
b) 5′-CAGAGATAGCGAATGGTTTCT-3′;
d) 5′-CCAGAAACCATTCGCTATCTCTGTT-3′;
and
d) 5′-AACAGAGATAGCGAATGGTTTCTGG-3′.

2) allele-specific probes for detecting rs2284216, where exemplary probes include, e.g.:

a) 5′-CTCTGGAGCATAAGTTTACCT-3′;
b) 5′-AGGTAAACTTATGCTCCAGAG-3′;
c) 5′-TTCTCTGGAGCATAAGTTTACCTTA-3′;
and
d) 5′-TAAGGTAAACTTATGCTCCAGAGAA-3′.

Such probes would be expected to detect the G-to-T substitution that characterizes rs2284216.

Probes that would be useful for detecting the corresponding “wild-type” or “normal” sequence corresponding to rs2284216 include, e.g.:

a) 5′-CTCTGGAGCAGAAGTTTACCT-3′
(nucleotides 22,425 to 22,445 of SEQ ID NO: 4);
b) 5′-AGGTAAACTTCTGCTCCAGAG-3′;
c) 5′-TTCTCTGGAGCAGAAGTTTACCTTA-3′;
and
d) 5′-TAAGGTAAACTTCTGCTCCAGAGAA-3′.

Those of ordinary skill in the art can readily design additional probes, given the information provided in the figures, and given the sequences provided above.

The present disclosure provides a panel of allele-specific probes that detect the following CRH-R1 SNPs: rs110402; rs16940674; rs171440; rs17689966; rs242924; rs242940; rs242948; rs4076452; rs4792887; rs4792888; rs7209436; rs3785877; rs4792825; rs4458044; rs12944712; rs17763104; rs2664008; rs17763658; rs242942; and rs11657992.

The present disclosure provides a panel of allele-specific probes that detect the following GR SNPs: rs6198; rs33388; rs2918419; rs10052957; rs10482633; rs12521436; rs12655166; rs17209258; and rs41423247.

### Primers

The design and use of allele-specific primers for analyzing polymorphisms are described by, e.g., WO 93/22456. Briefly, allele-specific primers are designed to hybridize to a site on target DNA overlapping a polymorphism and to prime DNA amplification according to standard PCR protocols only when the primer exhibits perfect complementarity to the particular allelic form. A single-base mismatch prevents DNA amplification and no detectable PCR product is formed. The method works best when the polymorphic site is at the extreme 3′-end of the primer, because this position is most destabilizing to elongation from the primer.

Suitable allele-specific primers for analyzing a depression-associated SNP in a human CRHR2 gene include the following.

Suitable allele-specific primers for analyzing an rs4723003 SNP include, e.g.:

a) 5′-GTCACTACCAGAAACCATTTGCT-3′;
b) 5′-TGTGTCACTACCAGAAACCATTTG-3′;
c) 5′-CAAACAAGGAACAGAGATAGCAAAT-3′;
and
d) 5′-ATACAAACAAGGAACAGAGATAGCAA-3′.

Such primers would detect the C-to-T substitution that characterizes rs4723003.

Corresponding primers that would be suitable for detecting the “normal” or “wild-type” sequence corresponding to rs4723003 include, e.g.:

a) 5′-GTCACTACCAGAAACCATTCGCT-3′;
b) 5′-TGTGTCACTACCAGAAACCATTCG-3′;
c) 5′-CAAACAAGGAACAGAGATAGCGAAT-3′;
and
d) 5′-ATACAAACAAGGAACAGAGATAGCGA-3′.

Suitable allele-specific primers for analyzing an rs2284216 SNP include, e.g.:

a) 5′-CAGGTTCTCTGGAGCATAAG-3′;
b) 5′-CAGCCCAGGTTCTCTGGAGCATA-3′;
c) 5′-ACAAAATAAGGTAAACTTATGC-3′;
and
d) 5′-TGTACAAAATAAGGTAAACTTAT-3′.

Such primers would detect the g-to-T substitution that characterizes rs2284216.

Corresponding primers that would be suitable for detecting the “normal” or “wild-type” sequence corresponding to rs2284216 include, e.g.:

a) 5′-CAGGTTCTCTGGAGCAGAAG-3′;
b) 5′-CAGCCCAGGTTCTCTGGAGCAGA-3′;
c) 5′-ACAAAATAAGGTAAACTTCTGC-3′;
and
d) 5′-TGTACAAAATAAGGTAAACTTCT-3′.

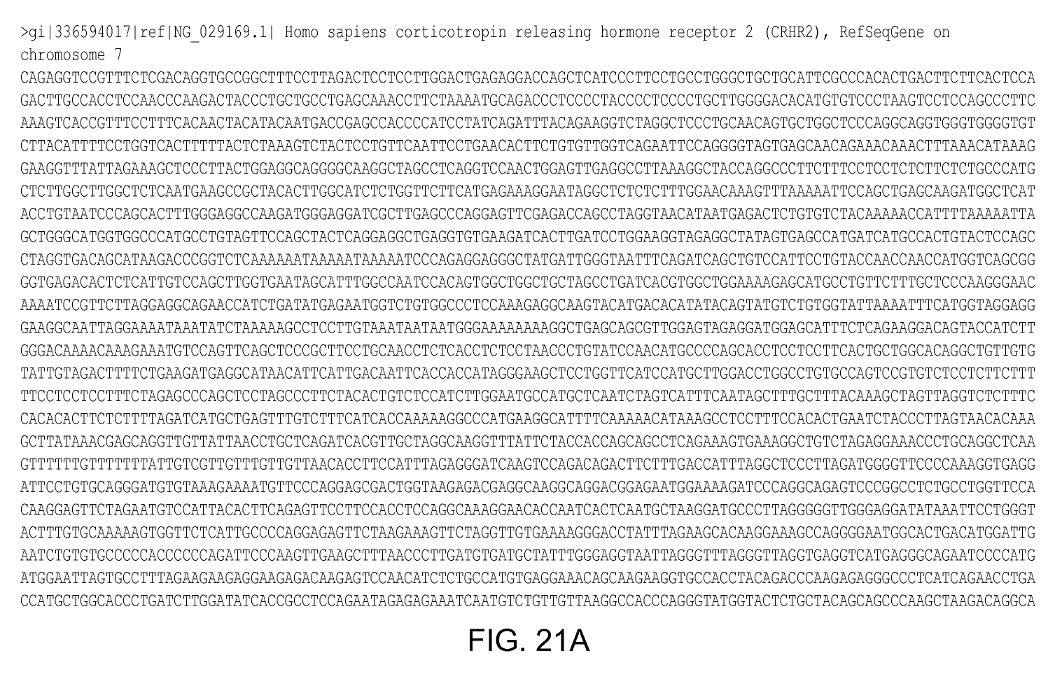

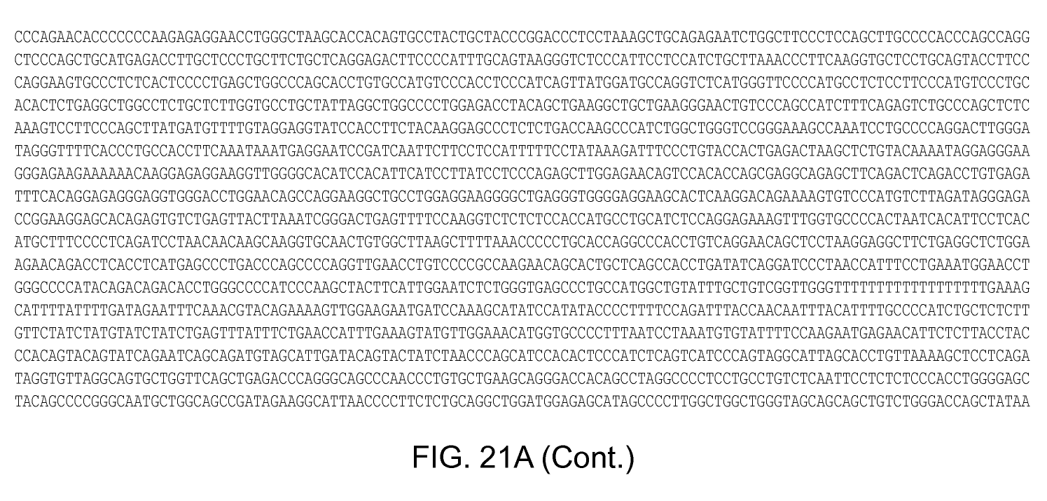

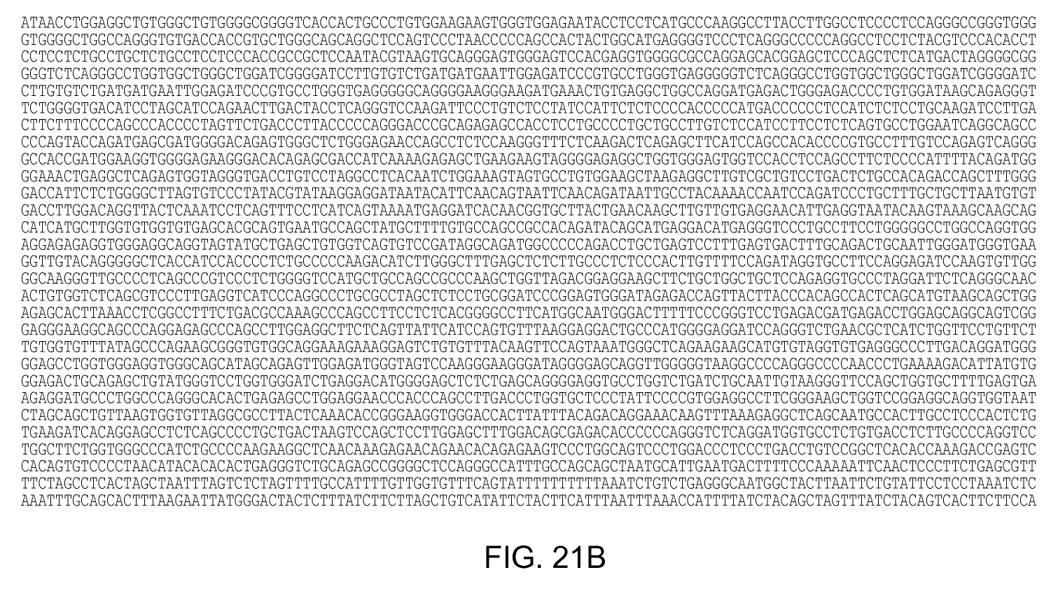

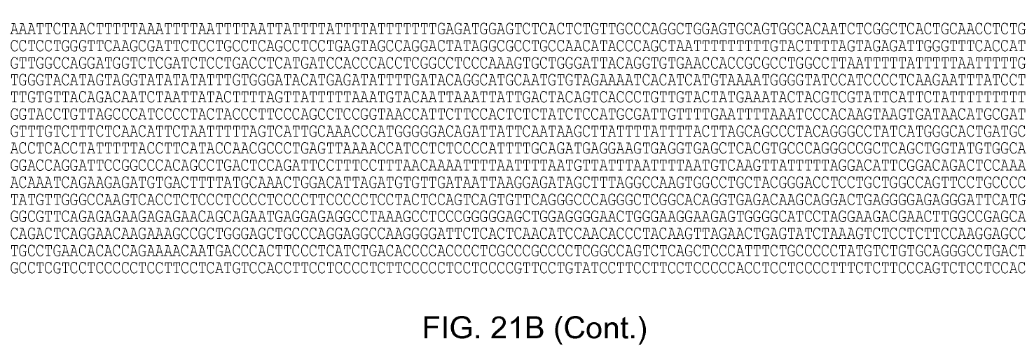

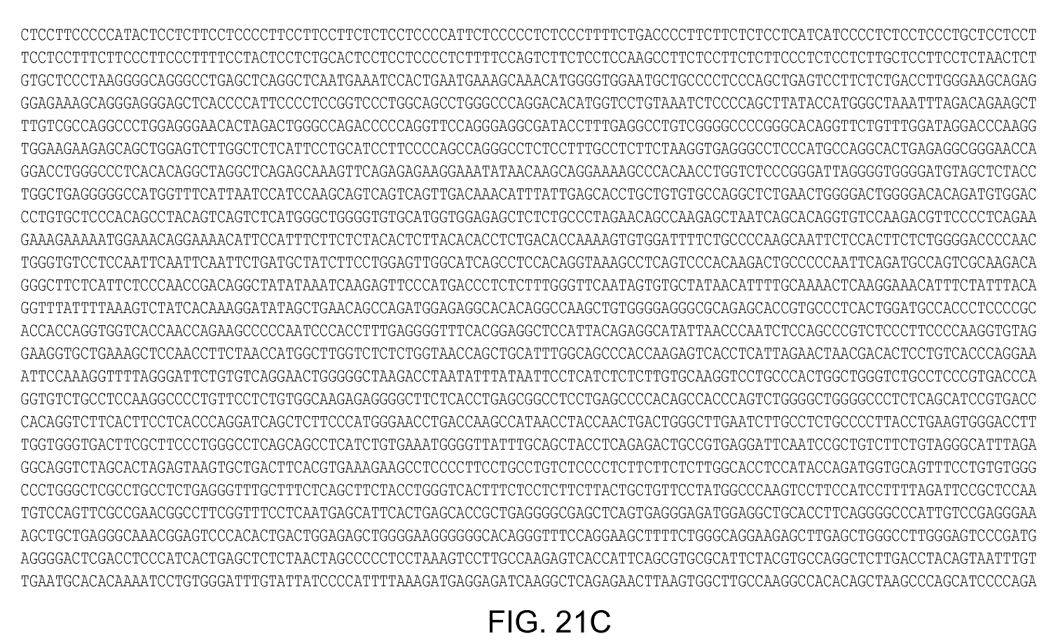

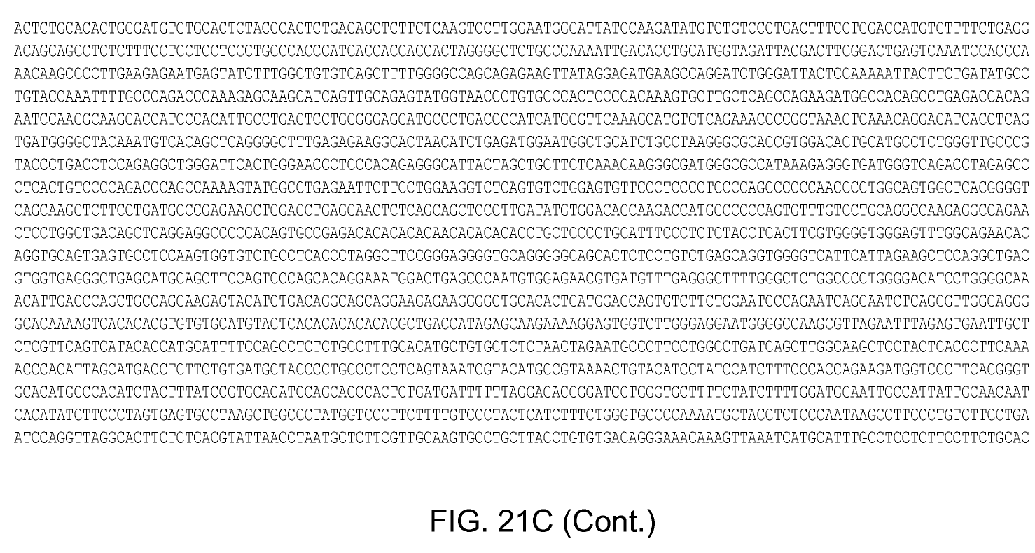

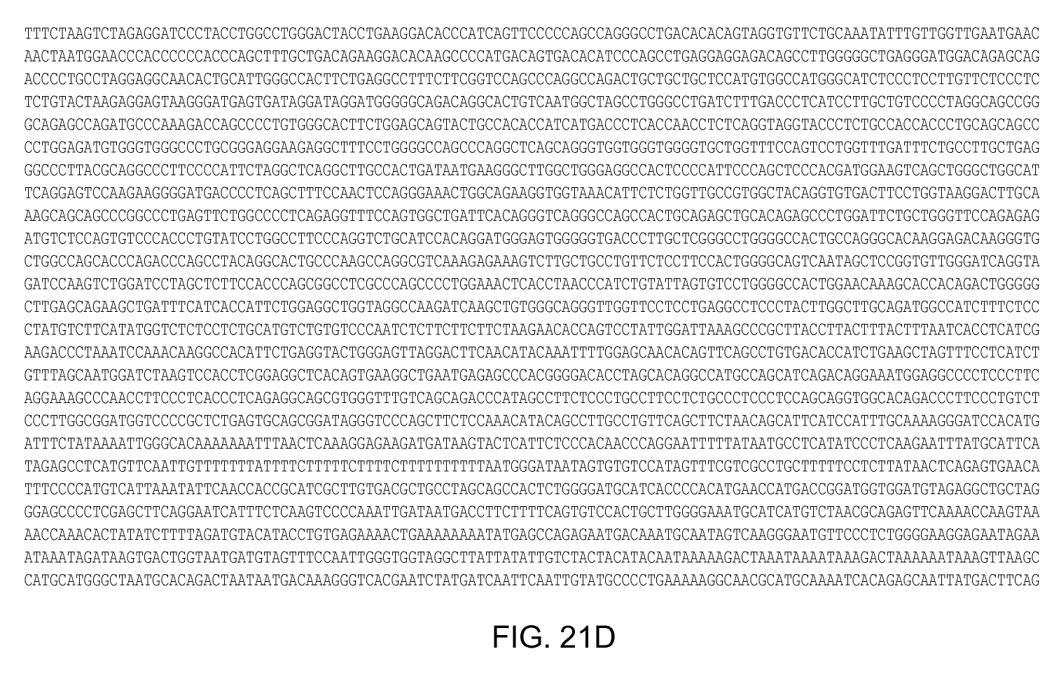

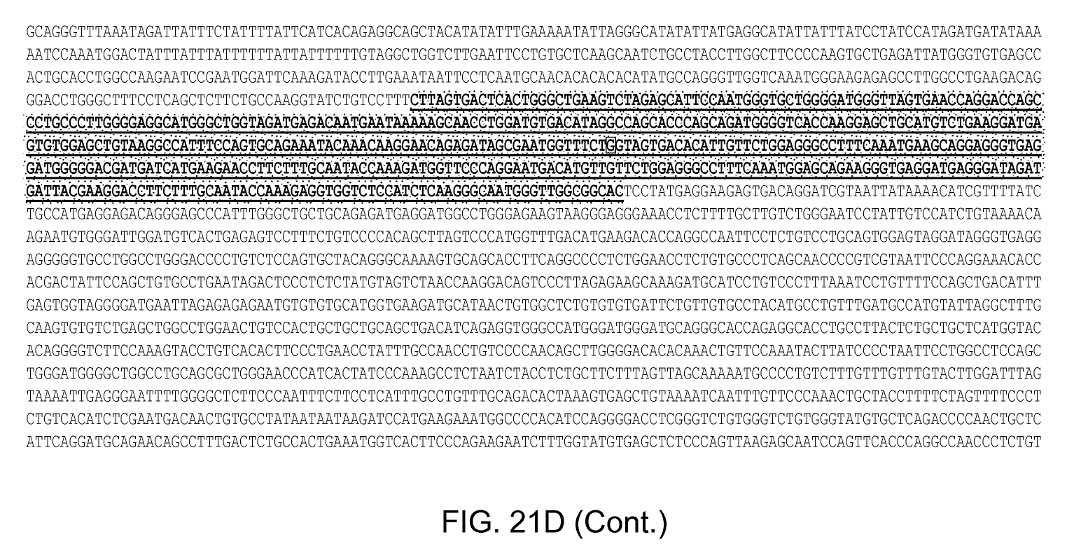

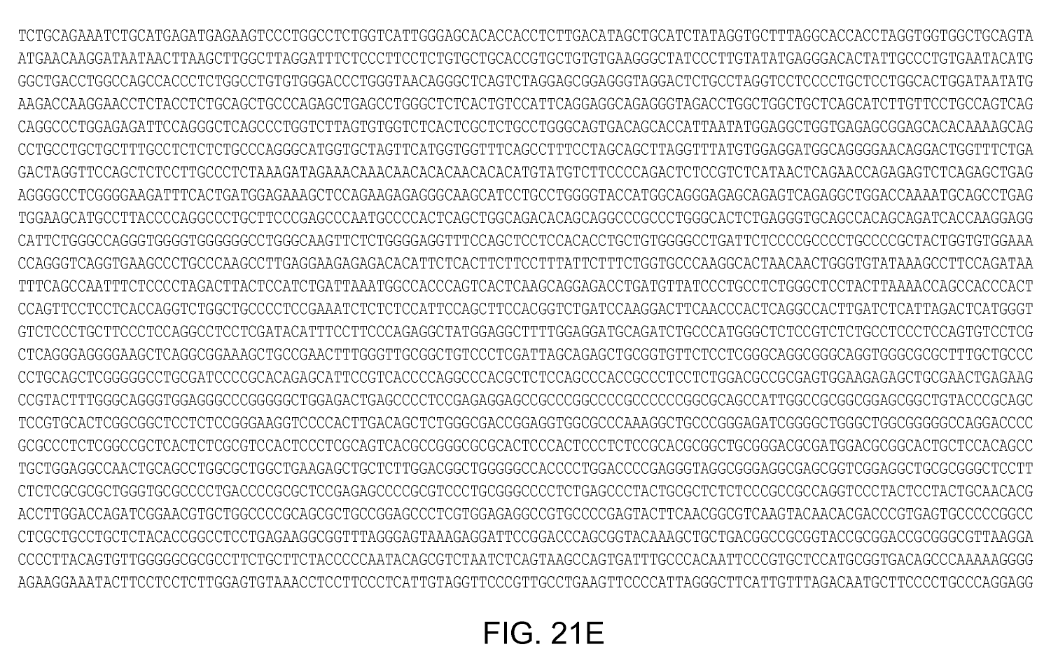

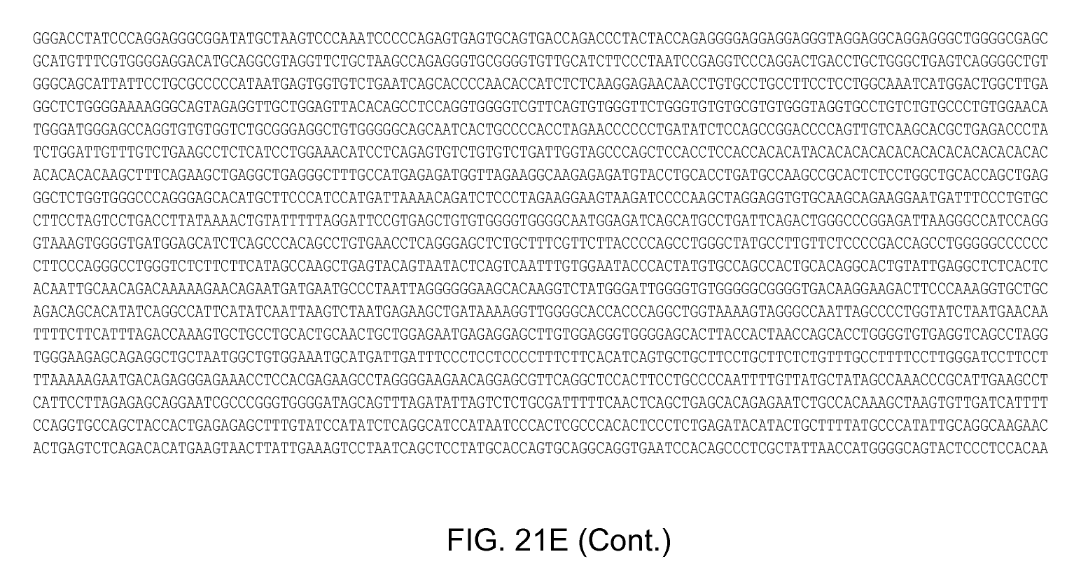

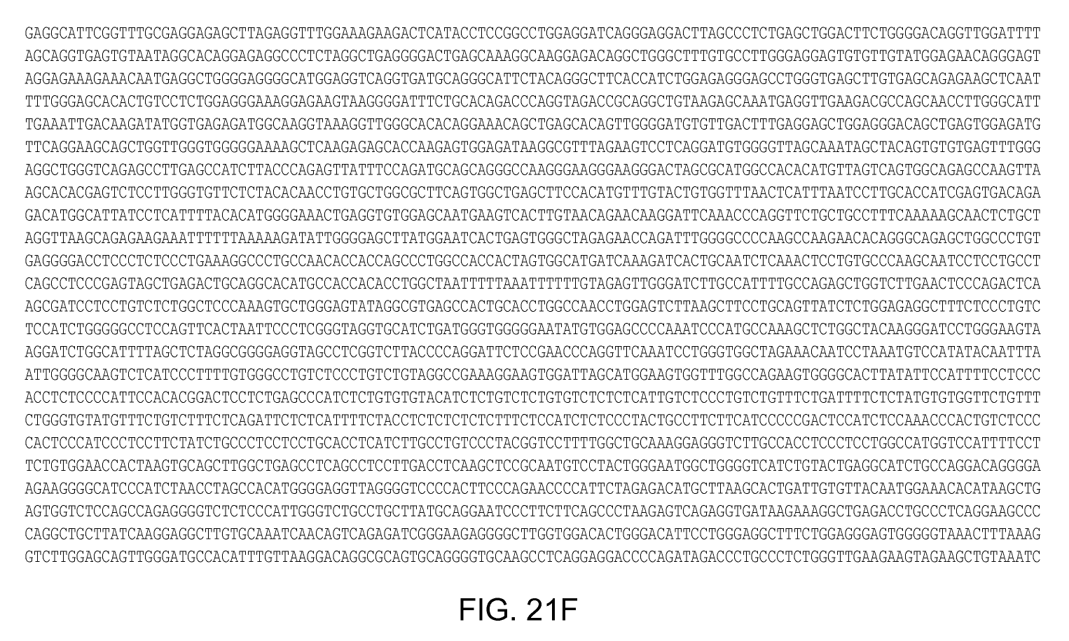

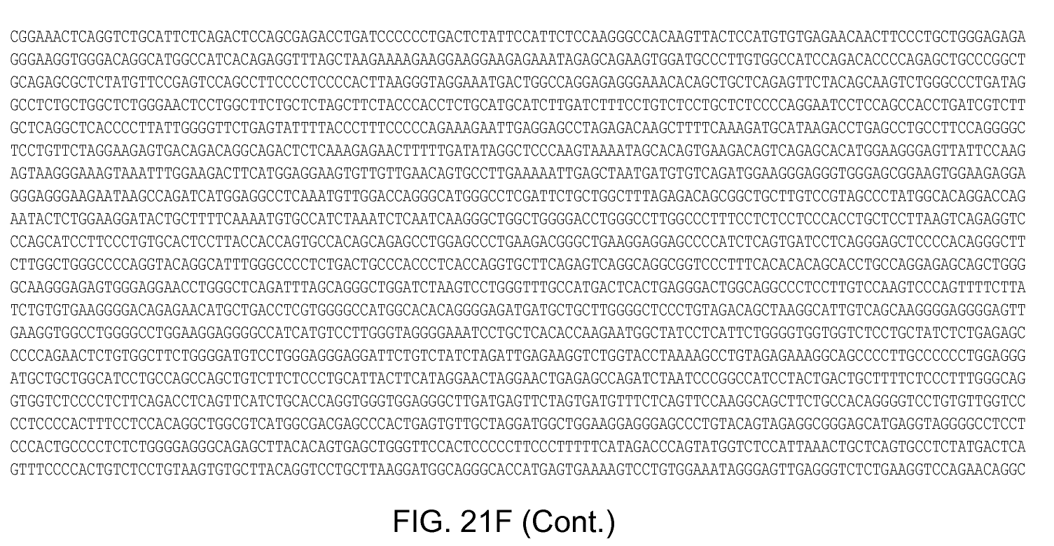

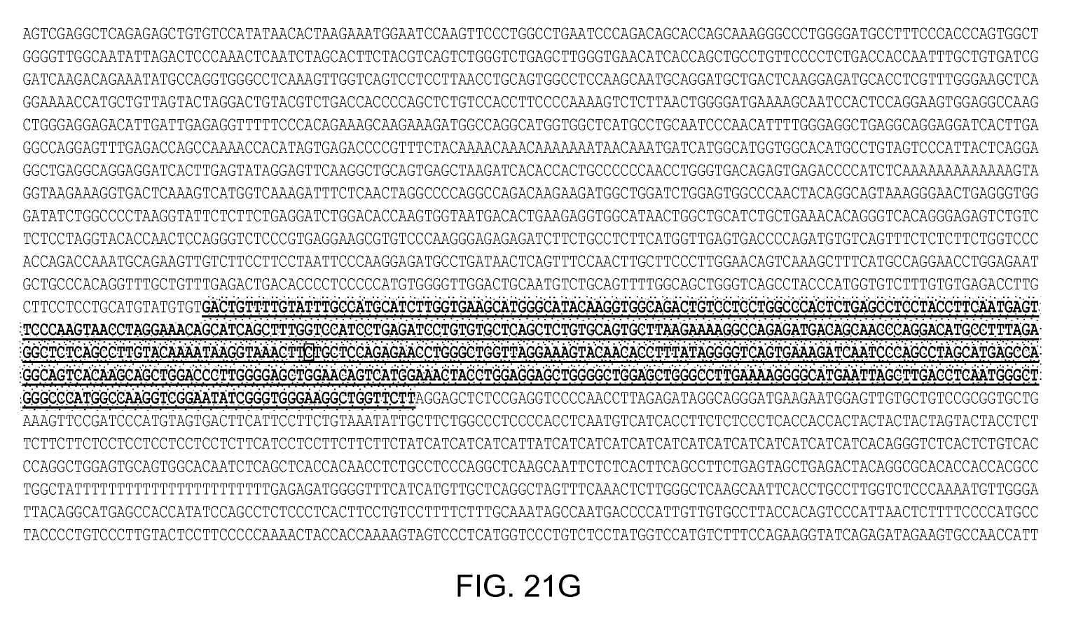

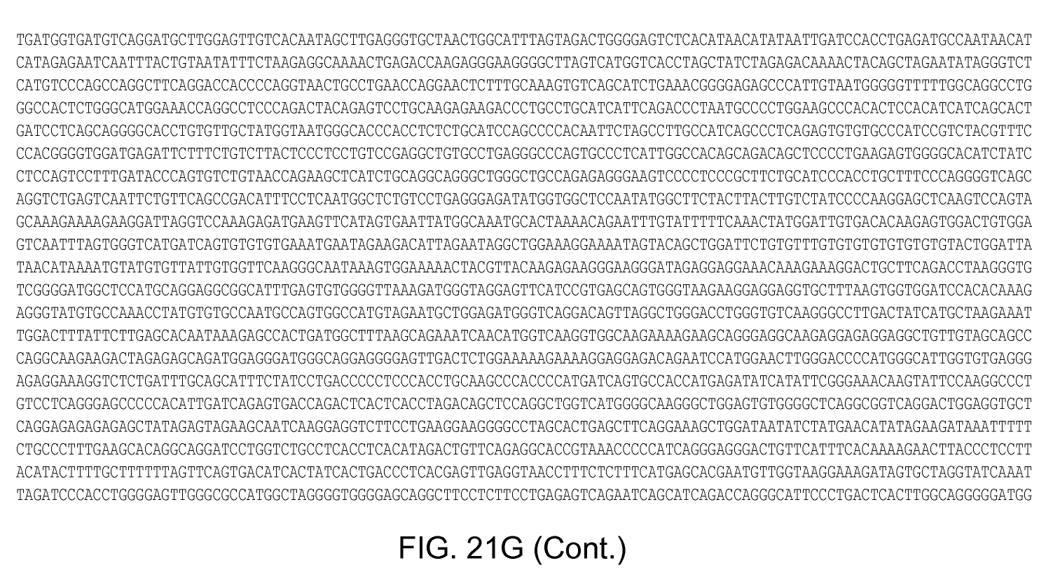

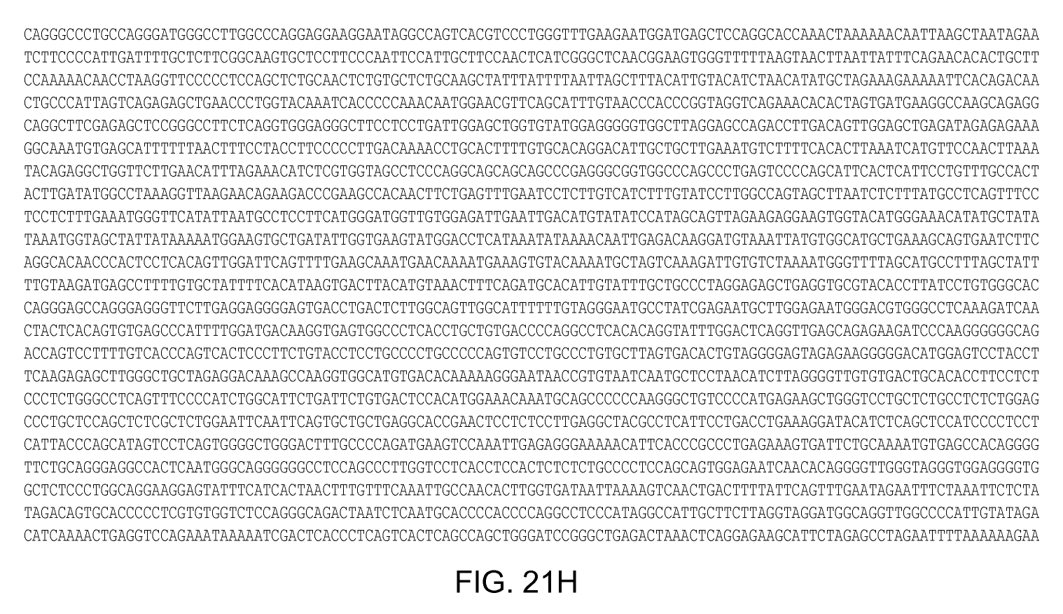

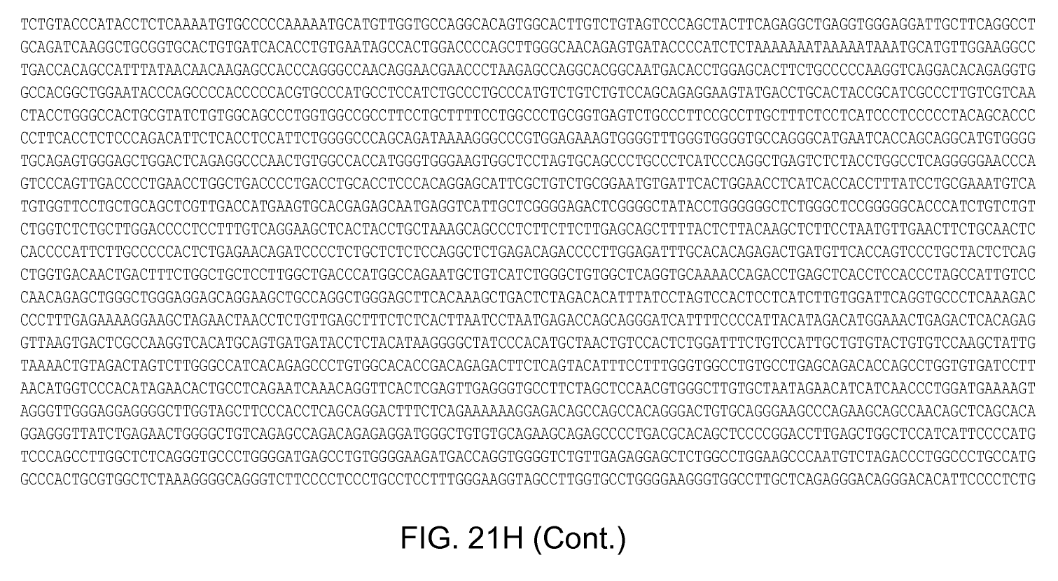

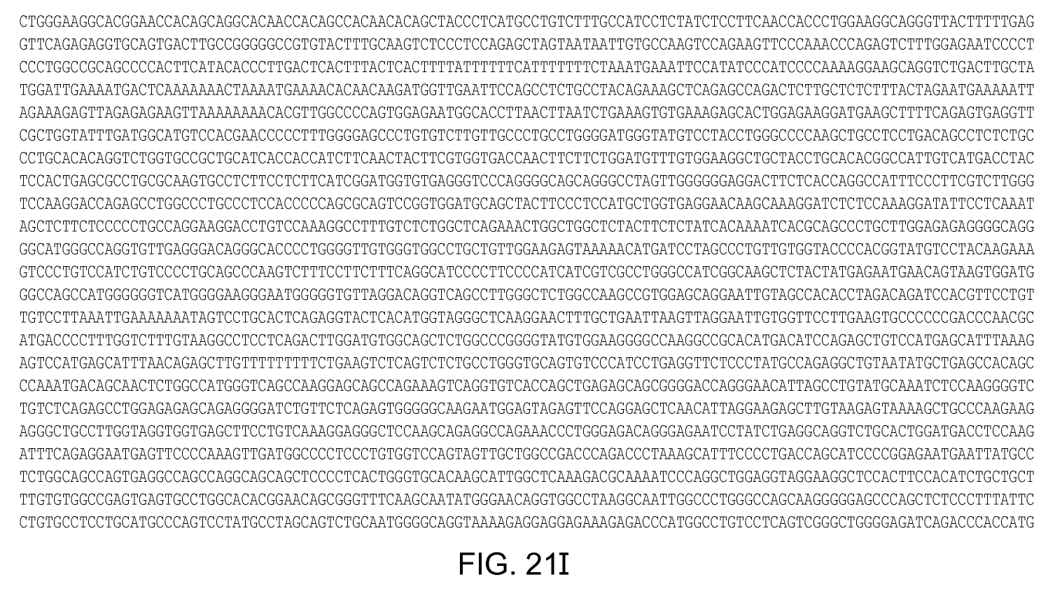

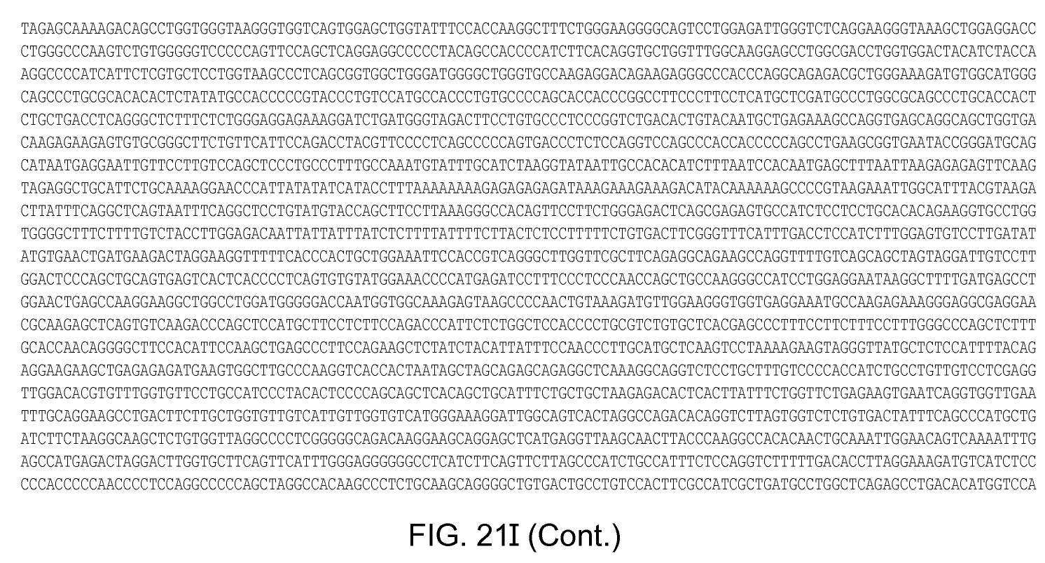

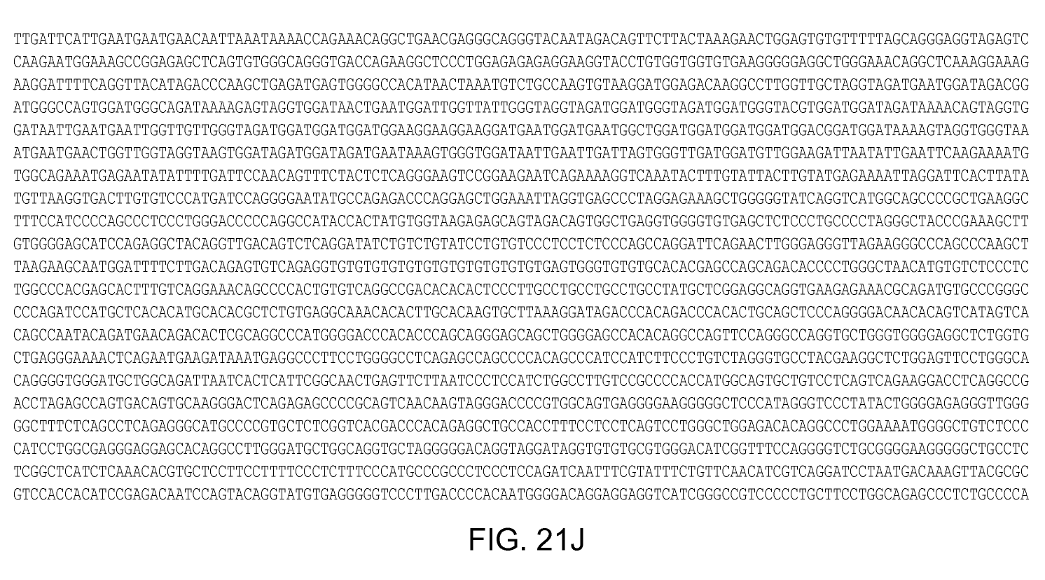

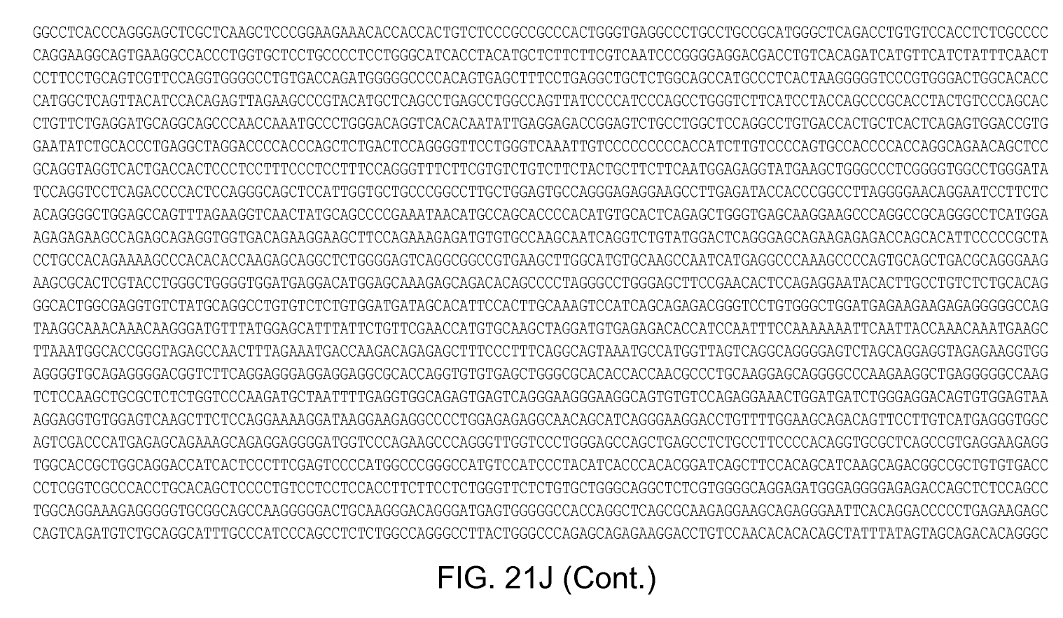

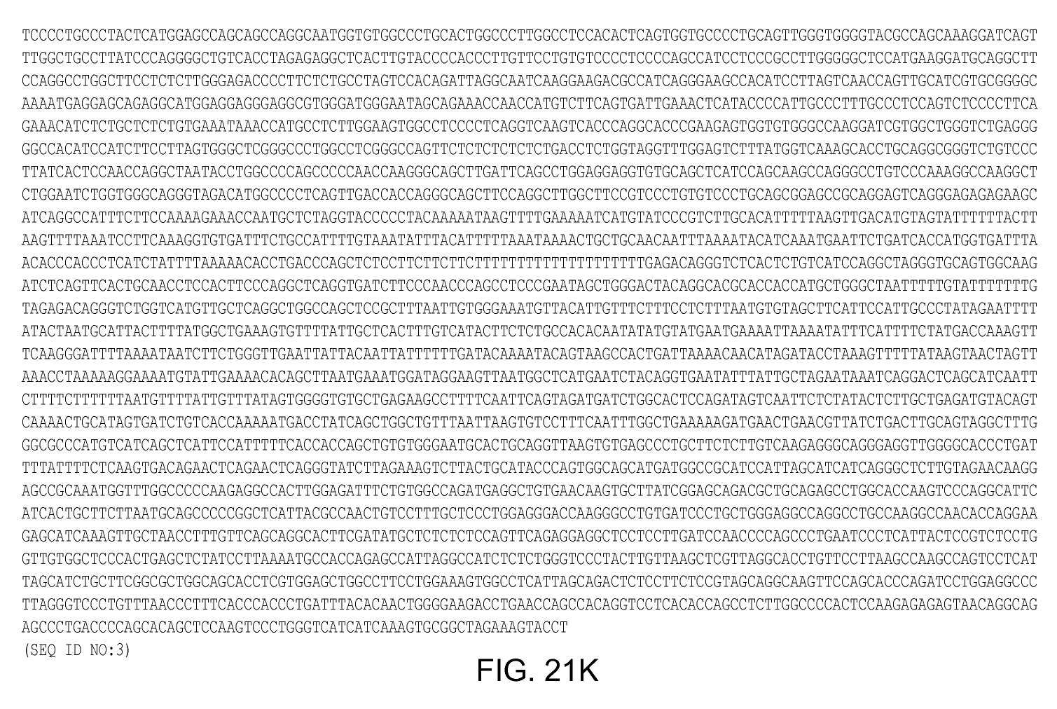

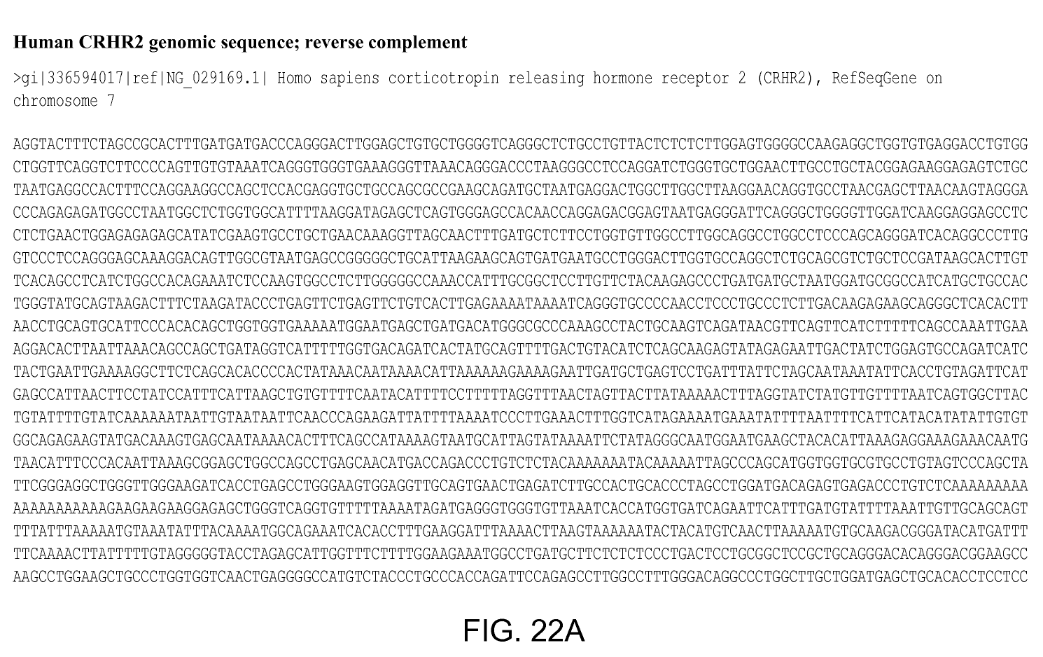

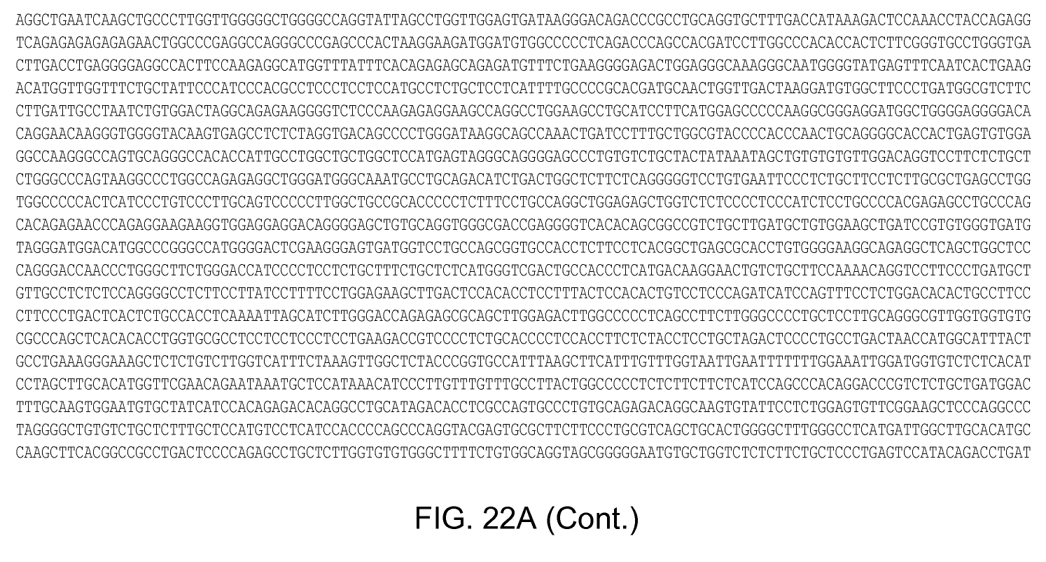

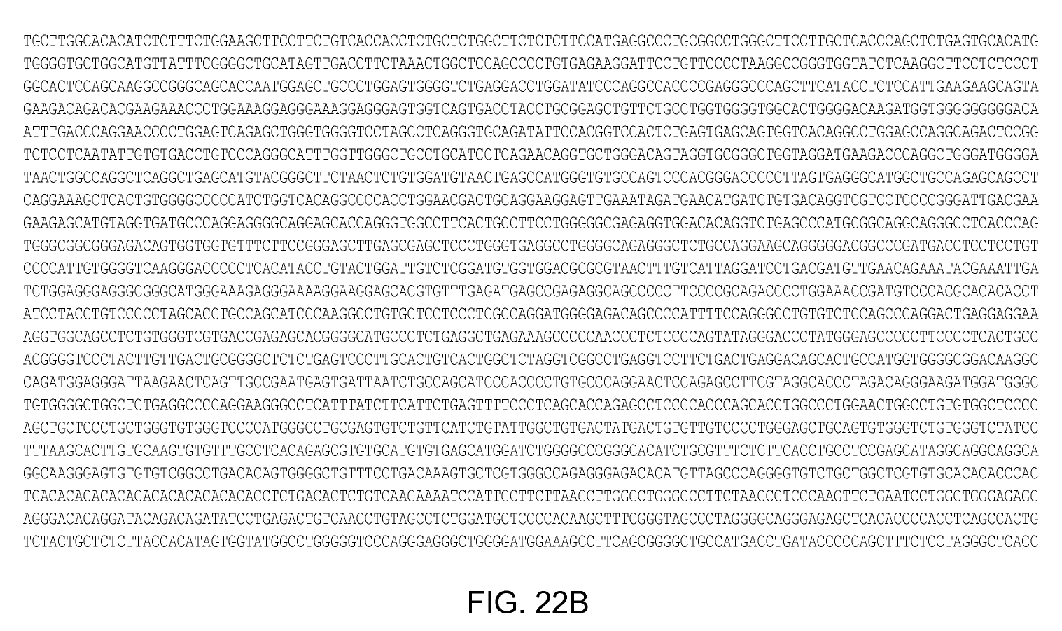

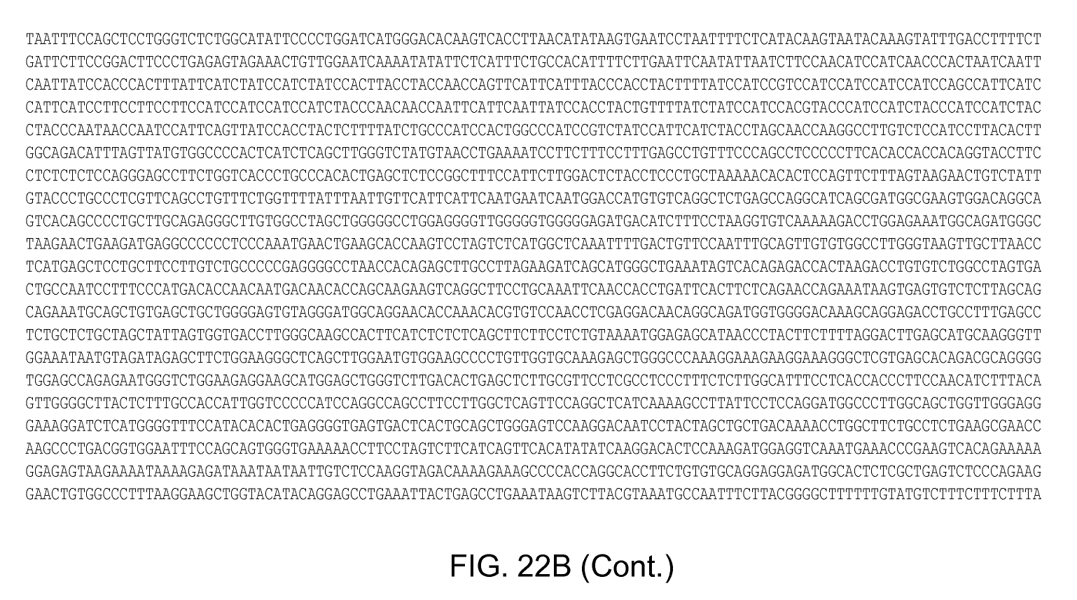

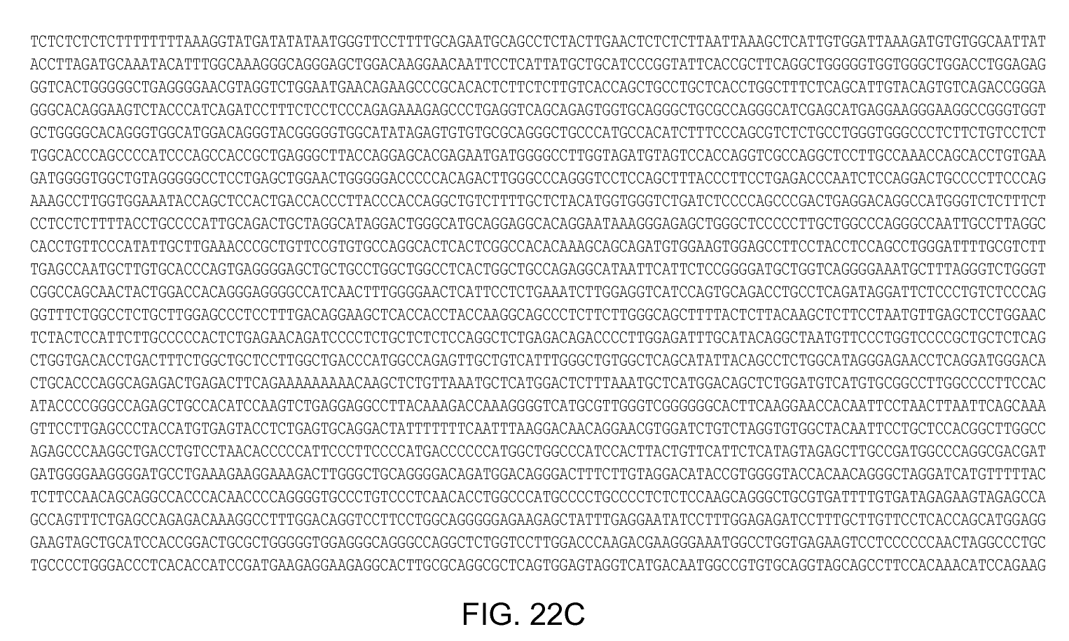

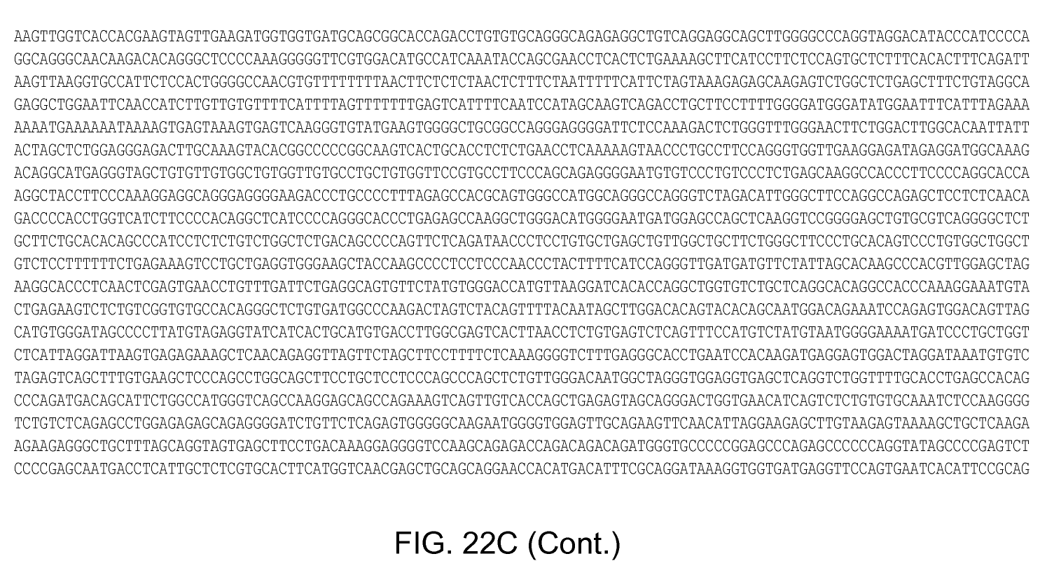

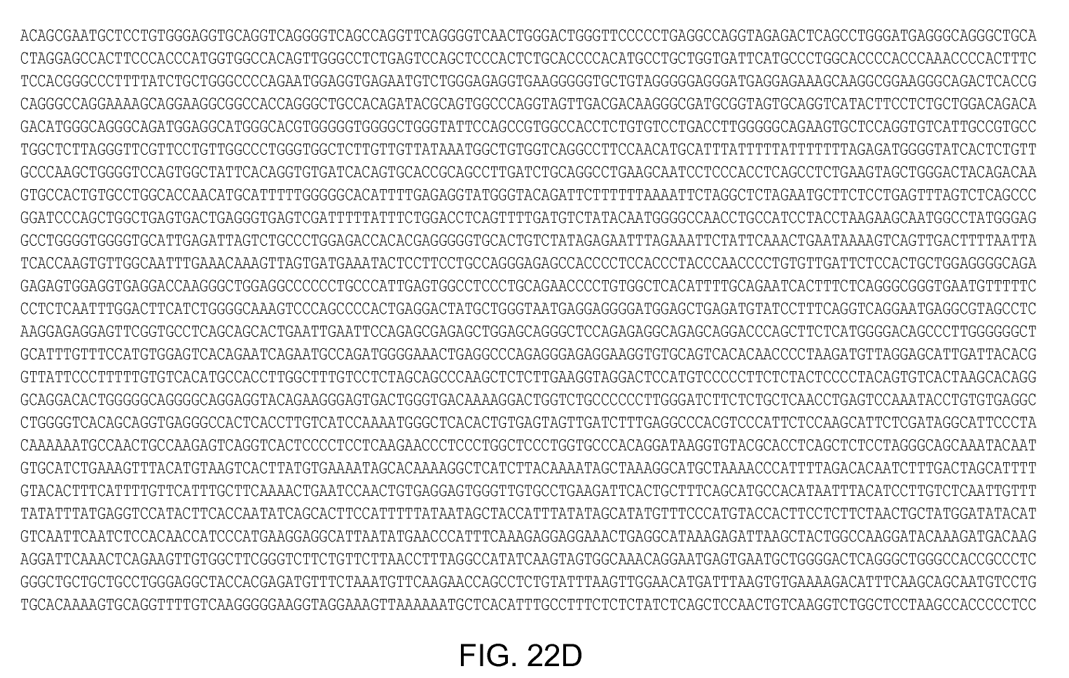

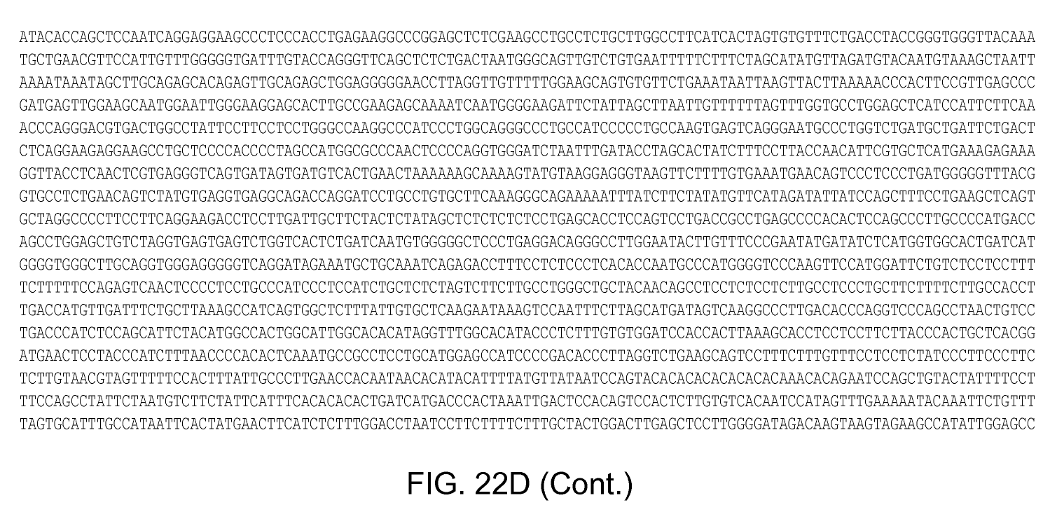

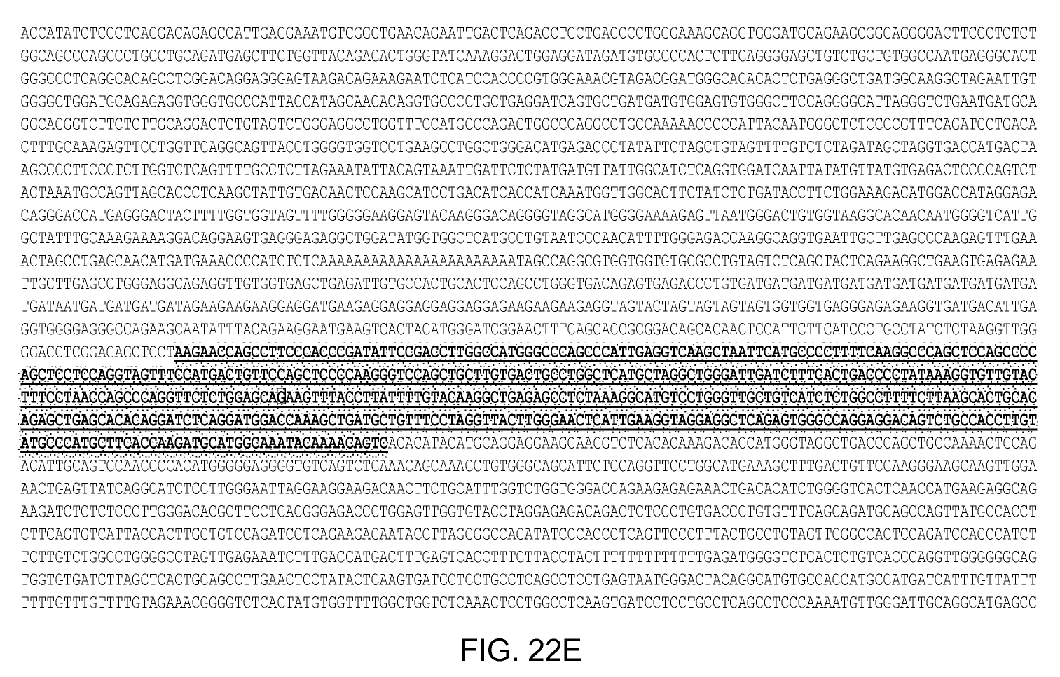

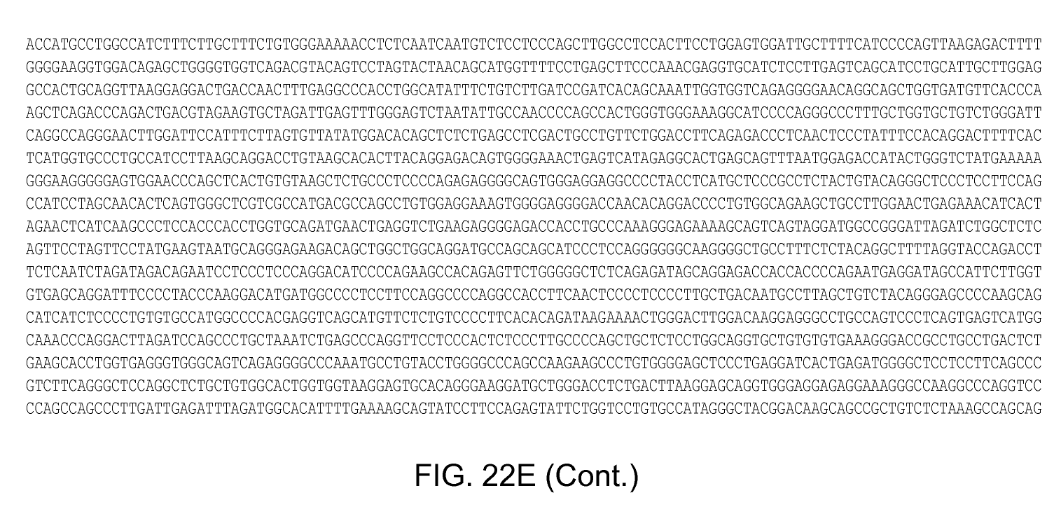

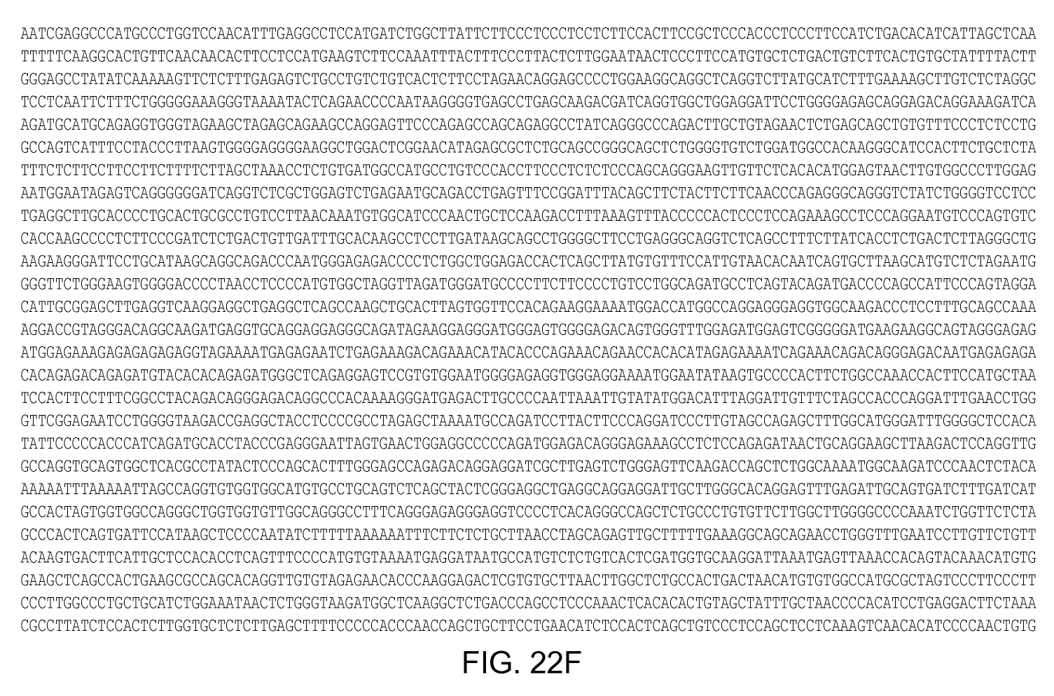

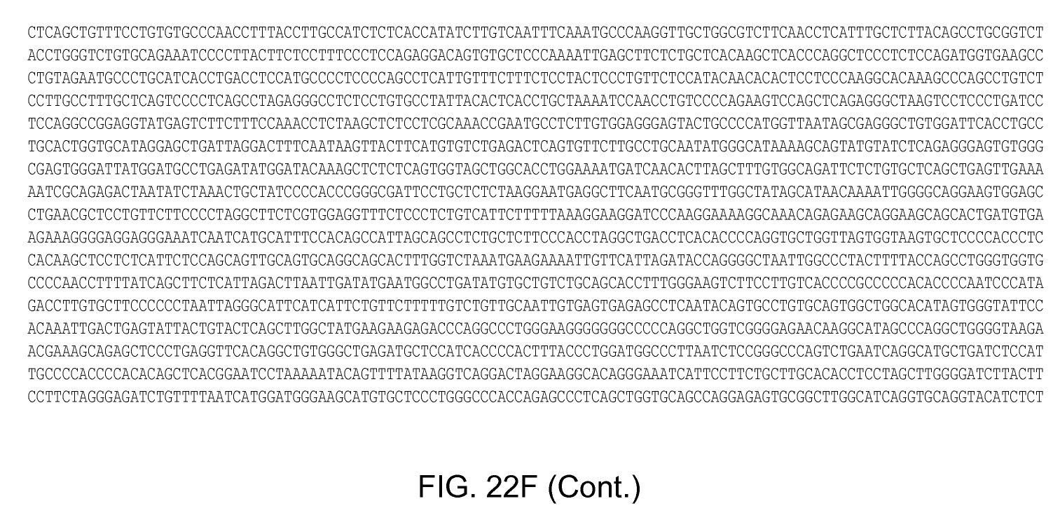

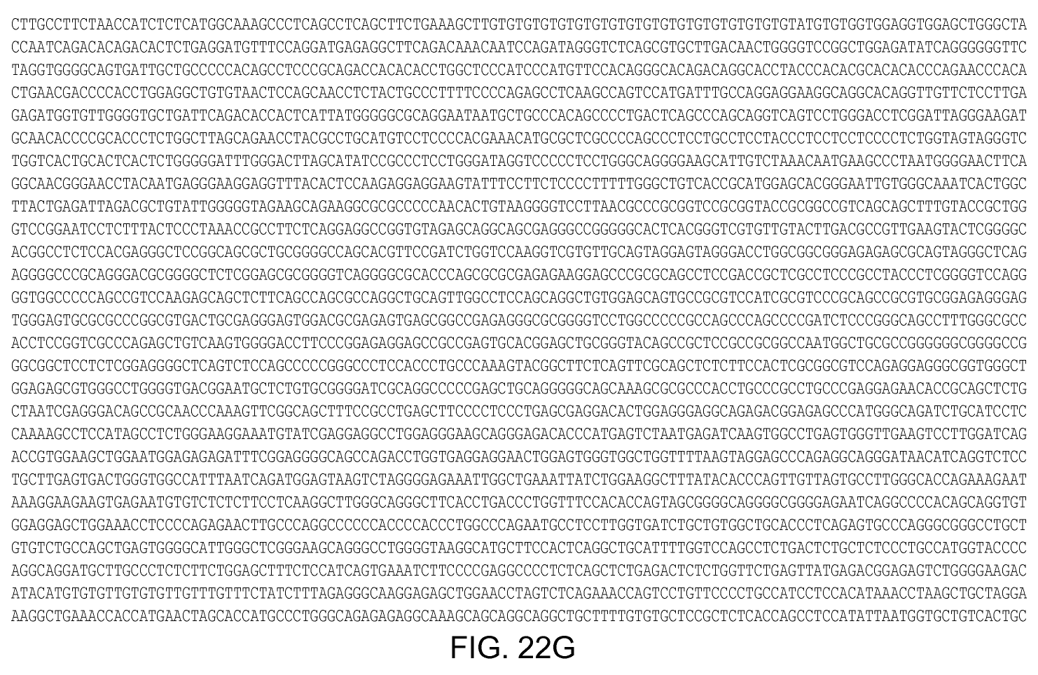

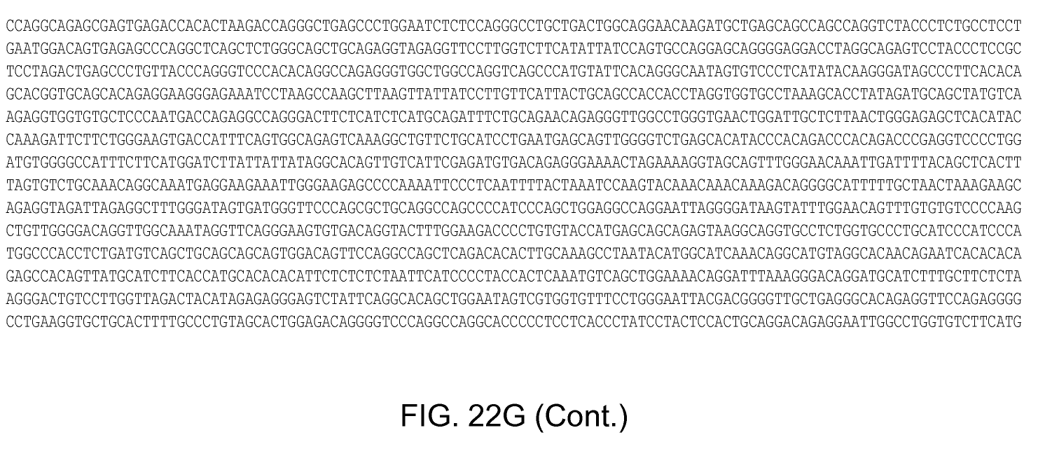

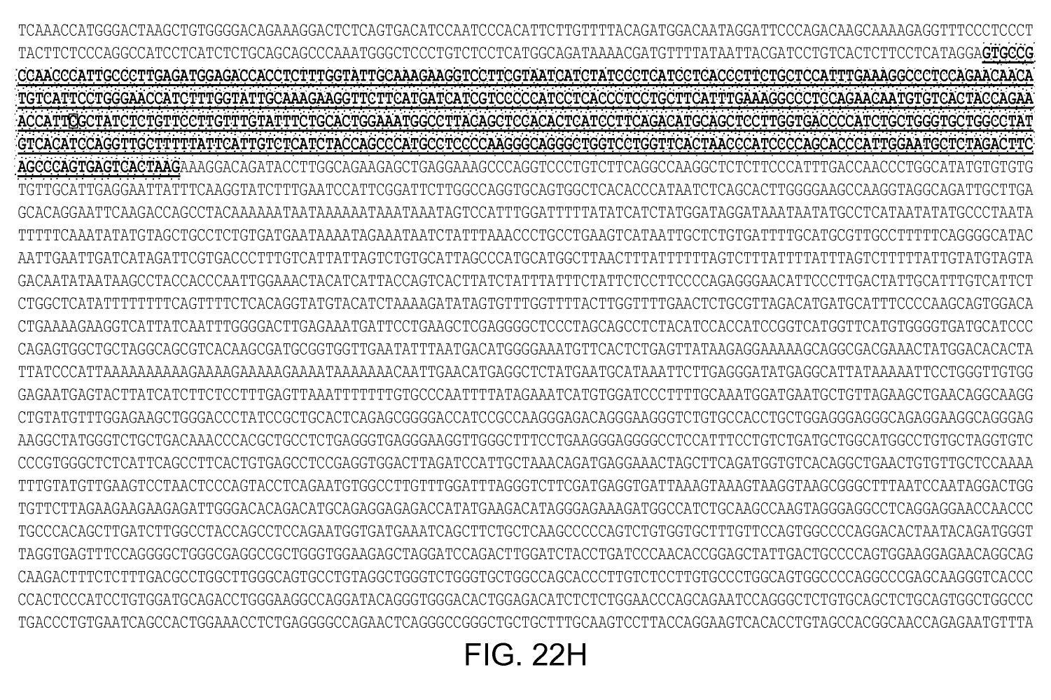

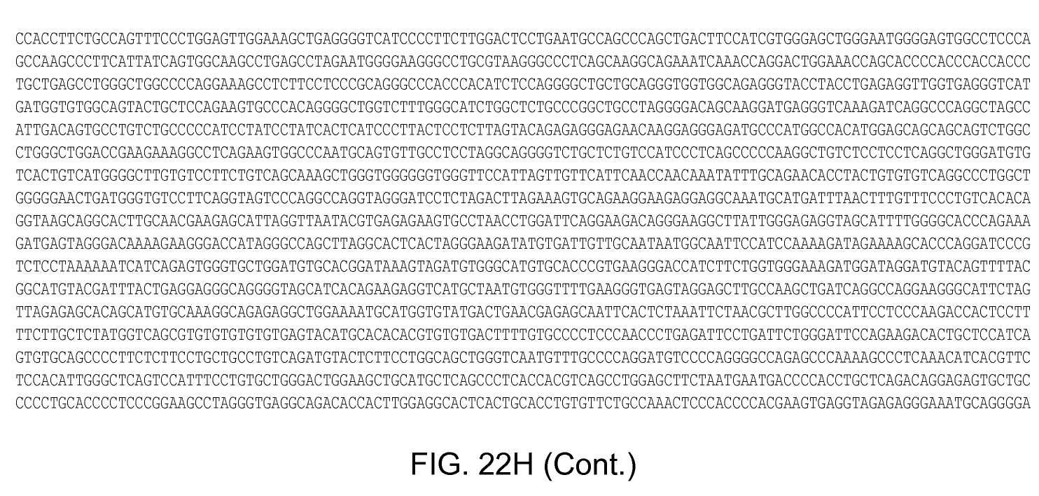

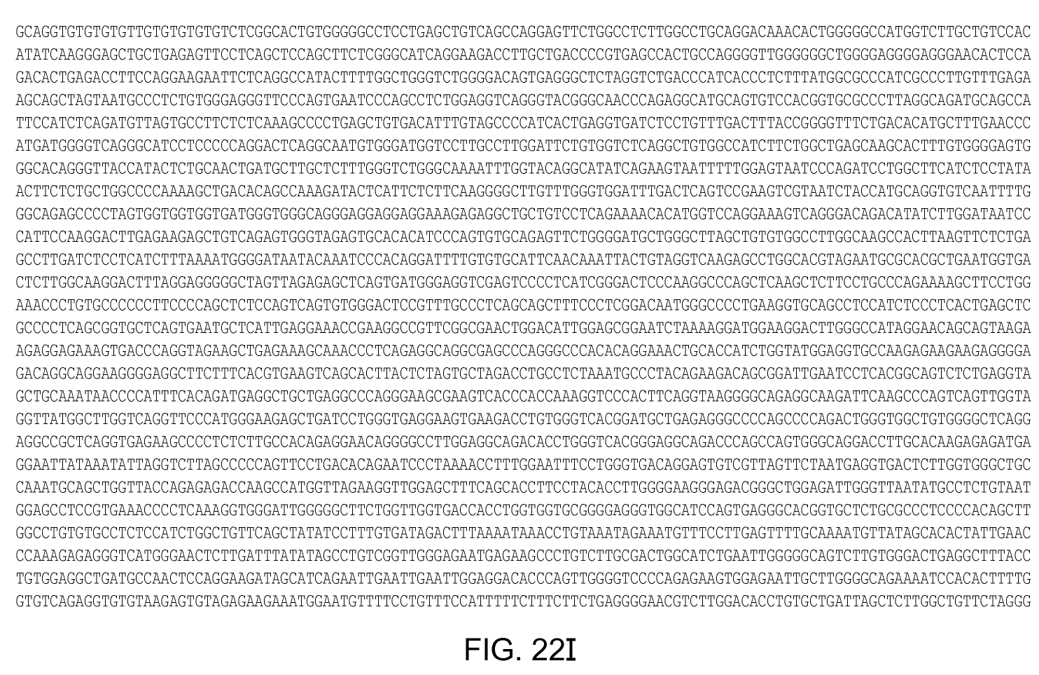

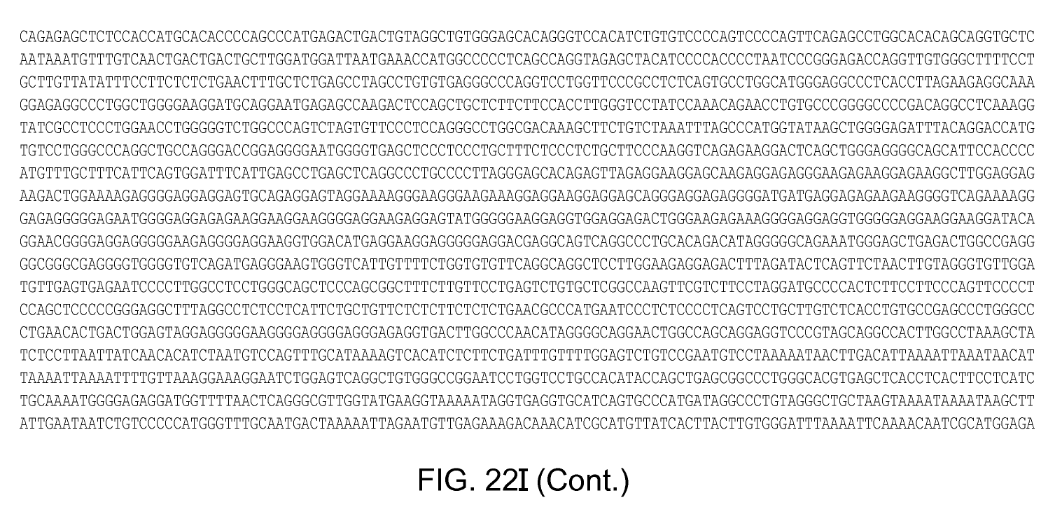

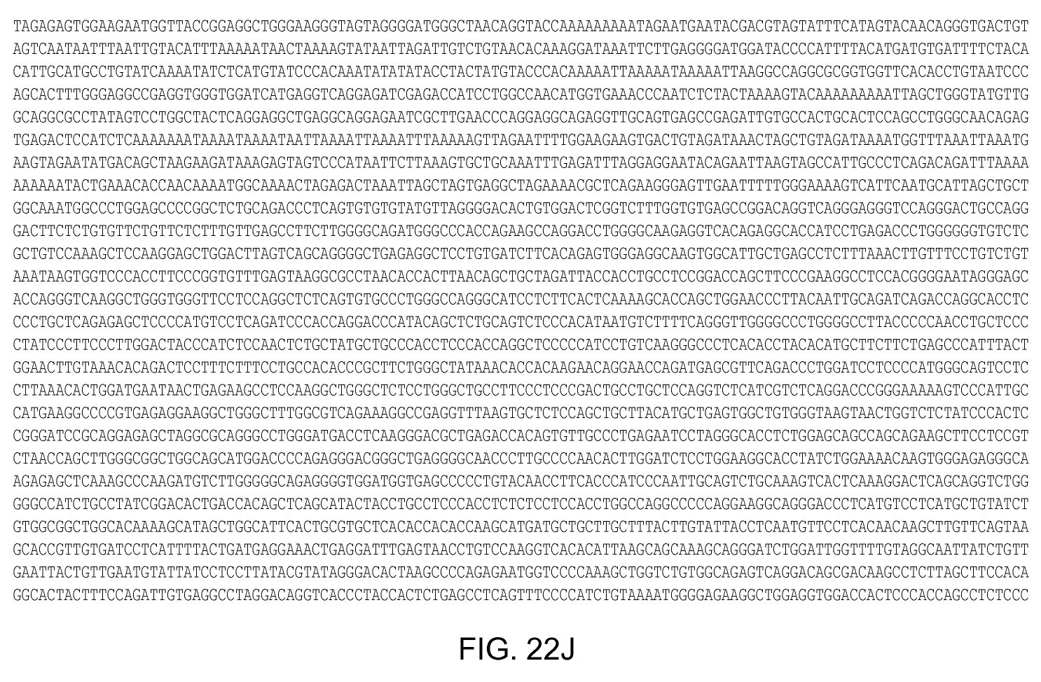

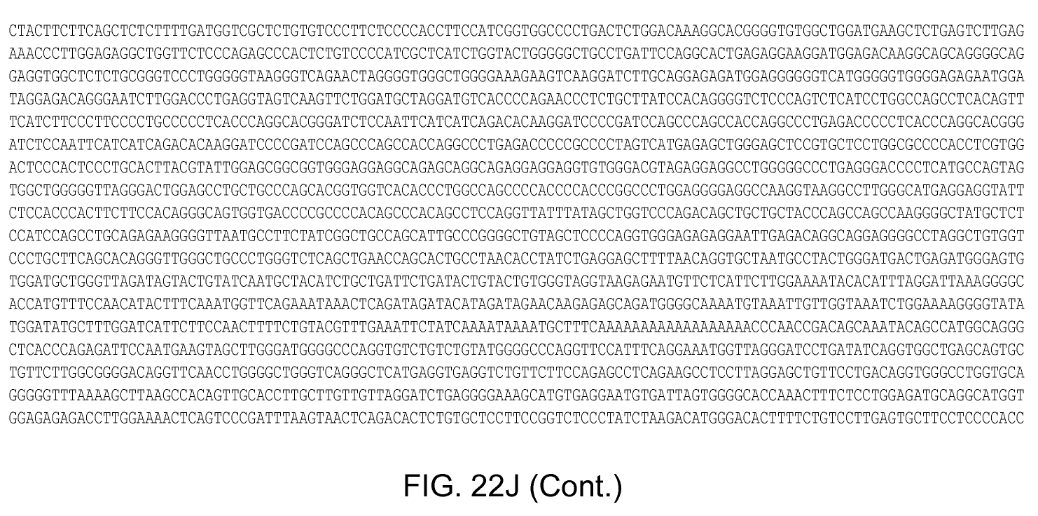

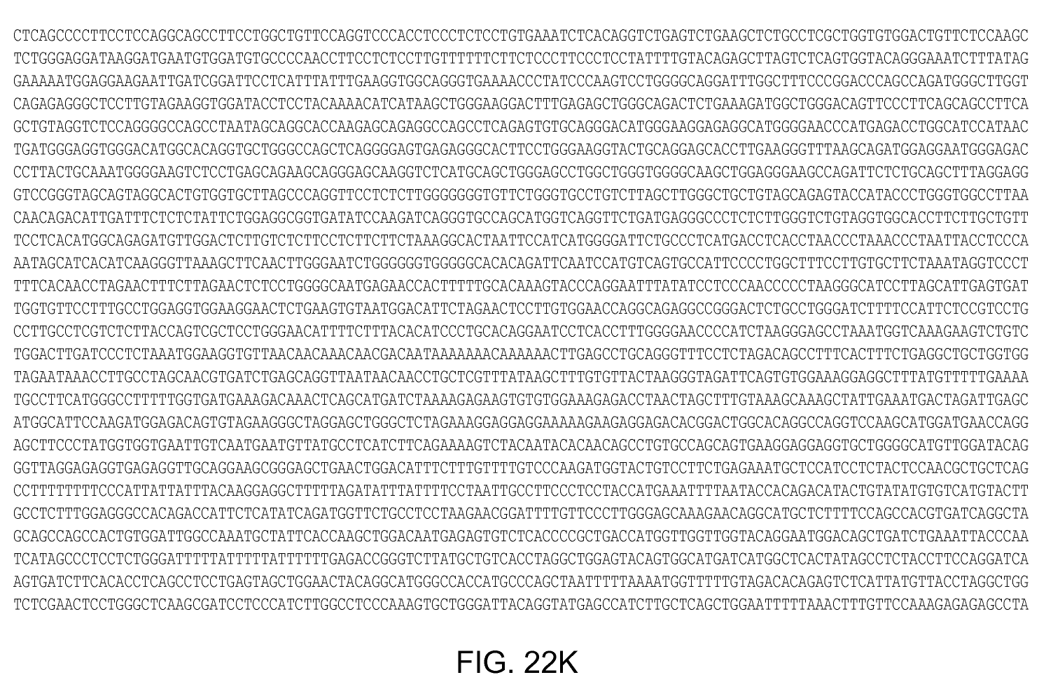

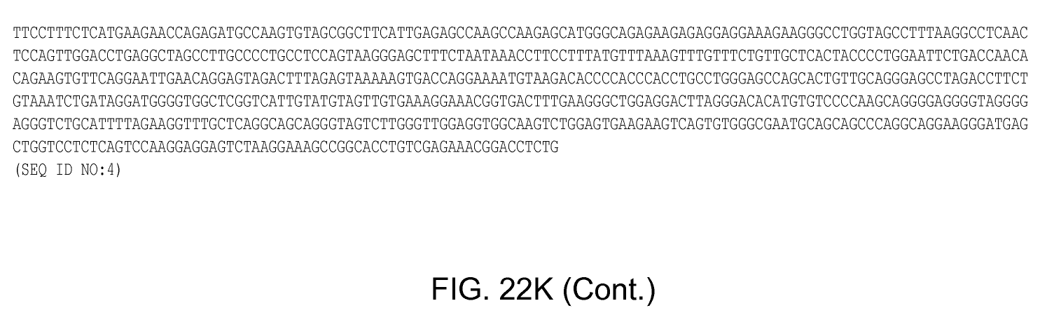

These primers are used in standard PCR protocols in conjunction with another common primer that hybridizes to the complementary strand of a human CRHR2 gene at a specified location from the polymorphism. The common primers are chosen such that the resulting PCR products can vary from about 100 to about 300 bases in length, or about 150 to about 250 bases in length, although smaller (about 50 to about 100 bases in length) or larger (about 300 to about 500 bases in length) PCR products are possible. The length of the primers can vary from about 10 to 30 bases in length, or about 15 to 25 bases in length. The sequences of the common primers can be determined by inspection of the human CRHR2 genomic sequence, which is found under GenBank accession number NG_{—}029169.1 and depicted in FIGS. 21A-5K; the reverse complement of the nucleotide sequence depicted in FIGS. 21A-K is depicted in FIGS. 22A-K.

Suitable methods for detecting polymorphisms include those that involve amplifying DNA or RNA from target samples (e.g., amplifying the segments of the CRHR2 gene of an individual using CRHR2-specific primers) and analyzing the amplified gene. This can be accomplished by standard polymerase chain reaction (PCR and RT-PCR) protocols or other methods known in the art. The amplifying may result in the generation of CRHR2 allele-specific oligonucleotides, which span the single nucleotide polymorphic sites in the CRHR2 gene. CRHR2-specific primer sequences and CRHR2 allele-specific oligonucleotides may be derived from the coding (exons) or non-coding (promoter, 5′ untranslated, introns or 3′ untranslated) regions of a human CRHR2 gene.

The present disclosure provides a panel of primer pairs that amplify nucleic acids comprising the following CRH-R1 SNPs: rs110402; rs16940674; rs171440; rs17689966; rs242924; rs242940; rs242948; rs4076452; rs4792887; rs4792888; rs7209436; rs3785877; rs4792825; rs4458044; rs12944712; rs17763104; rs2664008; rs17763658; rs242942; and rs11657992.

The present disclosure provides a panel of allele-specific probes that primer pairs that amplify nucleic acids comprising the following GR SNPs are detected: rs6198; rs33388; rs2918419; rs10052957; rs10482633; rs12521436; rs12655166; rs17209258; and rs41423247.

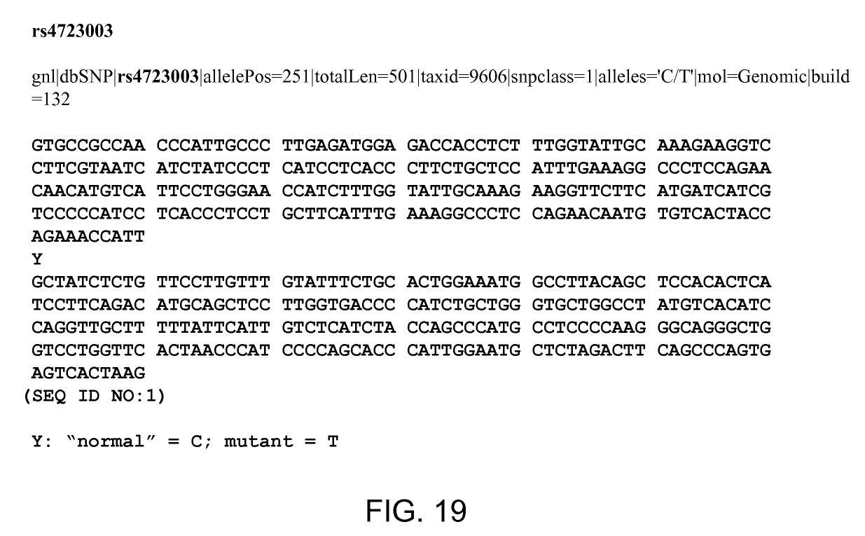

Exemplary, non-limiting, primer pairs that amplify a region including the rs4723003 polymorphic site include, e.g. (where the nucleotides (nt) correspond to the numbering depicted in FIG. 19):

| Forward primer | Reverse primer |
|---|---|
| 5′- TTCCTGGGAACCATCTTTGG -3′ | 5′- GGGTCACCAAGGAGCTGCAT -3′ |
| (nt 131-150) | (reverse complement of nt 322-341) |
| 5′- CCATCTTTGGTATTGCAAAG -3′ | 5′- CATTTCCAGTGCAGAAATAC -3′ |
| (nt 141-160) | (reverse complement of nt 272-291) |
| 5′- TCACCCTCCT GCTTCATTTG-3′ | 5′- GCAGAAATACAAACAAGGAA -3′ |
| (nt 191-210) | (reverse complement of nt 262-281) |

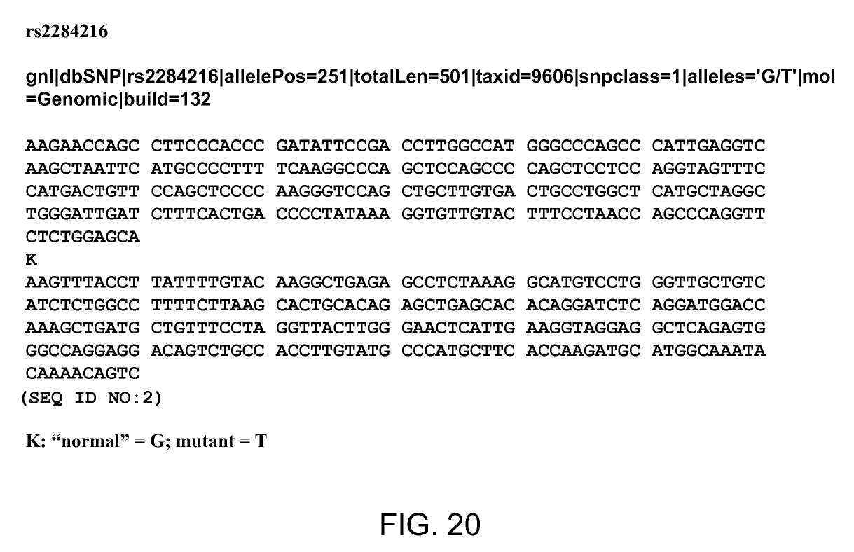

Exemplary, non-limiting, primer pairs that amplify a region including the rs2284216 polymorphic site include, e.g. (where the nucleotides (nt) correspond to the numbering depicted in FIG. 20):

| Forward primer | Reverse primer |
|---|---|
| 5′- CTGCCTGGCTCATGCTAGGC -3′ | 5′- CTGTGCAGTGCTTAAGAAAA -3′ |
| (nt 161-180) | (reverse complement of nt 322-341) |
| 5′- TGGGATTGATCTTTCACTGA -3′ | 5′- GGCCAGAGATGACAGCAACC -3′ |
| (nt 181-200) | (reverse complement of nt 302-321) |
| 5′- CCCCTATAAAGGTGTTGTAC -3′ | 5′- CAGGACATGCCTTTTAGAGGC -3′ |
| (nt 201-220) | (reverse complement of nt 282-301) |

### Analytic Methods

Amplification products generated using PCR can be analyzed by the use of denaturing gradient gel electrophoresis (DGGE). Different alleles can be identified based on sequence-dependent melting properties and electrophoretic migration in solution. See Erlich, ed., PCR Technology, Principles and Applications for DNA Amplification, Chapter 7 (W.H. Freeman and Co, New York, 1992).

Alleles of target sequences can be differentiated using single-strand conformation polymorphism (SSCP) analysis. Different alleles can be identified based on sequence- and structure-dependent electrophoretic migration of single stranded PCR products. Amplified PCR products can be generated according to standard protocols, and heated or otherwise denatured to form single stranded products, which may refold or form secondary structures that are partially dependent on base sequence.

Alleles of target sequences can be differentiated using denaturing high performance liquid chromatography (DHPLC) analysis. Different alleles can be identified based on base differences by alteration in chromatographic migration of single stranded PCR products. Amplified PCR products can be generated according to standard protocols, and heated or otherwise denatured to form single stranded products, which may refold or form secondary structures that are partially dependent on the base sequence.

Direct sequence analysis of polymorphisms can be accomplished using DNA sequencing procedures that are well-known in the art. See Sambrook et al., Molecular Cloning, A Laboratory Manual (2nd Ed., Cold Spring Harbor Press, New York 1989) and Zyskind et al., Recombinant DNA Laboratory Manual (Acad. Press, 1988).

A wide variety of other methods are known in the art for detecting polymorphisms in a biological sample. See, e.g., Ullman et al. “Methods for single nucleotide polymorphism detection” U.S. Pat. No. 6,632,606; Shi, 2002, “Technologies for individual genotyping: detection of genetic polymorphisms in drug targets and disease genes” Am J Pharmacogenomics 2:197-205; Kwok et al., 2003, “Detection of single nucleotide polymorphisms” Curr Issues Biol. 5:43-60).

### Methods of Determining Risk of Depression

The present disclosure provides methods of determining an increased risk of depression in an individual. The methods generally involve genotyping genomic nucleic acid from the individual to detect the presence of a depression-associated SNP in for the presence of a depression-associated SNP (or collection of such SNPs) in one or more of a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene, wherein the SNP is associated with depression, and where the presence of the polymorphism is indicative of an increased risk of developing depression, compared to a normal control. A “normal control” can include an individual, or a group of individuals, that are considered not to have depression. In some cases, the depression is major depression. In some cases, the depression is major depression with psychotic features. In some cases, the depression is major depression with non-psychotic features. In some cases, the depression is major depression with endogenous features.

### CRH-R1 SNPs

In some cases, a subject method involves genotyping genomic nucleic acid (DNA) from an individual for the presence of a SNP in a CRHR1 gene, wherein the SNP is associated with depression, and where the presence of the polymorphism is indicative of increased risk of developing depression, compared to the risk in a normal control individual or a normal control population. In some cases, the depression is major depression. In some cases, the depression is major depression with psychotic features. In some cases, the depression is major depression with non-psychotic features. In some cases, the depression is major depression with endogenous features.

Depression-associated SNPs in a CRHR1 gene include those depicted in FIG. 1, e.g.,

Rs110402;

Rs16940674;

Rs171440;

Rs17689966;

Rs242924;

Rs242940;

Rs242948;

Rs4076452;

Rs4792887;

Rs4792888;

Rs7209436;

Rs37858771;

Rs4792825;

Rs4458044;

Rs12944712;

Rs17763104;

Rs2664008;

Rs17763658;

Rs242942; and

Rs11657992.

In some embodiments, all 21 of the above-listed CRH-R1 SNPs are detected. In other embodiments, 1, 2, 3, 4, 5, 6, 7, 8, 9, 10, 11, 12, 13, 14, 15, 16, 17, 18, 19, or 20 of the above-listed CRH-R1 SNPs are detected. In some cases, rs17763104 alone is detected. In some cases, rs3785877 alone is detected. In some cases, rs4458044 alone is detected.

### Glucocorticoid Receptor SNPs

In some cases, a subject method involves genotyping genomic nucleic acid (DNA) from an individual for the presence of a SNP in a GR gene, wherein the SNP is associated with depression, and where the presence of the polymorphism is indicative of increased risk of developing depression, compared to the risk in a normal control individual or a normal control population.

Depression-associated SNPs in a GR gene include those depicted in FIG. 5, e.g.,

Rs6195;

Rs6198;

Rs33388;

Rs2918419;

Rs10052957;

Rs10482633;

Rs12521436;

Rs12655166;

Rs17209258;

Rs41423247.

In some cases, all 10 of the above-listed GR SNPs are detected. In other instances, 1, 2, 3, 4, 5, 6, 7, 8, or 9 of the above-listed GR SNPs are detected.

### Dopamine 2 Receptor SNPs

In some cases, a subject method involves genotyping genomic nucleic acid (DNA) from an individual for the presence of a SNP in a DRD2 gene, wherein the SNP is associated with depression, and where the presence of the polymorphism is indicative of increased risk of developing depression, compared to the risk in a normal control individual or a normal control population.

Depression-associated SNPs in a DRD2 gene include those depicted in FIG. 9, e.g.,

Rs1799978;

Rs10789944;

Rs7116768;

Rs34735140.

In some cases, all 4 of the above-listed DRD2 SNPs are detected. In other instances, 1, 2, or 3 of the above-listed DRD2 SNPs are detected.

### CRH-R2 SNPs

In some embodiments, the methods involve genotyping genomic nucleic acid from an individual to detect the presence of a single nucleotide polymorphism (SNP) in the individual's corticotropin releasing hormone receptor-2 (CRHR-2) gene, where the SNP is rs4723003, rs228416, or both rs4723003 and rs228416.

The SNP referred to as rs4723003 is a C-to-T substitution, as shown in FIG. 19. The SNP referred to as rs228416 is a G to T substitution. The SNP referred to as rs2284216 is a G-to-T substitution, as shown in FIG. 20. Detection of either or both of rs4723003 and rs228416 indicates that the individual is at increased risk of developing depression, compared to a normal control. A “normal control” can include an individual, or a group of individuals, that are considered not to have depression.

In some instances, both rs4723003 and rs228416 are detected. These two SNPs may be in linkage disequilibrium.

### Linkage Disequilibrium

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

The presence of a SNP that is associated with a propensity in an individual to develop depression indicates that the individual has an at least about 25%, at least about 50%, at least about 2-fold, at least about 5-fold, or at least about 10-fold, increased risk of developing depression, compared to the risk that an individual who does not have the SNP will develop depression.

In some cases, a subject method further involves treating a subject for depression. For example, if an individual has been determined to have an increased risk of developing depression, the subject can be treated with a treatment regimen for depression.

### Detecting a Depression-Associated SNP

A nucleic acid sample obtained from an individual being tested for a predisposition to developing depression can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary suitable SNP genotyping methods are described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues Mol. Biol. 2003 April; 5(2):43-60; Shi, “Technologies for individual genotyping: detection of genetic polymorphisms in drug targets and disease genes”, Am J. Pharmacogenomics. 2002; 2(3):197-205; and Kwok, “Methods for genotyping single nucleotide polymorphisms”, Annu Rev Genomics Hum Genet. 2001; 2:235-58. Exemplary techniques for high-throughput SNP genotyping are described in Marnellos, “High-throughput SNP analysis for genetic association studies”, Curr Opin Drug Discov Devel. 2003 May; 6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, oligonucleotide ligation assay (OLA) (U.S. Pat. No. 4,883,750; and U.S. Pat. No. 5,912,148), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al, Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and S1 protection or chemical cleavage methods.

In one exemplary embodiment, SNP genotyping is performed using a TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Suitable TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. Also suitable for use are modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

Another exemplary method suitable for genotyping a depression-associated SNP is the use of two oligonucleotide probes in an OLA. In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

Another exemplary method suitable for genotyping a depression-associated SNP is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization—Time of Flight) mass spectrometry technology is preferred for extremely precise determinations of molecular mass, such as SNPs. Numerous approaches to SNP analysis have been developed based on mass spectrometry. Suitable mass spectrometry-based methods of SNP genotyping include primer extension assays, which can also be utilized in combination with other approaches, such as traditional gel-based formats and micro arrays.

As a non-limiting example, a primer extension assay can involve designing and annealing a primer to a template PCR amplicon upstream (5′) from a target SNP position. A mix of dideoxynucleotide triphosphates (ddNTPs) and/or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer.

The extended primers can then be purified and analyzed by MALDI-TOF mass spectrometry to determine the identity of the nucleotide present at the target SNP position. In one method of analysis, the products from the primer extension reaction are combined with light absorbing crystals that form a matrix. The matrix is then hit with an energy source such as a laser to ionize and desorb the nucleic acid molecules into the gas-phase. The ionized molecules are then ejected into a flight tube and accelerated down the tube towards a detector. The time between the ionization event, such as a laser pulse, and collision of the molecule with the detector is the time of flight of that molecule. The time of flight is precisely correlated with the mass-to-charge ratio (m/z) of the ionized molecule. Ions with smaller m/z travel down the tube faster than ions with larger m/z and therefore the lighter ions reach the detector before the heavier ions. The time-of-flight is then converted into a corresponding, and highly precise, m/z. In this manner, SNPs can be identified based on the slight differences in mass, and the corresponding time of flight differences, inherent in nucleic acid molecules having different nucleotides at a single base position. For further information regarding the use of primer extension assays in conjunction with MALDI-TOF mass spectrometry for SNP genotyping, see, e.g., Wise et al., “A standard protocol for single nucleotide primer extension in the human genome using matrix-assisted laser desorption/ionization time-of-flight mass spectrometry”, Rapid Commun Mass Spectrom. 2003; 17(11):1195-202.

The following references provide further information describing mass spectrometry-based methods for SNP genotyping: Bocker, “SNP and mutation discovery using base-specific cleavage and MALDI-TOF mass spectrometry”, Bioinformatics. 2003 July; 19 Suppl 1: 144-153; Storm et al., “MALDI-TOF mass spectrometry-based SNP genotyping”, Methods Mol. Biol. 2003; 212:241-62; Jurinke et al., “The use of Mass ARRAY technology for high throughput genotyping”, Adv Biochem Eng Biotechnol. 2002; 77:57-74; and Jurinke et al., “Automated genotyping using the DNA MassArray technology”, Methods Mol. Biol. 2002; 187:179-92.

A depression-associated SNP can also be detected by direct DNA sequencing. A variety of automated sequencing procedures can be utilized ((1995) Biotechniques 19:448), including sequencing by mass spectrometry (see, e.g., WO94/16101; Cohen et al., Adv. Chromatogr. 36:127-162 (1996); and Griffin et al., Appl. Biochem. Biotechnol. 38:147-159 (1993)). The nucleic acid sequences disclosed herein enable one of ordinary skill in the art to readily design sequencing primers for such automated sequencing procedures. Commercial instrumentation, such as the Applied Biosystems 377, 3100, 3700, 3730, and 3730.times.1 DNA Analyzers (Foster City, Calif.), is commonly used in the art for automated sequencing.

Other methods that can be used to genotype a depression-associated SNP include single-strand conformational polymorphism (SSCP), and denaturing gradient gel electrophoresis (DGGE) (Myers et al., Nature 313:495 (1985)). SSCP identifies base differences by alteration in electrophoretic migration of single stranded PCR products. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel (Erlich, ed., PCR Technology, Principles and Applications for DNA Amplification, W.H. Freeman and Co, New York, 1992, Chapter 7).

Sequence-specific ribozymes (U.S. Pat. No. 5,498,531) can also be used to detect a depression-associated SNP based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis.

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

### Generating a Report

The results of a test (e.g., a test to determine an individual's risk for developing depression, based detection of a depression-associated SNP, as described above), and/or any other information pertaining to a test, can be provided in the form of a “report”. A tangible report can optionally be generated as part of a testing process (which may be interchangeably referred to herein as “reporting”, or as “providing” a report, “producing” a report, or “generating” a report). Examples of tangible reports may include, but are not limited to, reports on paper (such as computer-generated printouts of test results) or equivalent formats and reports stored on computer readable medium (such as a compact disk (CD), computer hard drive, or computer network server, etc.). Reports, e.g., those stored on computer readable medium, can be part of a database (such as a database of patient records, which may be a “secure database” that has security features that limit access to the report, such as to allow only the patient and the patient's medical practitioner(s) to view the report, for example). In addition to, or as an alternative to, generating a tangible report, reports can also be displayed on a computer screen (or the display of another electronic device or instrument).

A report can further be “transmitted” or “communicated” (these terms may be used herein interchangeably), such as to the individual who was tested, a medical practitioner (e.g., a doctor, nurse, clinical laboratory practitioner, psychiatric counselor, etc.), a healthcare organization, a clinical laboratory, and/or any other party intended to view or possess the report. The act of “transmitting” or “communicating” a report can be by any means known in the art, based on the form of the report. Furthermore, “transmitting” or “communicating” a report can include delivering a report (“pushing”) and/or retrieving (“pulling”) a report. For example, reports can be transmitted/communicated by such means as being physically transferred between parties (such as for reports in paper format), such as by being physically delivered from one party to another, or by being transmitted electronically or in signal form (e.g., via e-mail or over the internet, by facsimile, and/or by any wired or wireless communication methods known in the art), such as by being retrieved from a database stored on a computer network server, etc.

### Counseling and Treatment

Based on the results of a subject method, an individual may be identified as predisposed to developing depression. In such instances, the individual may be counseled by a medical professional to undergo psychological evaluation and/or counseling; and/or to undergo drug treatment for depression. The individual may also be monitored periodically by a medical professional.

Drugs suitable for treating depression include, but are not limited to, a selective serotonin reuptake inhibitor, a serotonin and norepinephrine reuptake inhibitor, a dopamine reuptake inhibitor, a tetracyclic antidepressant, a combined reuptake inhibitor, a receptor blocker, tricyclic antidepressant, a monoamine oxidase inhibitor (MAOI), a benzodiazepine, a beta-blocker, and a non-benzodiazepine hypnotic.

Drugs suitable for treating depression include, e.g., serotonin reuptake inhibitors, selective norepinephrine reuptake inhibitors, combined action SSRI/SNR1, serotonin-2 antagonist/reuptake inhibitors, an antidepressant with alpha-2 antagonism plus serotonin-2 and serotonin-3 antagonism, an antidepressant with serotonin/norepinephrine/dopamine reuptake inhibition, an antidepressant with norepinephrine and dopamine reuptake inhibition, 5-HT-1alpha antagonist, 5-HT-1beta antagonist, 5-HT1A receptor agonists, 5-HT1A receptor agonists and antagonists, 5-HT2 receptor antagonists, viloxazine hydrochloride, dehydroepiandosterone, NMDA receptor antagonists, AMPA receptor potentiators, substance P antagonists/neurokinin-1 receptor antagonists, nonpeptide Substance P antagonist, neurokinin 2 antagonists, neurokinin 3 antagonists, corticotropin-releasing factor receptor antagonists, antiglucocorticoid medications, glucocorticoid receptor antagonists, cortisol blocking agents, nitric oxide synthesize inhibitors, inhibitors of phosphodiesterase, enkephalinase inhibitors, GABA-A receptor agonists, free radical trapping agents, atypical MAOI's, selective MAOI inhibitors, hormones, folinic acid, leucovorin, tramadol, and tryptophan.

Examples of drugs suitable for treating depression include fluoxetine, norfluoxetine, paroxetine, sertraline, fluvoxamine, citalopram, escitalopram, bupropion, nefazodone, mirtazapine, venlafaxine, duloxetine, milnacipran, reboxetine, zimelidine, indalpine, gepirone, femoxetine, alaproclate and pharmaceutically acceptable salts thereof.

### Treatment Methods

The present disclosure provides methods of treating an individual who has been determined by a subject method to have an increased risk of depression. A subject treatment method includes: a) genotyping genomic nucleic acid from an individual for the presence of a depression-associated SNP in one or more of in one or more of a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene; (b) determining from said genotyping an increased risk for developing depression; and (c) based on the results of said determining step, treating the individual for depression. For example, if the determining step indicates that the individual is at increased risk of developing depression, the individual is treated for depression.

Drugs suitable for treating depression include, but are not limited to, a selective serotonin reuptake inhibitor, a serotonin and norepinephrine reuptake inhibitor, a dopamine reuptake inhibitor, a tetracyclic antidepressant, a combined reuptake inhibitor, a receptor blocker, tricyclic antidepressant, a monoamine oxidase inhibitor, a benzodiazepine, a beta-blocker, and a non-benzodiazepine hypnotic.

### Reagents, Devices, and Kits

The present disclosure provides reagents (e.g., nucleic acid reagents such as synthetic probes and synthetic primers), devices, and kits for carrying out a subject detection method. The present disclosure provides synthetic nucleic acids for use in detecting a depression-associated SNP described herein. The nucleic acids are useful in a subject method of determining an increased risk of depression.

A subject detection reagent in the form of a polynucleotide may optionally contain modified base analogs, intercalators or minor groove binders. Multiple detection reagents such as probes may be, for example, affixed to a solid support (e.g., arrays or beads) or supplied in solution (e.g., probe/primer sets for enzymatic reactions such as PCR, RT-PCR, TaqMan assays, or primer-extension reactions) to form a SNP detection kit.

### SNP Detection Reagents

The present disclosure provides SNP detection reagents for detecting a depression-associated SNP in one or more of a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene. A subject SNP detection reagent includes an allele-specific probe, and allele-specific primer, and a primer pair that specifically amplifies a region in a human gene (a CRHR1 gene, a CRHR2 gene, a GR gene, a DRD2 gene, etc.) that contains a depression-associated SNP.

A subject SNP detection reagent can have a length of from about 15 nucleotides (nt) to about 250 nt, e.g., from about 15 nt to about 20 nt, from about 20 nt to about 25 nt, from about 25 nt to about 30 nt, from about 30 nt to about 40 nt, from about 40 nt to about 50 nt, from about 50 nt to about 75 nt, from about 75 nt to about 100 nt, from about 100 nt to about 150 nt, from about 150 nt to about 200 nt, or from about 200 nt to about 250 nt.

In some embodiments, a subject SNP detection reagent is an allele-specific probe.

Exemplary allele-specific probes include the following.

Exemplary allele-specific probes for analyzing a CRHR2 SNP that is predictive of depression include:

1) allele-specific probes for detecting rs4723003, where exemplary probes include, e.g.:

(nucleotides 36,229-36,249 of SEQ ID NO: 4)
a) 5′-AGAAACCATTTGCTATCTCTG-3′;
b) 5′-CAGAGATAGCAAATGGTTTCT-3′;
c) 5′-CCAGAAACCATTTGCTATCTCTGTT-3′;
and
d) 5′-AACAGAGATAGCAAATGGTTTCTGG-3′.

Such probes would be expected to detect the C-to-T substitution that characterizes rs4723003.

2) allele-specific probes for detecting rs2284216, where exemplary probes include, e.g.:

a) 5′-CTCTGGAGCATAAGTTTACCT-3′;
b) 5′-AGGTAAACTTATGCTCCAGAG-3′;
c) 5′-TTCTCTGGAGCATAAGTTTACCTTA-3′;
and
d) 5′-TAAGGTAAACTTATGCTCCAGAGAA-3′.

Such probes would be expected to detect the G-to-T substitution that characterizes rs2284216.

Those of ordinary skill in the art can readily design additional probes, given the information provided in the figures, and given available sequence information.

A subject SNP detection reagent can include a pair of allele-specific probes, e.g., where the first member (the “reference” member) of the pair of allele-specific probes detects the “wild-type” or “normal” allele (e.g., the allele not associated with increased risk of developing depression); and the second member of the pair of allele-specific probes detects the depression-associated allele.

Probes that would be useful as “reference” probes for detecting the corresponding “wild-type” or “normal” sequence corresponding to rs4723003 include, e.g.:

a) 5′-AGAAACCATTCGCTATCTCTG-3′;
b) 5′-CAGAGATAGCGAATGGTTTCT-3′;
d) 5′-CCAGAAACCATTCGCTATCTCTGTT-3′;
and
d) 5′-AACAGAGATAGCGAATGGTTTCTGG-3′.

Probes that would be useful as “reference” probes for detecting the corresponding “wild-type” or “normal” sequence corresponding to rs2284216 include, e.g.:

(nucleotides 22,425 to 22,445 of SEQ ID NO: 4)
a) 5′-CTCTGGAGCAGAAGTTTACCT-3′;
b) 5′-AGGTAAACTTCTGCTCCAGAG-3′;
c) 5′-TTCTCTGGAGCAGAAGTTTACCTTA-3′;
and
d) 5′-TAAGGTAAACTTCTGCTCCAGAGAA-3′.

Exemplary, non-limiting allele-specific probe pairs, e.g., for detecting rs4723003 and the corresponding “normal” sequence, include:

a) 5′-AGAAACCATTTGCTATCTCTG-3′;
and
5'-AGAAACCATTCGCTATCTCTG-3′;
b) 5′-CAGAGATAGCAAATGGTTTCT-3′;
and
5′-CAGAGATAGCGAATGGTTTCT-3′;
c) 5′-CCAGAAACCATTTGCTATCTCTGTT-3′;
and
5′-CCAGAAACCATTCGCTATCTCTGTT-3′;
and
d) 5′-AACAGAGATAGCAAATGGTTTCTGG-3′;
and
5′-AACAGAGATAGCGAATGGTTTCTGG-3′.

Exemplary, non-limiting allele-specific probe pairs, e.g., for detecting rs2284216 and the corresponding “normal” sequence, include, e.g.:

a) 5′-CTCTGGAGCATAAGTTTACCT-3′;
and
5′-CTCTGGAGCAGAAGTTTACCT-3′;
b) 5′-AGGTAAACTTATGCTCCAGAG-3′;
and
5′-AGGTAAACTTCTGCTCCAGAG-3′;
c) 5′-TTCTCTGGAGCATAAGTTTACCTTA-3′;
and
5′-TTCTCTGGAGCAGAAGTTTACCTTA-3′;
and
d) 5′-TAAGGTAAACTTATGCTCCAGAGAA-3′;
and
5′-TAAGGTAAACTTCTGCTCCAGAGAA-3′.

In some embodiments, a subject SNP detection reagent is an allele-specific primer. Exemplary, non-limiting allele-specific primers for analyzing a depression-associated SNP in a human CRHR2 gene include the following.

Suitable allele-specific primers for analyzing a depression-associated SNP in a human CRHR2 gene include the following.

Suitable allele-specific primers for analyzing an rs4723003 SNP include, e.g.:

a) 5′-GTCACTACCAGAAACCATTTGCT-3′;
b) 5′-TGTGTCACTACCAGAAACCATTTG-3′;
c) 5′-CAAACAAGGAACAGAGATAGCAAAT-3′;
and
d) 5′-ATACAAACAAGGAACAGAGATAGCAA-3′.

Such primers would detect the C-to-T substitution that characterizes rs4723003.

Suitable allele-specific primers for analyzing an rs2284216 SNP include, e.g.:

a) 5′-CAGGTTCTCTGGAGCATAAG-3′;
b) 5′-CAGCCCAGGTTCTCTGGAGCATA-3′;
c) 5′-ACAAAATAAGGTAAACTTATGC-3′;
and
d) 5′-TGTACAAAATAAGGTAAACTTAT-3′.

Such primers would detect the g-to-T substitution that characterizes rs2284216.

Also suitable for use would be the complement of any of the forgoing.

In some embodiments, a subject SNP detection reagent comprises sets of allele-specific primer pairs, where the set includes a first primer pair (the “reference” primer pair) that amplifies a “normal” CRHR2 allele that does not include a depression-associated SNP; and the second primer pair amplifies a CRHR2 allele that includes a depression-associated SNP.

For example, primers that would be suitable as “reference” primers for detecting the “normal” or “wild-type” sequence corresponding to rs4723003 include, e.g.:

a) 5′-GTCACTACCAGAAACCATTCGCT-3′;
b) 5′-TGTGTCACTACCAGAAACCATTCG-3′;
c) 5′-CAAACAAGGAACAGAGATAGCGAAT-3′;
and
d) 5′-ATACAAACAAGGAACAGAGATAGCGA-3′.

Suitable primers that would be suitable as “reference” primers for detecting the “normal” or “wild-type” sequence corresponding to rs2284216 include, e.g.:

a) 5′-CAGGTTCTCTGGAGCAGAAG-3′;
b) 5′-CAGCCCAGGTTCTCTGGAGCAGA-3′;
c) 5′-ACAAAATAAGGTAAACTTCTGC-3′;
and
d) 5-TGTACAAAATAAGGTAAACTTCT-3′.

Also suitable would be a complement of any of the foregoing.

In some embodiments, a subject SNP detection reagent comprises a pair of nucleic acids, where the first member of the pair is an allele-specific primer, as described above, and the second member of the pair hybridizes to a human CRHR2 gene at a location upstream or downstream of the location to which the first member hybridizes, such that, under standard polymerase chain reaction conditions, the first and the second members of the primer pair amplify a segment of the human CRHR2 gene comprising a depression-associated SNP. For example, the first and the second members of the primer pair amplify a segment of a human CRHR2 gene that includes from 1 nt to 300 nt (e.g., from 1 nt to 5 nt, from 5 nt to 10 nt, from 10 nt to 15 nt, from 15 nt to 20 nt from 20 nt to 25 nt, from 25 nt to 50 nt, from 50 nt to 100 nt, from 100 nt to 200 nt, from 100 nt to 300 nt) 5′ of a depression-associated SNP in the CRHR2 gene, and from 1 nt to 300 nt (e.g., from 1 nt to 5 nt, from 5 nt to 10 nt, from 10 nt to 15 nt, from 15 nt to 20 nt from 20 nt to 25 nt, from 25 nt to 50 nt, from 50 nt to 100 nt, from 100 nt to 200 nt, from 100 nt to 300 nt) 3′ of the depression-associated SNP. The PCR product thus produced has a length of from about 15 to about 600 nt.

A subject SNP detection reagent can comprise a detectable label, e.g., a radiolabel, a fluorogenic dye, etc. For example, in some embodiments, a subject SNP detection reagent is labeled with a fluorogenic reporter dye that emits a detectable signal. Suitable reporter dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red. Suitable fluorogenic dyes include, e.g., 5-carboxyfluorescein, 6-carboxyfluorescein, 2′,7′-dimethoxy-4′,5′-dichloro-6-carboxyfluorescein, N,N,N′,N′-tetramethyl-6-carboxy rhodamine, 6-carboxyrhodamine X, 4,7,2′,4′,5′,7′-hexachloro-6-carboxyfluorescein, 4,7,2′,4′,5′,7′-hexachloro-5-carboxyfluorescein, 2′,4′,5′,7′-tetrachloro-5-carboxyfluorescein, 4,7,2′,7′-tetrachloro-6-carboxyfluorescein, 1′,2′,7′,8′-dibenzo-4,7-dichloro-5-carboxyfluorescein, and 1′,2′,7′,8′-dibenzo-4,7-dichloro-6-carboxyfluorescein.

A subject SNP detection reagent can be further labeled with a quencher dye such as Tamra, e.g., when the reagent is used as a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl. 4:357-362; Tyagi et al., 1996, Nature Biotechnology 14: 303-308; Nazarenko et al., 1997, Nucl. Acids Res. 25:2516-2521; U.S. Pat. Nos. 5,866,336 and 6,117,635).

A subject SNP detection reagent is in some embodiments immobilized on a substrate. Suitable substrates include, e.g., glass; plastic; paper, nylon, nitrocellulose, or other type of membrane (e.g., which membrane may be in the form of a test strip); a filter; a chip; or any other suitable solid support.

### SNP Detection Kits

Subject reagents include, e.g., allele-specific primers, primer pairs for amplification, allele-specific probes, and combinations thereof. Such reagents may be contained in separate containers in a subject kit. In an embodiment, the kit contains a first container containing a probe, primer, or primer pair for a depression-associated SNP present in a CRHR1 gene, a CRHR2 gene, a GR gene, or a DRD2 gene, as described above, and a second container containing a reference probe, primer, or primer pair, e.g., for detecting the reference allele corresponding to the depression-associated SNP.

In one embodiment, the invention provides kits comprising an allele-specific oligonucleotide that hybridizes to a human CRHR1 gene, a CRHR2 gene, a GR gene, or a DRD2 gene comprising a depression-associated SNP. The kits may contain one or more pairs of allele-specific oligonucleotides hybridizing to different forms of a polymorphism. The allele-specific oligonucleotides may include sequences derived from the coding (exons) or non-coding (promoter, 5′ untranslated, introns or 3′ untranslated) region of a human CRHR1 gene, a CRHR2 gene, a GR gene, or a DRD2 gene. The allele-specific oligonucleotides may be provided immobilized on a substrate.

A subject kit can include at least one gene-specific primer that hybridizes to a region spanning or adjacent to a depression-related polymorphism in a human CRHR1 gene, a CRHR2 gene, a GR gene, or a DRD2 gene. The gene-specific primers may include sequences derived from the coding (exons) or non-coding (promoter, 5′ untranslated, introns or 3′ untranslated) region of a CRHR1 gene, a CRHR2 gene, a GR gene, or a DRD2 gene. A subject kit can contain one or more pairs of gene-specific primers that hybridize to opposite strands of nucleic acid adjacent to a depression-associated polymorphism in a CRHR1 gene, a CRHR2 gene, a GR gene, or a DRD2 gene. In the presence of appropriate buffers and enzymes, the gene-specific primer pairs are useful in amplifying specific polymorphisms in a CRHR1 gene, a CRHR2 gene, a GR gene, or a DRD2 gene.

A subject kit can include, in addition to a SNP detection reagent, one or more biochemical reagents, containers, packages such as packaging intended for commercial sale, substrates to which SNP detection reagents are attached, electronic hardware components, etc.

In some embodiments, a subject SNP detection kit contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A subject kit can further contain means for determining the amount of a target nucleic acid, and means for comparing the amount with a standard, and can comprise instructions for using the kit to detect the SNP-containing nucleic acid.

A subject SNP detection kit can include components that are used to prepare nucleic acids from a test sample for the subsequent amplification and/or detection of a SNP-containing nucleic acid molecule. Such sample preparation components can be used to produce nucleic acid extracts (including DNA and/or RNA) from any bodily fluids (such as blood, serum, plasma, urine, saliva, phlegm, gastric juices, semen, tears, sweat, etc.), skin, hair, cells (especially nucleated cells; e.g., peripheral blood mononuclear cells), biopsies, buccal swabs or tissue specimens. The test samples used in a subject method will vary based on such factors as the assay format, nature of the detection method, and the specific tissues, cells or extracts used as the test sample to be assayed. Methods of preparing nucleic acids and cell extracts are well known in the art and can be readily adapted to obtain a sample that is compatible with the system utilized. Automated sample preparation systems for extracting nucleic acids from a test sample are commercially available.

In some embodiments, a subject SNP detection kit is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting a subject SNP, one or more containers which contain wash reagents (such as phosphate buffered saline, Tris-buffers, etc.), and one or more containers which contain the reagents used to reveal the presence of the bound probe or other SNP detection reagents. The kit can optionally further comprise compartments and/or reagents for, for example, nucleic acid amplification or other enzymatic reactions such as primer extension reactions, hybridization, ligation, electrophoresis (e.g., capillary electrophoresis), mass spectrometry, and/or laser-induced fluorescent detection. The kit may also include instructions for using the kit. Exemplary compartmentalized kits include microfluidic devices known in the art (see, e.g., Weigl et al., “Lab-on-a-chip for drug development”, Adv Drug Deliv Rev. 2003 Feb. 24; 55(3):349-77). In such microfluidic devices, the containers may be referred to as, for example, microfluidic “compartments”, “chambers”, or “channels.”

Microfluidic devices, which can also be referred to as “lab-on-a-chip” systems, biomedical micro-electro-mechanical systems (bioMEMs), or multicomponent integrated systems, are suitable for inclusion in a subject SNP detection kit. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect a subject SNP. One example of a microfluidic system is disclosed in U.S. Pat. No. 5,589,136, which describes the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. Nos. 6,153,073, and 6,156,181.

For genotyping SNPs, an exemplary microfluidic system may integrate, for example, nucleic acid amplification, primer extension, capillary electrophoresis, and a detection method such as laser induced fluorescence detection. In a first step of an exemplary process for using such an exemplary system, nucleic acid samples are amplified, e.g., by PCR. Then, the amplification products are subjected to automated primer extension reactions using ddNTPs (specific fluorescence for each ddNTP) and the appropriate oligonucleotide primers to carry out primer extension reactions which hybridize just upstream of the targeted SNP. Once the extension at the 3′ end is completed, the primers are separated from the unincorporated fluorescent ddNTPs by capillary electrophoresis. The separation medium used in capillary electrophoresis can be, for example, polyacrylamide, poly(ethylene glycol) or dextran. The incorporated ddNTPs in the single nucleotide primer extension products are identified by laser-induced fluorescence detection.

### Subjects Suitable for Testing

Subjects suitable for testing using a subject assay method include individuals who are suspected of having depression; and individuals who have not been diagnosed as having depression, but who may, due to various factors such as family history, be considered suitable for testing for an increased risk of developing depression. Also suitable for testing include individuals in the general population. Also suitable for testing include individuals who are being considered for inclusion in a clinical trial.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

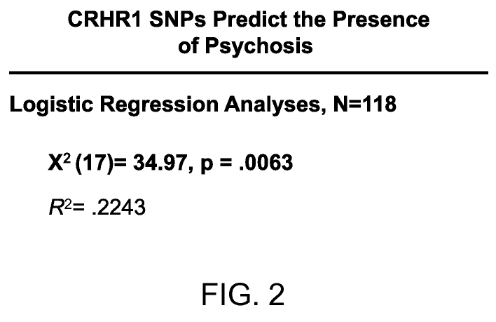

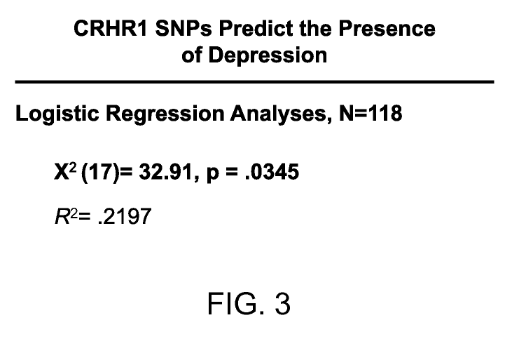

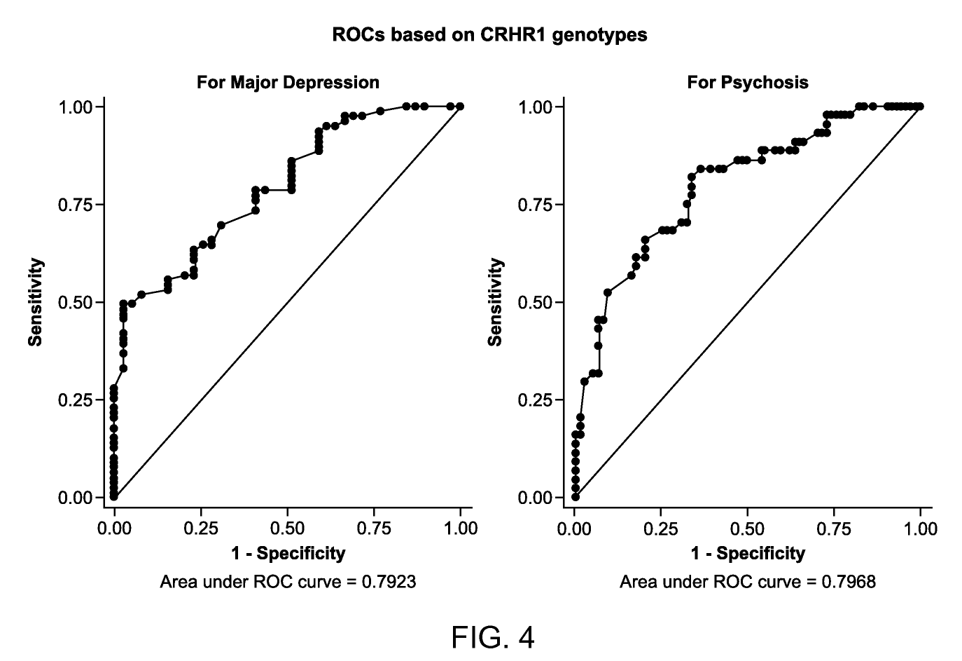

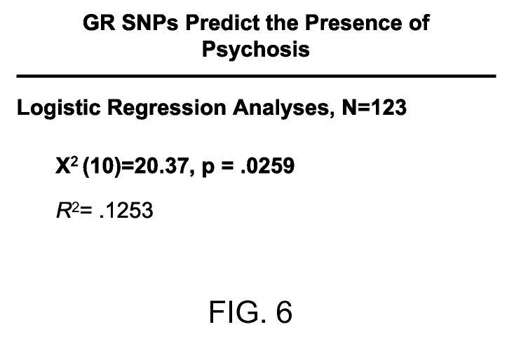

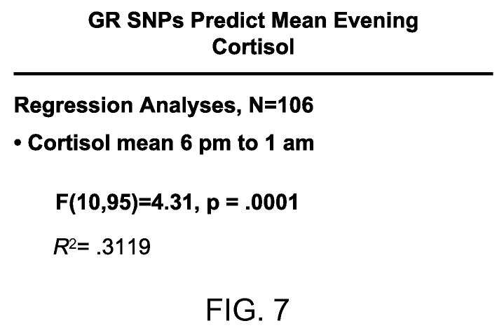

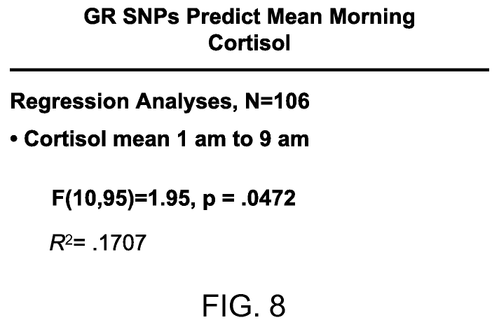

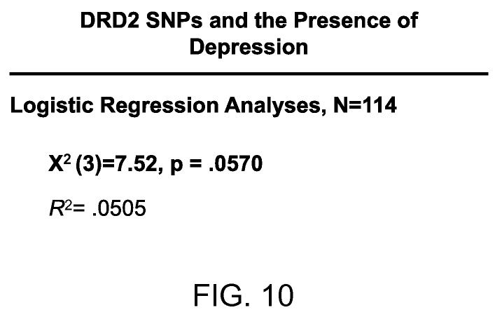

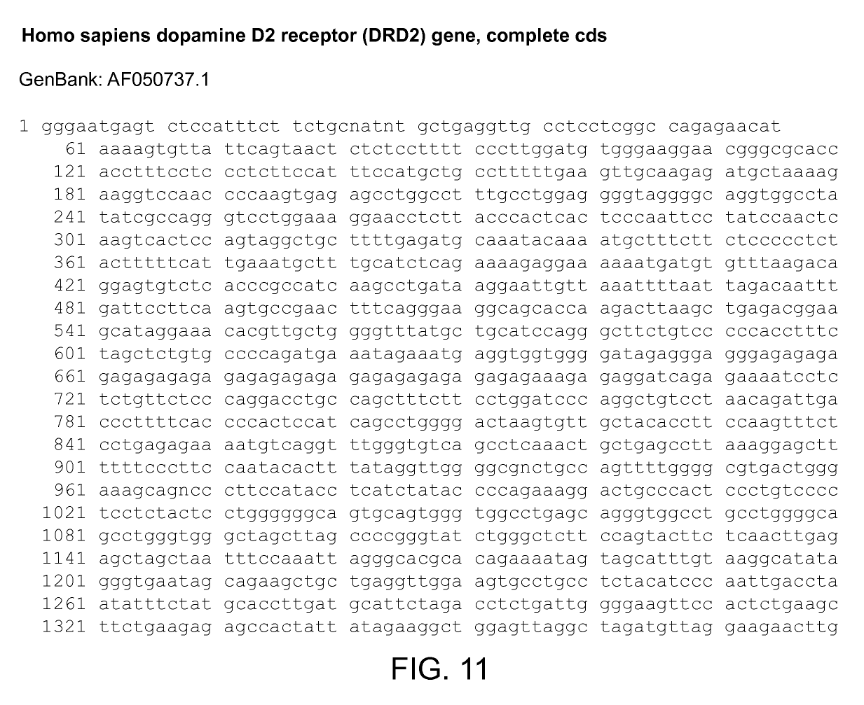

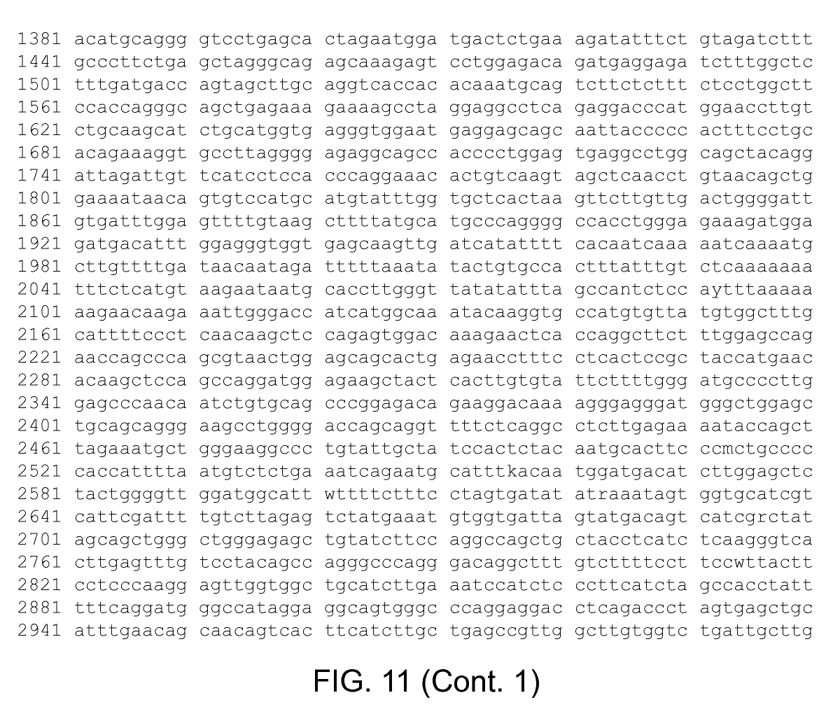

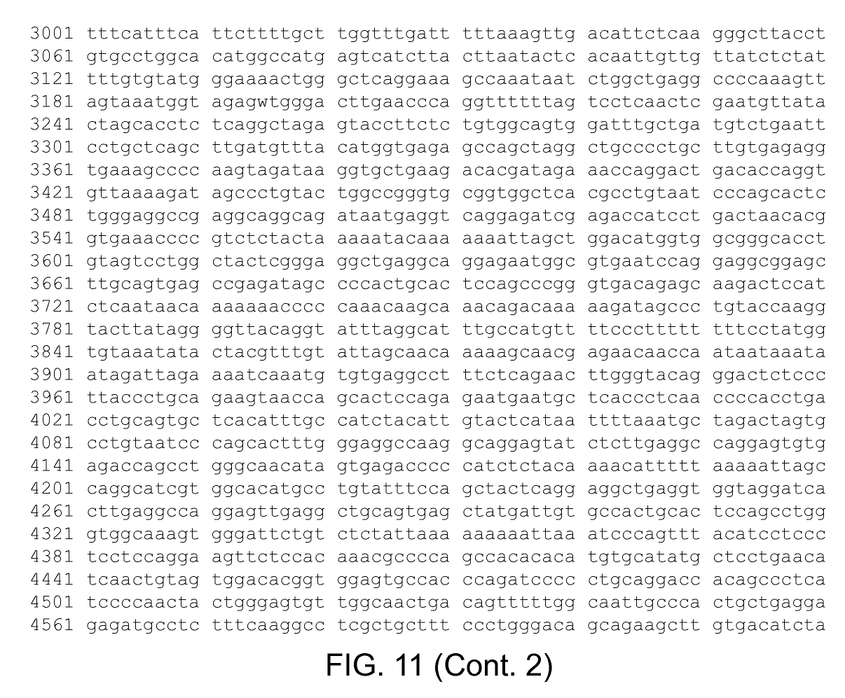

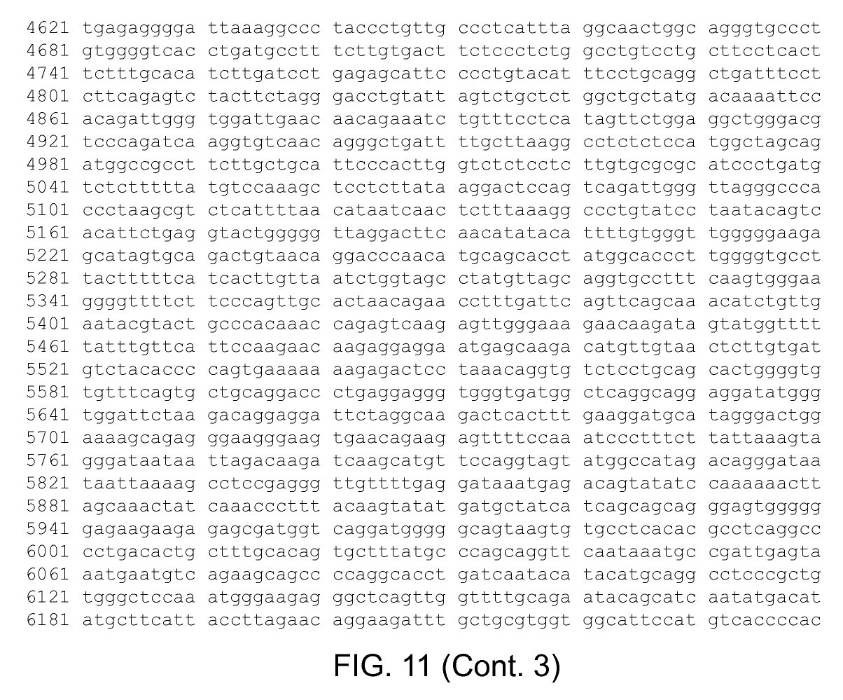

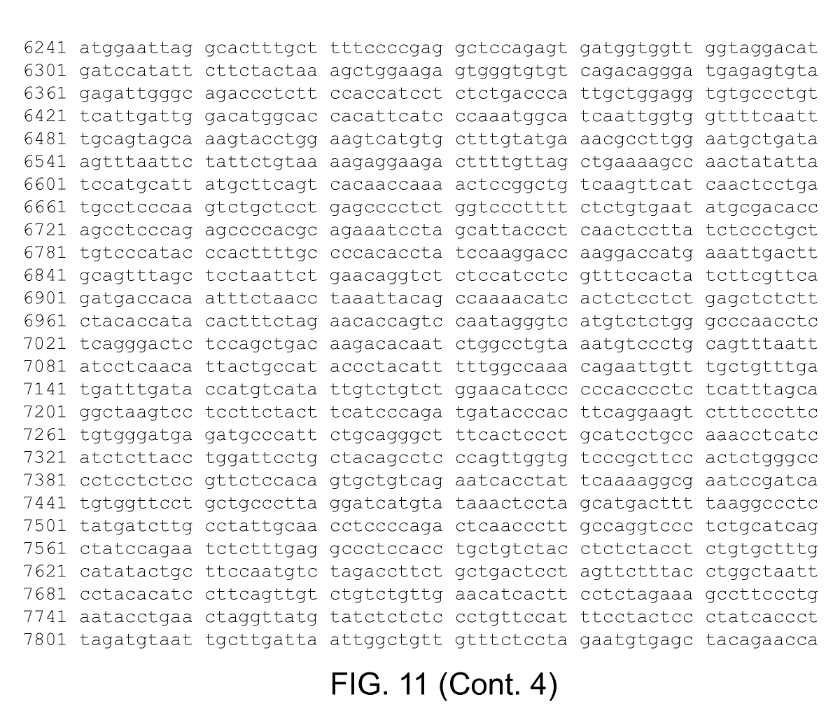

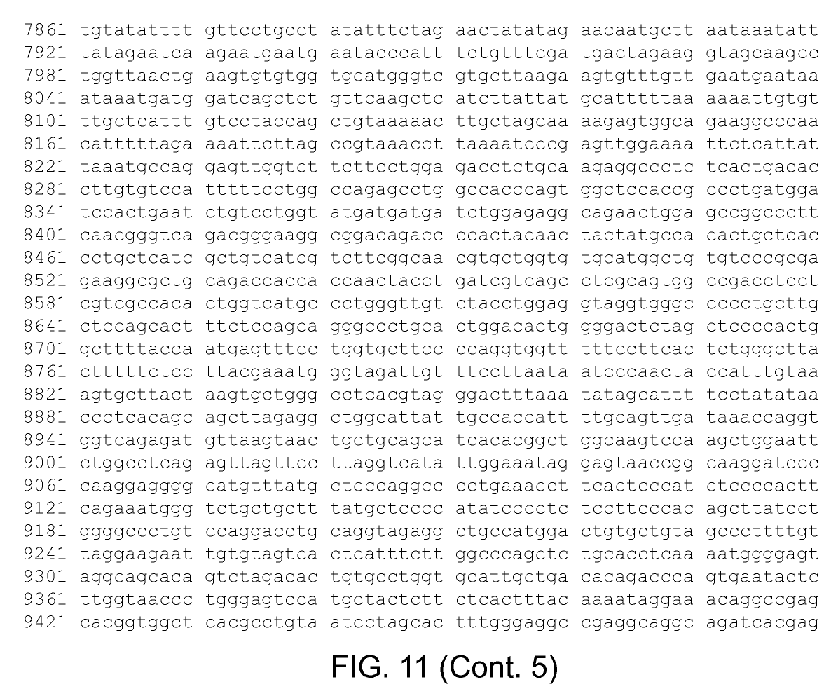

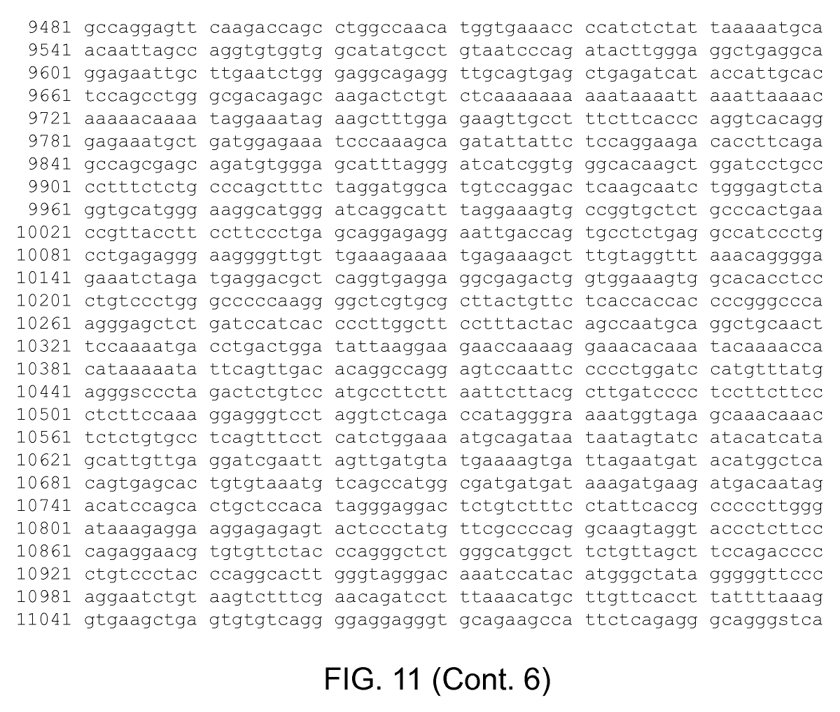

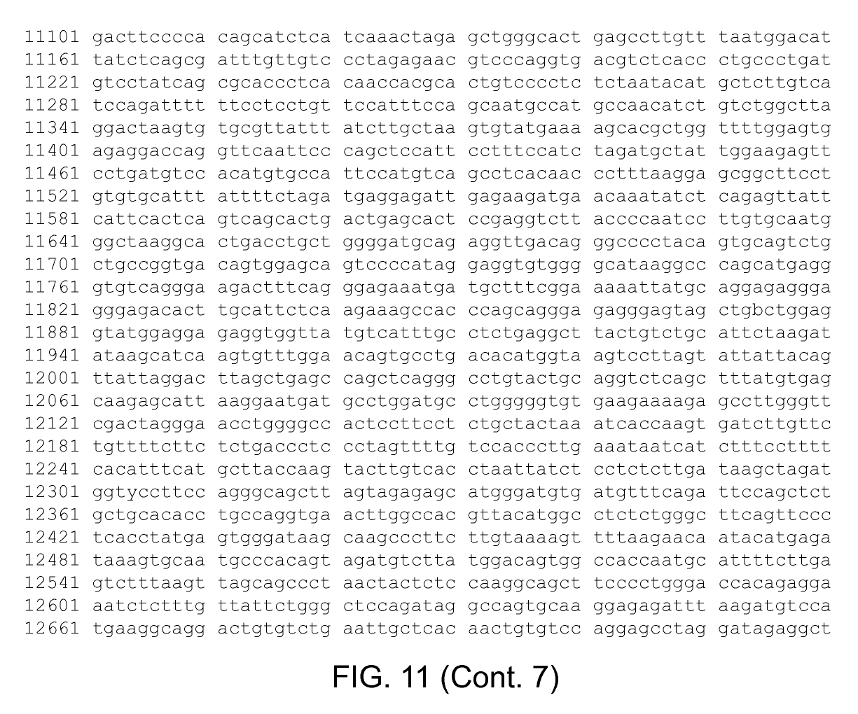

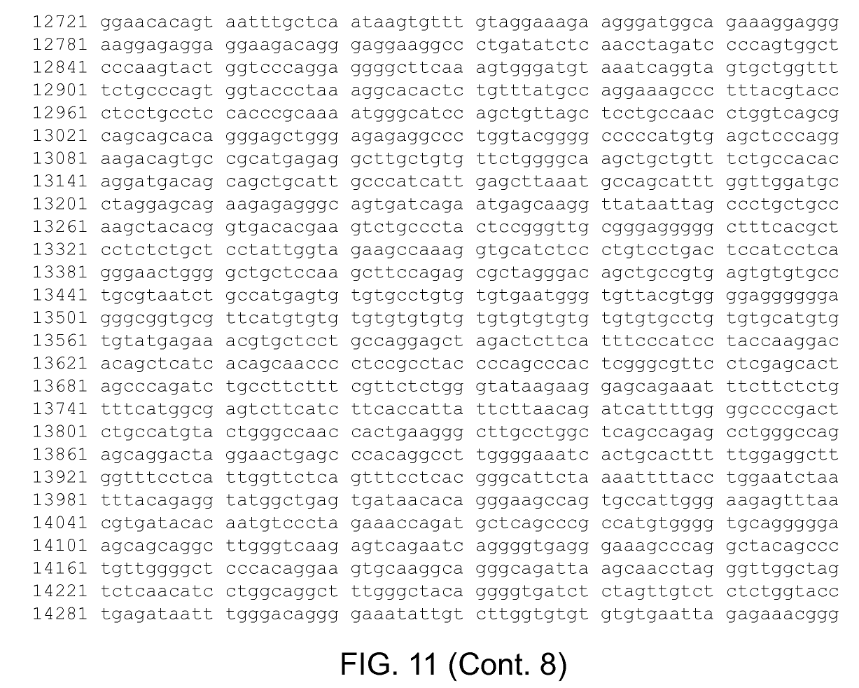

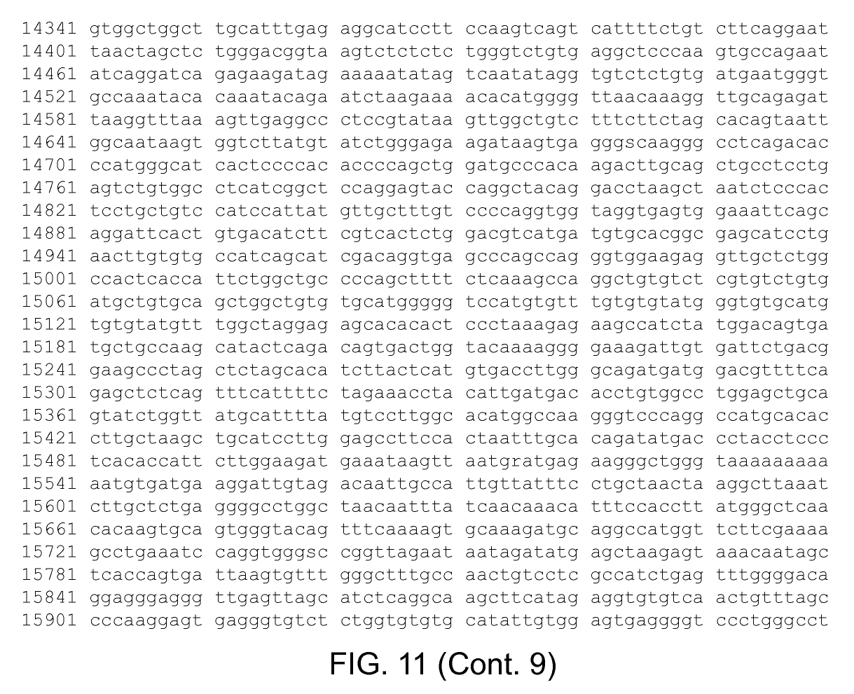

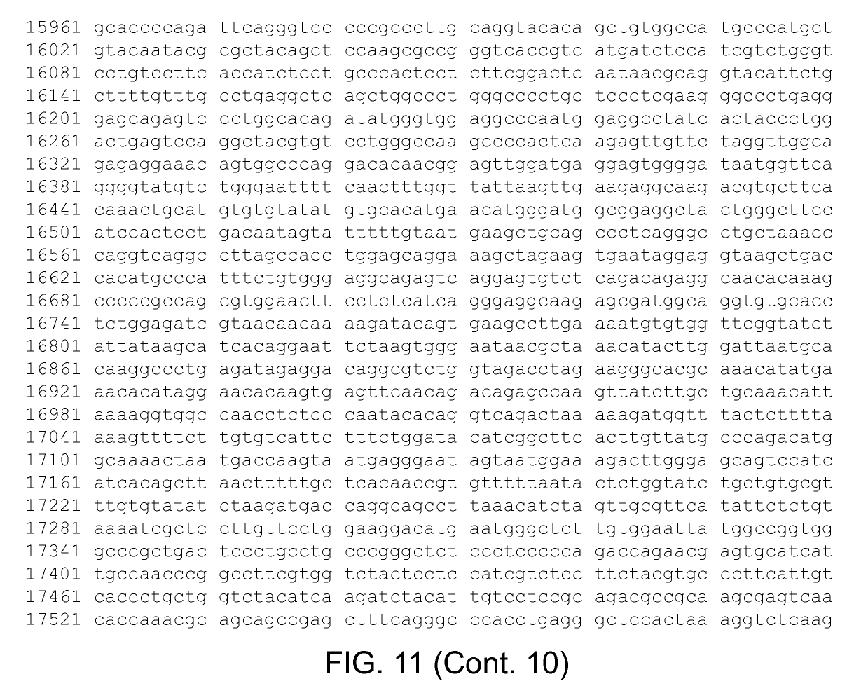

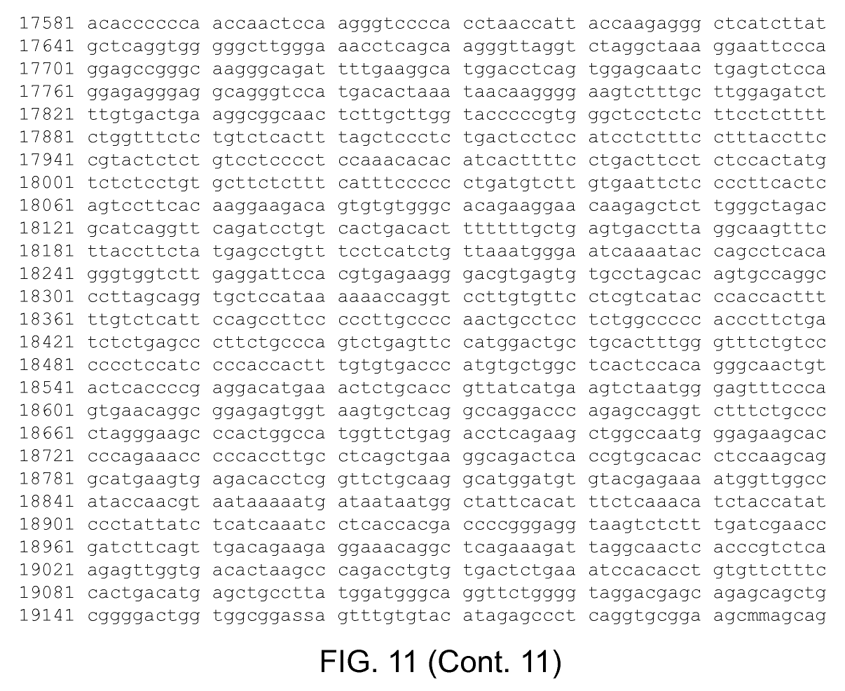

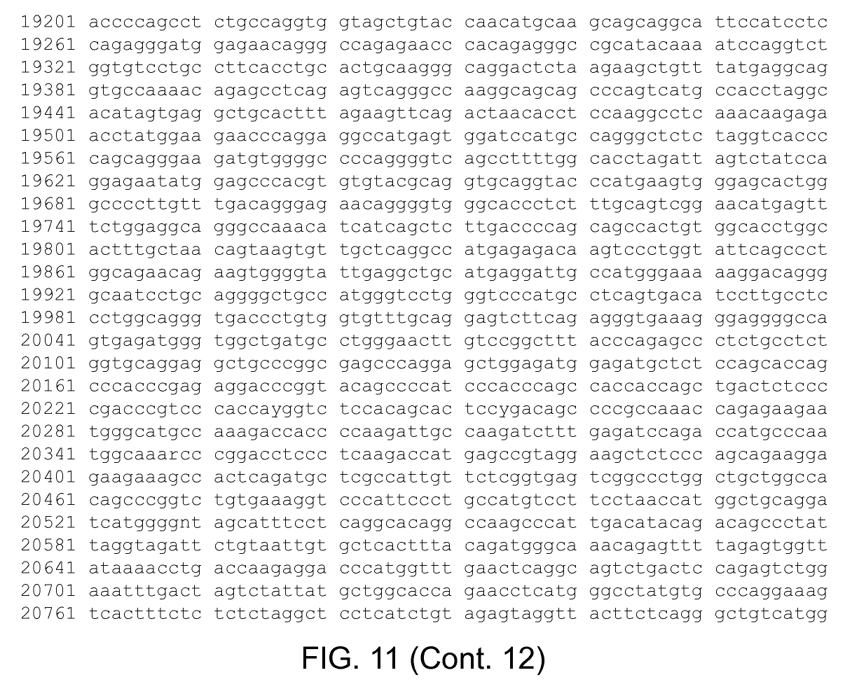

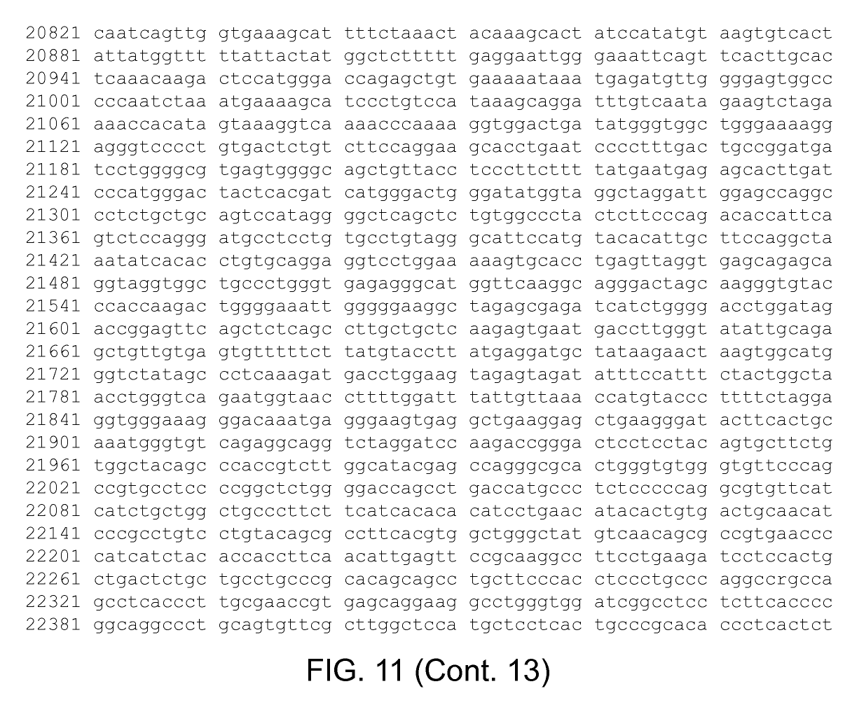

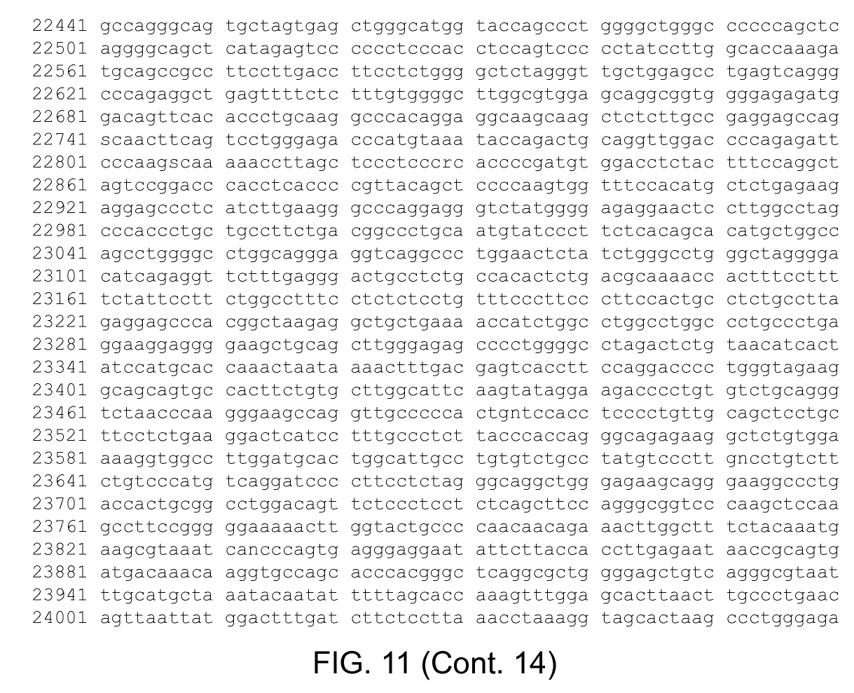

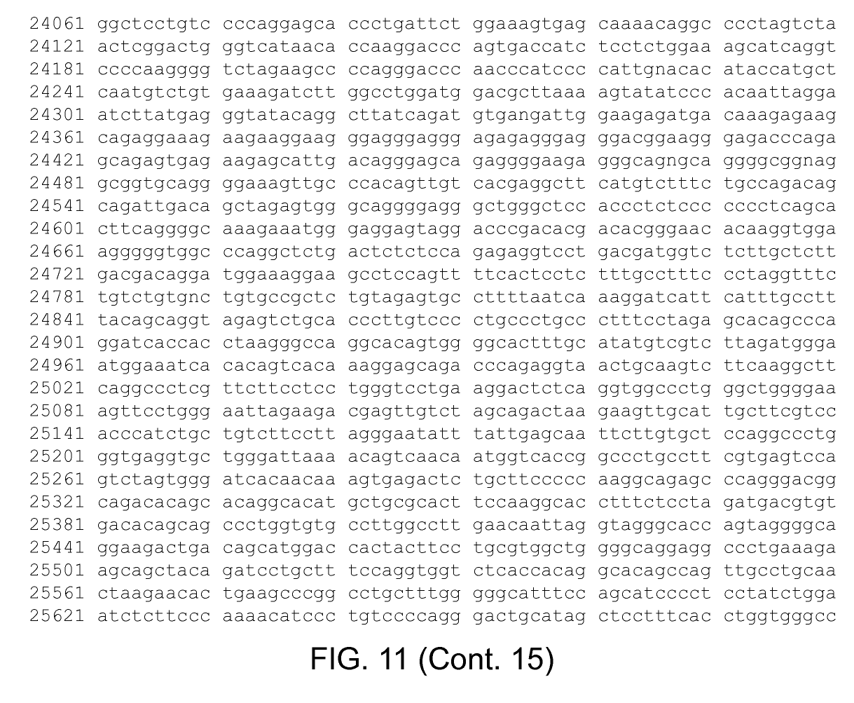

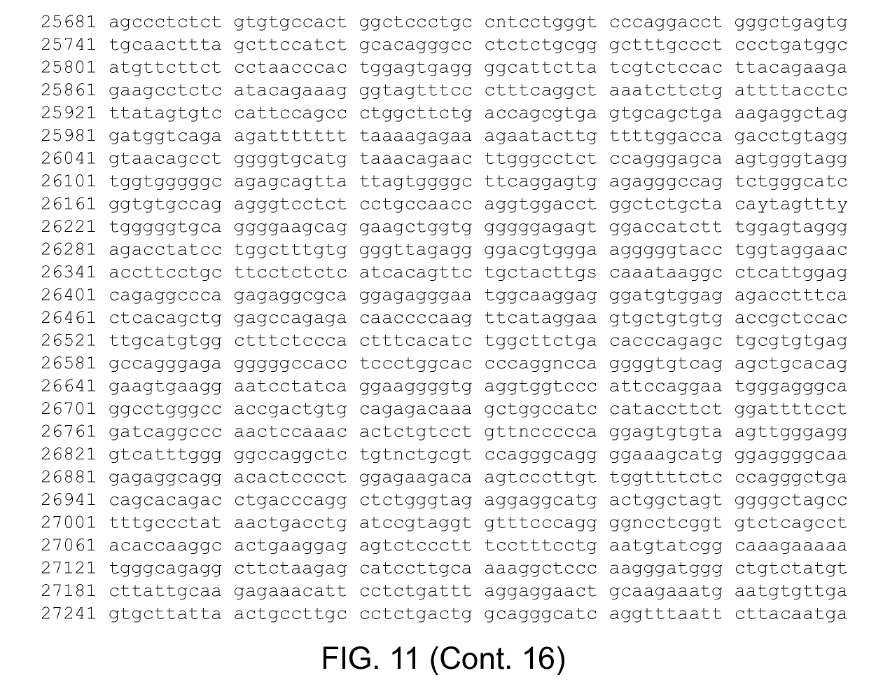

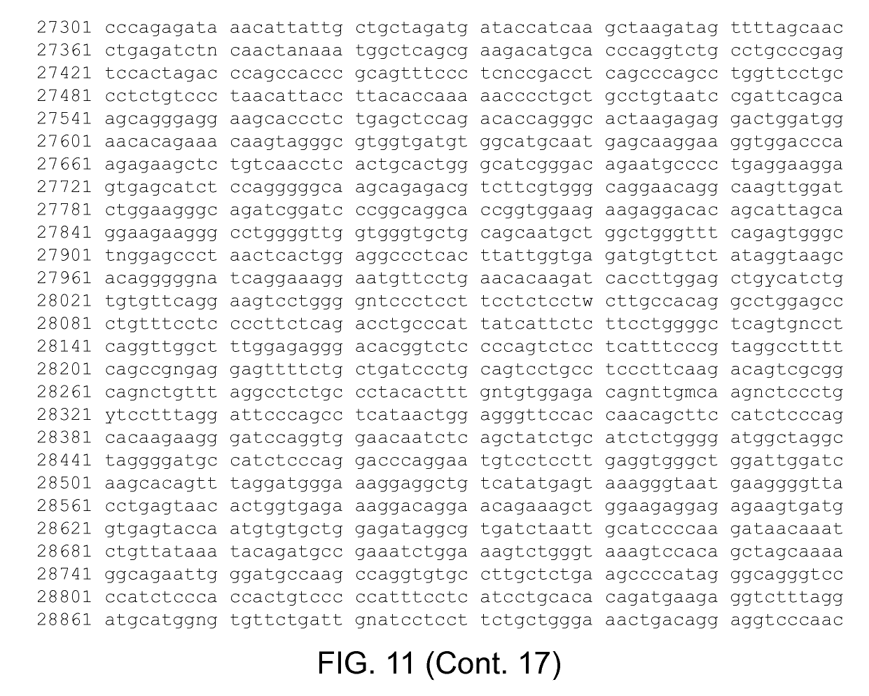

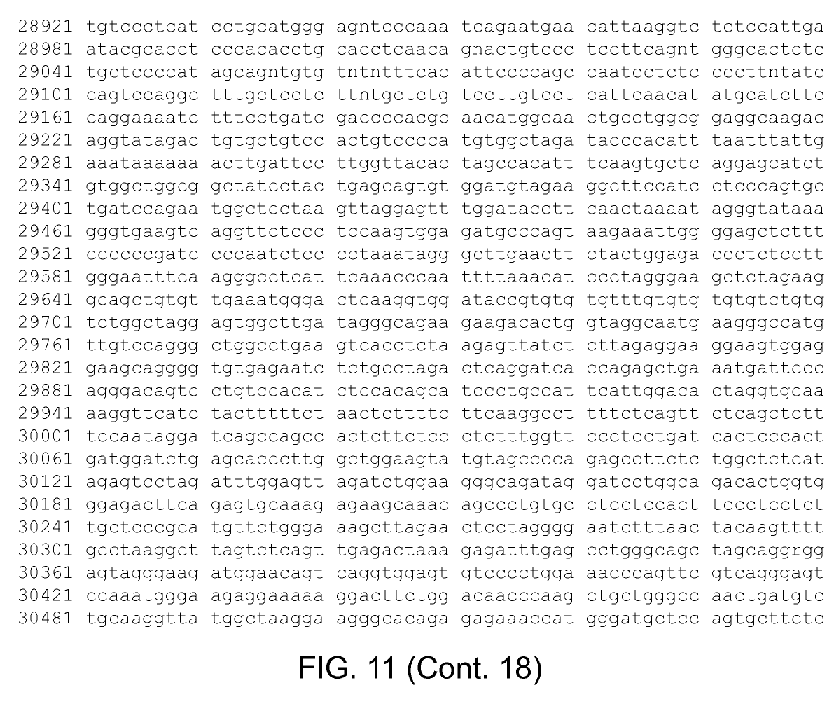

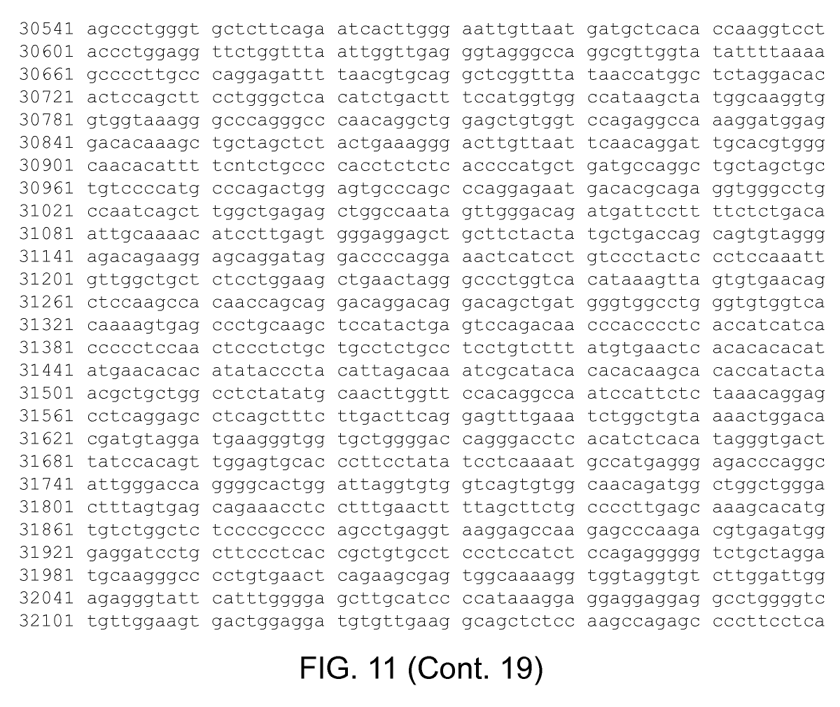

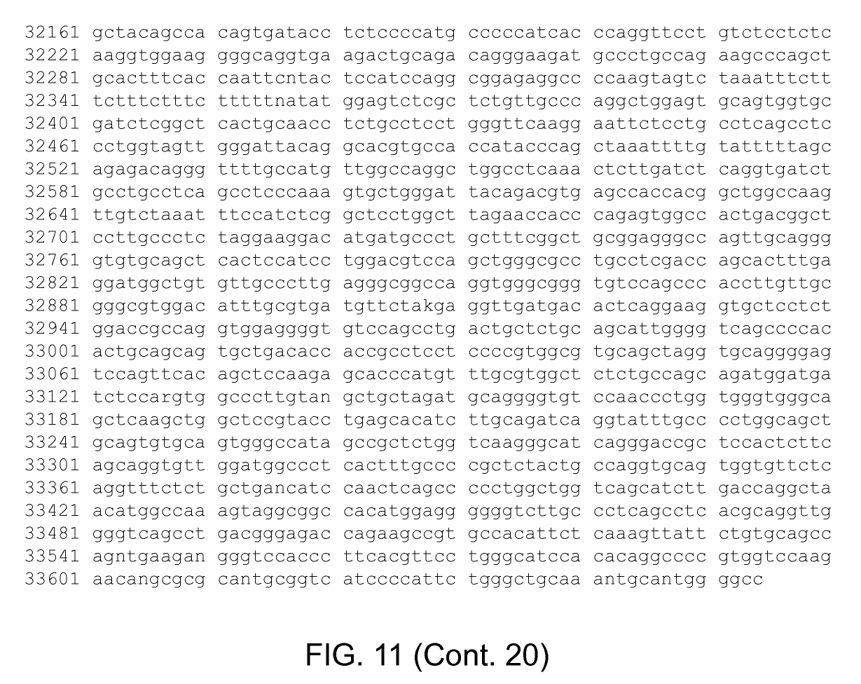

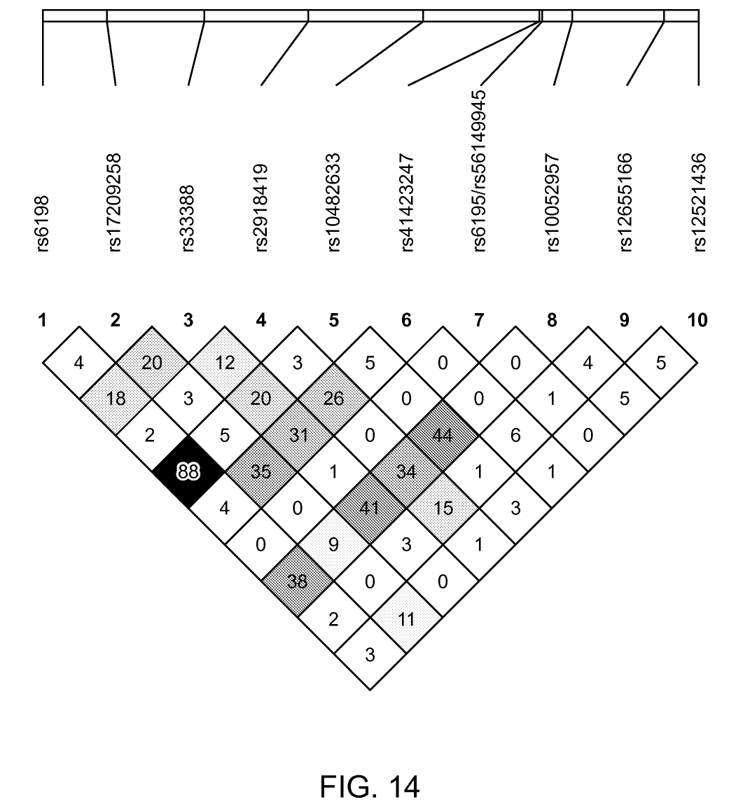

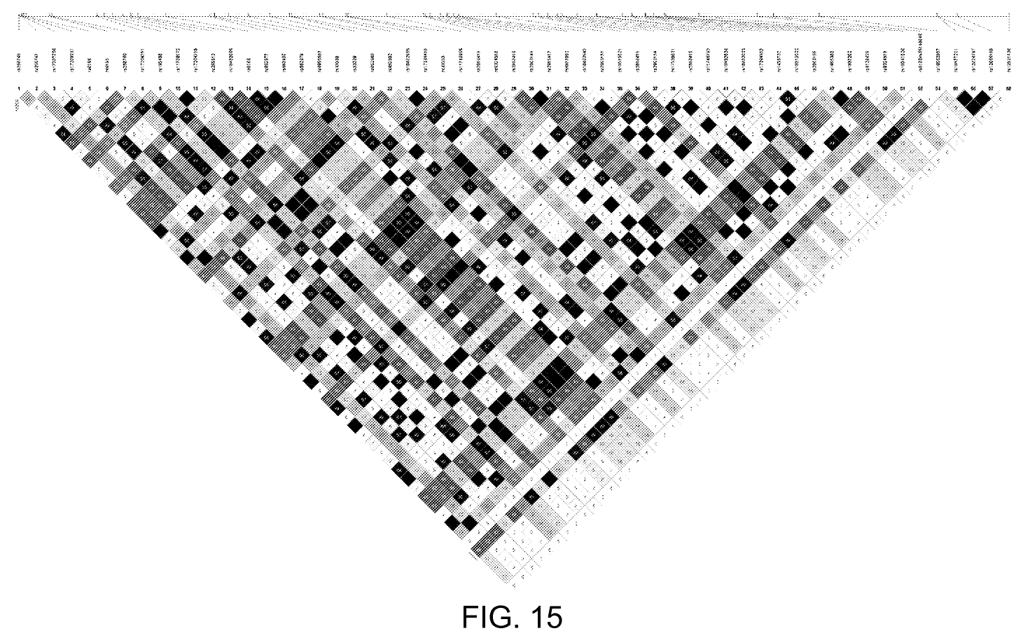

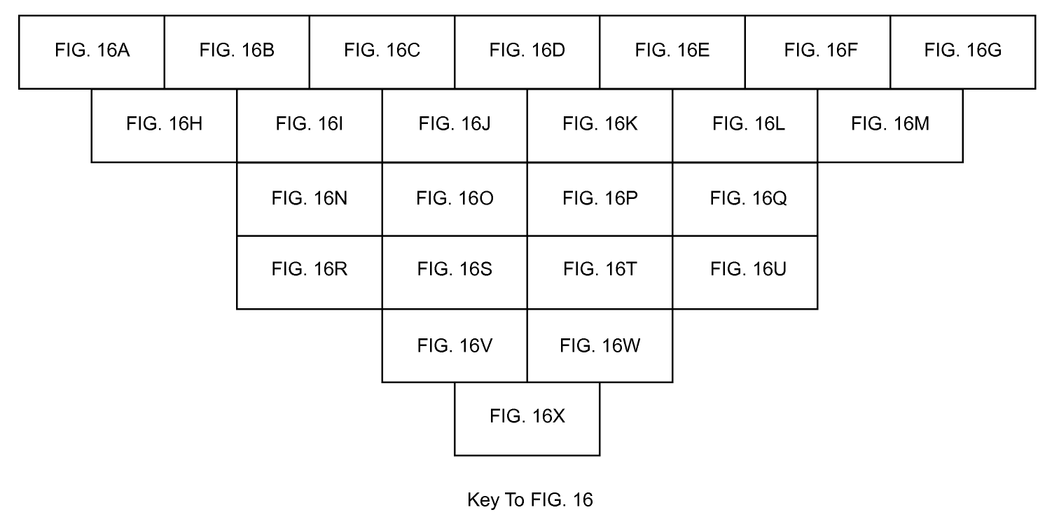

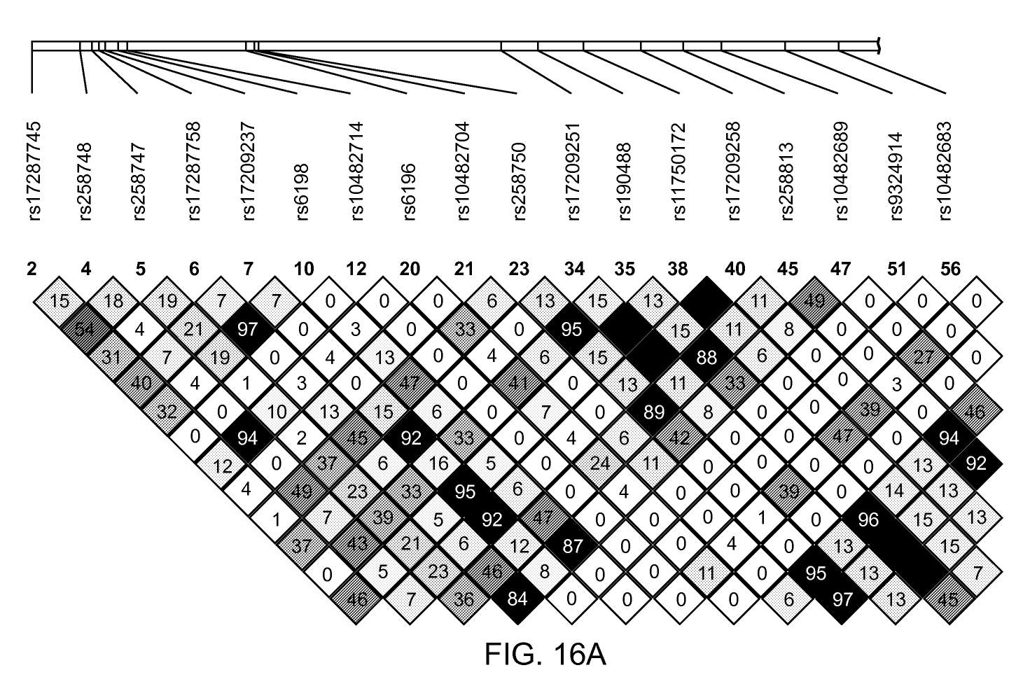

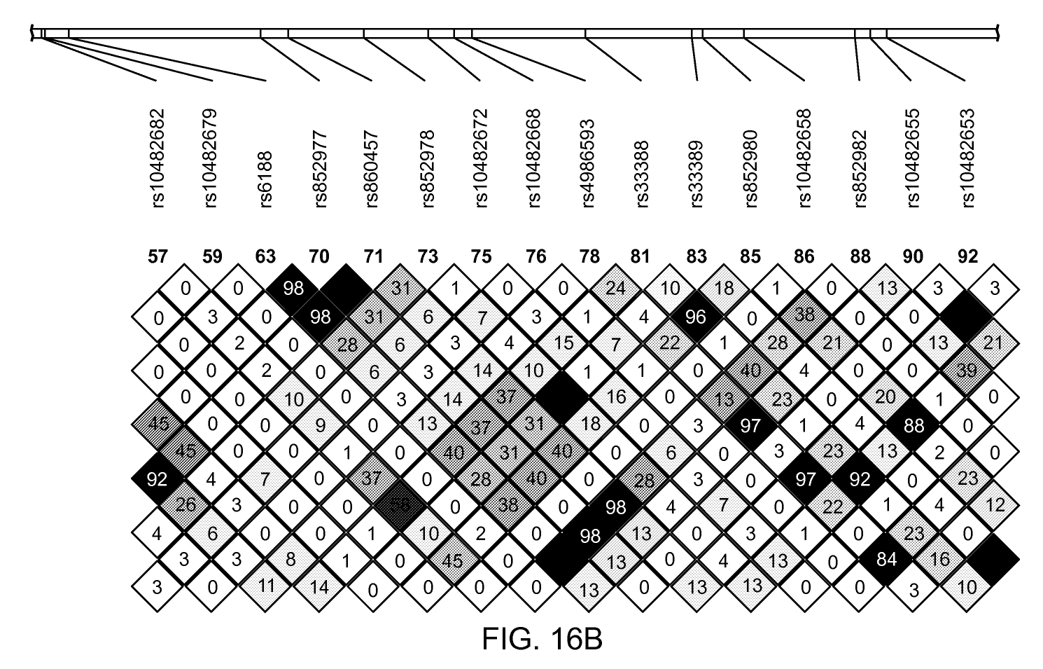

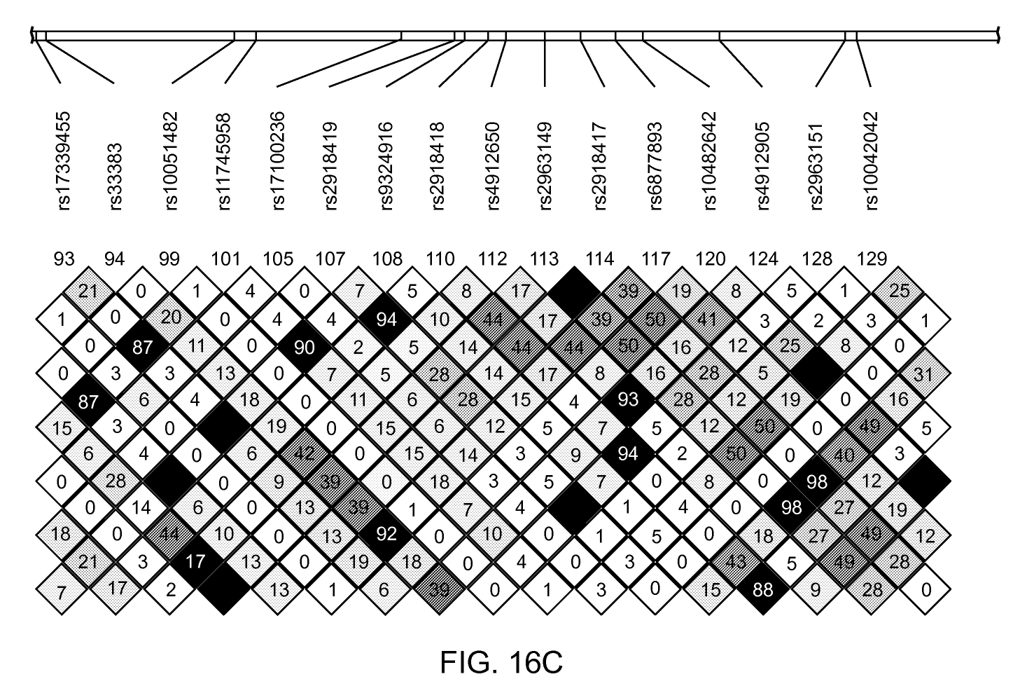

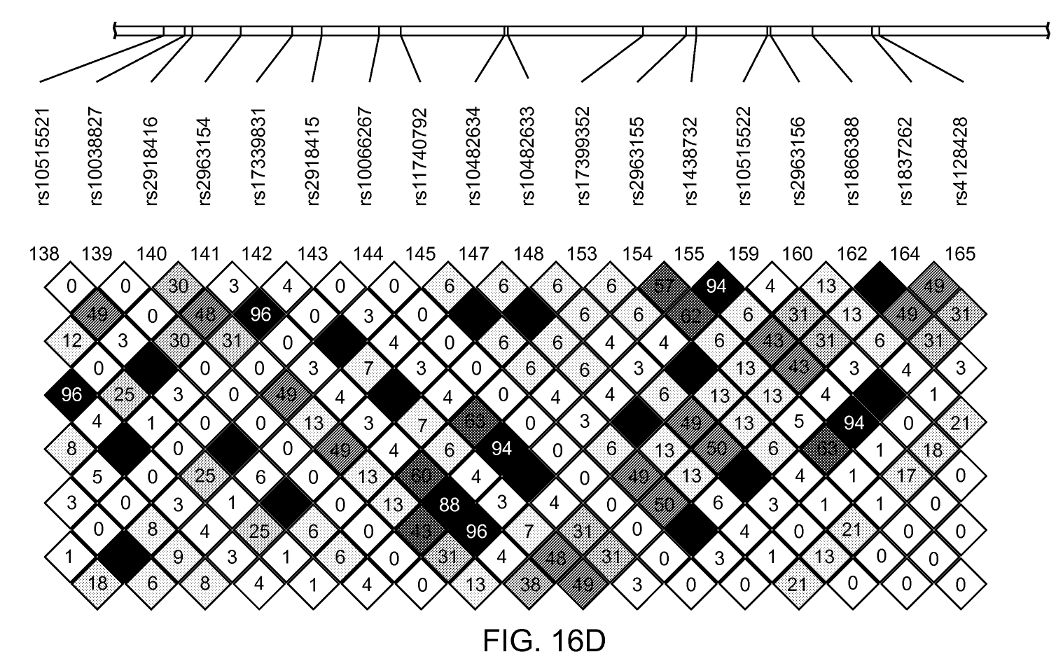

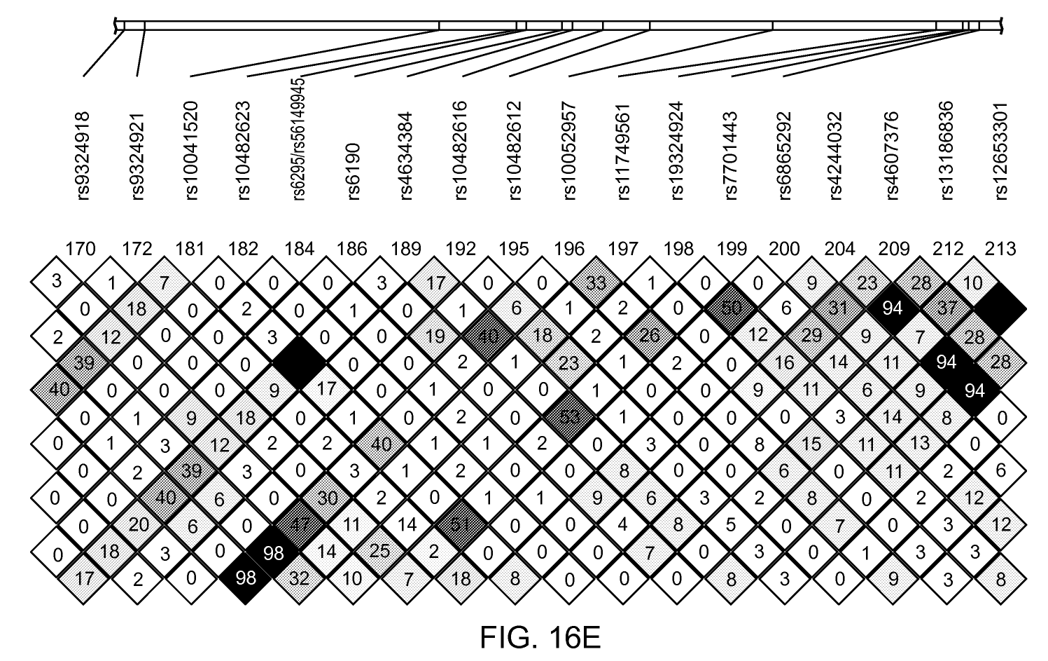

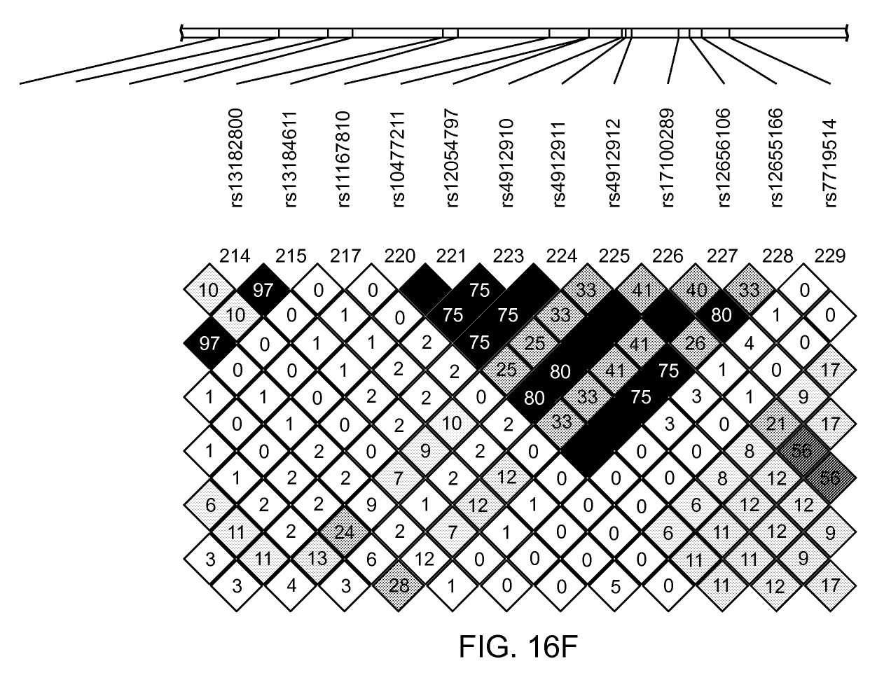

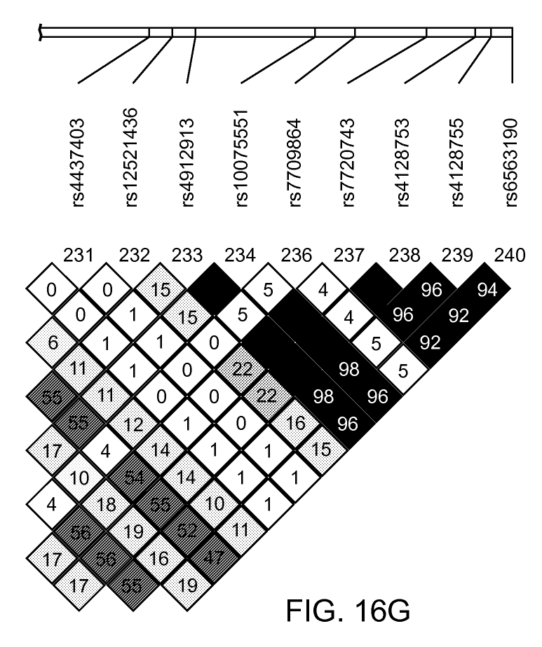

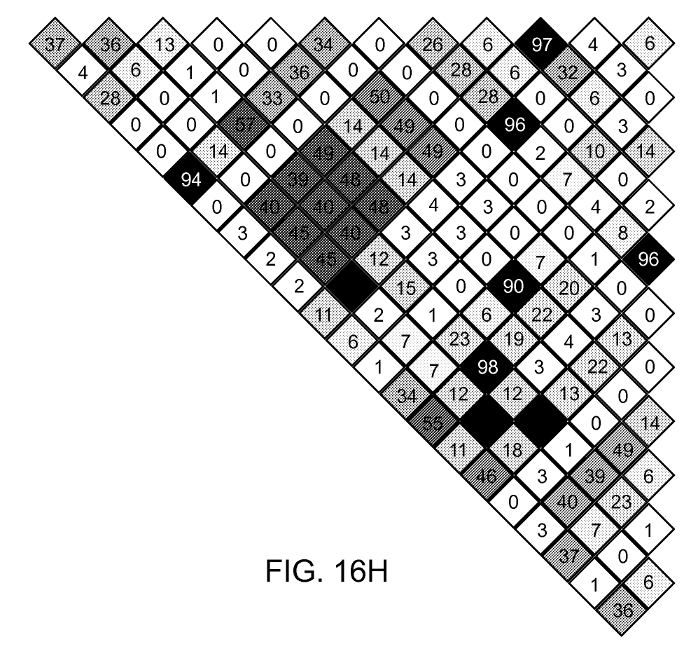

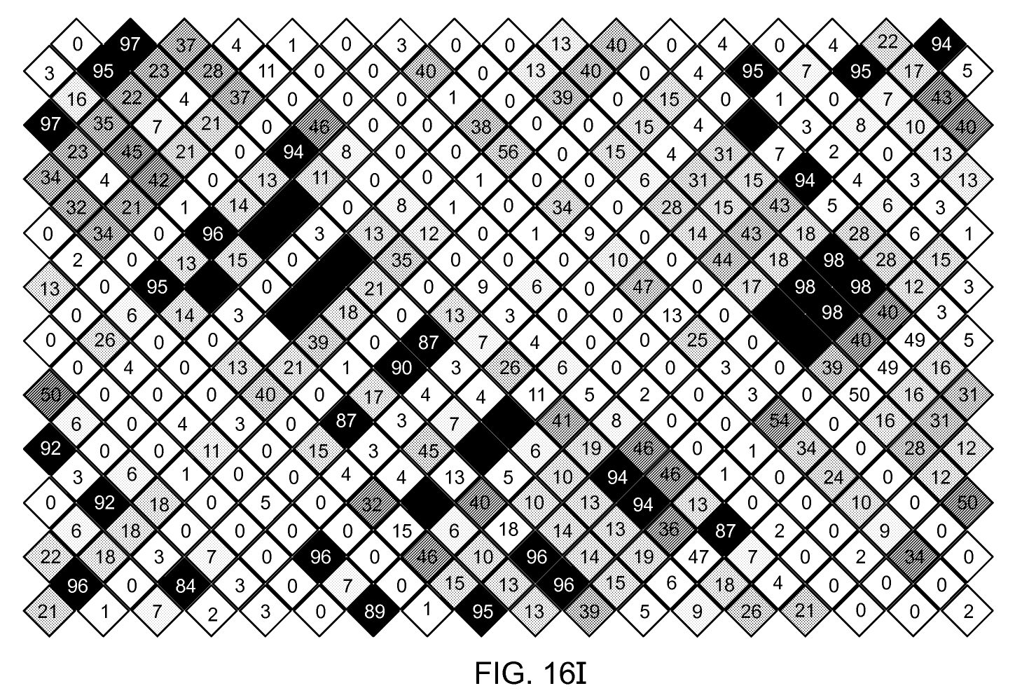

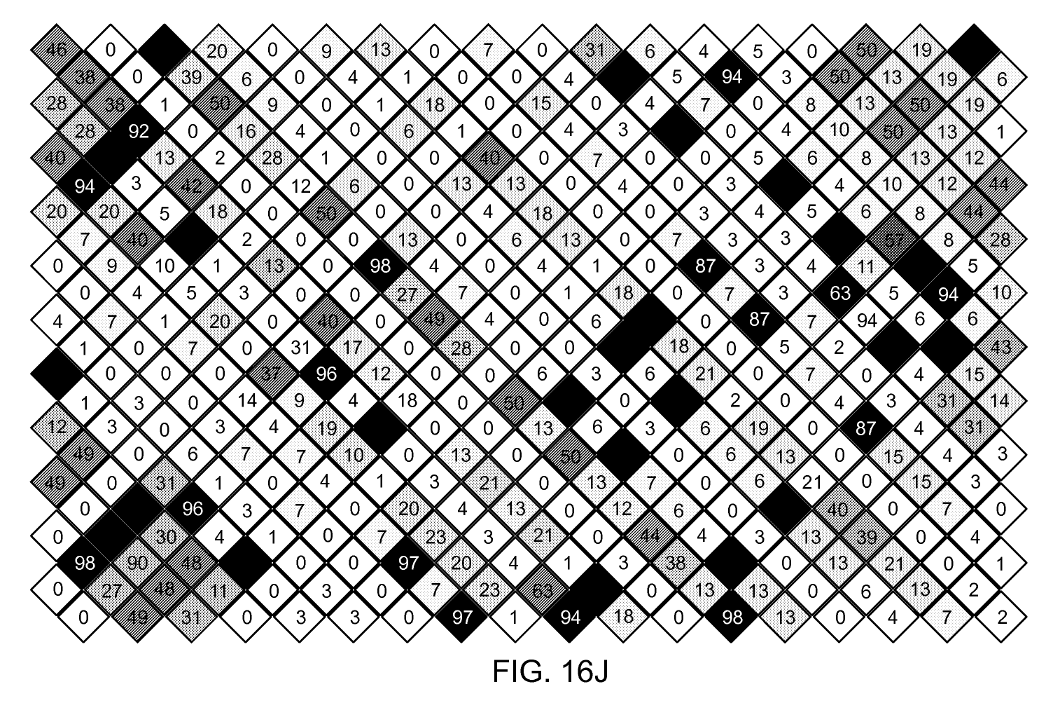

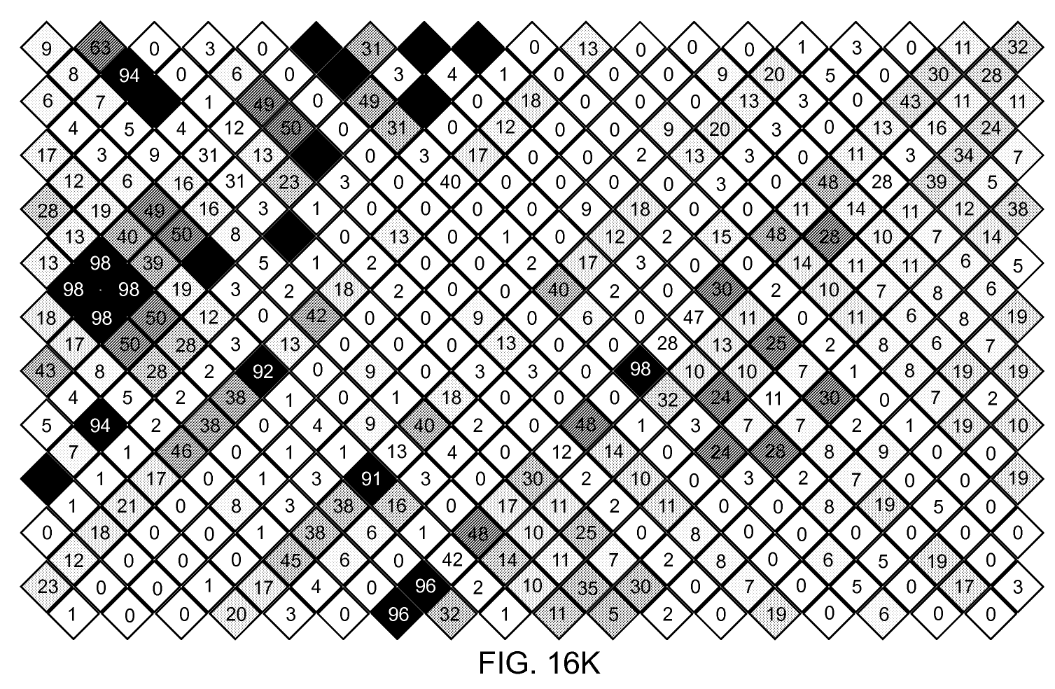

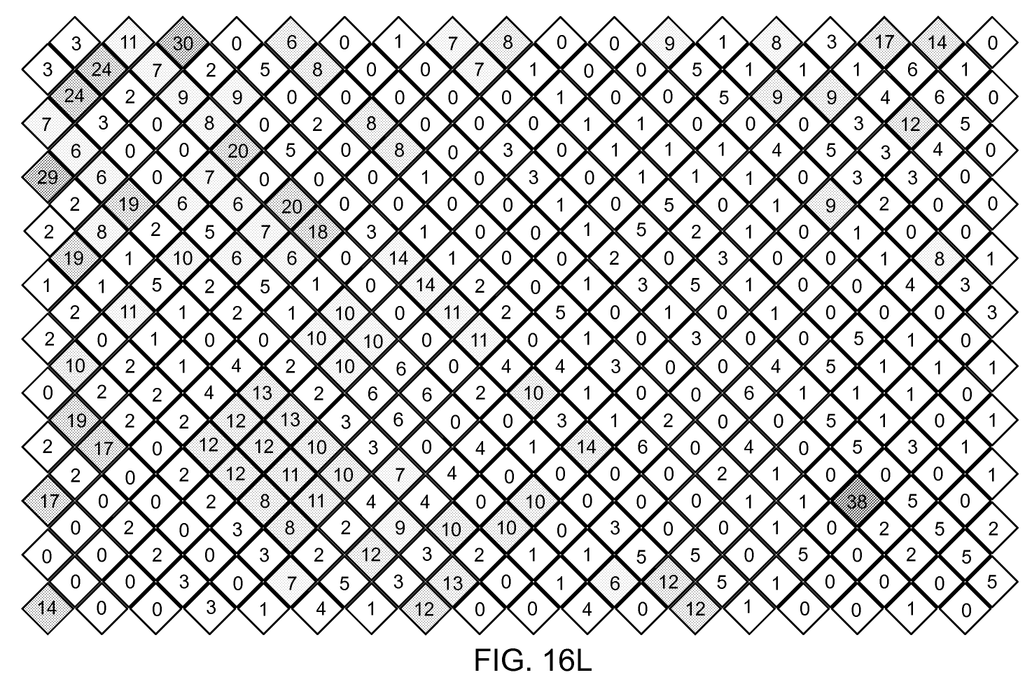

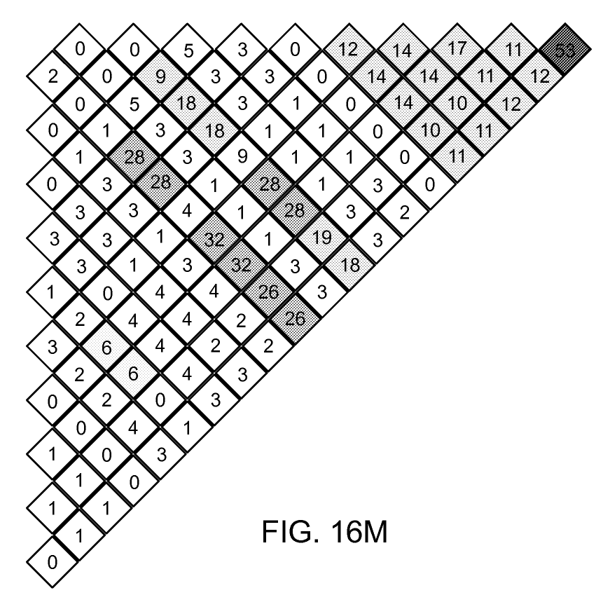

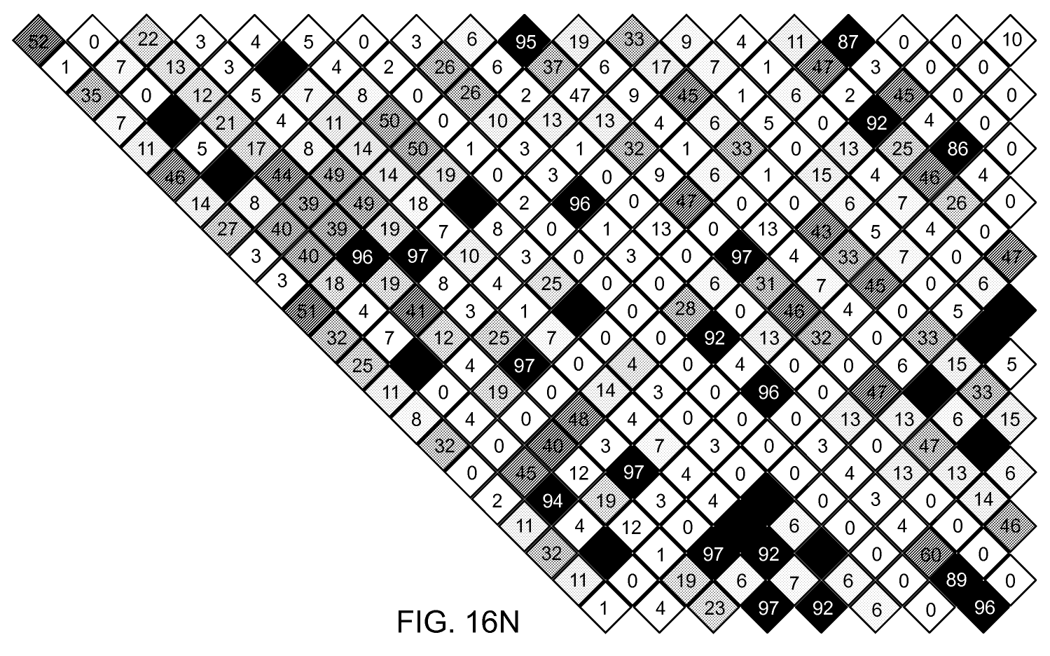

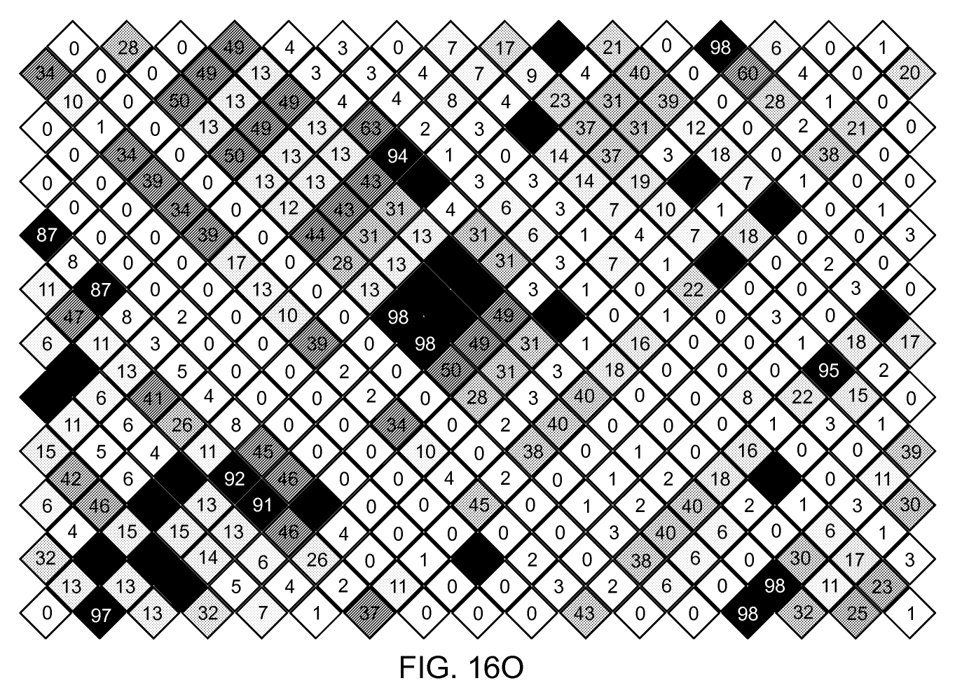

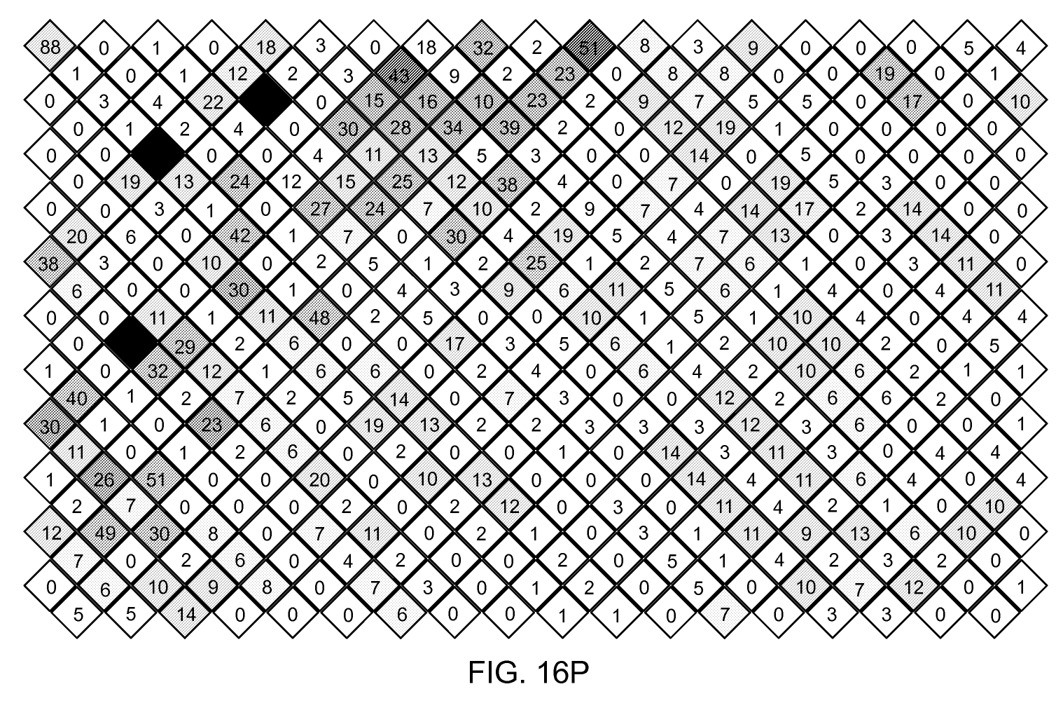

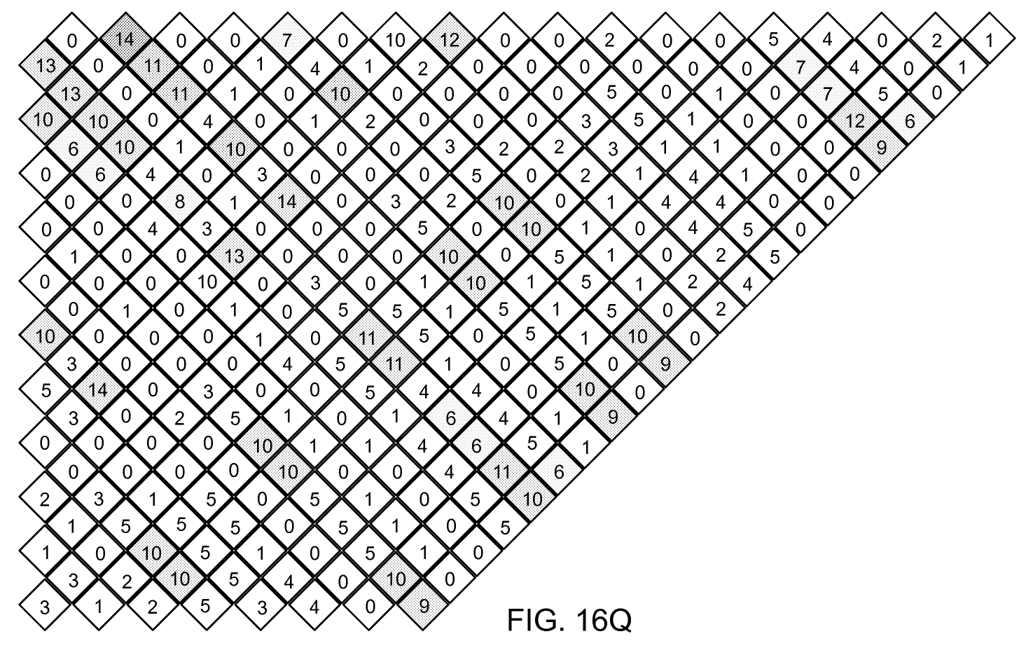

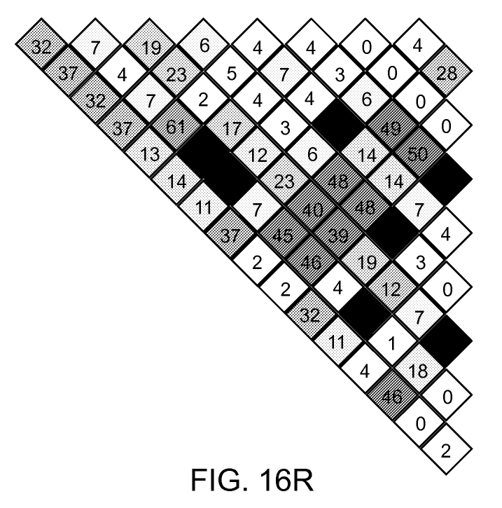

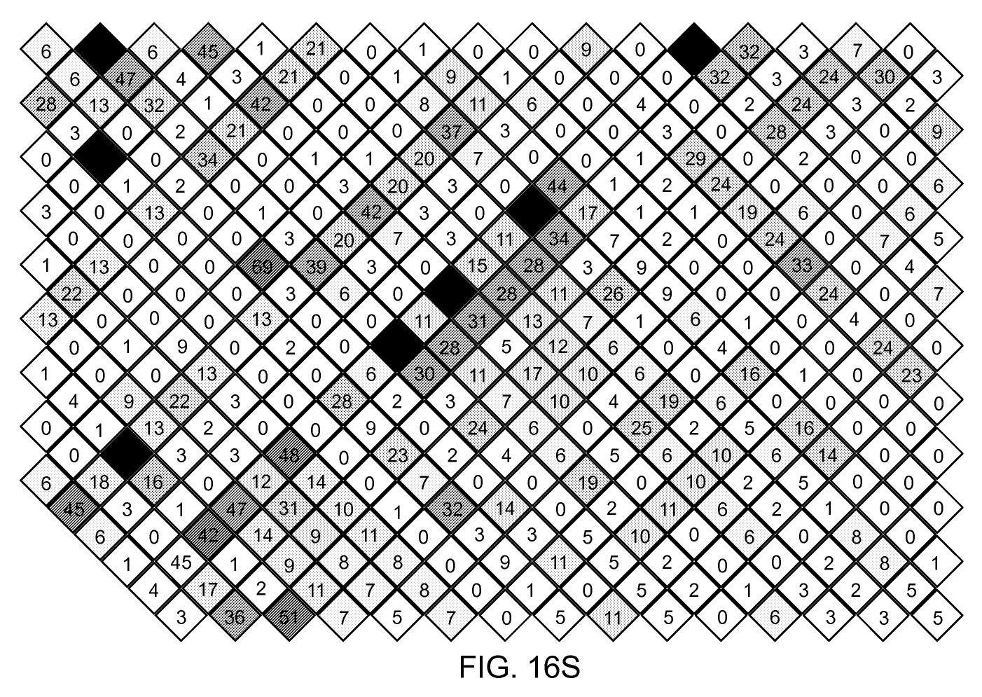

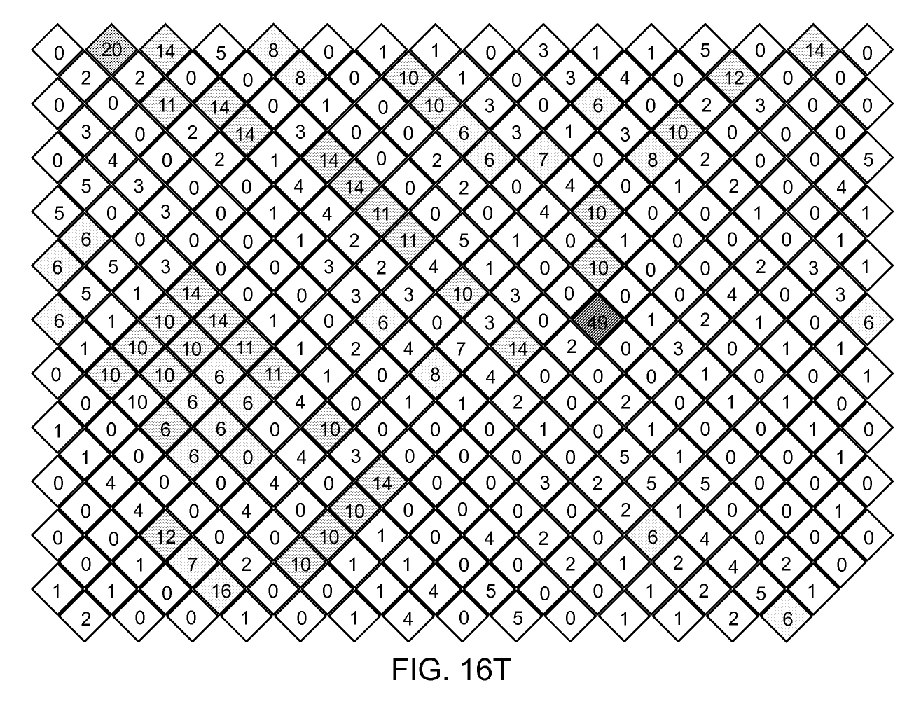

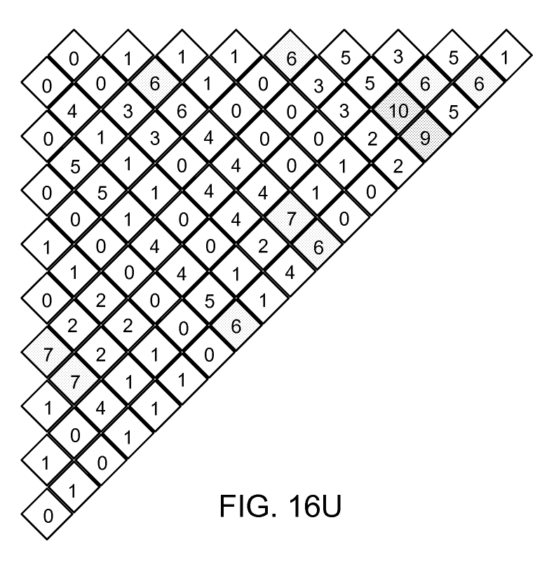

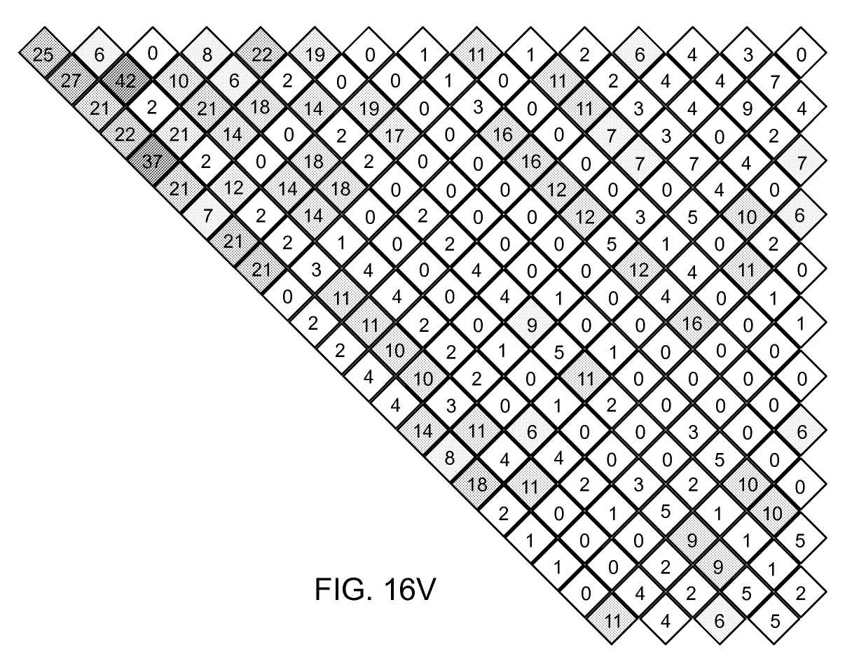

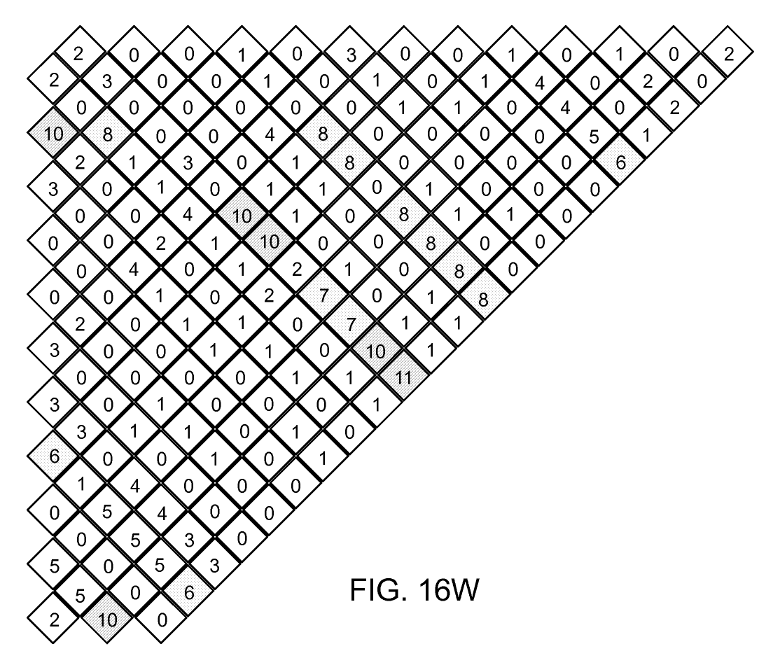

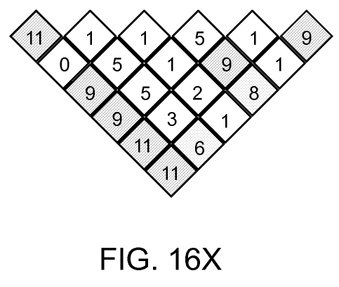

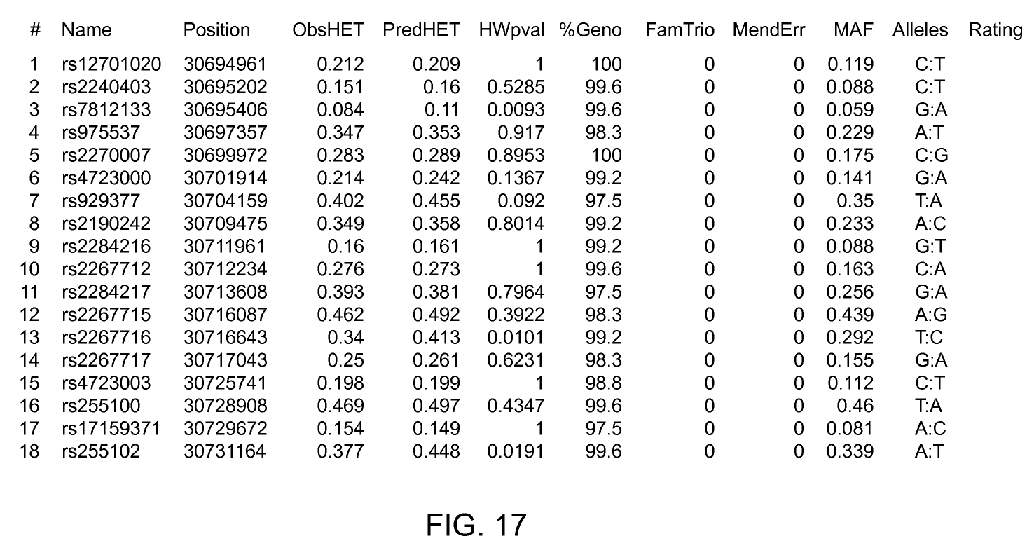

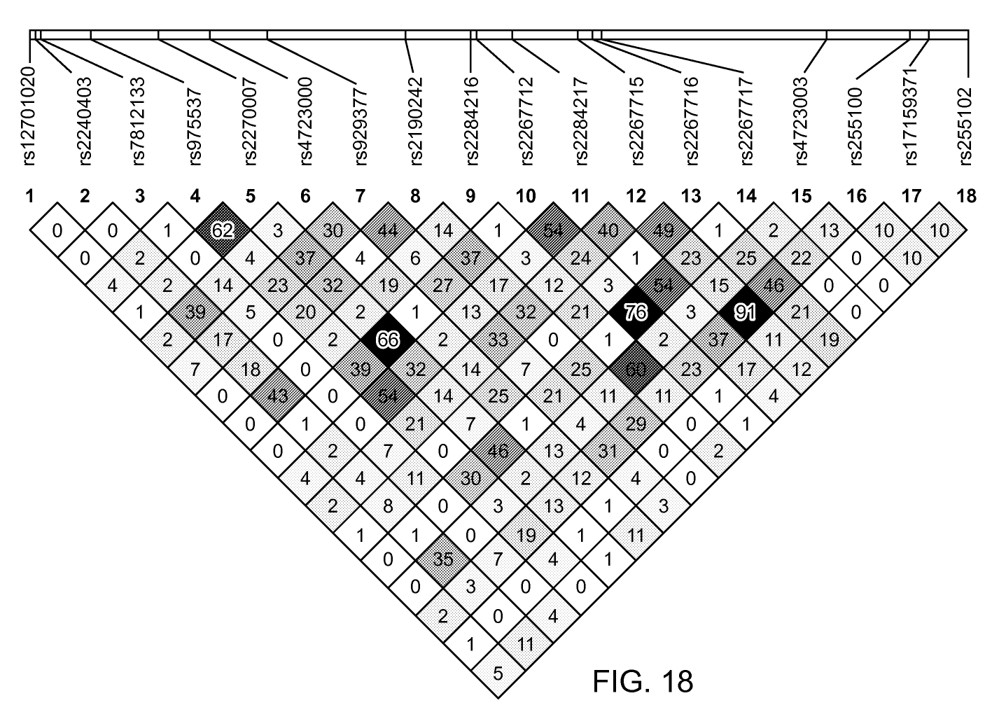

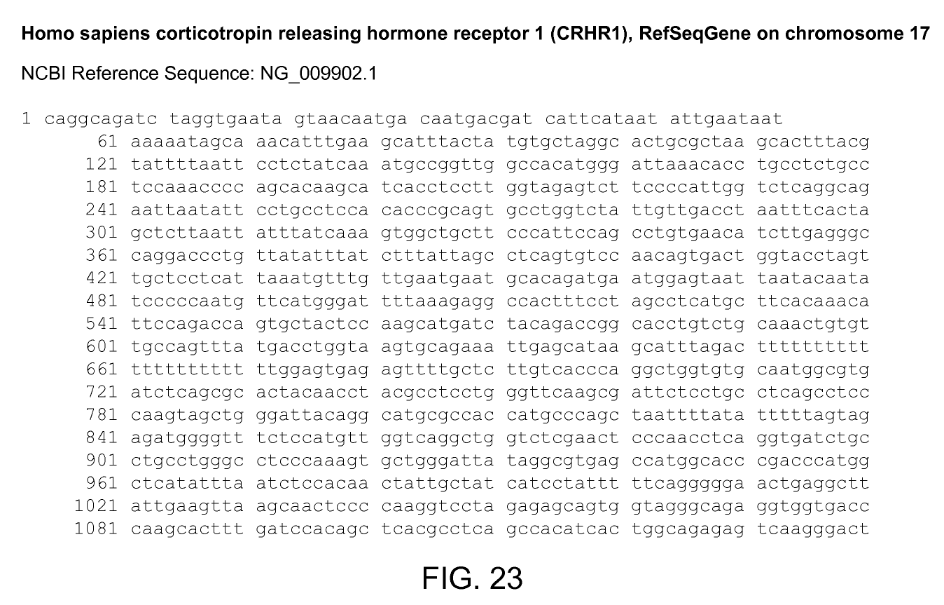

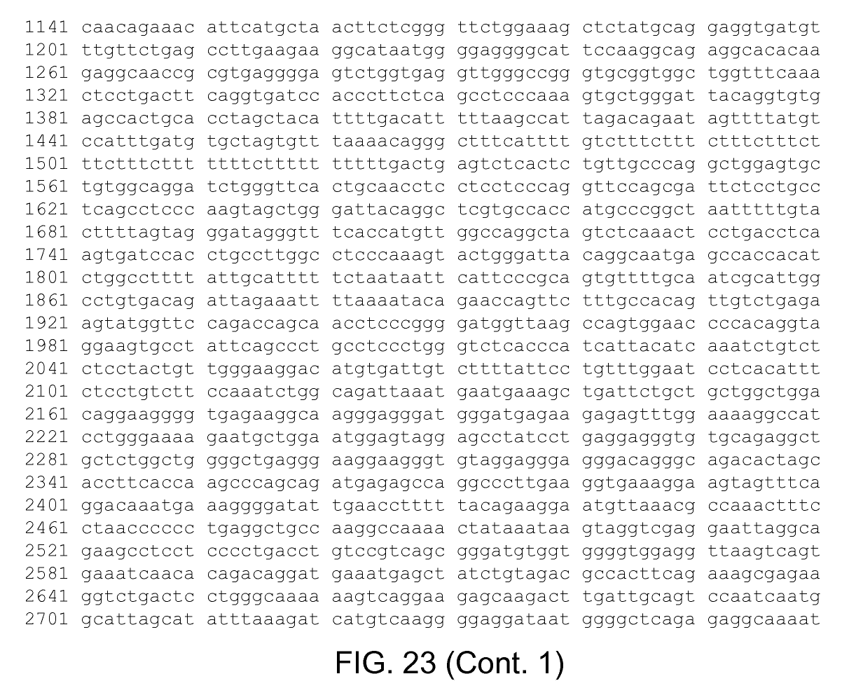

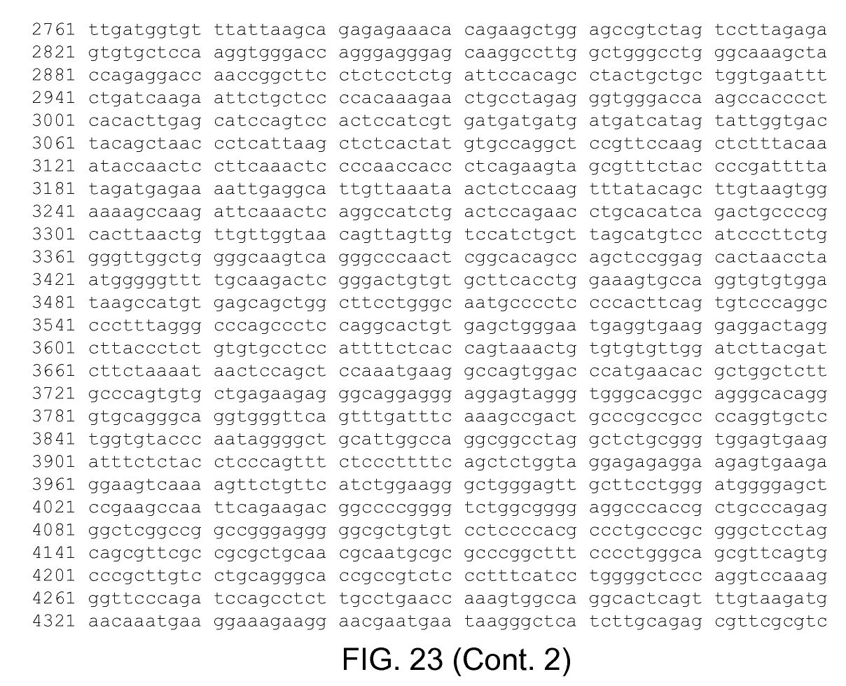

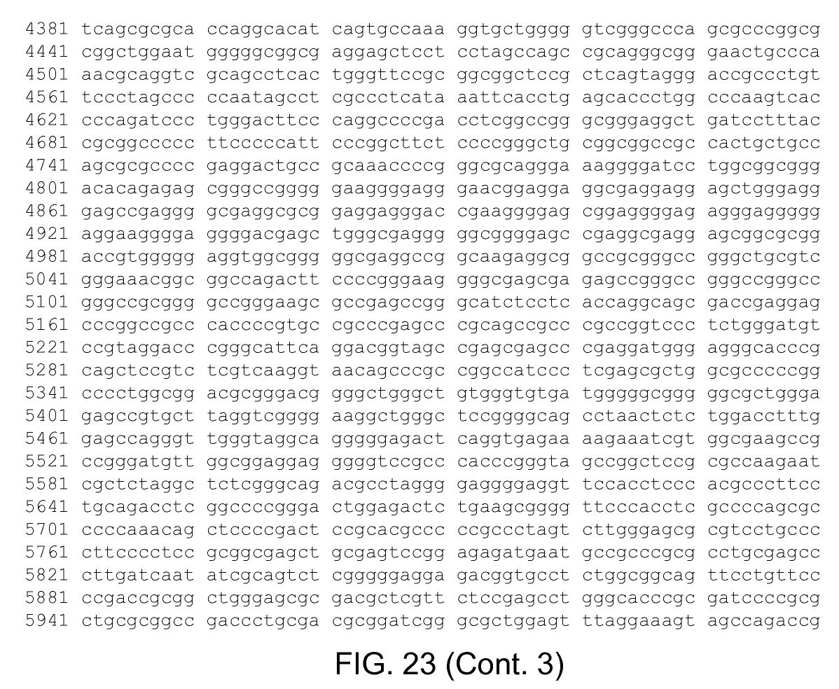

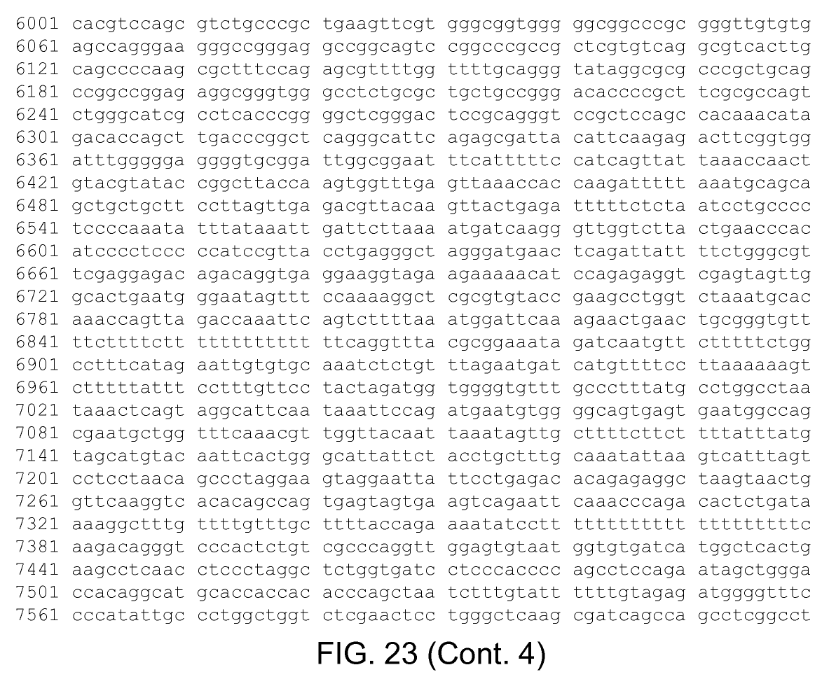

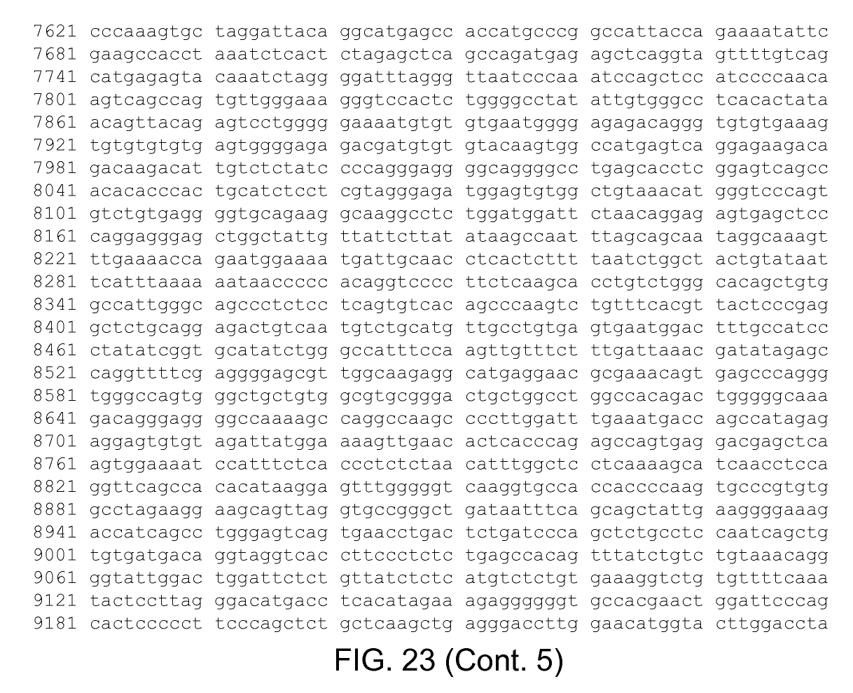

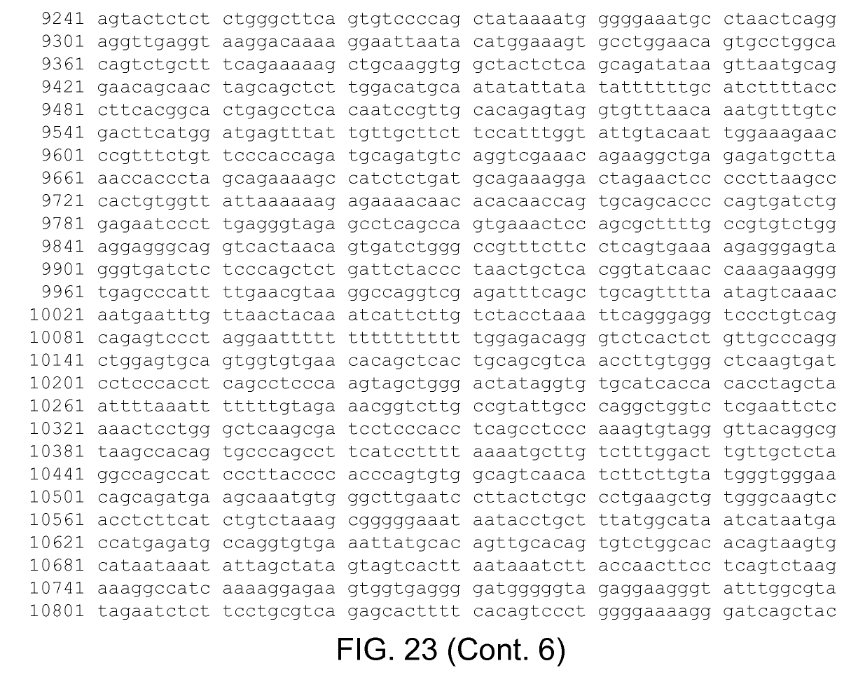

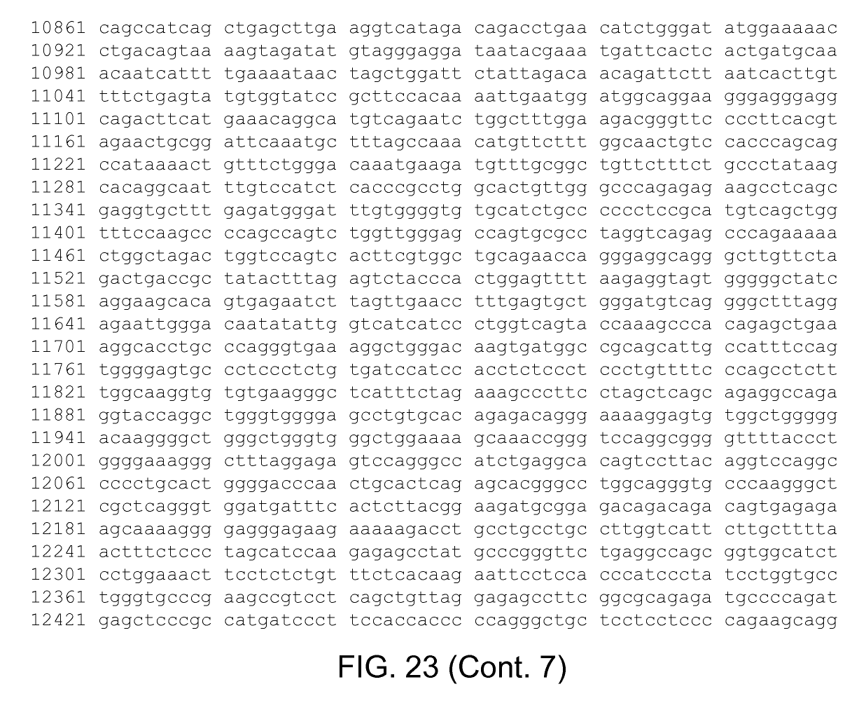

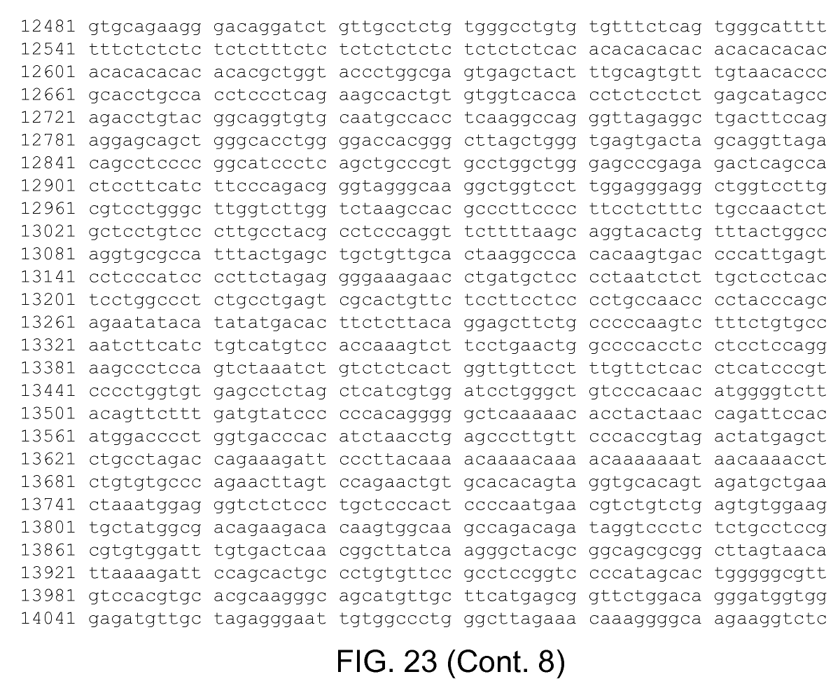

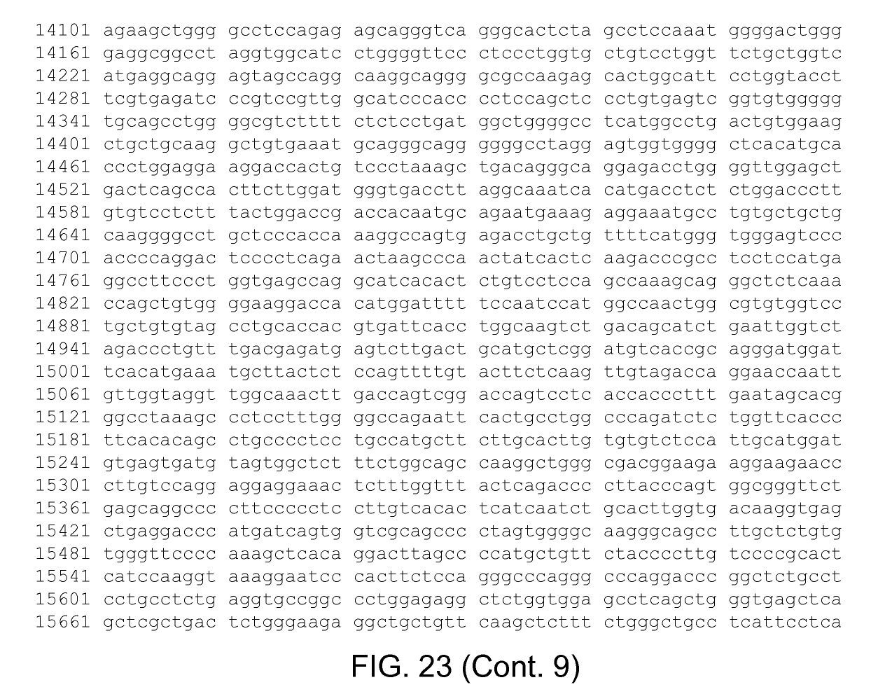

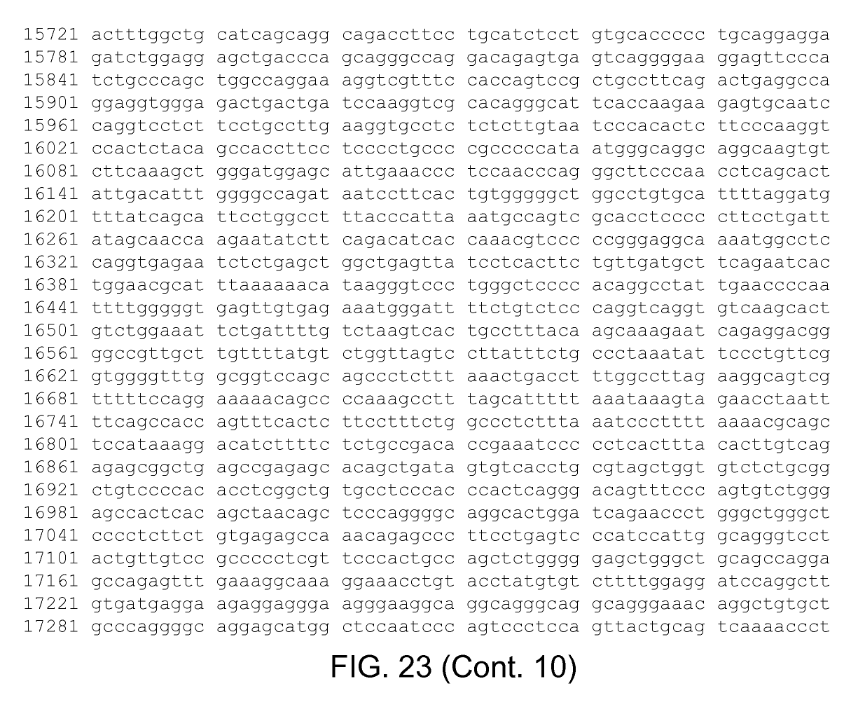

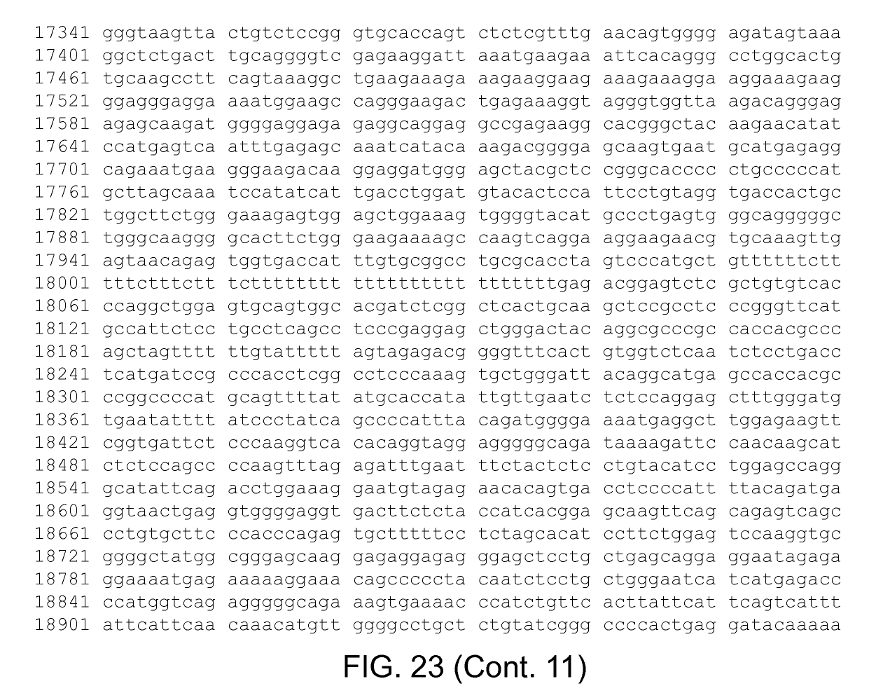

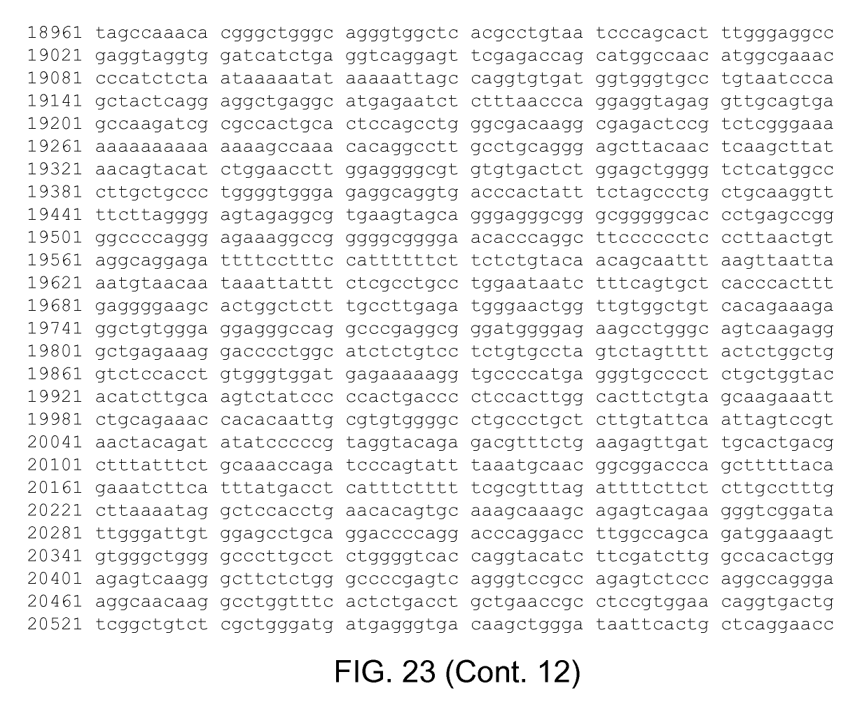

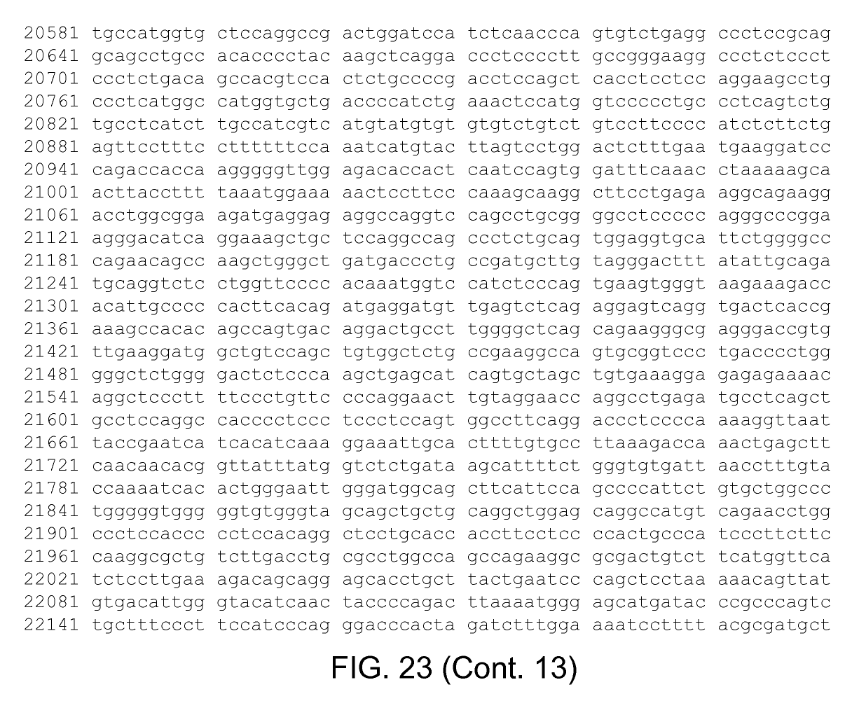

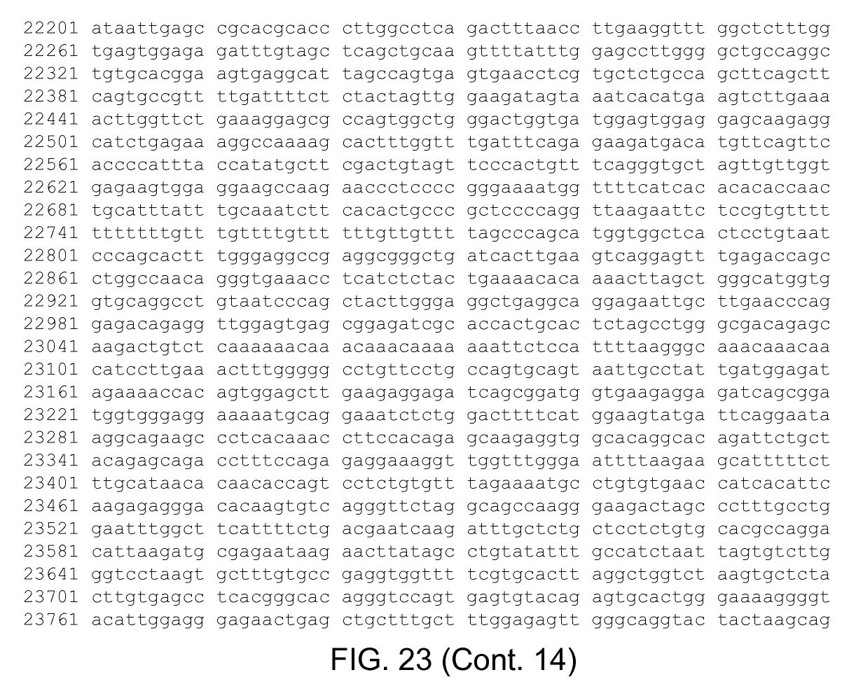

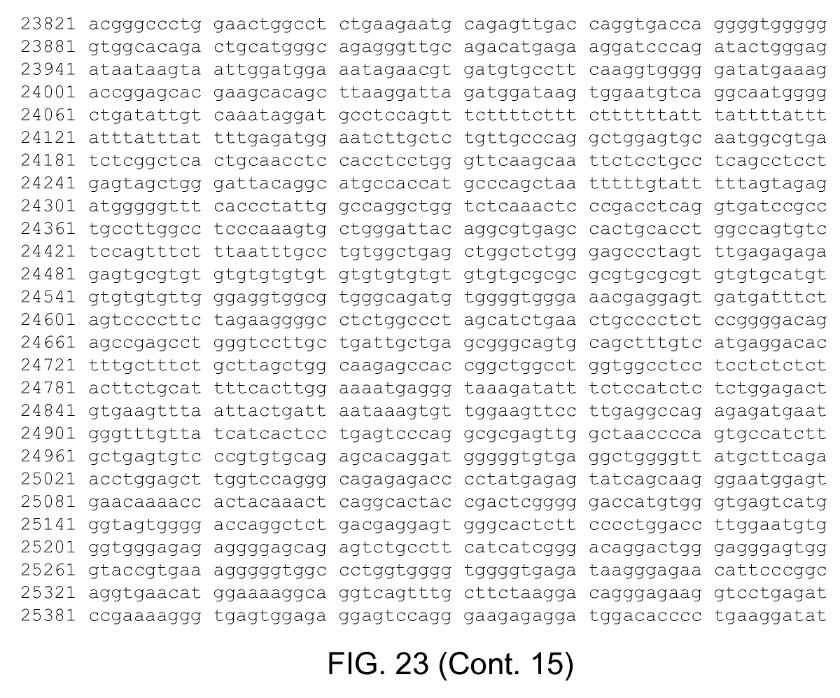

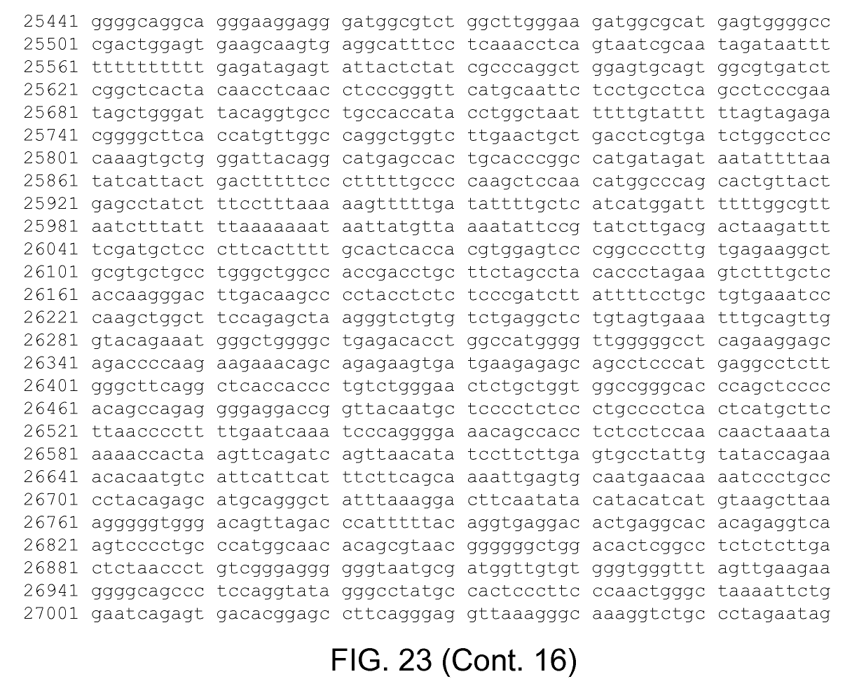

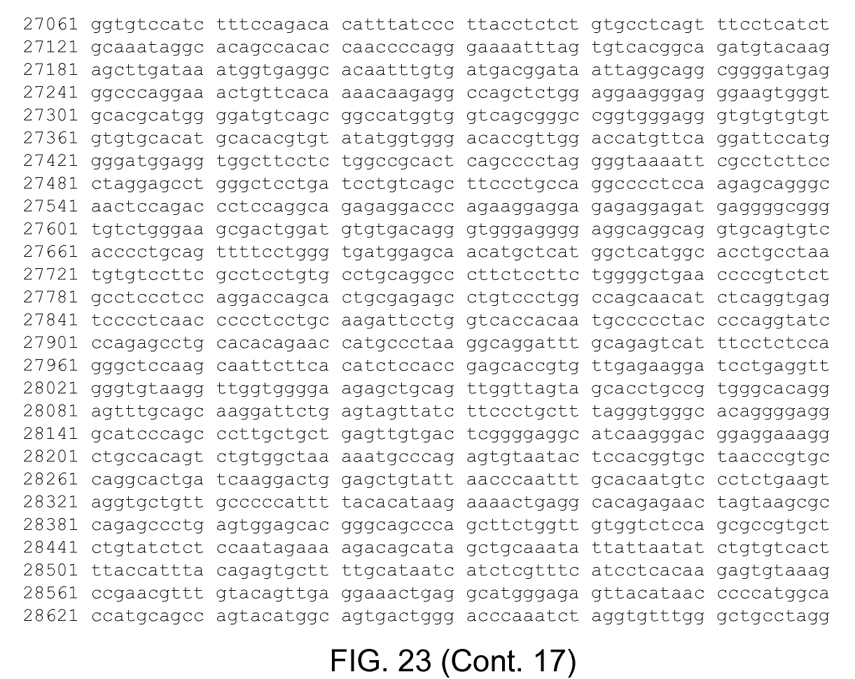

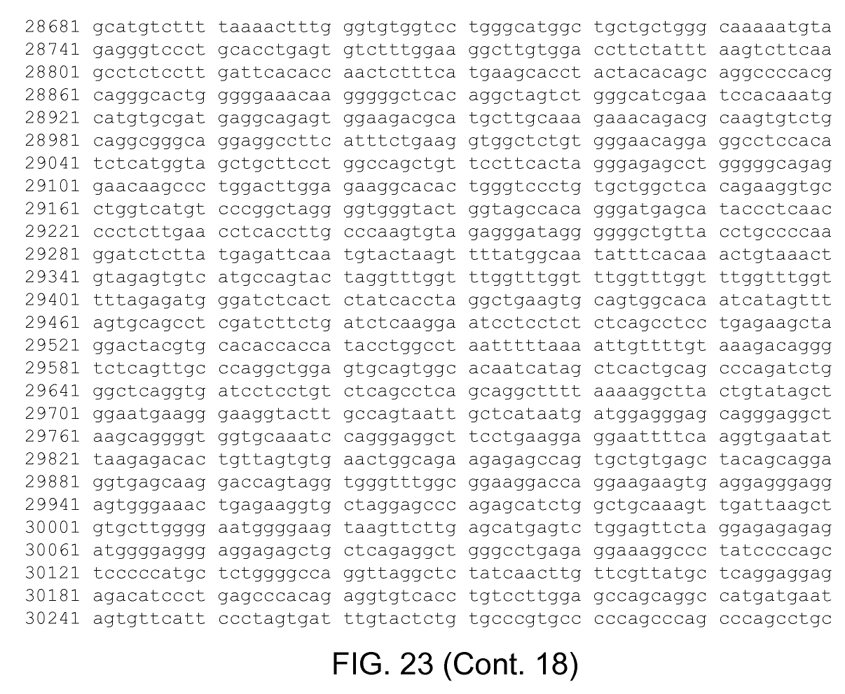

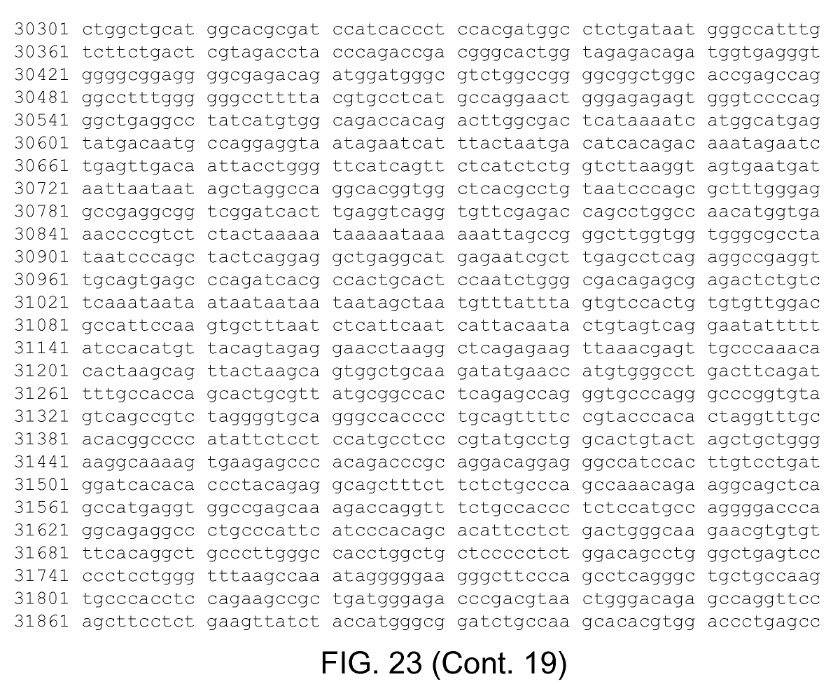

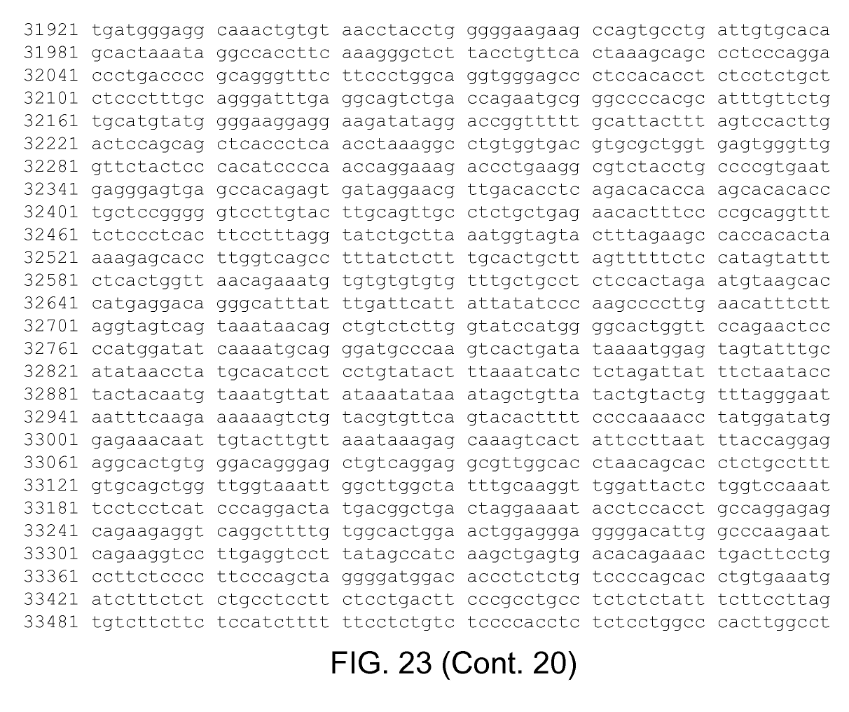

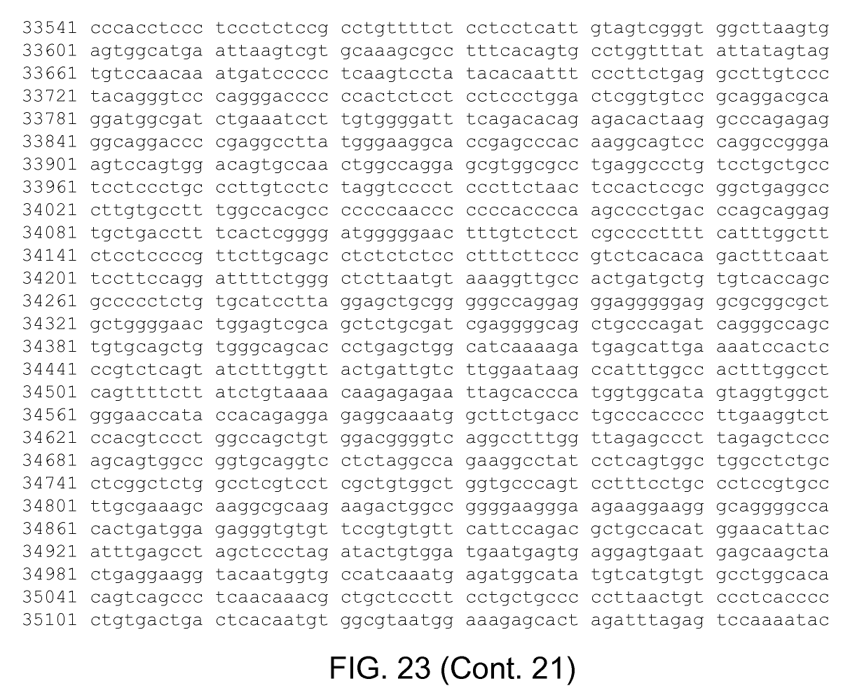

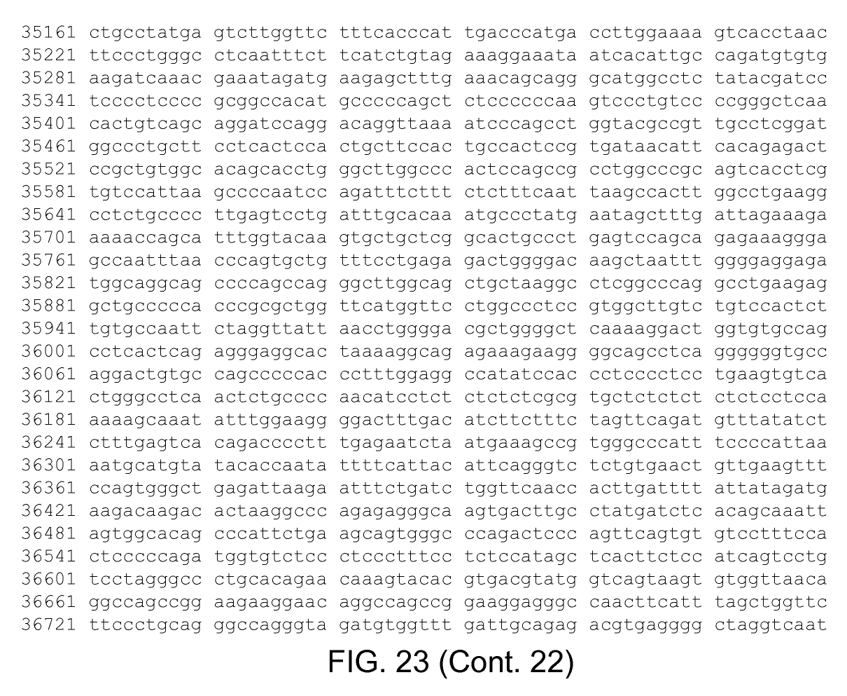

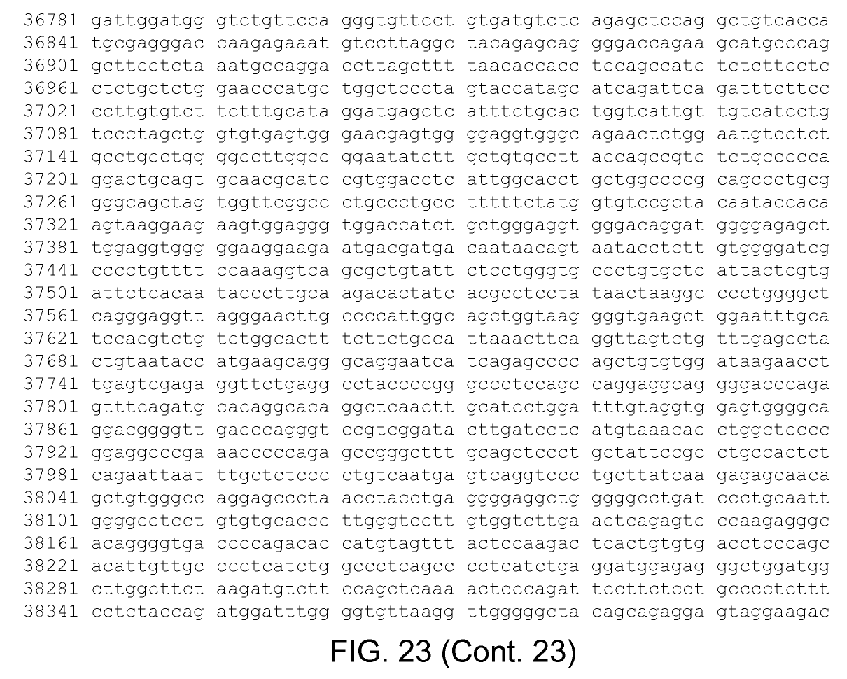

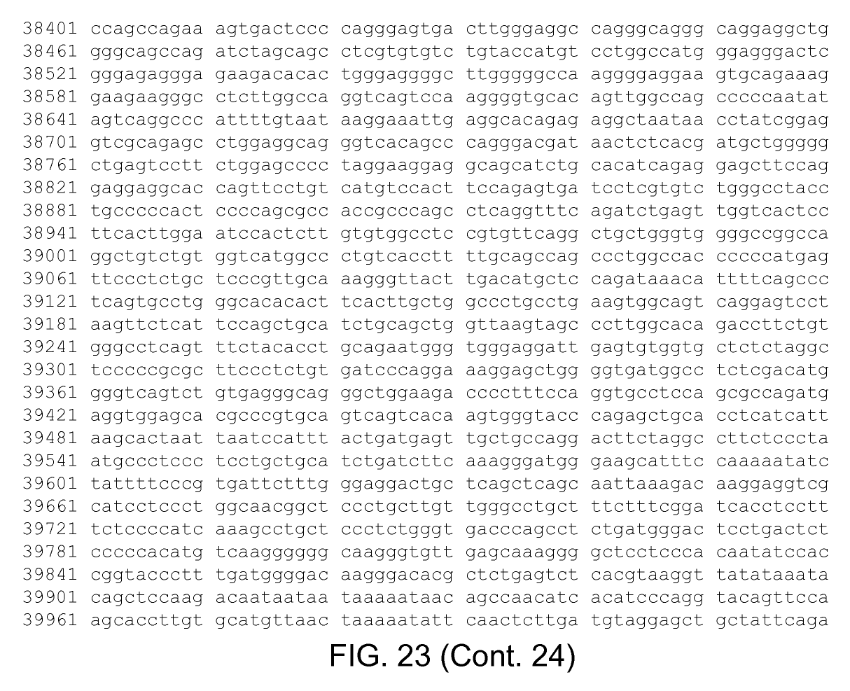

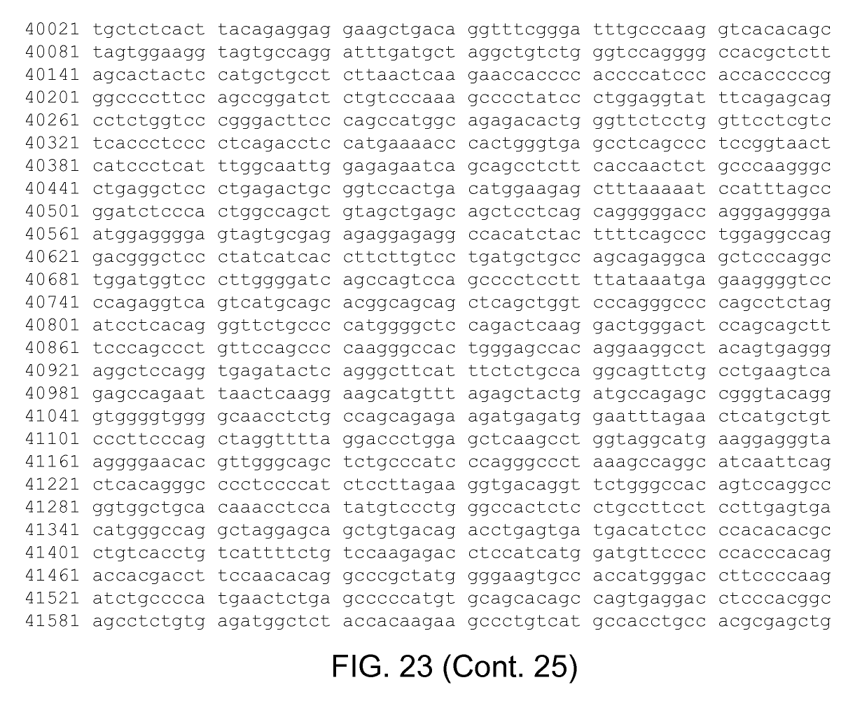

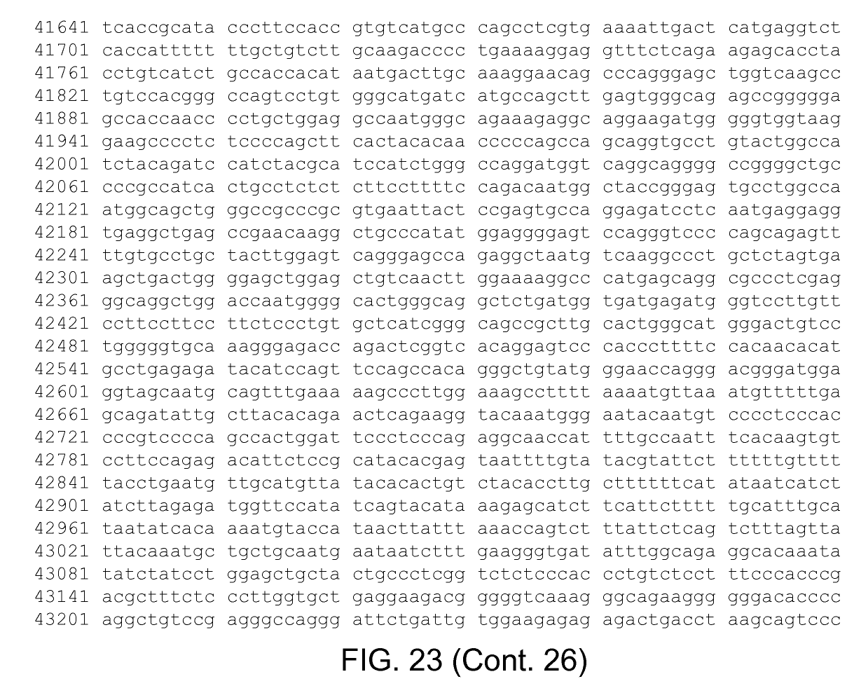

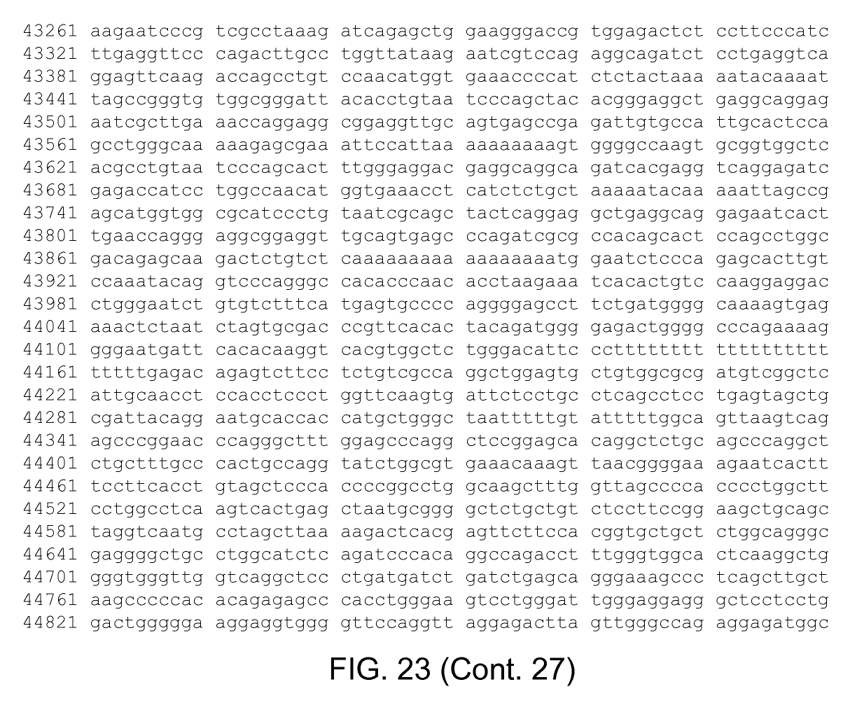

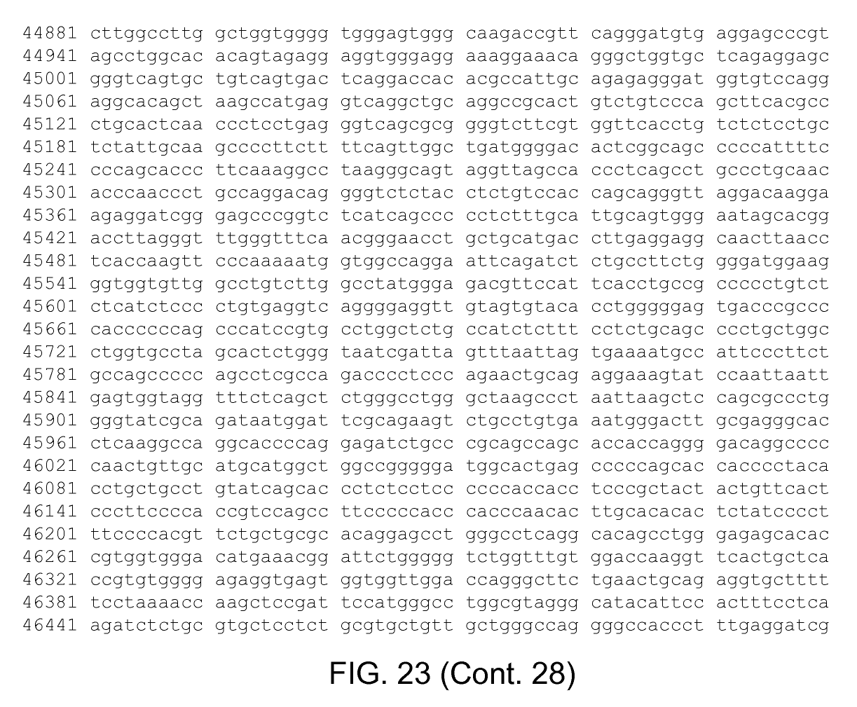

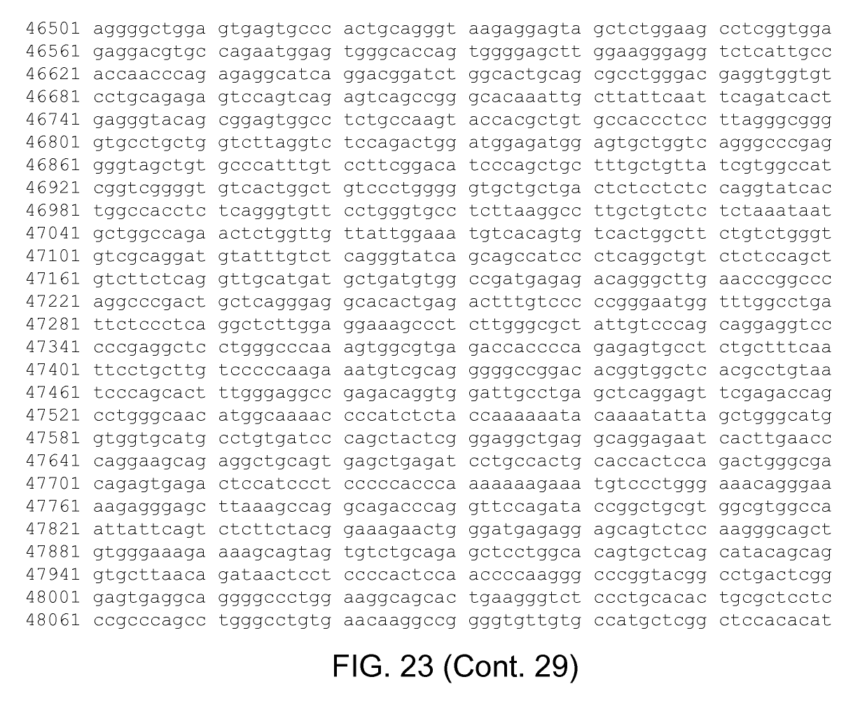

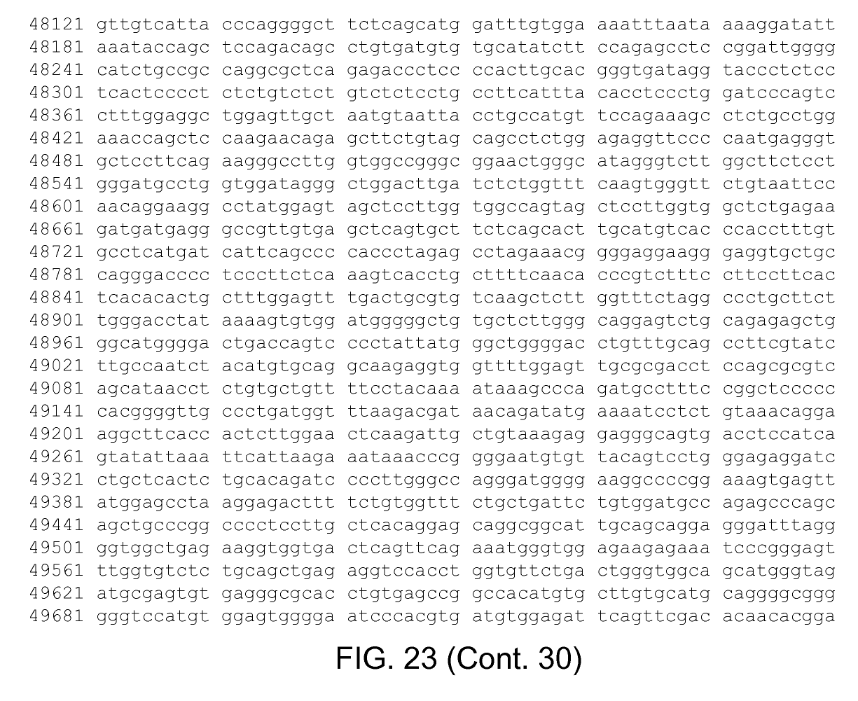

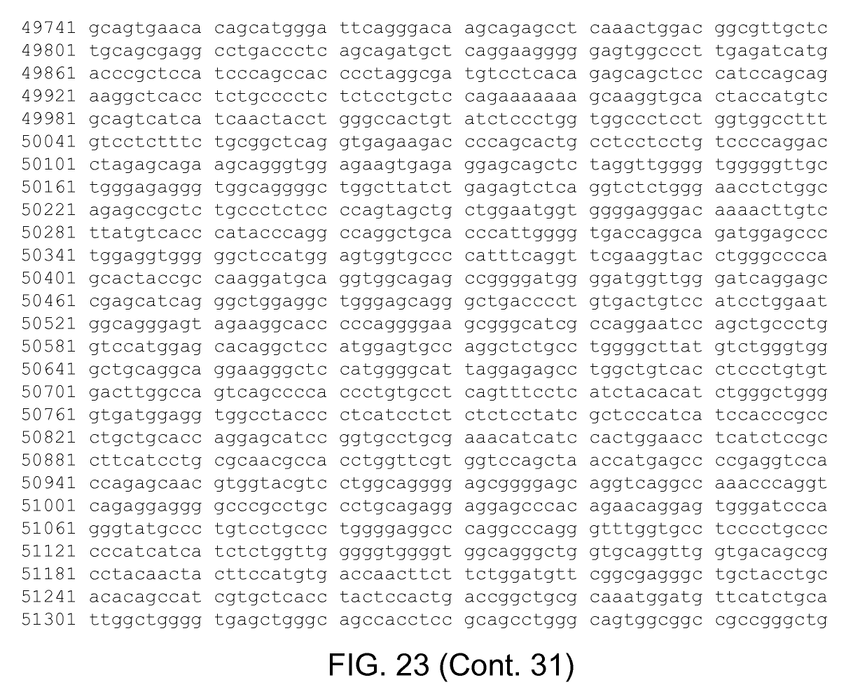

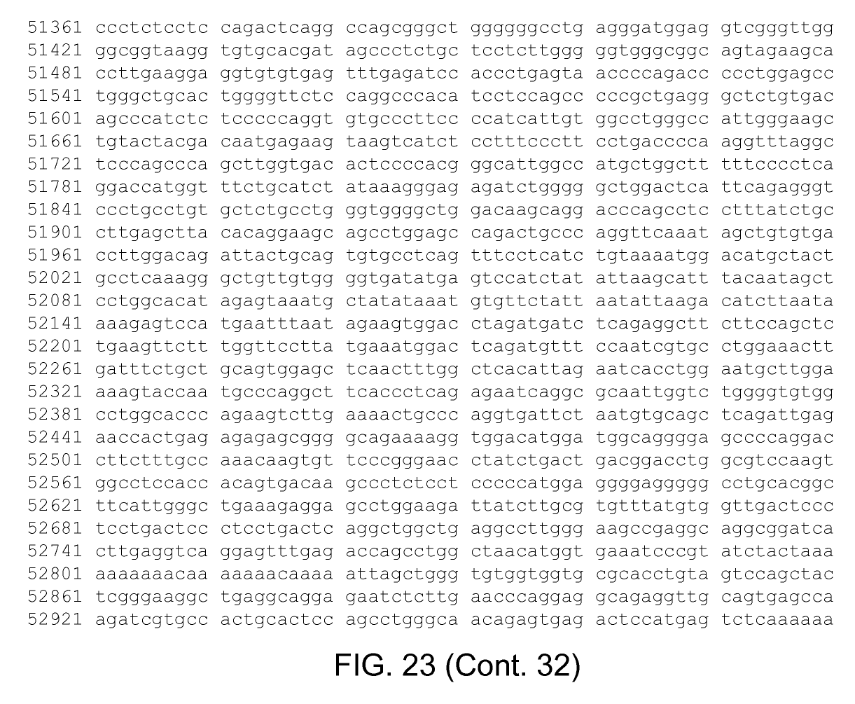

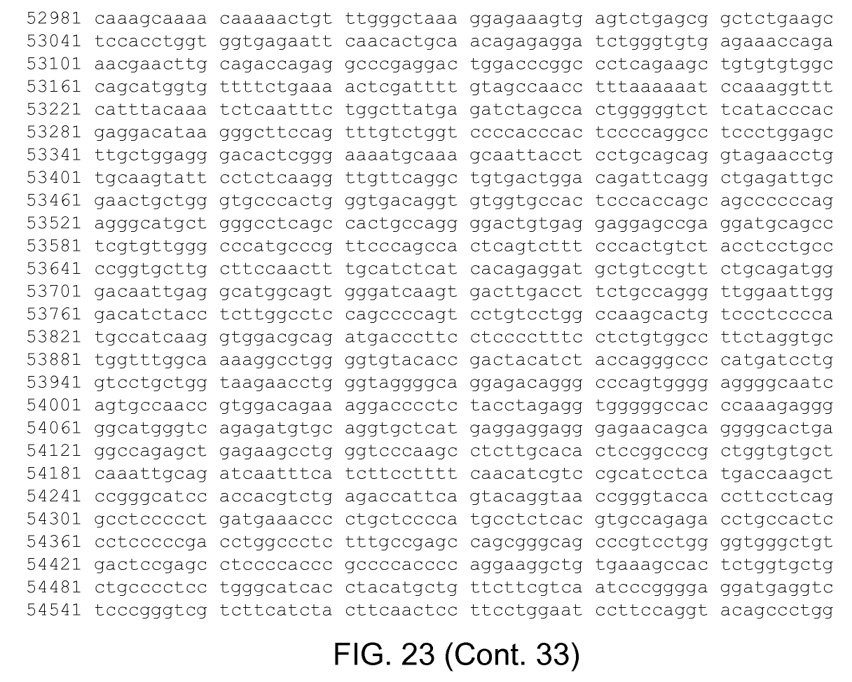

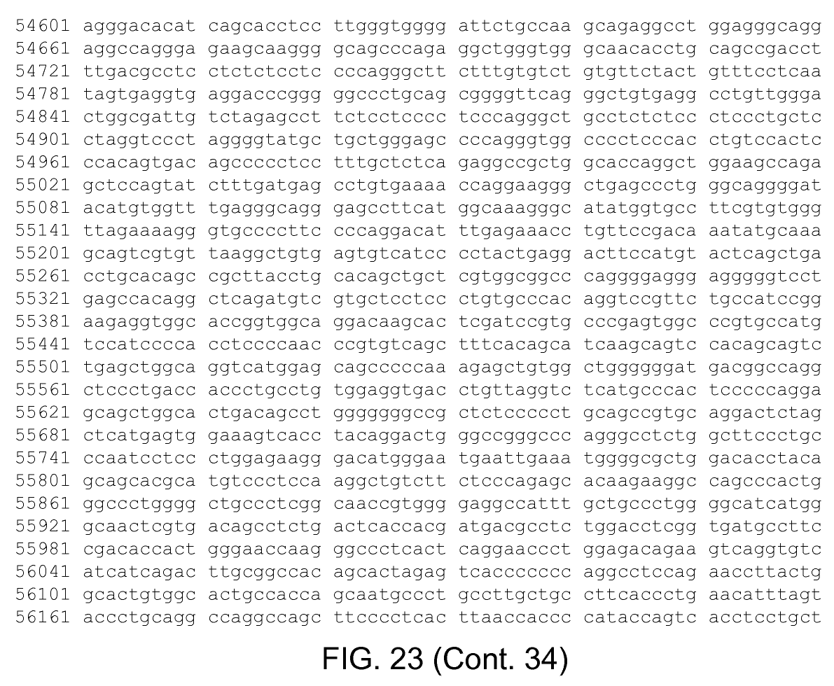

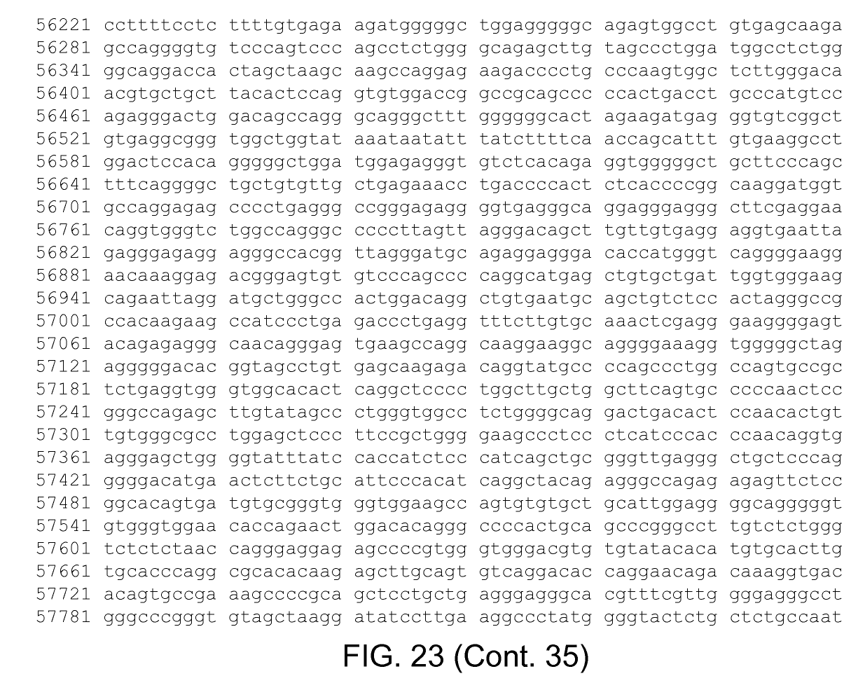

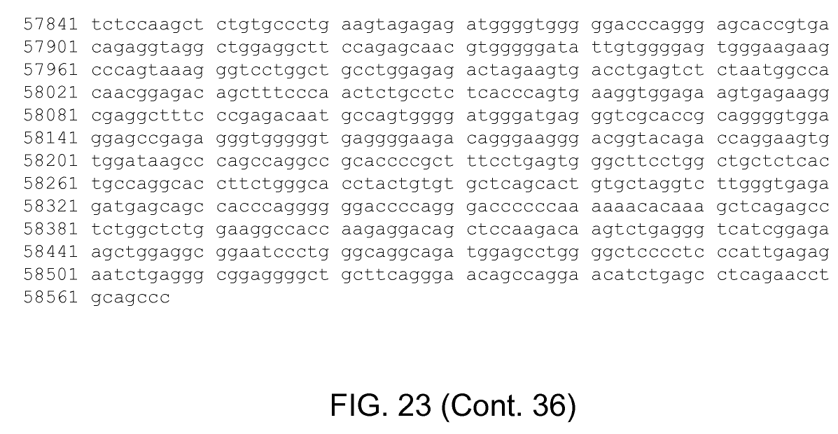

The present disclosure is exemplified by FIGS. 1-23.

### Example 2

### HPA Axis Genetic Variation, Cortisol, and Psychosis in Major Depression

The data presented below show that GR contributes significantly to cortisol activity as well as to risk for psychosis, and that CRH-R1 contributes significantly to risk for major depression with endogenous features, with and without psychosis.

### Methods

Participants were recruited either from patient facilities at Stanford University or self-referred via online and print advertisements in two waves of study (HPA-2 and HPA-3 collected over 10 years). One hundred and twenty-two patients and controls were recruited for this genetics study: forty-three patients with psychotic depression, 35 patients with nonpsychotic depression, and 44 healthy controls participated in the study and provided usable data.

All patients met DSM-IV criteria for a current unipolar major depressive episode, with or without psychotic features (see Table 1). Participants were diagnosed with the Structured Clinical Interview for DSM-IV (SCID) (23). Diagnoses were confirmed by consultation with the subjects' treating psychiatrists. Depressed patients with and without psychosis were required to have a minimum score of 18 on the 21-item Hamilton Depression Rating Scale (HAM-D) (24). There was also a minimum required score on the 7-item Thase Core Endogenomorphic Scale (25), an assessment scale derived from the HAM-D. In HPA2 the minimum Thase score required was 7 and in HPA3, the minimum Thase score required was a 6, with the exception that one psychotically depressed patient in HPA3 was included with a Thase score of 5. The minimum Thase cut-off scores were required to ensure that patients with major depression without psychosis demonstrated considerable endogenous, or melancholic, symptoms. Subjects with major depression with psychosis were also required to have a minimum score of 5 on the positive symptom subscale of the Brief Psychiatric Rating Scale (BPRS (26)), which consists of the following four items: conceptual disorganization, suspiciousness, hallucinations, and unusual thought content. A score of 4 indicates no positive symptoms. In addition, we included 5 patients who were delusional at the time of screening but not at the point of study (termed partially improved) in the psychotic group. Subjects with major depression without psychosis had no history of psychotic symptoms. Participants were allowed to continue their psychiatric medication but were required to maintain a stable medication regimen for at least 1 week prior to the start of the study.

Forty-four healthy comparison subjects were recruited through online and print advertisements. Comparison subjects were required to have a score of less than 6 on the HAM-D and have no psychotic symptoms. Additionally, they were required to have no current or past axis I psychiatric disorders, assessed via the SCID criteria. Cortisol data were not available on 11 of the 44 controls.

Exclusion criteria for all three groups were major medical illnesses, history of seizures or major head trauma, abnormal clinical laboratory tests, pregnant or lactating women, and individuals under age 18. Participants were excluded if they had unstable or untreated hypertension, cardiovascular disease, or endocrine disorders. Subjects being treated with any steroid-altering agents, including estrogen or hormone replacement therapy (HRT) were not included in the cortisol analyses. Additionally, patients who were actively suicidal or met DSM-IV criteria for obsessive-compulsive disorder or bipolar disorder were excluded as well as those patients with a history of substance abuse or electroconvulsive therapy within the 6 months prior to the start of the study. All participants were compensated $250.

### Procedure

The study was approved by the Stanford University Institutional Review Board, and all subjects gave written informed consent before screening. Eligibility screening procedures included the following: SCID, HAM-D, BPRS, clinical laboratory tests (with complete blood count examination), comprehensive metabolic panel, urine drug screening, and urine screening for pregnancy in female subjects. If participants met inclusion criteria at the eligibility screening, they were asked to return for baseline procedures. Participants underwent structural and functional MRI and a neuropyschological battery. In addition, subjects participated in blood sampling overnight on the Stanford University Hospital GCRC. An intravenous line was inserted at 4 PM and blood collected hourly for cortisol from 6 pm to 9 am the next day. Cortisol data on a subset of the patients (N=43) used in these data analyses were previously published (4).

### Cortisol Determination

The blood samples were kept frozen at −80 degrees C. until analysis. Cortisol assays were conducted by the Brigham Women's Hospital, General Clinical Research Laboratory in Boston. The analytic sensitivity for cortisol was 0.4 micrograms/dl with a coefficient of variation of less than 7.9%. As previously described (4) we calculated a mean cortisol level for each patient for the periods of 6 pm to 1 am as well as for 1 am to 9 am and used these means in the analyses.

### Genetics

Blood was collected for assessment of genetic markers at the following HPA axis loci: CRH, CRHR1, CRHR2, NR3C1 (glucocorticoid receptor, GR), NR3C2 (mineralocorticoid receptor, MR), and FKBP5. Genetic markers were selected using a standard protocol (27) that utilizes these criteria: 1) The marker has demonstrated (based on literature) or predicted (based on bioinformatic analysis) effects on protein abundance or function; 2) The marker has been associated in the literature with depression, psychosis, memory performance or the HPA axis regulation; 3) The marker is located in a region of interest: exons, 3′UTR, intron boundaries (28), or one of the many regulatory elements involved in gene expression; 4) The marker is a good predictor (“tagging SNP”) for the other polymorphisms in the gene due to high LD; 5) The tagging marker complements other selected markers to provide good LD coverage of the gene.

LD bins and the selection of tagging SNPs were predicted using Haploview and the unrelated individuals from the HapMap CEU (N=60 individuals of European descent collected in UTAH) (29), which was the closest match to our study sample. De Bakker and co-workers (30) showed that ‘tagging’ SNPs selected using the CEPH cohort retained a reasonable level of effectiveness across multiple non-African populations. However, in some cases, HapMap either does not contain a particular gene or genomic region, or a particular marker of interest was not present within the HapMap CEU cohort. In these cases, we utilized other SNP databases such as dbSNP, UCSC, Illumina, Seattle SNPs and SNPper. In general only SNPs with a minor allele frequency of 5% or greater were considered.

DNA was extracted from EDTA-treated whole blood using the Gentra Puregene kit (Qiagen, Valencia, Calif.). Genotyping was performed using Taqman real-time PCR (Applied Biosystems, Foster City, Calif.). All genotypes were tested for deviation from Hardy Weinberg equilibrium.

Tables 2 and 3 summarize the SNPs and their frequencies that were assayed for each HPA Axis gene. A total of 70 SNPs were assessed, distributed in the following manner: CRH (6 SNPs), CRHR1 (21 SNPs), CRH-R2 (18 SNPs), NR3C2 (MR; 15 SNPs), NR3C1 (GR; 10 SNPs), and FKBP5 (2 SNPs). SNPs that had no or minimal variability in our sample were excluded from the analysis. If SNPs had fewer than 5 subjects across all 3 groups homozygous for the rare allele, they were collapsed into the heterozygous group.

### Data Analyses

We used a gene-centric testing strategy in lieu of a SNP-specific approach. Gene-wide tests have several advantages. When the primary scientific interest is in the gene as a whole and its impact on a particular pathway, inconsistencies among individual SNP results can be difficult to interpret. In addition, performing a single global test of a gene first eliminates the need for a multiple testing penalty, which would impose a more stringent significance threshold and reduce power. Individual SNPs can still be evaluated individually once the gene as a whole has been found to have a significant association.

All analyses were conducted using SPSS statistical software. In this study, we examined the relationship between HPA axis variation and individual genes controlling for the well-known effects of age on serum cortisol levels. As oral contraceptives and hormone replacement treatments dramatically increase cortisol, all subjects on these medications were removed from the cortisol analyses. In addition, we had several subjects who had missing or incomplete cortisol data. In toto, 27 cases were excluded from the cortisol analyses. For each gene, we conducted stepwise, multiple regressions for cortisol response comparing a model with age alone as a predictor to a model with age and all available SNPs in the gene to obtain a single gene-wide test. Individual SNPs in genes with significant overall associations with cortisol response were evaluated based on coefficients and p-values for that SNP in the presence of age and all other SNPs.

We next performed a series of logistic regression analyses to assess the contribution of variation in each gene to clinical status—depression or psychosis. All SNPs for a specific gene were again entered as independent predictors and a gene-wide test was performed based on the significance of the entire model. Individual SNP contributions were assessed based on the same multi-SNP model, with the effect of each SNP adjusted for all other SNPs in the gene.

### Results

Demographic characteristics are summarized in Table 1. The three groups were well matched on age and gender. Patients with PMD had significantly higher total HDRS scores than did the NPMD's or HC's. Differences between the patients primarily reflected psychosis-related symptoms in the PMD group. Mean scores on the Thase Endogenomorphic Subscale were also significantly higher in the PMD patients, although, the differences between clinical groups were relatively small. On the BPRS, PSS scores were higher in the PMD group than in the other two groups.

TABLE 1
Table 1. Demographic and Clinical Measures in Subjects

| | PMD | NPMD | | | Post-hoc |
|---|---|---|---|---|---|
| | (N = 43) | (N = 35) | HC (N = 44) | Test Value | comparison |
| Age | 39.1 (13.6) | 40.31 (14.1) | 35.00 (14.1) | F(2,119) = 1.63, ns | |
| Gender | | | | χ²(2) = 2.67, ns | |
| Female | 23 | 25 | 26 | | |
| Male | 20 | 10 | 18 | | |
| Ethnicity\hat{ } | | | | χ²(8) = 4.25, ns | |
| Caucasian | 33 | 27 | 31 | | |
| African-American | 2 | 2 | 2 | | |
| Asian | 4 | 2 | 8 | | |
| Latino | 3 | 2 | 2 | | |
| Other | 1 | 2 | 1 | | |
| Education | 15.16 (2.8) | 15.40 (1.7) | 15.85 (2.0) | F(2,119) = 1.09, ns | |
| HDRS Total | 29.02 (5.4) | 23.40 (3.1) | .61 (.92) | F(2,119) = 728.3, | PMD > NPMD > |
| | | | | p < .001 | HC |
| HDRS | 9.09 (2.1) | 8.23 (1.6) | .16 (.43) | F(2,119) = 440.2, | PMD > NPMD > |
| Endogenormophic | | | | p < .001 | HC |
| Subscale | | | | | |
| BPRS Total* | 45.26 (7.2) | 33.34 (4.2) | 18.67 (1.3) | F(2,109) = 257.8, | PMD > NPMD > |
| | | | | p < .001 | HC |
| BPRS PSS* | 10.13 (4.0) | 4.31 (.83) | 4.09 (.38) | F(2,109) = 72.20, | PMD > (NPMD = |
| | | | | p < .001 | HC) |
| Cortisol 6pm-1 am | N = 40 | N = 26 | N = 29 | F(2,92) = 4.19, | PMD > (NPMD = |
| | 4.94 (2.7) | 3.65 (1.7) | 3.65 (1.5) | p = .018 | HC) |
| Cortisol 1 am-9 am | N = 40 | N = 26 | N = 29 | F(2,92) = .800, ns | |
| | 9.73 (4.7) | 8.68 (2.6) | 8.93 (2.2) | | |

*10 Control patients were not administered the BPRS.
\hat{ }Ethnicity was assessed because SNP prevalence may differ between ethnic groups.

Only one of the 6 genes—NR3C1 (GR)—significantly predicted evening cortisol from 6 pm-1 am. The overall model with age and NR3C1 genetic variation together significantly accounted for 26.6% of the variance, F(10,84)=3.05, p=0.002. Variation in NR3C1 accounted for an additional 20.1% of the variance (F(9,84)=2.56, p=0.012), on top of the 6.5% accounted for by age. Four of the 9 individual SNPs attained statistical significance when added separately and last to the model (rs33888, p=0.042; rs2918419 p=0.003; rs10052957, p=0.001; and rs10482633, p=0.025).

Similarly, only NR3C1 significantly predicted early morning cortisol from 1 am to 9 am. Together, age and NR3C1 account for 32.2% of the variance (F(10,84)=3.99, p<0.001). The NR3C1 gene alone accounted for 18.2% of the variance (F(9,84)=2.50, p=0.014), on top of the 14.0% explained by age. Four NR3C1 SNPs attained significance when added last (rs2918419, p=0.011; rs10052957, p=0.001; rs10482633, p=0.027; the fourth was rs41423247, p=0.042). Three of the SNPs, rs2918419, rs10052957, and rs10482633, predicted both evening and early morning cortisol. The significant SNPs are generally in low LD with one another, other than an r² of 0.51 observed between rs10052957 and rs1048263.

We performed binomial logistic regression for separating depressed patients (combined PMD and NPMD) from HC's as well as for separating psychotic patients (PMD) from the others (NPMD combined with HC's.) Only variation in CRHR1 predicted depression, X² (20)=33.91, p=0.027. CRHR1 markers correctly classified 72.2% of the subjects and accounted for 35.1% of the variance. Only one CRHR1 SNP was of trend significance when added to the model last, rs4458044, p=0.076. No variants in other genes significantly predicted depression.

Of the 6 genes examined, both NR3C1 and CRHR1 significantly predicted psychosis. For NR3C1, X²(9)=21.02, p=0.013, with SNPs accounting for 22.5% of the variance and correctly classifying 71.4% of the sample. Only one SNP significantly predicted psychosis when added last to the model—rs33388, p=0.043; however, two other SNPs were of trend significance, rs12655166, p=0.092 and rs41423247, p=0.064.

CRHR1 SNPs also predicted psychosis X²(20)=36.02, p=0.015. CRHR1 SNPs correctly classified 75.7% of subjects and accounted for 37.2% of the variance. One SNP attained significance when added last, rs11657992, p=0.018, and one additional SNP was of trend significance, rs1690674, p=0.07.

For all analyses, results were similar for the full sample (N=122) and for the subsample with European Caucasians only (N=88). In the Caucasian-only sample, the overall prediction of evening cortisol by NR3C1, with age accounted for, held strong (change r²=48.8, p<0.001). Similar results were found for early morning cortisol (change r²=30.0, p=0.02). Logistic regression results predicting depression and psychosis were similar with the exception that CRHR2 variation overall significantly predicted psychosis (r²=40.5, p<0.05).

TABLE 2
Table 2. HPA axis Genes Assessed

| Gene | Function | Brain Location in humans | SNPs Studied |
|---|---|---|---|
| CRH | Stimulates HPA axis; mediates | See below for CRH--R₁ | N = 6 |
| | stress responses in amygdala | and CRH-R₂ | **rs10098823 |
| | | | rs3176921 |
| | | | \hat{ }rs5030875 |
| | | | rs5030877 |
| | | | rs6999100 |
| | | | \hat{ }rs7350113 |
| CRHR1 | Mediates stress responses and | Distributed widely | N = 21 |
| | HPA axis stimulation. Binds | throughout brain with | rs110402 |
| | primarily to CRH. | particular concentration in | *rs12938031 |
| | | the amygdala, | rs16940674 |
| | | hippocampus, and | rs171440 |
| | | prefrontal cortex, | rs17689966 |
| | | | rs242924 |
| | | | rs242940 |
| | | | rs242948 |
| | | | **rs4076452 |
| | | | **rs4792887 |
| | | | **rs4792888 |
| | | | rs7209436 |
| | | | \hat{ }rs3785877 |
| | | | \hat{ }rs4792825 |
| | | | rs4458044 |
| | | | rs12944712 |
| | | | **rs17763104 |
| | | | **rs2664008 |
| | | | **rs17763658 |
| | | | **rs242942 |
| | | | **rs11657992 |
| CRHR2 | Involved in appetitive behaviors | Little is known about | N = 18 |
| | and stress; binds to both CRH and | human CRH-R2 | **rs2240403 |
| | urocortin | distribution | rs2267712 |
| | | | rs2267715 |
| | | | rs2267717_coded |
| | | | rs2270007 |
| | | | rs255100 |
| | | | **rs4723003 |
| | | | **rs7812133 |
| | | | rs255102 |
| | | | rs975537 |
| | | | rs2190242 |
| | | | rs2267716 |
| | | | **rs2284216 |
| | | | rs2284217 |
| | | | **rs4723000 |
| | | | **rs12701020 |
| | | | **rs17159371 |
| | | | rs929377 |
| NR3C1 | Feedback inhibition of HPA axis; | Cortex, hypothalamus, | N = 10 |
| (GR) | cognition; immune response | amygdala, hippocampus | *rs6195 |
| | | | **rs6198 |
| | | | rs33388 |
| | | | \hat{ }rs2918419 |
| | | | rs10052957 |
| | | | rs10482633 |
| | | | **rs12521436 |
| | | | **rs12655166 |
| | | | **rs17209258 |
| | | | rs41423247 |
| NR3C2 | Inhibitory control of HPA axis; | Hypothalamus, | N = 13 |
| (MR) | memory; blood pressure | hippocampus, amygdala | **rs5525 |
| | | | *rs5530 |
| | | | rs1879829 |
| | | | rs2070951 |
| | | | **rs2272089 |
| | | | rs3910052 |
| | | | rs4835488 |
| | | | rs6535578 |
| | | | rs7658048 |
| | | | **rs7694064 |
| | | | **rs10213471 |
| | | | *rs17024360 |
| | | | **rs17484245 |
| | | | rs2070950 |
| | | | **rs5522 |
| FKBP5 | Co-chaperone to heat shock | See GR | N = 2 |
| | protein for GR; stabilizes GR | | rs1360780 |
| | confirmation | | rs3800373 |

*Little or no variance in the SNPs; SNP not used in analyses
**Less than 5 total in the rare homozygous, collapsed into the heterozygous SNP group
\hat{ }Only 2 SNP variations present

TABLE 3
Allele Frequencies

| | | Minor allele | | | |
|---|---|---|---|---|---|
| | | (major | | | |
| | Position | allele) | MAF | HWP | Role |
| rs6198 | 142657621 | C(t) | 14.6% | 0.94 | 3′UTR |
| | | | (n = 240)* | | |
| rs17209258 | 142673397 | G(a) | 17.4% | 0.51 | Intron |
| | | | (n = 242) | | |
| rs33388 | 142697295 | T(a) | 45.8% | 0.34 | Intron |
| | | | (n = 240) | | |
| rs2918419 | 142722353 | G(a) | 10.2% | 0.51 | Intron |
| | | | (n = 244) | | |
| rs10482633 | 142750533 | C(a) | 17.6% | 0.60 | Intron |
| | | | (n = 244) | | |
| rs41423247 | 142778575 | C(g) | 31.8% | 1.00 | Intron |
| | | | (n = 242) | | |
| rs6195 aka | 142779317 | G(a) | 1.2% | 1.00 | Asn > Ser |
| rs56149945 | | | (n = 244) | | at codon |
| | | | | | 363 |
| | | | | | (exon 2) |
| rs10052957 | 142786701 | A(g) | 27.9% | 0.94 | Promoter |
| | | | (n = 244) | | or Intron |
| rs12655166 | 142809272 | C(t) | 18.4% | 0.34 | Intron |
| | | | (n = 244) | | |
| rs12521436 | 142817607 | A(g) | 18.9% | 0.68 | Promoter |
| | | | (n = 244) | | |

*Number of chromosomes assayed.

### REFERENCES

1. Anton R F. Urinary free cortisol in psychotic depression. Biol Psychiatry. 1987; 22(1):24-34.
2. Posener J A, DeBattista C, Williams G H, Kraemer H C, Kalehzan B M, Schatzberg A F. 24-Hour Monitoring of Cortisol and Corticotropin Secretion in Psychotic and Nonpsychotic Major Depression. Arch Gen Psychiatry. 2000; 57(8):755-60.
3. Belanoff J K, Kalehzan M, Sund B, Fleming Ficek S K, Schatzberg A F. Cortisol activity and cognitive changes in psychotic major depression. Am J. Psychiatry. 2001; 158(10):1612-6.
4. Keller J, Flores B, Gomez R G, Solvason H B, Kenna H, Williams G H, et al. Cortisol circadian rhythm alterations in psychotic major depression. Biol Psychiatry. 2006; 60(3):275-81.
5. Lembke A, Gomez R, Tenakoon L, Keller J, Cohen G, Williams G H, et al. The mineralocorticoid receptor agonist, fludrocortisone, differentially inhibits pituitary-adrenal activity in humans with psychotic major depression. Psychoneuroendocrinology. 2012.
6. Arana G W, Barreira P J, Cohen B M, Lipinski J F, Fogelson D. The dexamethasone suppression test in psychotic disorders. Am J. Psychiatry. 1983; 140(11):1521-3.
7. Arana G W, Wilens T E, Baldessarini R J. Plasma corticosterone and cortisol following dexamethasone in psychiatric patients. Psychoneuroendocrinology. 1985; 10(1):49-60.
8. Evans D L, Burnett G B, Nemeroff C B. The dexamethasone suppression test in the clinical setting. Am J. Psychiatry. 1983; 140(5):586-9.
9. Nelson J C, Davis J M. DST studies in psychotic depression: a meta-analysis. Am J. Psychiatry. 1997; 154(11):1497-503.
10. Schatzberg A F, Rothschild A J, Stahl J B, Bond T C, Rosenbaum A H, Lofgren S B, et al. The dexamethasone suppression test: identification of subtypes of depression. Am J. Psychiatry. 1983; 140(1):88-91.
11. Keck M E, Kern N, Erhardt A, Unschuld P G, Ising M, Salyakina D, et al. Combined effects of exonic polymorphisms in CRHR1 and AVPR1B genes in a case/control study for panic disorder. Am J Med Genet B Neuropsychiatr Genet. 2008; 147B(7):1196-204.
12. Liu Z, Zhu F, Wang G, Xiao Z, Wang H, Tang J, et al. Association of corticotropin-releasing hormone receptor1 gene SNP and haplotype with major depression. Neurosci Lett. 2006; 404(3):358-62.
13. Bradley R G, Binder E B, Epstein M P, Tang Y, Nair H P, Liu W, et al. Influence of child abuse on adult depression: moderation by the corticotropin-releasing hormone receptor gene. Arch Gen Psychiatry. 2008; 65(2):190-200.
14. Ressler K J, Bradley B, Mercer K B, Deveau T C, Smith A K, Gillespie C F, et al. Polymorphisms in CRHR1 and the serotonin transporter loci: gene x gene x environment interactions on depressive symptoms. Am J Med Genet B Neuropsychiatr Genet. 2010; 153B(3):812-24.
15. Ressler S, Radhi J, Aigner T, Loo C, Zwerschke W, Sergi C. Insulin-like growth factor-binding protein-3 in osteosarcomas and normal bone tissues. Anticancer Res. 2009; 29(7):2579-87.
16. Binder E B, Owens M J, Liu W, Deveau T C, Rush A J, Trivedi M H, et al. Association of polymorphisms in genes regulating the corticotropin-releasing factor system with antidepressant treatment response. Arch Gen Psychiatry. 2010; 67(4):369-79.
17. El-Hage W, Phillips M L, Radua J, Gohier B, Zelaya F O, Collier D A, et al. Genetic modulation of neural response during working memory in healthy individuals: interaction of glucocorticoid receptor and dopaminergic genes. Mol. Psychiatry. 2011.
18. Krishnamurthy P, Romagni P, Torvik S, Gold P W, Charney D S, Detera-Wadleigh S, et al. Glucocorticoid receptor gene polymorphisms in premenopausal women with major depression. Horm Metab Res. 2008; 40(3):194-8.
19. Binder E B, Salyakina D, Lichtner P, Wochnik G M, Ising M, Putz B, et al. Polymorphisms in FKBP5 are associated with increased recurrence of depressive episodes and rapid response to antidepressant treatment. Nat. Genet. 2004; 36(12):1319-25.
20. Mehta D, Gonik M, Klengel T, Rex-Haffner M, Menke A, Rubel J, et al. Using polymorphisms in FKBP5 to define biologically distinct subtypes of posttraumatic stress disorder: evidence from endocrine and gene expression studies. Arch Gen Psychiatry. 2011; 68(9):901-10.
21. Zimmermann P, Bruckl T, Nocon A, Pfister H, Binder E B, Uhr M, et al. Interaction of FKBP5 gene variants and adverse life events in predicting depression onset: results from a 10-year prospective community study. Am J. Psychiatry. 2011; 168(10):1107-16.
22. Binder E B, Bradley R G, Liu W, Epstein M P, Deveau T C, Mercer K B, et al. Association of FKBP5 polymorphisms and childhood abuse with risk of posttraumatic stress disorder symptoms in adults. JAMA. 2008; 299(11):1291-305.
23. First M B, Spitzer R L, Gibbone M, Williams J B W. Structured Clinical Interview for the DSM-IV-TR Axis I Disorders—Patient Edition. 1997.
24. Hamilton M. A rating scale for depression. Journal of Neurology, Neurosurgery, and Psychiatry. 1980; 23:49-65.
25. Thase M E, Hersen M, Bellack A S, Himmelhoch J M, Kupfer D J. Validation of a Hamilton subscale for endogenomorphic depression. J Affect Disord. 1983; 5(3):267-78.
26. Overall J E, Gorham D E. The brief psychiatric rating scale. Psychological Reports. 1961; 10:799-812.
27. Pettersson F H, Anderson C A, Clarke G M, Barrett J C, Cardon L R, Morris A P, et al. Marker selection for genetic case-control association studies. Nat. Protoc. 2009; 4(5):743-52.
28. Lomelin D, Jorgenson E, Risch N. Human genetic variation recognizes functional elements in noncoding sequence. Genome Res. 2010; 20(3):311-9.
29. Frazer K A, Ballinger D G, Cox D R, Hinds D A, Stuve L L, Gibbs R A, et al. A second generation human haplotype map of over 3.1 million SNPs. Nature. 2007; 449(7164):851-61.
30. De Bakker P I, Graham R R, Altshuler D, Henderson B E, Haiman C A. Transferability of tag SNPs to capture common genetic variation in DNA repair genes across multiple populations. Pac Symp Biocomput. 2006:478-86.
31. Kumsta R, Entringer S, Koper J W, van Rossum E F, Hellhammer D H, Wust S. Sex specific associations between common glucocorticoid receptor gene variants and hypothalamus-pituitary-adrenal axis responses to psychosocial stress. Biol Psychiatry. 2007; 62(8):863-9.
32. van Rossum E F, Binder E B, Majer M, Koper J W, Ising M, Modell S, et al. Polymorphisms of the glucocorticoid receptor gene and major depression. Biol Psychiatry. 2006; 59(8):681-8.
33. Sinclair D, Fullerton J M, Webster M J, Shannon Weickert C. Glucocorticoid receptor 1B and 1C mRNA transcript alterations in schizophrenia and bipolar disorder, and their possible regulation by GR gene variants. PLoS One. 2012; 7(3):e31720.
34. Gomez R G, Fleming S H, Keller J, Flores B, Kenna H, DeBattista C, et al. The neuropsychological profile of psychotic major depression and its relation to cortisol. Biol Psychiatry. 2006; 60(5):472-8.
35. Schatzberg A F, Posener J A, DeBattista C, Kalehzan B M, Rothschild A J, Shear P K. Neuropsychological deficits in psychotic versus nonpsychotic major depression and no mental illness. Am J. Psychiatry. 2000; 157(7):1095-100.
36. Garrett A, Kelly R, Gomez R, Keller J, Schatzberg A F, Reiss A L. Aberrant brain activation during a working memory task in psychotic major depression. Am J. Psychiatry. 2011; 168(2):173-82.
37. Kelly R, Garrett A, Cohen J, Gomez R G, Lembke A, Keller J, et al. Altered brain function underlying verbal memory encoding and retrieval in psychotic major depression. Psychiatry Research: Neuroimaging. 2012.
38. Szczepankiewicz A, Rybakowski J K, Suwalska A, Hauser J. Glucocorticoid receptor polymorphism is associated with lithium response in bipolar patients. Neuro Endocrinol Lett. 2011; 32(4):545-51.
39. Syed A A, Halpin C G, Irving J A, Unwin N C, White M, Bhopal R S, et al. A common intron 2 polymorphism of the glucocorticoid receptor gene is associated with insulin resistance in men. Clin Endocrinol (Oxf). 2008; 68(6):879-84.
40. Funk C K, O'Dell L E, Crawford E F, Koob G F. Corticotropin-releasing factor within the central nucleus of the amygdala mediates enhanced ethanol self-administration in withdrawn, ethanol-dependent rats. J. Neurosci. 2006; 26(44):11324-32.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for determining an increased risk for developing depression in a subject, the method comprising:
(a) genotyping genomic nucleic acid from the subject for the presence of a single nucleotide polymorphism (SNP) in one or more of a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene; and
(b) determining from said genotyping an increased risk for developing depression.

2. The method of claim 1, wherein the genotyping is by a method selected from the group consisting of: allele-specific probe hybridization, allele-specific primer extension, allele-specific amplification, nucleic acid sequencing, 5′ nuclease digestion, a molecular beacon assay, an oligonucleotide ligation assay, size analysis, single-stranded conformation polymorphism analysis, and denaturing gradient gel electrophoresis.

3. The method of claim 1, wherein said nucleic acid is a nucleic acid extract from a biological sample from said subject.

4. The method of claim 3, wherein said biological sample is blood, saliva, or buccal cells.

5. The method of claim 1, further comprising generating a report based on the determined risk.

6. The method of claim 1, further comprising treating the individual for depression.

7. The method of claim 6, wherein said treating comprises administration of a drug selected from a selective serotonin reuptake inhibitor, a serotonin and norepinephrine reuptake inhibitor, a dopamine reuptake inhibitor, a tetracyclic antidepressant, a combined reuptake inhibitor, a receptor blocker, tricyclic antidepressant, a monoamine oxidase inhibitor, a benzodiazepine, a beta-blocker, and a non-benzodiazepine hypnotic.

8. The method of claim 1, wherein the depression-associated SNP is in the subject's corticotropin releasing hormone type 1 (CRHR1) gene, and wherein the depression-associated CRHR1 gene SNP is one or more of: rs110402, Rs16940674, rs171440, rs17689966, rs242924, rs242940, rs242948, rs4076452, rs4792887, rs4792888, rs7209436, rs37858771, rs4792825, rs4458044, rs12944712, rs17763104, rs2664008, rs17763658, rs242942, and rs11657992.

9. The method of claim 8, wherein rs17763104, rs4792887, rs4458044 individually identify an individual with depression, or an individual at increased risk of depression.

10. The method of claim 9, wherein the depression-associated SNP in the subject's GR gene, and wherein the depression-associated GR gene SNP is one or more of: rs6195, rs6198, rs33388, rs2918419, rs10052957, rs10482633, rs12521436, rs12655166, rs17209258, and rs41423247.

11. The method of claim 1, wherein the depression-associated SNP is in the subject's dopamine 2 receptor (DRD2) gene, and wherein the depression-associated DRD2 gene SNP is one or more of: rs1799978, rs10789944; rs7116768, and rs34735140.

12. The method of claim 1, further comprising using the information to select a subject population for a clinical trial.

13. A method of selecting a human subject for inclusion or exclusion in a clinical trial, the method comprising:
(a) genotyping genomic nucleic acid the subject for the presence of a depression-associated single nucleotide polymorphism (SNP) in one or more of a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene;
(b) determining from said genotyping an increased risk for developing depression; and
(c) including or excluding the subject if the genotyping indicates that the subject has an increased risk of developing depression.

14. The method of claim 13, wherein the clinical trial is of a treatment for depression.

15. A method of detecting a single nucleotide polymorphism (SNP) associated with depression in an individual, the method comprising analyzing a nucleic acid sample from said individual for the present of a depression-associated SNP in one or more of a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene, wherein said SNP is associated with increased risk of developing depression.

16. A synthetic nucleic acid consisting of from about 18 consecutive bases to about 100 consecutive bases of a gene selected from a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene, wherein said synthetic nucleic acid includes a depression-associated single nucleotide polymorphism.

17. The synthetic nucleic acid of claim 16, wherein the nucleic acid comprises a detectable label.

18. The synthetic nucleic acid of claim 17, wherein the detectable label is a fluorogenic dye.

19. The synthetic nucleic acid of claim 16, wherein the nucleic acid is immobilized on an insoluble support.

20. A pair of isolated nucleic acid primers, each from about 10 to 200 nucleotides in length,
the first nucleic acid primer of said pair comprising a sequence of at least 10 contiguous nucleotides having 100% sequence identity to a nucleic acid sequence of a gene selected from a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene; and
the second nucleic acid primer of said pair comprising a sequence of at least 10 contiguous nucleotides having 100% sequence identity to the reverse complement of the nucleic acid sequence, wherein said sequence of said second nucleic acid primer is located 3′ of the nucleic acid sequence of the first nucleic acid.

21. The pair of claim 20, wherein said pair of nucleic acid molecules primes amplification of a portion of a gene selected from a CRHR1 gene, a GR gene, a DRD2 gene, and a CRHR2 gene comprising a nucleotide position corresponding to a depression-associated single nucleotide polymorphism when used in a polymerase chain reaction with a CRHR1 gene, a GR gene, a DRD2 gene, or a CRHR2 nucleic acid as a template.

22. The pair of claim 20, wherein the first or the second primer is an allele-specific primer.

23. The pair of claim 20, wherein the first and/or the second primer comprises a detectable label.

24. A kit comprising:
a) a nucleic acid reagent for detecting a depression-associated single nucleotide polymorphism (SNP) in a gene selected from a corticotropin releasing hormone receptor type 1 (CRHR1) gene, a corticotropin releasing hormone receptor type 2 (CRHR2) gene, a glucocorticoid receptor (GR) gene, and a dopamine 2 receptor (DRD2) gene; and
b) instructions for detecting the SNP.

25. The kit of claim 24, wherein the nucleic acid reagent comprises:
a) an allele-specific probe that detects a depression-associated SNP-containing region of a gene selected from a CRHR1 gene, a CRHR2 gene, a GR gene, and a DRD2 gene;
b) an allele-specific primer or primer pair that provides for detection of a depression-associated SNP-containing region of a gene selected from a CRHR1 gene, a CRHR2 gene, a GR gene, and a DRD2 gene; or
c) a nucleic acid primer that amplifies a depression-associated SNP-containing region of a gene selected from a CRHR1 gene, a CRHR2 gene, a GR gene, and a DRD2 gene.

26. The kit of claim 24, further comprising one or more of:
a) a buffer;
b) a DNA polymerase; and
c) deoxyribonucleotides.

27. The kit of claim 26, wherein the buffer is a hybridization buffer.
